(19) United States
(12) Patent Application Publication
(10) Pub. No.: US 2025/0382623 A1
(43) Pub. Date: Dec. 18, 2025

(54) METHODS AND MICROORGANISMS FOR THE FERMENTATION OF METHANE TO MULTI-CARBON COMPOUNDS

(71) Applicant: BioVerde Tech LLC, Houston, TX (US)

(72) Inventors: Jefrey David ORTH, Houston, TX (US); Louis A. CLARK, Houston, TX (US); Lily Yuin CHAO, Houston, TX (US); Na My TRINH, Houston, TX (US); Christopher Cheyney FARWELL, Houston, TX (US); Xinhua ZHAO, Houston, TX (US); Matthias Helmut SCHMALISCH, Houston, TX (US); Grayson Thomas WAWRZYN, Houston, TX (US); Xuezhi LI, Houston, TX (US); Mark Anton HELD, Houston, TX (US); Kevin Lee DIETZEL, Houston, TX (US); James KEALEY, Houston, TX (US)

(21) Appl. No.: 19/061,255

(22) Filed: Feb. 24, 2025

Related U.S. Application Data
(63) Continuation of application No. 17/811,776, filed on Jul. 11, 2022, now Pat. No. 12,234,465.
Application No. 17/811,776 is a continuation of application No. 16/604,425, filed on Oct. 10, 2019, now Pat. No. 11,421,235.
(60) Provisional application No. 62/491,683, filed on Apr. 28, 2017.
(60) Provisional application No. 62/512,315, filed on May 30, 2017.

(51) Int. Cl. C12N 15/52; C12N 9/04; C12N 9/10; C12N 9/12; C12N 9/88; C12N 15/70; C12P 7/16; C12P 7/24

(52) U.S. Cl. CPC C12N 15/52; C12N 9/0006; C12N 9/1022; C12N 9/1205; C12N 9/88; C12N 15/70; C12P 7/16; C12P 7/24; C12Y 101/01001; C12Y 101/01086; C12Y 202/01006; C12Y 401/01072; C12Y 402/01009

(57) ABSTRACT

Genetically modified microorganisms that have the ability to convert carbon substrates into chemical products such as isobutanol are disclosed. For example, genetically modified methanotrophs that are capable of generating isobutanol at high titers from a methane source are disclosed. Methods of making these genetically modified microorganisms and methods of using them are also disclosed.

### CROSS-REFERENCE TO RELATED APPLICATIONS

This application is a continuation of U.S. application Ser. No. 17/811,776, filed Jul. 11, 2022, which in turn is a continuation of U.S. application Ser. No. 16/604,425 (now U.S. Pat. No. 11,421,235), which is a national stage entry of International Application No. PCT/US2018/029688, filed Apr. 27, 2018, which claims the priority benefit of U.S. Provisional Application Nos. 62/491,683, filed Apr. 28, 2017, and 62/512,315, filed May 30, 2017. Each of these are hereby incorporated by reference in their entireties.

### REFERENCE TO SEQUENCE LISTING

The instant application contains a Sequence Listing which has been submitted electronically in XML format and is hereby incorporated by reference in its entirety. The XML copy was created on Feb. 24, 2025, is named 17811776CONSL.xml and is 179,666 bytes in size.

### BACKGROUND OF THE DISCLOSURE

As crude oil becomes very expensive, there has been a push to use alternative methods to produce fuels and fuel additives. Alternative methods, including fermentation, have been pursued in recent years; however, most of these methods require a feedstock that consumes our food supply. For example, sugar (usually in the form of corn) is used to produce ethanol and isobutanol.

A feedstock that is relatively cheap and does not decrease overall food supply is natural gas. The methane (CH₄) contained in natural gas has great value as a chemical feedstock for the production of chemicals and food additives. Methane can be obtained from shale gas, oil drilling, municipal solid waste, biomass gasification/conversion, and methanogenic archaea. Wellhead natural gas varies in composition from 40% to 95% methane, wherein the other components include ethane, propane, butane, pentane, and heavier hydrocarbons, along with hydrogen sulfide, carbon dioxide, helium and nitrogen.

One chemical that has recently received a great deal of attention is isobutanol. Isobutanol (also known as 2-methylpropan-1-ol) is an organic compound with the formula (CH₃)₂CHCH₂OH. Since isobutanol is a higher-chain alcohol, it has an energy density that is close to gasoline. Currently, ethanol is used to supplement gasoline, and is added up to 10%. However, isobutanol has several advantageous properties that make it an attractive alternative to ethanol as a gasoline additive or biofuel. For example, isobutanol is not as volatile or corrosive as ethanol, and does not readily absorb water. Furthermore, branched-chain alcohols, such as isobutanol, have higher-octane numbers, resulting in less knocking in engines. Thus, isobutanol is fully compatible with gasoline combusting engines as well as in jet engines.

Other uses of isobutanol include, but are not limited to, its use as: a feedstock chemical in the manufacture of isobutyl acetate (which is used in the production of lacquer and similar coatings, and in the food industry as a flavoring agent); a precursor of derivative esters—isobutyl esters such as diisobutyl phthalate (DIBP) (used as plasticizers in plastics, rubbers, and other dispersions); a precursor of p-xylene (a building block for plastic bottles, textiles and clothing); a paint solvent; a varnish remover; an ink ingredient; a paint additive (to reduce viscosity, improve brush flow, and retard formation of oil residues (blush) on painted surfaces); a gasoline additive (to reduce carburetor icing); an automotive polish additive; an automotive paint cleaner additive; a chemical extractant in production of organic compounds; and a mobile phase in thin layer chromatography.

The present inventors have developed a way of using genetically modified microorganisms, such as methanotrophs, bacteria, or yeast, in order to dramatically improve the production of multi-carbon compounds, such as isobutyraldehyde and isobutanol, from cheap carbon compounds, such as methane.

### INCORPORATION BY REFERENCE

All publications, patents, and patent applications cited herein are hereby incorporated by reference in their entirety and to the same extent as if each individual publication, patent, or patent application was specifically and individually indicated to be incorporated by reference. In the event of a conflict between a term herein and a term in an incorporated reference, the term herein controls.

### SUMMARY OF THE INVENTION

Isobutanol and other alcohols are valuable chemicals that can be used in a variety of ways, such as for fuels and solvents. Disclosed herein are methods and microorganisms that can be used to generate valuable alcohols such as isobutanol.

Aldehydes, such as isobutyraldehyde and isovaleraldehyde, can also be produced by the methods and microorganisms disclosed herein. These aldehydes can be used to generate alcohols and can be converted into different useful polymers.

Disclosed herein are genetically modified microorganisms capable of converting a C₁ carbon to a multicarbon product. These microorganisms can comprise a gene encoding an acetolactate synthase (AlsS); a ketol-acid reductoisomerase; a dihydroxy-acid dehydratase (DHAD); and/or a 2-keto acid decarboxylase (KDC). In some cases, the genes encoding for the acetolactate synthase (AlsS); ketol-acid reductoisomerase (KARI); dihydroxy-acid dehydratase (DHAD); and/or 2-keto acid decarboxylase (KDC) is under the control of a rare earth metal switch. In some cases, the rare earth metal switch can be a lanthanum switch.

In one example, disclosed herein is a genetically modified microorganism capable of converting a C₁ carbon to a multicarbon product comprising a gene encoding: an acetolactate synthase (AlsS); a ketol-acid reductoisomerase (KARI); a dihydroxy-acid dehydratase (DHAD); and a 2-keto acid decarboxylase (KDC), where the gene encoding the 2-keto acid decarboxylase (KDC) comprises a polynucleotide that is at least 60% identical to SEQ ID NO: 9.

In another example, disclosed herein is a genetically modified microorganism capable of converting a C₁ carbon to a multicarbon product comprising a gene encoding for an acetolactate synthase (AlsS); a ketol-acid reductoisomerase (KARI); a dihydroxy-acid dehydratase (DHAD); a 2-keto acid decarboxylase (KDC); and an alcohol dehydrogenase (ADH), where the gene encoding the alcohol dehydrogenase (ADH) comprises a polynucleotide that is at least 60% identical to SEQ ID NO: 17.

The genetically modified microorganism can produce multicarbon products such as aldehydes. For example, the aldehyde can be isobutyraldehyde. In some cases, the genetically modified microorganism can produce an alcohol as a multicarbon product. The alcohol can be ethanol, methanol, and/or isobutanol. In some cases, isobutanol is produced.

The acetolactate synthase (AlsS) gene used can be a gram positive bacterial AlsS gene. In some cases, the AlsS gene can comprise a polynucleotide that is at least 60% identical SEQ ID NO: 1. In some cases, the AlsS gene can encode for a polypeptide that comprises an amino acid sequence that is at least 90% identical to SEQ ID NO: 2. In some cases, the AlsS gene can comprise a polynucleotide that is at least 60% identical SEQ ID NO: 100. In some cases, the AlsS gene can encode for a polypeptide that comprises an amino acid sequence that is at least 90% identical to SEQ ID NOs: 99.

The ketol-acid reductoisomerase (KARI) gene can be from a gram negative bacterial ketol-acid reductoisomerase gene. In some cases, the gene encoding for a ketol-acid reductoisomerase (KARI) comprises a polynucleotide that is at least 85% identical to SEQ ID NO: 3. In some cases, the ketol-acid reductoisomerase gene can encode for a polypeptide that comprises an amino acid sequence that is at least 90% identical to SEQ ID NO: 4.

The dihydroxy-acid dehydratase (DHAD) gene can be a gram negative bacterial dihydroxy-acid dehydratase (DHAD) gene or a methanotrophic dihydroxy-acid dehydratase (DHAD) gene. In some cases, the gene encoding a dihydroxy-acid dehydratase (DHAD) can comprise a polynucleotide that is at least 82% identical to SEQ ID NO: 5 or can comprise a polynucleotide that is 90% identical to SEQ ID NO: 7. In some cases, the gene encoding for a dihydroxy-acid dehydratase (DHAD) can encode for a polypeptide that comprises an amino acid sequence that is at least 90% identical to SEQ ID NOs: 6 or 8.

The KDC gene used in these microorganisms can comprise a polynucleotide that is at least 60% identical to SEQ ID NO: 9. In some cases, the genetically modified microorganism can further comprise one or more additional genes encoding for a 2-keto acid decarboxylase (KDC), e.g., a second KDC gene. In some cases, the 2-keto acid decarboxylase (KDC) (e.g., the second KDC) can be from a microorganism that is capable of converting a C₁ carbon to a multicarbon product. In some cases, the additional gene encoding for a 2-keto acid decarboxylase (KDC) can be a methanotroph KDC gene. In some cases, the additional gene encoding for a 2-keto acid decarboxylase (KDC) can be a Methylococcus capsulatus KDC gene. In some cases, the additional gene encoding for a 2-keto acid decarboxylase (KDC) can comprise a polynucleotide that is at least 60% identical to SEQ ID NO: 9. In some cases, the additional gene encoding for a 2-keto acid decarboxylase (KDC) can comprise a polynucleotide that is at least 60% identical to SEQ ID NO: 11. In some cases, the additional gene encoding for a 2-keto acid decarboxylase (KDC) can encode for a polypeptide comprising an amino acid sequence at least 90% identical to the amino acid sequence of any one of SEQ ID NOs: 10, 12, 55, 57, 59, 61, 63, 65, 67, 69, 71, 73, 75, 77, 79, 81, 83, 85, 87, 89, 91, 93, 95, or 97.

Also disclosed herein are microorganisms that can produce an alcohol, such as ethanol, methanol, or isobutanol (or other alcohols such as isopentanol). In these cases, the microorganism can further comprise an alcohol dehydrogenase (ADH) gene. The ADH gene can be from a gram negative or a gram positive bacteria ADH or a yeast. The ADH can be under the control of a rare earth metal switch. In some cases, the rare earth metal switch can be a lanthanum switch. In some cases, the ADH gene can encode for a polynucleotide that comprises at least 60% identical to any one of SEQ ID NOs: 13, 15, or 17. In some cases, the ADH gene encodes for a polypeptide that comprises an amino acid sequence that is at least 90% identical to any one of SEQ ID NOs: 14, 16, or 18. In some cases, the ADH gene comprises a polynucleotide that is substantially similar to any one of SEQ ID NOs: 13, 15, 17, 24, 26, 28, 30, 32, 34, 36, 38, 40, 42, 44, 46, 48, 50, 52, or 54. In some cases, the ADH gene encodes for a polypeptide that comprises an amino acid sequence that is at least 90% identical to the amino acid sequence of any one of SEQ ID NOs: 14, 16, 18, 23, 25, 27, 29, 31, 33, 35, 37, 39, 41, 43, 45, 47, 49, 51, or 53.

In some cases, the ADH gene can be an E. coli ADH gene, a S. cerevisiae ADH gene, or both. The ADH gene can also be from the genus Clostridium, Geobacillus, and/or Lactococcus. In some cases, when the ADH gene is an E. coli ADH gene, the E. coli ADH gene can comprise a polynucleotide that is at least 60% identical to SEQ ID NO: 17. In some cases, when the ADH gene is an S. cerevisiae ADH gene, the S. cerevisiae ADH gene can comprise a polynucleotide that is at least 60% identical to SEQ ID NO: 13. In some cases, both a S. cerevisiae ADH gene and an E. coli ADH gene is used, and the S. cerevisiae ADH gene can comprise a polynucleotide that is at least 60% identical to SEQ ID NO: 13, whereas the E. coli ADH gene can comprise a polynucleotide that is at least 60% identical to SEQ ID NO: 17. Additionally, the genetically modified microorganism can comprise a second ADH gene. The second ADH gene can be from E. coli, S. cerevisiae, or both. Additional ADH genes can be used as well (e.g., a third, fourth, or fifth, etc.).

In order to increase the efficiency of aldehyde or alcohol production, the genetically modified microorganism can further comprises a sugar permease gene. The sugar permease gene can be a LacY gene. In some cases, the sugar permease gene is used for gene expression. In some cases, the LacY gene can be under the control of a rare earth metal switch. In some cases, the rare earth metal switch can be a lanthanum switch.

The genetically modified microorganism can use different C₁ carbons as a carbon source, such as carbon monoxide (CO), carbon dioxide (CO₂), methane (CH₄), or any combination thereof. In some cases, the genetically modified microorganism uses CH₄ as the C₁ carbon source.

In some cases, the genetically modified microorganism can be a methanotroph, for example, from the genera Methylobacter, Methylomicrobium, Methylomonas, Methylocaldum, Methylococcus, Methylosoma, Methylosarcina, Methylothermus, Methylohalobius, Methylogaea, Methylovulum, Crenothrix, Clonothrix, Methylosphaera, Methylocapsa, Methylocella, Methylosinus, Methylocystis, Methyloferula, Methylomarinum, or Methyloacidoiphilum. In particular, methanotrophs that can be used can be from the genera Methylococcus, e.g., Methylococcus capsulatus.

In some instances, one or more of the acetolactate synthase, ketol-acid reductoisomerase, dihydroxy-acid dehydratase, 2-keto acid decarboxylase (KDC), and alcohol dehydrogenase (ADH) genes can be heterologous to the microorganism. In some cases, one or more of those genes can be endogenous to the microorganism. Further, one or more of the genes can be overexpressed. In some cases, the microorganism can comprise multiple copies of one or more of the genes.

Also disclosed herein is a genetically modified microorganism capable of converting a C₁ carbon source to a multicarbon product comprising a sugar permease gene. In some cases, the sugar permease gene can be under the control of rare earth metal switch. In some cases, the rare earth metal switch is a lanthanum switch. The sugar permease genes can be a LacY gene. In some cases, the LacY gene can be a gram negative bacterial LacY gene. In some cases, the LacY gene can comprise a polynucleotide that is at least 80% identical to SEQ ID NO: 19. In some cases, the LacY gene can encode for a polypeptide that comprises an amino acid sequence that is at least 90% identical to SEQ ID NO: 20. In some cases, the genetically modified microorganism can further comprise one or more genes encoding for: (i) acetolactate synthase (AlsS); (ii) ketol-acid reductoisomerase (KARI); (iii) dihydroxy-acid dehydratase (DHAD); (iv) 2-keto acid decarboxylase (KDC); (v) alcohol dehydrogenase (ADH); or (vi) any combination thereof. In some cases, one or more of these additional genes can be under the control of a rare earth metal switch, e.g., a lanthanum switch. One or more of these genes can be heterologous, endogenous, overexpressed, and/or comprise multiple copies (e.g., LacY, AlsS, KARI, DHAD, KDC, and/or ADH).

Further disclosed herein is a vector comprising a polynucleotide that is at least 60% identical to SEQ ID NO: 9. Additionally disclosed is a vector comprising a polynucleotide that is at least 60% identical to SEQ ID NO: 11. Also disclosed is a vector comprising a polynucleotide that is at least 60% identical to SEQ ID NO: 100.

The vector can further comprise an operably linked promoter. The vector can also further comprise one or more genes encoding for: (i) acetolactate synthase (AlsS); (ii) a ketol-acid reductoisomerase (KARI); (iii) a dihydroxy-acid dehydratase (DHAD); (iv) a 2-keto acid decarboxylase (KDC); (v) an alcohol dehydrogenase (ADH); or (vi) any combination thereof. In some cases, the one or more gene encoding for (i) an acetolactate synthase (AlsS); (ii) a ketol-acid reductoisomerase (KARI); (iii) a dihydroxy-acid dehydratase (DHAD); (iv) 2-keto acid decarboxylase (KDC); (v) an alcohol dehydrogenase (ADH); or (vi) any combination thereof, can be under the control of a rare earth metal switch, e.g., a lanthanum switch. In some cases, the vector can comprise a sugar permease gene. In some instances, the vector can comprise two or more genes encoding for the same enzyme. The two or more genes encoding for the same enzyme can be non-identical genes or in some cases, the two or more gene can be identical genes.

Additionally disclosed herein is a method of making a genetically modified microorganism capable of converting a C₁ carbon source to a multicarbon product comprising contacting a microorganism with a polynucleotide encoding for an acetolactate synthase (AlsS); a ketol-acid reductoisomerase (KARI); a dihydroxy-acid dehydratase (DHAD); and/or a 2-keto acid decarboxylase (KDC). In some cases, the 2-keto acid decarboxylase (KDC) can comprise a polynucleotide that is at least 60% identical to SEQ ID NO: 9. In some cases, the microorganism is further contacted with a second polynucleotide encoding for a 2-keto acid decarboxylase (KDC). In some cases, the microorganism is further contacted with a polynucleotide encoding for an alcohol dehydrogenase (ADH). In some cases, the genes can be under the control of a rare earth metal switch, such as a lanthanum switch. One or more of these genes can be heterologous, endogenous, overexpressed, and/or comprise multiple copies (e.g., LacY, AlsS, KARI, DHAD, KDC, and/or ADH). In some cases, the microorganism can be contacted with a sugar permease gene.

In some cases, the microorganism is contacted with a single vector or nucleic acid comprising the acetolactate synthase (AlsS) gene, the ketol-acid reductoisomerase gene, the dihydroxy-acid dehydratase gene, and the 2-keto acid decarboxylase (KDC) gene. In some cases, the microorganism is contacted with the acetolactate synthase (AlsS) gene, the ketol-acid reductoisomerase gene, the dihydroxy-acid dehydratase gene, and the 2-keto acid decarboxylase (KDC) gene using multiple vectors or nucleic acids.

Also described herein is a method of making a genetically modified microorganism capable of converting a C₁ carbon source to a multicarbon product comprising contacting a microorganism with a polynucleotide encoding for a sugar permease. The method can further comprise contacting the microorganism with one or more genes encoding for: (i) acetolactate synthase; (ii) ketol-acid reductoisomerase; (iii) dihydroxy-acid dehydratase; (iv) 2-keto acid decarboxylase; (v) alcohol dehydrogenase; or (vi) any combination thereof.

Further disclosed herein is a method of making an aldehyde from a C₁ carbon comprising: (a) contacting the C₁ carbon with a genetically modified microorganism capable of converting the C₁ carbon into a multicarbon product, where the genetically modified microorganism comprises a polynucleotide encoding for an acetolactate synthase (AlsS), a ketol-acid reductoisomerase; a dihydroxy-acid dehydratase; and a 2-keto acid decarboxylase (KDC), where the KDC comprises a polynucleotide that is at least 60% identical to SEQ ID NO: 9; and (b) growing the genetically modified microorganism to produce the aldehyde. In some cases, one or more of the genes can be under the control of a rare earth metal switch, such as a lanthanum switch.

This method can also further comprise (c) isolating the aldehyde. In some cases, the aldehyde can be isobutyraldehyde. The method can result in isobutyraldehyde being produced at a level of at least 1 g/L. The isobutyraldehyde can be isolated and can also be substantially pure.

In some cases, the microorganism can further comprise a second gene encoding for a 2-keto acid decarboxylase (KDC). In some cases, the KDC can comprise a polynucleotide that is at least 60% identical to SEQ ID NOs: 9 or 11. In some cases, the KDC can encode for a polypeptide that comprises an amino acid sequence that is at least 90% identical to any one of SEQ ID NOs: 10, 12, 55, 57, 59, 61, 63, 65, 67, 69, 71, 73, 75, 77, 79, 81, 83, 85, 87, 89, 91, 93, 95, or 97.

In some cases, the microorganism used in the method can further comprise a nucleic acid encoding for an ADH. In this case, the genetically modified microorganism can produce an alcohol, such as isobutanol.

In some cases, one or more of the genes can be under the control of a rare earth metal switch, such as a lanthanum switch.

Also disclosed herein is a method of making an alcohol from a C₁ carbon comprising: (a) contacting the C₁ carbon with a genetically modified microorganism capable of converting the C₁ carbon into a multicarbon product, where the genetically modified microorganism comprises a polynucleotide encoding for an acetolactate synthase (AlsS); a ketol-acid reductoisomerase; a dihydroxy-acid dehydratase; a 2-keto acid decarboxylase (KDC); and an alcohol dehydrogenase (ADH), where the KDC is encoded by a nucleotide sequence at least 60% identical to the nucleic acid sequence of SEQ ID NO: 9; and (b) growing the genetically modified microorganism to produce the alcohol. In some cases, the alcohol can be isobutanol.

The method can further comprise (c) isolating the alcohol. In some cases, the alcohol produced can be used as a gasoline additive, a gasoline substitute, or as jetfuel.

Also disclosed herein is an isolated polynucleotide comprising a nucleic acid sequence at least 84% identical to the nucleic acid sequence of SEQ ID NO: 1. Also disclosed herein is an isolated polynucleotide comprising a nucleic acid sequence at least 60% identical to the nucleic acid sequence of SEQ ID NO: 100. These nucleic acid sequences can encode for a protein that has acetolactate synthase activity.

Also disclosed herein is an isolated polynucleotide comprising a nucleic acid sequence at least 88% identical to the nucleic acid sequence of SEQ ID NO: 3. This nucleic acid sequence can encode for a protein that has ketol-acid reductoisomerase activity.

Further disclosed herein is an isolated polynucleotide comprising a nucleic acid sequence at least 88% identical to the nucleic acid sequence of SEQ ID NO: 5 or an isolated polynucleotide comprising a nucleic acid sequence at least 88% identical to the nucleic acid sequence of SEQ ID NO: 7. These nucleic acid sequences can encode for a protein that has dihydroxy-acid dehydratase activity.

Disclosed herein is an isolated polynucleotide comprising a nucleic acid sequence at least 85% identical to the nucleic acid sequence of SEQ ID NO: 9 or an isolated polynucleotide comprising a nucleic acid sequence at least 85% identical to the nucleic acid sequence of SEQ ID NO: 11. These nucleic acid sequences can encode for a protein that has 2-keto acid decarboxylase activity.

Further disclosed herein is an isolated polynucleotide comprising a nucleic acid sequence at least 85% identical to the nucleic acid sequence of SEQ ID NO: 13; an isolated polynucleotide comprising a nucleic acid sequence at least 85% identical to the nucleic acid sequence of SEQ ID NO: 15; and an isolated polynucleotide comprising a nucleic acid sequence at least 85% identical to the nucleic acid sequence of SEQ ID NO: 17. These nucleic acid sequences can encode for a protein that has alcohol dehydrogenase activity.

Also disclosed herein is an isolated polynucleotide comprising a nucleic acid sequence at least 84% identical to the nucleic acid sequence of SEQ ID NO: 19. This nucleic acid sequence can encode for a protein that has sugar permease activity.

Disclosed herein is an isolated polynucleotide comprising a nucleic acid sequence at least 84% identical to the nucleic acid sequence of SEQ ID NO: 21. This nucleic acid sequence can encode for a protein that has arabinose operon regulatory protein activity.

Disclosed herein is also a genetically modified microorganism capable of converting a C₁ carbon source to an aldehyde comprising one or more genes encoding for: (i) acetolactate synthase; (ii) ketol-acid reductoisomerase; (iii) dihydroxy-acid dehydratase; (iv) 2-keto acid decarboxylase; or (v) any combination thereof; where (a) the acetolactate synthase gene comprises a polynucleotide that is at least 60% identical to SEQ ID NO: 1 or 100; (b) the ketol-acid reductoisomerase gene comprises a polynucleotide that is at least 85% identical to SEQ ID NO: 3; (c) the dihydroxy-acid dehydratase gene comprises a polynucleotide that is at least 82% identical to SEQ ID NOs: 5 or 7; and/or (d) the 2-keto acid decarboxylase gene comprises a polynucleotide that is at least 60% identical to SEQ ID NOs: 9 or 11. The genetically modified microorganism can further comprise an ADH gene. The ADH gene can comprise (a) a polynucleotide that is at least 60% identical to SEQ ID NO: 13; (b) the nucleic acid sequence of SEQ ID NO: 15; and/or (c) a polynucleotide that is at least 60% identical to SEQ ID NO: 17.

Also described herein is a vector comprising one or more genes encoding for: (i) acetolactate synthase; (ii) ketol-acid reductoisomerase; (iii) dihydroxy-acid dehydratase; (iv) 2-keto acid decarboxylase; (v) alcohol dehydrogenase; or (vi) any combination thereof; where (i) the acetolactate synthase gene comprises a polynucleotide that is at least 60% identical to SEQ ID NO: 1 or 100; (ii) the ketol-acid reductoisomerase gene comprises a polynucleotide that is at least 85% identical to SEQ ID NO: 3; (iii) the dihydroxy-acid dehydratase gene comprises a polynucleotide that is at least 82% identical to SEQ ID NO: 5 and/or comprises the a polynucleotide sequence that is SEQ ID NO: 7; (iv) the 2-keto acid decarboxylase gene comprises a polynucleotide that is at least 60% identical to SEQ ID NO: 9 and/or comprises a polynucleotide that is SEQ ID NO: 11; and/or (v) the alcohol dehydrogenase gene comprises (a) a polynucleotide that is at least 60% identical to SEQ ID NO: 13; (b) a polynucleotide that is SEQ ID NO: 15; and/or (c) a polynucleotide that is at least 60% identical to SEQ ID NO: 17.

Further disclosed herein is a method of making a genetically modified microorganism capable of converting a C₁ carbon source to an aldehyde or an alcohol comprising contacting a microorganism with one or more genes encoding for: (i) an acetolactate synthase; (ii) a ketol-acid reductoisomerase; (iii) a dihydroxy-acid dehydratase; (iv) a 2-keto acid decarboxylase; (v) an alcohol dehydrogenase; or (vi) any combination thereof; where (i) the acetolactate synthase gene comprises a polynucleotide that is at least 60% identical to SEQ ID NO: 1 or 100; (ii) the ketol-acid reductoisomerase gene comprises a polynucleotide that is at least 85% identical SEQ ID NO: 3; (iii) the dihydroxy-acid dehydratase gene comprises a polynucleotide that is at least 82% identical to SEQ ID NO: 5 and/or comprises a polynucleotide that is SEQ ID NO: 7; (iv) the 2-keto acid decarboxylase gene comprises a polynucleotide that is at least 60% identical to SEQ ID NO: 9 and/or comprises a polynucleotide that is SEQ ID NO: 11; and/or (v) the alcohol dehydrogenase gene comprises (a) a polynucleotide that is at least 60% identical to SEQ ID NO: 13; (b) a polynucleotide that is SEQ ID NO: 15; and/or (c) a polynucleotide that is at least 60% identical to SEQ ID NO: 17.

Also disclosed herein is a method of making a useful product comprising: (a) contacting a genetically modified microorganism with a C₁ carbon substrate, where the microorganism comprises at least one heterologous gene encoding for: (i) an acetolactate synthase, (ii) a ketol-acid reductoisomerase, (iii) a dihydroxy-acid dehydratase, (iv) a 2-keto acid decarboxylase, (v) an alcohol dehydrogenase, or (vi) any combination thereof; and (b) growing the microorganism to produce the useful product, where the useful product comprises 2-acetolactate; 2,3-butanediol (2,3-BDO); diacetyl; 2,3-dihydroxy-2-methylbutanoic acid; 2,3-dihydroxyisovalerate; amino acids; ketoisovalerate; isobutyraldehyde; methyl methacrylate (MMA); isovaleraldehyde; isovalerate; isopentanol; isoamyl acetate; pentadecanoic acid; isobutene; or p-xylene.

Further disclosed is a genetically modified microorganism capable of converting a C₁ carbon to a multicarbon product, where the genetically modified microorganism comprises an acetolactate synthase gene; a ketol-acid reductoisomerase gene; a dihydroxy-acid dehydratase gene; and a 2-keto acid decarboxylase gene, where the acetolactate synthase gene comprises a polynucleotide that is at least 60% identical to SEQ ID NO: 100. In some cases, the genetically modified microorganism can comprise an alcohol dehydrogenase gene. In other cases, the acetolactate synthase gene, the ketol-acid reductoisomerase gene, the dihydroxy-acid dehydratase gene, the 2-keto acid decarboxylase gene, or the alcohol dehydrogenase gene is heterologous to the microorganism.

Further disclosed is a method of making a genetically modified microorganism capable of converting a C₁ carbon source to a multicarbon product, the method comprising contacting a microorganism with an acetolactate synthase gene, a ketol-acid reductoisomerase gene, a dihydroxy-acid dehydratase gene, and a 2-keto acid decarboxylase gene, where the acetolactate synthase gene comprises a polynucleotide that is at least 60% identical to SEQ ID NO: 100.

Also disclosed herein is a method of making 2-acetolactate comprising (a) contacting a C₁ carbon with a genetically modified microorganism capable of converting the C₁ carbon into a multicarbon product, where the genetically modified microorganism comprises an acetolactate synthase gene comprising a polynucleotide that is at least 60% identical to SEQ ID NO: 100; and (b) growing the genetically modified microorganism to produce the 2-acteolactate.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a metabolic pathway from sugar to pyruvate. Pyruvate can then be used to make various products such as fuels and chemicals.

FIG. 2 shows a metabolic pathway from methane (CH₄) to pyruvate. Pyruvate can then be used to make various products such as fuels and chemicals.

FIG. 3 shows a metabolic pathway from methane (CH₄) to isobutanol. Methane is converted to pyruvate by the pathway shown in FIG. 2, and pyruvate is converted into isobutanol through the action of at least five enzymes which include acetolactate synthase (gene name: AlsS); ketol-acid reductoisomerase (enzyme abbreviation: KARI; gene name: IlvC); dihydroxy-acid dehydratase (enzyme abbreviation: DHAD; gene name: IlvD); 2-keto acid decarboxylase (gene name: KDC); and alcohol dehydrogenase (gene name: ADH).

FIGS. 4A-4C show inducible expression vectors useful to express isobutanol pathway enzymes in microorganisms such as methanotrophs. FIG. 4A shows from left to right, a Kanamycin marker, an arabinose induction machinery (LacY and AraC driven by a J23115 promoter) and a BAD promoter (pBAD) driving the isobutanol pathway ((MCA0996=Kdc), Adh6, AlsS, IlvC and IlvD). FIG. 4B shows the vector from FIG. 4A with three differences: the KDC here comes from Carnobacterium divergens (Cdi), the IlvD comes from Methylococcus capsulatus, and an additional ADH gene has been added (YqhD from E. coli). FIG. 4C shows a vector that shares a similar architecture to the vector in FIG. 4A with many of the same genes. However, there are several key differences including: 1) two operons, one with pBAD (inducible by arabinose) and the second using pMxaF (strong endogenous promoter) driving the genes; and 2) the addition of a FucO alcohol dehydrogenase from E. coli.

FIG. 5 shows the arabinose induction machinery: LacY and AraC.

FIG. 6 shows the isobutanol operon expressed from the pBAD promoter. The pBAD promoter uses the pBAD ribosome binding site. In between each of the genes that follow MCA0996, the same RBS GTW0001 is used.

FIG. 7 shows a comparison of the isobutanol pathway in E. coli (see e.g., Atsumi., S., et al., “Non-fermentative pathways for synthesis of branch-chain higher alcohol as biofuels,” Nature, 451(7174); 86-9 (2008) and Atsumi, S., et al., “Engineering the isobutanol biosynthetic pathway in Escherichia coli by comparison of three aldehyde reductase/alcohol dehydrogenase genes,” Appl. Microbiol. Biotechnol., 85(3): 651-7 (2010)) versus the isobutanol pathway designed herein using an M. capsulatus KDC. The data shows that KivD (used in the E. coli pathway by Atsumi et al.) does not produce nearly as much isobutanol compared to the M. capsulatus KDC when expressed in a methanotroph. M. capsulatus KDC is better in the context of the full pathway from pyruvate to isobutanol. MH04 comprises KivD, and MH01 comprises M. capsulatus KDC.

FIG. 8 shows isobutanol titers when methanotrophs are transformed with plasmids expressing a KDC from Carnobacterium divergens (CDI) (MF1650, MF1651; MF 1654; MF1655). Methanotrophs that were not transformed (MF1659, MF1660) with plasmids expressing Carnobacterium divergens (CDI) showed an approximate 40% decrease of isobutanol production.

FIG. 9 shows the isobutanol production titer of methanotrophs that are transformed with Methylococcus capsulatus KDC or Lactococcus lactis KDC. KDC from Methylococcus capsulatus showed a vast improvement of isobutanol production (approximately 40%) compared to Lactococcus lactis KDC.

FIG. 10 shows isobutanol titers when methanotrophs are transformed with plasmids expressing either an Eco.fucO (MR2005; MF2006) or an Ec.YqdD (MF2003; MF2004) alcohol dehydrogenase. The methanotrophs expressing the Eco.fucO ADH showed increased isobutanol titers.

FIG. 11 shows isobutanol titers when methanotrophs are transformed with plasmids expressing different alcohol dehydrogenases and tested in a fermenter. Plasmids expressing Adh6, BdhB, FucO, and YqhD are represented. Methanotrophs expressing Adh6 produced the most isobutanol. BdhB and YqhD produced similar amounts of isobutanol.

FIG. 12 shows isobutanol production levels using KDCs from Andreprevotia chitinilytica (GW1035), Carnobacterium divergens (GW1049), Methylococcus capsulatus (GW1137), Methylomonas denitrificans (GW1145), and Methylobacter marinus (GW1151). KDCs from Andreprevotia chitinilytica or Carnobacterium divergens showed increased ability to produce isobutanol titers at high titers, approximately 0.8 g/L of isobutanol. Andreprevotia chitinilytica or Carnobacterium divergens KDCs produced approximately double that of Methylomonas denitrificans KDC.

FIG. 13 shows production titers of isobutyraldehyde, isovaleraldehyde, and isobutanol of two strains that do not express heterologous alcohol dehydrogenase. The strains expressed acetolactate synthase; ketol-acid reductoisomerase; dihydroxy-acid dehydratase; and 2-keto acid decarboxylase; and are designated GW692 (pBAD>Mc.KDC_Bs.AlsS_Ec.ILVC_Ec.ILVD) and SL691 (pBAD>Bsu.alsS-g.Cdi.kdc; p.mxaF>g.Mca.kdc-g.Eco.ilvC-g.Mca.ilvD). Strain GW692 produced approximately 7 g/L of isobutyraldehyde, whereas strain SL691 produced close to 10 g/L. Strain GW692 also produced approximately 5.8 g/L of isovaleraldehyde, whereas strain SL691 produced approximately 4.1 g/L.

FIG. 14 shows that strains expressing a Bacillus licheniformis AlsS exhibited significantly improved 2-acetolate production, as indicated by measuring 2,3-BDO titers. In one strain expressing Bacillus licheniformis AlsS (XZ562), the 2,3-BDO titers increased an average of 44.6% over the XZ58 strain over the course of the fermentation run. Another biological replicate (XZ561), produced also significantly higher average 2,3-BDO titers compared to the XZ58 strain. This data indicates that methanotrophs expressing Bacillus licheniformis AlsS produce significantly higher levels of 2-acetolactate compared to methanotrophs expressing other AlsS, including the Bacillus subtilis AlsS.

FIG. 15 shows the ability of various rare earth metals at a concentration of 10 μM, 35 μM, 70 μM, or 140 μM to activate or repress the pMxaF promoter as measured by mCherry. Scandium (Sc), yttrium (Y), samarium (Sm), europium (Eu), gadolinium (Gd), terbium (Tb), dysprosium (Dy), holmium (Ho), erbium (Er), thulium (Tm), and ytterbium (Yb) minimally activated or repressed the pMxaF promoter at 10 μM, 35 μM, 70 μM, or 140 μM. However, lanthanum (La), cerium (Ce), praseodymium (Pr), and neodymium (Nd) significantly repressed the expression of the pMxaF promoter at all concentrations. Maximal repression was observed starting at 35 μM for lanthanum, 70 μM for cerium, and 140 μM for praseodymium and neodymium.

FIG. 16 shows the ability of a M. capsulatus strain expressing isobutanol pathway genes under the control of a rare earth metal switch to produce isobutanol. The strains were grown up in the presence of 35 μM lanthanum in shake bottles. After 24 hours, lanthanum was diluted out and the strain was allowed to produce isobutanol. Strains expressing isobutanol pathway genes under the control of a rare earth metal switch were able to produce isobutanol while the control strain did not. Optical density was highest in control strains.

FIG. 17 shows the ability of M. capsulatus strain with an integrated copy of ADH6 to produce isobutanol. A significant amount of alcohol dehydrogenase activity from the integrated ADH was observed. Compared with the non-integrated ADH strains, there was no observable difference in total carbon, alcohol and aldehyde. Optimal density fared better with in strains with integrated copies of ADH6.

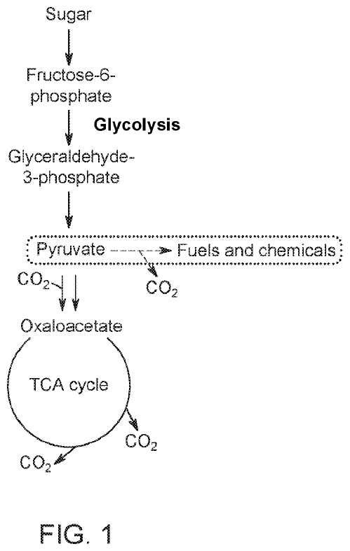

### DETAILED DESCRIPTION OF THE INVENTION

The following description and examples illustrate embodiments of the invention in detail. It is to be understood that this invention is not limited to the particular embodiments described herein and as such can vary. Those of skill in the art will recognize that there are numerous variations and modifications of this invention, which are encompassed within its scope.

Isobutanol is a high value chemical and fuel that is currently produced from the carbonylation of propylene. Two methods are currently practiced in the industry, including hydroformylation and reppe carbonylation. Hydroformylation is more common and generates a mixture of isobutyraldehydes, which are hydrogenated to the alcohols and then separated. There has been high interest in the biosynthesis of isobutanol. Fermentation typically involves taking a carbon source (usually sugar) and fermenting it using a microorganism that is capable of converting the carbon source into a desired product such as isobutanol.

Costs to produce chemicals, such as isobutanol, by fermentation typically depend on the cost of the carbon source used. Sugars are generally higher cost carbon sources that also result in a decrease of food supply. One carbon source that is currently extremely cost-effective and abundant is natural gas. The primary source of carbon within natural gas is methane. By using cheap carbon sources such as methane, alcohols, such as isobutanol, can be produced economically. However, the challenge lies in engineering fermentation methods and microorganisms to efficiently convert cheap carbon sources, such as methane, into alcohols, such as isobutanol, using a fermentation process.

Only a very few microorganisms are capable of producing isobutanol naturally at very low levels. (Felpeto-Santero, C., et al., “Engineering alternative isobutanol production platforms,” AMB Express, 5:32 (2015)) At these low titers, the cost of fermentation would be too great to be economically feasible. Thus, genetic engineering is required to produce isobutanol at an economically viable level.

Described herein are genetically modified microorganisms, e.g., methanotrophs, that can convert a carbon substrate, such as methane, into desired products. Some of the genetically modified microorganisms disclosed herein have been designed and altered to efficiently produce alcohols, such as isobutanol, or aldehydes, such as isobutyraldehyde or isovaleraldehyde, multiple folds over what is naturally produced or expected to be produced. Additionally some of the genetically modified microorganisms disclosed herein can be used to convert a carbon substrate (such as methane) into alcohols, such as isobutanol, and subsequently into fuels or other desired products. These genetically modified microorganisms and the novel methods of fermentation and uses thereof are described herein.

### Definitions

The term “alcohol” and its grammatical equivalents as used herein can refer to any and all any organic compounds whose molecule contains one or more hydroxyl groups (—OH) attached to a carbon atom. For example, ethanol and isobutanol are alcohols.

The term “aldehyde” and its grammatical equivalents as used herein can refer to any and all organic compounds whose molecule contains a function group with the structure —CHO, which has a carbonyl center (a carbon double-bonded to oxygen) with the carbon atom also bonded to hydrogen and to an R group (which can be any generic alkyl or side chain). For example, isobutyraldehyde and isovaleraldehyde are aldehydes.

The term “about” in relation to a reference numerical value and its grammatical equivalents as used herein can include the numerical value itself and a range of values plus or minus 10% from that numerical value. For example, the amount “about 10” includes 10 and any amounts from 9 to 11. For example, the term “about” in relation to a reference numerical value can also include a range of values plus or minus 10%, 9%, 8%, 7%, 6%, 5%, 4%, 3%, 2%, or 1% from that value. In some cases, the numerical disclosed throughout can be “about” that numerical value even without specifically mentioning the term “about.”

The term “gene” and its grammatical equivalents as used herein can refer to any sequence of DNA or RNA which codes for a molecule that has a function.

The terms “protein”, “peptide”, “polypeptide”, and “amino acid sequence” are used interchangeably herein to refer to polymers of amino acid residues of any length. The polymer can be linear or branched. The polymer can comprise modified amino acids or amino acid analogs, and it can be interrupted by chemical moieties other than amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling or bioactive component.

The terms “polynucleotide” and “polynucleic acid” are used interchangeably herein and refer to a polymer of ribonucleic acid (RNA) or deoxyribonucleic acid (DNA). A polynucleotide is made up of four bases: adenine, cytosine, guanine, and thymine/uracil (uracil is used in RNA). A coding sequence from a polynucleotide is indicative of the sequence of the protein encoded by the polynucleotide. The terms include various modifications and analogues.

The terms “nucleotide sequences” and “nucleic acid sequences” are used interchangeably herein and refer to deoxyribonucleic acid (DNA) or ribonucleic acid (RNA) sequences, including, without limitation, messenger RNA (mRNA), DNA/RNA hybrids, or synthetic nucleic acids. The nucleic acid can be single-stranded, or partially or completely double stranded (duplex). Duplex nucleic acids can be homoduplex or heteroduplex.

The term “genetic modification” or “genetically modified” and their grammatical equivalents as used herein can refer to one or more alterations of a nucleic acid, e.g., the nucleic acid within a microorganism's genome. For example, genetic modification can refer to alterations, additions, and/or deletion of nucleic acid (e.g., whole genes or fragments of genes).

The term “disrupting” and its grammatical equivalents as used herein can refer to a process of altering a gene, e.g., by deletion, insertion, mutation, rearrangement, or any combination thereof. For example, a gene can be disrupted by knockout. Disrupting a gene can be partially reducing or completely suppressing expression (e.g., mRNA and/or protein expression) of the gene. Disrupting can also include inhibitory technology, such as shRNA, siRNA, microRNA, dominant negative, or any other means to inhibit functionality or expression of a gene or protein.

The term “gene editing” and its grammatical equivalents as used herein can refer to genetic engineering in which one or more nucleotides are inserted, replaced, or removed from a genome. For example, gene editing can be performed using a nuclease (e.g., a natural-existing nuclease or an artificially engineered nuclease).

The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.”

The term “substantially pure” and its grammatical equivalents as used herein can mean that a particular substance does not contain a majority of another substance. For example, “substantially pure isobutanol” can mean at least 90% isobutanol. In some instances, “substantially pure isobutanol” can mean at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, 99.99%, 99.999%, or 99.9999% isobutanol. For example, substantially pure isobutanol can mean at least 70% isobutanol. In some cases, substantially pure isobutanol can mean at least 75% isobutanol. In some cases, substantially pure isobutanol can mean at least 80% isobutanol. In some cases, substantially pure isobutanol can mean at least 85% isobutanol. In some cases, substantially pure isobutanol can mean at least 90% isobutanol. In some cases, substantially pure isobutanol can mean at least 91% isobutanol. In some cases, substantially pure isobutanol can mean at least 92% isobutanol. In some cases, substantially pure isobutanol can mean at least 93% isobutanol. In some cases, substantially pure isobutanol can mean at least 94% isobutanol. In some cases, substantially pure isobutanol can mean at least 95% isobutanol. In some cases, substantially pure isobutanol can mean at least 96% isobutanol. In some cases, substantially pure isobutanol can mean at least 97% isobutanol. In some cases, substantially pure isobutanol can mean at least 98% isobutanol. In some cases, substantially pure isobutanol can mean at least 99% isobutanol.

The terms “heterologous” and “exogenous” and their grammatical equivalents as used herein can mean “from a different species.” For example, a “heterologous gene” can mean a gene that is from a different species. In some instances, as “a methanotroph comprising a heterologous gene” can mean that the methanotroph contains a gene that is not from the same methanotroph. The gene can be from a different microorganism such as yeast or from a different species such as a different methanotroph species. In some cases, the terms “heterologous” and “exogenous” and their grammatical equivalents as used herein can refer to polynucleotides and polypeptides.

The term “substantially similar” and its grammatical equivalents in reference to another sequence as used herein can mean at least 50% identical. In some instances, the term substantially similar refers to a sequence that is at least 55% identical. In some instances, the term substantially similar refers to a sequence that is at least 60% identical. In some instances, the term substantially similar refers to a sequence that is at least 65% identical. In some instances, the term substantially similar refers to a sequence that is at least 70% identical. In some instances, the term substantially similar refers to a sequence that is at least 75% identical. In some instances, the term substantially similar refers to a sequence that is at least 80% identical. In other instances, the term substantially similar refers to a sequence that is at least 85% identical. In some instances, the term substantially similar refers to a sequence that is at least 90% identical. In some instances, the term substantially similar refers to a sequence that is at least 91% identical. In some instances, the term substantially similar refers to a sequence that is at least 92% identical. In some instances, the term substantially similar refers to a sequence that is at least 93% identical. In some instances, the term substantially similar refers to a sequence that is at least 94% identical. In some instances, the term substantially similar refers to a sequence that is at least 95% identical. In some instances, the term substantially similar refers to a sequence that is at least 96% identical. In some instances, the term substantially similar refers to a sequence that is at least 97% identical. In some instances, the term substantially similar refers to a sequence that is at least 98% identical. In some instances, the term substantially similar refers to a sequence that is at least 99% identical. In some instances, the term substantially similar refers to a sequence that is 100% identical. In order to determine the percentage of identity between two sequences, the two sequences are aligned, using for example the alignment method of Needleman and Wunsch (J. Mol. Biol., 1970, 48: 443), as revised by Smith and Waterman (Adv. Appl. Math., 1981, 2: 482) so that the highest order match is obtained between the two sequences and the number of identical amino acids/nucleotides is determined between the two sequences. For example, methods to calculate the percentage identity between two amino acid sequences are generally art recognized and include, for example, those described by Carillo and Lipton (SIAM J. Applied Math., 1988, 48:1073) and those described in Computational Molecular Biology, Lesk, e.d. Oxford University Press, New York, 1988, Biocomputing: Informatics and Genomics Projects. Generally, computer programs will be employed for such calculations. Computer programs that can be used in this regard include, but are not limited to, GCG (Devereux et al., Nucleic Acids Res., 1984, 12: 387) BLASTP, BLASTN and FASTA (Altschul et al., J. Molec. Biol., 1990:215:403). A particularly preferred method for determining the percentage identity between two polypeptides involves the Clustal W algorithm (Thompson, J D, Higgines, D G and Gibson T J, 1994, Nucleic Acid Res 22(22): 4673-4680 together with the BLOSUM 62 scoring matrix (Henikoff S & Henikoff, J G, 1992, Proc. Natl. Acad. Sci. USA 89: 10915-10919 using a gap opening penalty of 10 and a gap extension penalty of 0.1, so that the highest order match obtained between two sequences where at least 50% of the total length of one of the two sequences is involved in the alignment.

The terms “acetolactate synthase” or “AlsS” and their grammatical equivalents as used herein can be interchangeably used with acetohydroxy acid synthase; acetolactate pyruvate-lyase; or acetolactate synthease. The terms “acetolactate synthase” or “AlsS” can include enzymes that are capable of converting pyruvate to 2-acetolactate. For example, the terms “acetolactate synthase” or “AlsS” can include an enzyme having an EC 2.2.1.6.

The terms “ketol acid reductoisomerase” or “IlvC” and their grammatical equivalents as used herein can be used interchangeably with acetohydroxy acid isomeroreductase, ketol acid reductoisomerase, alpha-keto-beta-hydroxylacyl reductoisomerase, acetohydroxy acid reductoisomerase, acetolactate reductoisomerase, dihydroxyisovalerate (isomerizing) dehydrogenase, isomeroreductase, and/or reductoisomerase. The terms “ketol acid reductoisomerase” or “IlvC” can include enzymes that are capable of converting 2-acetolactate into 2,3-dihydroxyisovalerate. For example, the terms “ketol acid reductoisomerase” or “IlvC” can include an enzyme having an EC 1.1.1.86.

The terms “dihydroxy-acid dehydratase” or “IlvD” and their grammatical equivalents as used herein can be used interchangeably with acetohydroxyacid dehydratase, alpha,beta-dihydroxyacid dehydratase, 2,3-dihydroxyisovalerate dehydratase, alpha,beta-dihydroxyisovalerate dehydratase, dihydroxy acid dehydrase, DHAD, or 2,3-dihydroxy-acid hydro-lyase. The terms “dihydroxy-acid dehydratase” or “IlvD” can include enzymes that are capable of converting 2,3-dihydroxyisovalerate into ketoisovalerate. For example, the terms “dihydroxy-acid dehydratase” or “IlvD” can include an enzyme having an EC 4.2.1.9.

The terms “2-keto acid decarboxylase” or “KDC” and their grammatical equivalents as used herein can include enzymes that are capable of converting ketoisovalerate into isobutyraldehyde. For example, the terms “2-keto acid decarboxylase” or “KDC” can include an enzyme having an EC 4.1.1.72.

The terms “alcohol dehydrogenase”, “ADH” or “Adh” and their grammatical equivalents as used herein can include enzymes that are capable of converting isobutyraldehyde into an alcohol such as isobutanol. For example, in some instances, the terms “alcohol dehydrogenase” or “Adh” can include an enzyme having an EC 1.1.1.1.

### I. Genetically Modified Microorganisms and Methods of Making the Same

Isobutanol and/or isobutyraldehyde is produced by some unmodified microorganisms; however, production levels are extremely low. Disclosed herein are genetically modified microorganisms that have dramatically improved isobutanol and/or isobutyraldehyde biosynthesis rates, and in some cases orders of magnitude higher than what could be naturally produced. For example, disclosed herein are microorganisms that do not normally produce isobutanol and/or isobutyraldehyde that can be genetically modified to synthesize isobutanol and/or isobutyraldehyde, including at significantly high levels.

### Microorganisms

The microorganisms described herein can use carbon substrates, such as, but not limited to CH₄, as carbon source to produce desired products. This however does not mean that these microorganisms use solely CH₄ as a carbon source. Some of the microorganisms disclosed herein can be made to utilize additional carbon substrates, including carbon substrates that the microorganism naturally uses in addition to other carbon substrates. For example, the microorganisms can be made to use two or more carbon substrates, such as CH₄ and sugar.

The microorganisms disclosed herein can be a prokaryote or eukaryote. In some cases, other microorganisms such as bacteria, yeast, or algae can be used.

Some microorganisms can use a C₁ carbon to generate a desired product. For example, some of the microorganisms that can convert C₁ carbon substrates into desired products can be a microorganism that is capable of using natural gas as a carbon substrate. In some cases, the microorganism can use the methane contained within the natural gas as a carbon source to make desired products. One type of microorganism that uses C₁ carbon substrates to form desired organic compounds are methanotrophs. The methanotrophs that can be particularly useful include methanotrophs from the genera Methylobacter, Methylomicrobium, Methylomonas, Methylocaldum, Methylococcus, Methylosoma, Methylosarcina, Methylothermus, Methylohalobius, Methylogaea, Methylovulum, Crenothrix, Clonothrix, Methylosphaera, Methylocapsa, Methylocella, Methylosinus, Methylocystis, Methyloferula, Methylomarinum, Methylacidiphilum, or any combinations thereof. Methanotrophs from the genus Methylococcus can be particularly useful. When a methanotroph from the genus Methylococcus is used, a methanotroph from the species Methylococcus capsulatus can be used.

Some microorganisms disclosed throughout are microorganisms that are capable of using CO₂ as a carbon substrate. For instance, the microorganisms can be a methanogen. Microorganisms that are capable of using CO₂ as a substrate can contain chlorophyll. One type of microorganism that uses CO₂ to form desired organic compounds are algae. Another type of microorganism that can use CO₂ as a substrate is a cyanobacterium.

Some microorganisms that can convert C₁ carbon substrates into desired products can be a microorganism that is capable of using CO as a carbon substrate. Anaerobic microorganisms can typically process CO and therefore can be used herein. One type of microorganism that naturally uses CO to form desired organic compounds are bacterium such as Clostridium. These microorganisms can be genetically modified into making substantial amounts of alcohols, such as isobutanol.

### Enzymes

In order to genetically engineer certain microorganisms to produce certain useful products such as isobutanol, microorganisms can be transformed with one or more genes that encode for specific enzymes. The genes encoding for these enzymes can be heterologous to the microorganism.

For example, in order to create a microorganism that can produce an alcohol, such as isobutanol, or an aldehyde, such as isobutyraldehyde, one or more genes (e.g., heterologous genes) can be transformed/transfected (i.e., inserted) into the microorganism (transiently or stably). The microorganism can contain an acetolactate synthase (gene name: AlsS), which is an enzyme that coverts two molecules of pyruvate into 2-acetolactate. The microorganism can in some cases comprise an ketol-acid reductoisomerase (gene name: ilvC) which is an enzyme that converts 2-acetolactate into 2,3-dihydroxy-isovalerate using NADPH as a reduced cofactor. The microorganism can also comprise an dihydroxy-acid dehydratase (gene name: ilvD), which is enzyme that converts 2,3-dihydroxy-isovalerate into 2-ketoisovalerate. The microorganism can in some cases comprise an 2-keto acid decarboxylase (gene name: KDC), which is enzyme that converts 2-ketoisovalerate into isobutyraldehyde. In order to produce isobutanol, the microorganism can in some cases comprise an alcohol dehydrogenase (gene name: ADH), which is enzyme that converts isobutyraldehyde into isobutanol.

Described throughout are microorganisms used to make alcohols, such as isobutanol, or an aldehyde, such as isobutyraldehyde, from a C₁ carbon (e.g., methane) or other multi-carbon source. In some cases, the microorganism herein can be transformed with a gene encoding for one or more of the following enzymes: (i) acetolactate synthase; (ii) ketol-acid reductoisomerase; (iii) dihydroxy-acid dehydratase; and/or (iv) 2-keto acid decarboxylase. Should the microorganism be directed to making an alcohol, the microorganism can be transformed with (v) an alcohol dehydrogenase. For example, the microorganism can be transformed with a gene encoding for an acetolactate synthase. The microorganism can be transformed with a gene encoding for a ketol-acid reductoisomerase. The microorganism can be transformed with a gene encoding for a dihydroxy-acid dehydratase. The microorganism can be transformed with a gene encoding for a 2-keto acid decarboxylase. The microorganism can be transformed with a gene encoding for an alcohol dehydrogenase. Any one of or more than one of these genes can be heterologous to the microorganism.

In some instances, the microorganism can be transformed with two or more genes selected from (i) acetolactate synthase; (ii) ketol-acid reductoisomerase; (iii) dihydroxy-acid dehydratase; (iv) 2-keto acid decarboxylase; and/or (v) alcohol dehydrogenase. One or more of the genes can be heterologous to the microorganism.

In one instance, the microorganism can be transformed with at least three or more genes selected from (i) acetolactate synthase; (ii) ketol-acid reductoisomerase; (iii) dihydroxy-acid dehydratase; (iv) 2-keto acid decarboxylase; and/or (v) alcohol dehydrogenase. One or more of the genes can be heterologous to the microorganism.

In other instances, the microorganism can be transformed with at least four or more genes selected from (i) acetolactate synthase; (ii) ketol-acid reductoisomerase; (iii) dihydroxy-acid dehydratase; (iv) 2-keto acid decarboxylase; and/or (v) alcohol dehydrogenase. One or more of the genes can be heterologous to the microorganism.

In some cases, the microorganism can be transformed with at least five or more genes encoding for enzymes such as an acetolactate synthase; ketol-acid reductoisomerase; dihydroxy-acid dehydratase; 2-keto acid decarboxylase; and alcohol dehydrogenase. In this case, the output will be an alcohol, such as isobutanol, unless the alcohol dehydrogenase is non-functional. If the alcohol dehydrogenase is non-functional, then the output will be an aldehyde, such as isobutyraldehyde. One or more of the genes can be heterologous to the microorganism.

In some cases, when an acetolactate synthase is used, the acetolactate synthase can be from a bacteria (e.g., a gram positive bacterium), such as from the genus Bacillus. For example, an acetolactate synthase can be from the species Bacillus subtilis. In some cases, the acetolactate synthase can be from the species Bacillus licheniformis.

The acetolactate synthase can comprise an amino acid sequence that is substantially similar to SEQ ID NO: 2 or 99. For example, the acetolactate synthase can comprise an amino acid sequence that is at least 60% identical to SEQ ID NO: 2 or 99. In some cases, the acetolactate synthase can comprise an amino acid sequence that is at least 65% identical to SEQ ID NO: 2 or 99. In some cases, the acetolactate synthase can comprise an amino acid sequence that is at least 70% identical to SEQ ID NO: 2 or 99. In some cases, the acetolactate synthase can comprise an amino acid sequence that is at least 75% identical to SEQ ID NO: 2 or 99. In some cases, the acetolactate synthase can comprise an amino acid sequence that is at least 80% identical to SEQ ID NO: 2 or 99. In some cases, the acetolactate synthase can comprise an amino acid sequence that is at least 85% identical to SEQ ID NO: 2 or 99. In some cases, the acetolactate synthase can comprise an amino acid sequence that is at least 90% identical to SEQ ID NO: 2 or 99. In some cases, the acetolactate synthase can comprise an amino acid sequence that is at least 91% identical to SEQ ID NO: 2 or 99. In some cases, the acetolactate synthase can comprise an amino acid sequence that is at least 92% identical to SEQ ID NO: 2 or 99. In some cases, the acetolactate synthase can comprise an amino acid sequence that is at least 93% identical to SEQ ID NO: 2 or 99. In some cases, the acetolactate synthase can comprise an amino acid sequence that is at least 94% identical to SEQ ID NO: 2 or 99. In some cases, the acetolactate synthase can comprise an amino acid sequence that is at least 95% identical to SEQ ID NO: 2 or 99. In some cases, the acetolactate synthase can comprise an amino acid sequence that is at least 96% identical to SEQ ID NO: 2 or 99. In some cases, the acetolactate synthase can comprise an amino acid sequence that is at least 97% identical to SEQ ID NO: 2 or 99. In some cases, the acetolactate synthase can comprise an amino acid sequence that is at least 98% identical to SEQ ID NO: 2 or 99. In some cases, the acetolactate synthase can comprise an amino acid sequence that is at least 99% identical to SEQ ID NO: 2 or 99. In some cases, the acetolactate synthase can comprise an amino acid sequence that is SEQ ID NO: 2 or 99.

When a ketol-acid reductoisomerase is used, the ketol-acid reductoisomerase can be from a bacteria (e.g., a gram negative bacterium), such as from the genus Escherichia. For example, the ketol-acid reductoisomerase can be from the species Escherichia coli.

The ketol-acid reductoisomerase can comprise an amino acid sequence that is substantially similar to SEQ ID NO: 4. For example, the ketol-acid reductoisomerase can comprise an amino acid sequence that is at least 60% identical to SEQ ID NO: 4. In some cases, the ketol-acid reductoisomerase can comprise an amino acid sequence that is at least 65% identical to SEQ ID NO: 4. In some cases, the ketol-acid reductoisomerase can comprise an amino acid sequence that is at least 70% identical to SEQ ID NO: 4. In some cases, the ketol-acid reductoisomerase can comprise an amino acid sequence that is at least 75% identical to SEQ ID NO: 4. In some cases, the ketol-acid reductoisomerase can comprise an amino acid sequence that is at least 80% identical to SEQ ID NO: 4. In some cases, the ketol-acid reductoisomerase can comprise an amino acid sequence that is at least 85% identical to SEQ ID NO: 4. In some cases, the ketol-acid reductoisomerase can comprise an amino acid sequence that is at least 90% identical to SEQ ID NO: 4. In some cases, the ketol-acid reductoisomerase can comprise an amino acid sequence that is at least 91% identical to SEQ ID NO: 4. In some cases, the ketol-acid reductoisomerase can comprise an amino acid sequence that is at least 92% identical to SEQ ID NO: 4. In some cases, the ketol-acid reductoisomerase can comprise an amino acid sequence that is at least 93% identical to SEQ ID NO: 4. In some cases, the ketol-acid reductoisomerase can comprise an amino acid sequence that is at least 94% identical to SEQ ID NO: 4. In some cases, the ketol-acid reductoisomerase can comprise an amino acid sequence that is at least 95% identical to SEQ ID NO: 4. In some cases, the ketol-acid reductoisomerase can comprise an amino acid sequence that is at least 96% identical to SEQ ID NO: 4. In some cases, the ketol-acid reductoisomerase can comprise an amino acid sequence that is at least 97% identical to SEQ ID NO: 4. In some cases, the ketol-acid reductoisomerase can comprise an amino acid sequence that is at least 98% identical to SEQ ID NO: 4. In some cases, the ketol-acid reductoisomerase can comprise an amino acid sequence that is at least 99% identical to SEQ ID NO: 4. In some cases, the ketol-acid reductoisomerase can comprise an amino acid sequence that is SEQ ID NO: 4.

When a dihydroxy-acid dehydratase is used, the dihydroxy-acid dehydratase can be from a bacterium (e.g., from a gram negative bacterium or a methanotroph), such as from the genus Escherichia and/or Methylococcus. More specifically, the dihydroxy-acid dehydratase can be from the species Escherichia coli and/or Methylococcus capsulatus.

The dihydroxy-acid dehydratase can comprise an amino acid sequence that is substantially similar to SEQ ID NO: 6 or 8. For example, the dihydroxy-acid dehydratase can comprise an amino acid sequence that is at least 60% identical to SEQ ID NO: 6 or 8. In some cases, the dihydroxy-acid dehydratase can comprise an amino acid sequence that is at least 65% identical to SEQ ID NO: 6 or 8. In some cases, the dihydroxy-acid dehydratase can comprise an amino acid sequence that is at least 70% identical to SEQ ID NO: 6 or 8. In some cases, the dihydroxy-acid dehydratase can comprise an amino acid sequence that is at least 75% identical to SEQ ID NO: 6 or 8. In some cases, the dihydroxy-acid dehydratase can comprise an amino acid sequence that is at least 80% identical to SEQ ID NO: 6 or 8. In some cases, the dihydroxy-acid dehydratase can comprise an amino acid sequence that is at least 85% identical to SEQ ID NO: 6 or 8. In some cases, the dihydroxy-acid dehydratase can comprise an amino acid sequence that is at least 90% identical to SEQ ID NO: 6 or 8. In some cases, the dihydroxy-acid dehydratase can comprise an amino acid sequence that is at least 91% identical to SEQ ID NO: 6 or 8. In some cases, the dihydroxy-acid dehydratase can comprise an amino acid sequence that is at least 92% identical to SEQ ID NO: 6 or 8. In some cases, the dihydroxy-acid dehydratase can comprise an amino acid sequence that is at least 93% identical to SEQ ID NO: 6 or 8. In some cases, the dihydroxy-acid dehydratase can comprise an amino acid sequence that is at least 94% identical to SEQ ID NO: 6 or 8. In some cases, the dihydroxy-acid dehydratase can comprise an amino acid sequence that is at least 95% identical to SEQ ID NO: 6 or 8. In some cases, the dihydroxy-acid dehydratase can comprise an amino acid sequence that is at least 96% identical to SEQ ID NO: 6 or 8. In some cases, the dihydroxy-acid dehydratase can comprise an amino acid sequence that is at least 97% identical to SEQ ID NO: 6 or 8. In some cases, the dihydroxy-acid dehydratase can comprise an amino acid sequence that is at least 98% identical to SEQ ID NO: 6 or 8. In some cases, the dihydroxy-acid dehydratase can comprise an amino acid sequence that is at least 99% identical to SEQ ID NO: 6 or 8. In some cases, the dihydroxy-acid dehydratase can comprise an amino acid sequence that is SEQ ID NO: 6 or 8.

When a 2-keto acid decarboxylase (KDC) is used, the (KDC can be from a bacterium (e.g., a gram positive bacterium) such as from the genus Carnobacterium and/or a methanotroph such as from the genus Methylococcus. More specifically, the KDC can be from the species Carnobacterium divergens and/or Methylococcus capsulatus.

The KDC can be from other bacterium, such as those listed in Table 4. For example, the KDC can be from the genus Methylocaldum, Methylosarcina, Methylomonas, Methylohalobius, Methylobacter, Lamprocystis, Andreprevotia, Lactococcus, Streptococcus, Enterococcus, Brochothrix, Carnobacterium, Helicobacter, Staphylococcus, and/or Fictibacillus. For example, KDC from the following species can be particularly useful: Methylocaldum szegediense, Methylosarcina lacus, Methylomonas denitrificans, Methylomonas methanica, Methylohalobius crimeensis, Methylobacter marinus, Methylobacter luteus, Lamprocystis purpurea, Andreprevotia chitinilytica, Lactococcus lactis, Streptococcus didelphis, Enterococcus caccae, Enterococcus haemoperoxidus, Enterococcus moraviensis, Carnobacterium maltaromaticum, Brochothrix thermosphacta, Carnobacterium gallinarum, Carnobacterium divergens, Helicobacter bizzozeronii, Staphylococcus aureus subsp. aureus CIG290, and/or Fictibacillus macauensis.

The 2-keto acid decarboxylase can comprise an amino acid sequence that is substantially similar to any one of SEQ ID NOs: 10, 12, 55, 57, 59, 61, 63, 65, 67, 69, 71, 73, 75, 77, 79, 81, 83, 85, 87, 89, 91, 93, 95, or 97. For example, the 2-keto acid decarboxylase can comprise an amino acid sequence that is at least 60% identical to any one of SEQ ID NOs: 10, 12, 55, 57, 59, 61, 63, 65, 67, 69, 71, 73, 75, 77, 79, 81, 83, 85, 87, 89, 91, 93, 95, or 97. In some cases, the 2-keto acid decarboxylase can comprise an amino acid sequence that is at least 65% identical to any one of SEQ ID NOs: 10, 12, 55, 57, 59, 61, 63, 65, 67, 69, 71, 73, 75, 77, 79, 81, 83, 85, 87, 89, 91, 93, 95, or 97. In some cases, the 2-keto acid decarboxylase can comprise an amino acid sequence that is at least 70% identical to any one of SEQ ID NOs: 10, 12, 55, 57, 59, 61, 63, 65, 67, 69, 71, 73, 75, 77, 79, 81, 83, 85, 87, 89, 91, 93, 95, or 97. In some cases, the 2-keto acid decarboxylase can comprise an amino acid sequence that is at least 75% identical to any one of SEQ ID NOs: 10, 12, 55, 57, 59, 61, 63, 65, 67, 69, 71, 73, 75, 77, 79, 81, 83, 85, 87, 89, 91, 93, 95, or 97. In some cases, the 2-keto acid decarboxylase can comprise an amino acid sequence that is at least 80% identical to any one of SEQ ID NOs: 10, 12, 55, 57, 59, 61, 63, 65, 67, 69, 71, 73, 75, 77, 79, 81, 83, 85, 87, 89, 91, 93, 95, or 97. In some cases, the 2-keto acid decarboxylase can comprise an amino acid sequence that is at least 85% identical to any one of SEQ ID NOs: 10, 12, 55, 57, 59, 61, 63, 65, 67, 69, 71, 73, 75, 77, 79, 81, 83, 85, 87, 89, 91, 93, 95, or 97. In some cases, the 2-keto acid decarboxylase can comprise an amino acid sequence that is at least 90% identical to any one of SEQ ID NOs: 10, 12, 55, 57, 59, 61, 63, 65, 67, 69, 71, 73, 75, 77, 79, 81, 83, 85, 87, 89, 91, 93, 95, or 97. In some cases, the 2-keto acid decarboxylase can comprise an amino acid sequence that is at least 91% identical to any one of SEQ ID NOs: 10, 12, 55, 57, 59, 61, 63, 65, 67, 69, 71, 73, 75, 77, 79, 81, 83, 85, 87, 89, 91, 93, 95, or 97. In some cases, the 2-keto acid decarboxylase can comprise an amino acid sequence that is at least 92% identical to any one of SEQ ID NOs: 10, 12, 55, 57, 59, 61, 63, 65, 67, 69, 71, 73, 75, 77, 79, 81, 83, 85, 87, 89, 91, 93, 95, or 97. In some cases, the 2-keto acid decarboxylase can comprise an amino acid sequence that is at least 93% identical to any one of SEQ ID NOs: 10, 12, 55, 57, 59, 61, 63, 65, 67, 69, 71, 73, 75, 77, 79, 81, 83, 85, 87, 89, 91, 93, 95, or 97. In some cases, the 2-keto acid decarboxylase can comprise an amino acid sequence that is at least 94% identical to any one of SEQ ID NOs: 10, 12, 55, 57, 59, 61, 63, 65, 67, 69, 71, 73, 75, 77, 79, 81, 83, 85, 87, 89, 91, 93, 95, or 97. In some cases, the 2-keto acid decarboxylase can comprise an amino acid sequence that is at least 95% identical to any one of SEQ ID NOs: 10, 12, 55, 57, 59, 61, 63, 65, 67, 69, 71, 73, 75, 77, 79, 81, 83, 85, 87, 89, 91, 93, 95, or 97. In some cases, the 2-keto acid decarboxylase can comprise an amino acid sequence that is at least 96% identical to any one of SEQ ID NOs: 10, 12, 55, 57, 59, 61, 63, 65, 67, 69, 71, 73, 75, 77, 79, 81, 83, 85, 87, 89, 91, 93, 95, or 97. In some cases, the 2-keto acid decarboxylase can comprise an amino acid sequence that is at least 97% identical to any one of SEQ ID NOs: 10, 12, 55, 57, 59, 61, 63, 65, 67, 69, 71, 73, 75, 77, 79, 81, 83, 85, 87, 89, 91, 93, 95, or 97. In some cases, the 2-keto acid decarboxylase can comprise an amino acid sequence that is at least 98% identical to any one of SEQ ID NOs: 10, 12, 55, 57, 59, 61, 63, 65, 67, 69, 71, 73, 75, 77, 79, 81, 83, 85, 87, 89, 91, 93, 95, or 97. In some cases, the 2-keto acid decarboxylase can comprise an amino acid sequence that is at least 99% identical to any one of SEQ ID NOs: 10, 12, 55, 57, 59, 61, 63, 65, 67, 69, 71, 73, 75, 77, 79, 81, 83, 85, 87, 89, 91, 93, 95, or 97. In some cases, the 2-keto acid decarboxylase can comprise an amino acid sequence that is any one of SEQ ID NOs: 10, 12, 55, 57, 59, 61, 63, 65, 67, 69, 71, 73, 75, 77, 79, 81, 83, 85, 87, 89, 91, 93, 95, or 97.

In some cases, two or more KDCs can be used. In some cases, two KDC genes can be used to increase the production of aldehydes, such as isobutyraldehyde, and/or alcohols, such as isobutanol. In other cases, three KDCs can be used to increase the production of isobutyraldehyde and/or isobutanol. In some cases, four, five, or six KDCs can be used to increase the production of isobutyraldehyde and/or isobutanol.

When an alcohol dehydrogenase is used, the alcohol dehydrogenase can be from a yeast such as from the genus Saccharomyces or a bacterium (e.g., a gram negative or gram positive bacterium) such as from the genus Escherichia. More specifically, the alcohol dehydrogenase can be from the species Saccharomyces cerevisiae and/or Escherichia coli.

Other ADHs that can be used can be from the genus Clostridium, Geobacillus, Lactococcus, Oenococcus, Pectobacterium, and/or Psychrobacter. For example, ADHs from the following species can be particularly useful: Clostridium acetobutylicum, Geobacillus stearothermophilus, Geobacillus thermoglucosidas, Lactococcus lactis, Oenococcus oeni, Pectobacterium atrosepticum, and/or Psychrobacter cryohalolentis.

The alcohol dehydrogenase can comprise an amino acid sequence that is substantially similar to any one of SEQ ID NOs: 14, 16, 18, 23, 25, 27, 29, 31, 33, 35, 37, 39, 41, 43, 45, 47, 49, 51, or 53. For example, the alcohol dehydrogenase can comprise an amino acid sequence that is at least 60% identical to any one of SEQ ID NOs: 14, 16, 18, 23, 25, 27, 29, 31, 33, 35, 37, 39, 41, 43, 45, 47, 49, 51, or 53. In some cases, the alcohol dehydrogenase can comprise an amino acid sequence that is at least 65% identical to any one of SEQ ID NOs: 14, 16, 18, 23, 25, 27, 29, 31, 33, 35, 37, 39, 41, 43, 45, 47, 49, 51, or 53. In some cases, the alcohol dehydrogenase can comprise an amino acid sequence that is at least 70% identical to any one of SEQ ID NOs: 14, 16, 18, 23, 25, 27, 29, 31, 33, 35, 37, 39, 41, 43, 45, 47, 49, 51, or 53. In some cases, the alcohol dehydrogenase can comprise an amino acid sequence that is at least 75% identical to any one of SEQ ID NOs: 14, 16, 18, 23, 25, 27, 29, 31, 33, 35, 37, 39, 41, 43, 45, 47, 49, 51, or 53. In some cases, the alcohol dehydrogenase can comprise an amino acid sequence that is at least 80% identical to any one of SEQ ID NOs: 14, 16, 18, 23, 25, 27, 29, 31, 33, 35, 37, 39, 41, 43, 45, 47, 49, 51, or 53. In some cases, the alcohol dehydrogenase can comprise an amino acid sequence that is at least 85% identical to any one of SEQ ID NOs: 14, 16, 18, 23, 25, 27, 29, 31, 33, 35, 37, 39, 41, 43, 45, 47, 49, 51, or 53. In some cases, the alcohol dehydrogenase can comprise an amino acid sequence that is at least 90% identical to any one of SEQ ID NOs: 14, 16, 18, 23, 25, 27, 29, 31, 33, 35, 37, 39, 41, 43, 45, 47, 49, 51, or 53. In some cases, the alcohol dehydrogenase can comprise an amino acid sequence that is at least 91% identical to any one of SEQ ID NOs: 14, 16, 18, 23, 25, 27, 29, 31, 33, 35, 37, 39, 41, 43, 45, 47, 49, 51, or 53. In some cases, the alcohol dehydrogenase can comprise an amino acid sequence that is at least 92% identical to any one of SEQ ID NOs: 14, 16, 18, 23, 25, 27, 29, 31, 33, 35, 37, 39, 41, 43, 45, 47, 49, 51, or 53. In some cases, the alcohol dehydrogenase can comprise an amino acid sequence that is at least 93% identical to any one of SEQ ID NOs: 14, 16, 18, 23, 25, 27, 29, 31, 33, 35, 37, 39, 41, 43, 45, 47, 49, 51, or 53. In some cases, the alcohol dehydrogenase can comprise an amino acid sequence that is at least 94% identical to any one of SEQ ID NOs: 14, 16, 18, 23, 25, 27, 29, 31, 33, 35, 37, 39, 41, 43, 45, 47, 49, 51, or 53. In some cases, the alcohol dehydrogenase can comprise an amino acid sequence that is at least 95% identical to any one of SEQ ID NOs: 14, 16, 18, 23, 25, 27, 29, 31, 33, 35, 37, 39, 41, 43, 45, 47, 49, 51, or 53. In some cases, the alcohol dehydrogenase can comprise an amino acid sequence that is at least 96% identical to any one of SEQ ID NOs: 14, 16, 18, 23, 25, 27, 29, 31, 33, 35, 37, 39, 41, 43, 45, 47, 49, 51, or 53. In some cases, the alcohol dehydrogenase can comprise an amino acid sequence that is at least 97% identical to any one of SEQ ID NOs: 14, 16, 18, 23, 25, 27, 29, 31, 33, 35, 37, 39, 41, 43, 45, 47, 49, 51, or 53. In some cases, the alcohol dehydrogenase can comprise an amino acid sequence that is at least 98% identical to any one of SEQ ID NOs: 14, 16, 18, 23, 25, 27, 29, 31, 33, 35, 37, 39, 41, 43, 45, 47, 49, 51, or 53. In some cases, the alcohol dehydrogenase can comprise an amino acid sequence that is at least 99% identical to any one of SEQ ID NOs: 14, 16, 18, 23, 25, 27, 29, 31, 33, 35, 37, 39, 41, 43, 45, 47, 49, 51, or 53. In some cases, the alcohol dehydrogenase can comprise an amino acid sequence that is any one of SEQ ID NOs: 14, 16, 18, 23, 25, 27, 29, 31, 33, 35, 37, 39, 41, 43, 45, 47, 49, 51, or 53.

In some cases, two or more ADHs can be used. In some cases, two ADHs can be used to increase the production of alcohols, such as isobutanol. In other cases, three ADHs can be used to increase the production of isobutanol. In some cases, four, five, or six ADHs can be used to increase the production of isobutanol.

Additional enzymes can be placed inside the microorganism in order to make the process more efficient and/or to produce other desired end products.

For example, a sugar permease can be placed within the microorganism in order to increase production of the desired end product, such as an alcohol, e.g., isobutanol, or an aldehyde, e.g., isobutyraldehyde. In some cases, the sugar permease can be from a bacterium (e.g., a gram negative bacterium) such as from the genus Escherichia. More specifically, the sugar permease can be from the species Escherichia coli.

The sugar permease can comprise an amino acid sequence that is substantially similar to SEQ ID NO: 20. For example, the sugar permease can comprise an amino acid sequence that is at least 60% identical to SEQ ID NO: 20. In some cases, the sugar permease can comprise an amino acid sequence that is at least 65% identical to SEQ ID NO: 20. In some cases, the sugar permease can comprise an amino acid sequence that is at least 70% identical to SEQ ID NO: 20. In some cases, the sugar permease can comprise an amino acid sequence that is at least 75% identical to SEQ ID NO: 20. In some cases, the sugar permease can comprise an amino acid sequence that is at least 80% identical to SEQ ID NO: 20. In some cases, the sugar permease can comprise an amino acid sequence that is at least 85% identical to SEQ ID NO: 20. In some cases, the sugar permease can comprise an amino acid sequence that is at least 90% identical to SEQ ID NO: 20. In some cases, the sugar permease can comprise an amino acid sequence that is at least 91% identical to SEQ ID NO: 20. In some cases, the sugar permease can comprise an amino acid sequence that is at least 92% identical to SEQ ID NO: 20. In some cases, the sugar permease can comprise an amino acid sequence that is at least 93% identical to SEQ ID NO: 20. In some cases, the sugar permease can comprise an amino acid sequence that is at least 94% identical to SEQ ID NO: 20. In some cases, the sugar permease can comprise an amino acid sequence that is at least 95% identical to SEQ ID NO: 20. In some cases, the sugar permease can comprise an amino acid sequence that is at least 96% identical to SEQ ID NO: 20. In some cases, the sugar permease can comprise an amino acid sequence that is at least 97% identical to SEQ ID NO: 20. In some cases, the sugar permease can comprise an amino acid sequence that is at least 98% identical to SEQ ID NO: 20. In some cases, the sugar permease can comprise an amino acid sequence that is at least 99% identical to SEQ ID NO: 20. In some cases, the sugar permease can comprise an amino acid sequence that is SEQ ID NO: 20.

Another peptide that can be placed within the microorganism in order to increase production of the desired end product, such as an alcohol, e.g., isobutanol, or an aldehyde, e.g., isobutyraldehyde, is an arabinose operon regulatory protein (AraC). In some cases, the arabinose operon regulatory protein can be from a bacterium (e.g., a gram negative bacterium) such as from the genus Escherichia. More specifically, the arabinose operon regulatory protein can be from the species Escherichia coli.

The arabinose operon regulatory protein can comprise an amino acid sequence that is substantially similar to SEQ ID NO: 22. For example, the arabinose operon regulatory protein can comprise an amino acid sequence that is at least 60% identical to SEQ ID NO: 22. In some cases, the arabinose operon regulatory protein can comprise an amino acid sequence that is at least 65% identical to SEQ ID NO: 22. In some cases, the arabinose operon regulatory protein can comprise an amino acid sequence that is at least 70% identical to SEQ ID NO: 22. In some cases, the arabinose operon regulatory protein can comprise an amino acid sequence that is at least 75% identical to SEQ ID NO: 22. In some cases, the arabinose operon regulatory protein can comprise an amino acid sequence that is at least 80% identical to SEQ ID NO: 22. In some cases, the arabinose operon regulatory protein can comprise an amino acid sequence that is at least 85% identical to SEQ ID NO: 22. In some cases, the arabinose operon regulatory protein can comprise an amino acid sequence that is at least 90% identical to SEQ ID NO: 22. In some cases, the arabinose operon regulatory protein can comprise an amino acid sequence that is at least 91% identical to SEQ ID NO: 22. In some cases, the arabinose operon regulatory protein can comprise an amino acid sequence that is at least 92% identical to SEQ ID NO: 22. In some cases, the arabinose operon regulatory protein can comprise an amino acid sequence that is at least 93% identical to SEQ ID NO: 22. In some cases, the arabinose operon regulatory protein can comprise an amino acid sequence that is at least 94% identical to SEQ ID NO: 22. In some cases, the arabinose operon regulatory protein can comprise an amino acid sequence that is at least 95% identical to SEQ ID NO: 22. In some cases, the arabinose operon regulatory protein can comprise an amino acid sequence that is at least 96% identical to SEQ ID NO: 22. In some cases, the arabinose operon regulatory protein can comprise an amino acid sequence that is at least 97% identical to SEQ ID NO: 22. In some cases, the arabinose operon regulatory protein can comprise an amino acid sequence that is at least 98% identical to SEQ ID NO: 22. In some cases, the arabinose operon regulatory protein can comprise an amino acid sequence that is at least 99% identical to SEQ ID NO: 22. In some cases, the arabinose operon regulatory protein can comprise an amino acid sequence that is SEQ ID NO: 22.

The amino acid sequences can also be optimized based on the microorganism in which the enzymes will be expressed. In other words, conservative amino acids substitutions can be made based on whether the respective microorganism typically uses a specific amino acid or how much of that particular amino acid is available for use within the microorganism.

### Vectors

Polynucleotide constructs prepared for introduction into a prokaryotic or eukaryotic host can typically, but not always, comprise a replication system (i.e. vector) recognized by the host, including the intended polynucleotide fragment encoding the desired polypeptide, and can, but not necessarily, also include transcription and translational initiation regulatory sequences operably linked to the polypeptide-encoding segment. Expression systems (such as expression vectors) can include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, mRNA stabilizing sequences, nucleotide sequences homologous to host chromosomal DNA, and/or a multiple cloning site. Signal peptides can also be included where appropriate, for example, from secreted polypeptides of the same or related species, which allow the protein to cross and/or lodge in cell membranes or be secreted from the cell.

The vectors can be constructed using standard methods (see, e.g., Sambrook et al., Molecular Biology: A Laboratory Manual, Cold Spring Harbor, N.Y. 1989; and Ausubel, et al., Current Protocols in Molecular Biology, Greene Publishing, Co. N.Y, 1995).

The manipulation of polynucleotides that encode the enzymes disclosed herein is typically carried out in recombinant vectors. Numerous vectors are publicly available, including bacterial plasmids, bacteriophage, artificial chromosomes, episomal vectors and gene expression vectors, which can all be employed. A vector can be selected to accommodate a polynucleotide encoding a protein of a desired size. Following recombinant modification of a selected vector, a suitable host cell (e.g., the microorganisms described herein) is transfected or transformed with the vector. Each vector contains various functional components, which generally include a cloning site, an origin of replication and at least one selectable marker gene. A vector can additionally possess one or more of the following elements: an enhancer, promoter, and transcription termination and/or other signal sequences. Such sequence elements can be optimized for the selected host species. Such sequence elements can be positioned in the vicinity of the cloning site, such that they are operatively linked to the gene encoding a preselected enzyme.

Vectors, including cloning and expression vectors, can contain nucleic acid sequences that enable the vector to replicate in one or more selected microorganisms. For example, the sequence can be one that enables the vector to replicate independently of the host chromosomal DNA and can include origins of replication or autonomously replicating sequences. Such sequences are known for a variety of bacteria, yeast and viruses. For example, the origin of replication from the plasmid pBR322 is suitable for most Gram-negative bacteria, the 2 micron plasmid origin is suitable for yeast, and various viral origins (e.g.SV40, adenovirus) are useful for cloning vectors.

A cloning or expression vector can contain a selection gene (also referred to as a selectable marker). This gene encodes a protein necessary for the survival or growth of transformed microorganisms in a selective culture medium. Microorganisms not transformed with the vector containing the selection gene will therefore not survive in the culture medium. Typical selection genes encode proteins that confer resistance to antibiotics and other toxins, e.g. ampicillin, neomycin, methotrexate, hygromycin, thiostrepton, apramycin or tetracycline, complement auxotrophic deficiencies, or supply critical nutrients not available in the growth media.

The replication of vectors can be performed in E. coli. An E. coli-selectable marker, for example, the β-lactamase gene that confers resistance to the antibiotic ampicillin, can be of use. These selectable markers can be obtained from E. coli plasmids, such as pBR322 or a pUC plasmid such as pUC18 or pUC19, or pUC119.

### Promoters

Vectors can contain a promoter that is recognized by the host microorganism. The promoter can be operably linked to a coding sequence of interest. Such a promoter can be inducible or constitutive. Polynucleotides are operably linked when the polynucleotides are in a relationship permitting them to function in their intended manner.

Different promoters can be used to drive the expression of the genes. For example, if temporary gene expression (i.e., non-constitutively expressed) is desired, expression can be driven by inducible promoters. For example, the inducible or repressible promoters that can be used include but are not limited to: (a) sugars such as arabinose and lactose (or non metabolizable analogs, e.g., isopropyl β-D-1-thiogalactopyranoside (IPTG)); (b) metals such as lanthanum, copper, and calcium; (c) temperature; (d) nitrogen-source; (e) oxygen; (f) cell state (growth or stationary); (g) micronutrients such as phosphate, magnesium, and sulfur; (h) CRISPRi; (i) jun; (j) fos; (k) metallothionein and/or (1) heat shock. These promoters can be used in a methanotroph systems. For example, examples of a promoter that can be used within the methanotrophs are a pBAD promoter, a pMxaF promoter, and/or a pTrc promoter.

Constitutively expressed promoters can also be used in the vector systems herein. For example, the promoters that can be used include but are not limited to p.Bba.J23111, p.Bba.J23115, p.Bba.J61111, p.Bba.J61103, p.Bba.J61104, p.Bba.J61105, p.Bba.J61102, p.Bba.J61106, p.Bba.J61107, p.Bba.J61116, p.Bba.J61113, p.Bba.J61101, p.Bba.J61109, p.Bba.J61100, p.Bba.J61114, p.Bba.J61108, p.Bba.J61115, p.Bba.J61110, p.Bba.J61112, uMc.GlgC, uMc_IlvC, uGTW0001, uMc.IlvD, uMCA0996, uMc.IlvK, uMc.pmoB, iIlvE, uMc.IlvC, RL122pM, uMc.MCA, or uMc.GrosES promoters. Other promoters that can be used include but are not limited to pXoxF, pMxaF, pTRC, J12100, J23102, pBAD, J23110, lacO, J23116, J23106, J23105, J23108, J23107, J23115, J23114, J23118, J23104, J23101, J23119, and uMCA3034.

Promoters suitable for use with prokaryotic hosts can include, for example, the a-lactamase and lactose promoter systems, alkaline phosphatase, the tryptophan (trp) promoter system, the erythromycin promoter, apramycin promoter, hygromycin promoter, methylenomycin promoter and hybrid promoters such as the tac promoter. Promoters for use in bacterial systems will also generally contain a Shine-Dalgarno sequence operably linked to the coding sequence.

Generally, a strong promoter can be employed to provide for high level transcription and expression of the desired product.

One or more promoters of a transcription unit can be an inducible promoter. For example, a green fluorescent protein (GFP) can be expressed from a constitutive promoter while an inducible promoter drives transcription of a gene coding for one or more enzymes as disclosed herein and/or the amplifiable selectable marker.

Some vectors can contain prokaryotic sequences that facilitate the propagation of the vector in bacteria. Thus, the vectors can have other components such as an origin of replication (e.g., a nucleic acid sequence that enables the vector to replicate in one or more selected microorganisms), antibiotic resistance genes for selection in bacteria, and/or an amber stop codon which can permit translation to read through the codon. Additional selectable gene(s) can also be incorporated. Generally, in cloning vectors the origin of replication is one that enables the vector to replicate independently of the host chromosomal DNA, and includes origins of replication or autonomously replicating sequences. Such sequences can include the ColEl origin of replication in bacteria or other known sequences.

### Rare Earth Metal Molecular Switches

In some cases, the gene expression during the fermentation of high value chemicals (such as those described throughout e.g., isobutyraldehyde and isobutanol), requires precise control/timing of gene expression. In these cases, a molecular switch can be used. Switches that are particularly useful can be a rare earth metal switch. For example, a rare earth metal can be used to control gene expression, including but not limited to cerium (Ce), dysprosium (Dy), erbium (Er), europium (Eu), gadolinium (Gd), holmium (Ho), lanthanum (La), lutetium (Lu), neodymium (Nd), praseodymium (Pr), promethium (Pm), samarium (Sm), scandium (Sc), terbium (Tb), thulium (Tm), ytterbium (Yb), yttrium (Y), or any combination thereof. Any of the genes disclosed throughout or any combination thereof, can be controlled by a rare earth metal switch.

### Lanthanum

In cases where a switch is used, the media can comprise a molecule that induces or represses the switch. For example, when a lanthanum sensitive switch is used to repress the expression of one or more of the genes described herein, the media can comprise lanthanum, which will repress expression of the one or more genes under the control of the switch. In the case of a lanthanum switch any one of the following concentrations can be used to effectively repress expression of the one or more genes that are under the control of a lanthanum switch: 0.1 μM; 0.5 μM; 1 μM; 2 μM; 3 μM; 4 μM; 5 μM; 6 μM; 7 μM; 8 μM; 9 μM; 10 μM; 12.5 μM; 15 μM; 17.5 μM; 20 μM; 25 μM; 50 μM; 100 μM or more.

In some cases, the lanthanum in the media can be diluted to turn on expression of the one or more lanthanum repressed genes. For example, in some cases, the dilution of lanthanum containing media can be 1:1 (1 part lanthanum containing media to 1 part non-lanthanum containing media). In some cases, the dilution can be at least 1:2; 1:3; 1:4; 1:5; 1:7.5; 1:10; 1:15; 1:20; 1:25; 1:30; 1:35; 1:40; 1:45; 1:50; 1:75; 1:100; 1:200; 1:300; 1:400; 1:500; 1:1,000; or 1:10,000.

In some cases, the microorganism can be grown in media comprising lanthanum. The media can then be diluted to effectively turn on the expression of the lanthanum repressed genes. The microorganism can be then grown to produce desired products, such as the multicarbon products (or others disclosed throughout).

### Cerium, Praseodymium and Neodymium

In certain cases, a cerium, praseodymium, and/or neodymium sensitive switch can be used to repress or induce the expression of one or more of the genes described herein. In some cases, the media can comprise cerium, praseodymium, and/or neodymium, which can in some instances repress expression of the one or more genes under the control of the switch. In the case of cerium, praseodymium, and/or neodymium any one of the following concentrations can effectively repress expression of the one or more genes under the control of the cerium, praseodymium, and/or neodymium switch: 1 μM; 2 μM; 3 μM; 4 μM; 5 μM; 6 μM; 7 μM; 8 μM; 9 μM; 10 μM; 12.5 μM; 15 μM; 17.5 μM; 20 μM; 25 μM; 30 μM; 35 μM; 40 μM; 45 μM; 50 μM; 55 μM; 60 μM; 65 μM; 70 μM; 75 μM; 80 μM; 85 μM; 90 μM; 95 μM; 100 μM; 105 μM; 110 μM; 115 μM; 120 μM; 125 μM; 130 μM; 135 μM; 140 μM; 145 μM; 150 μM; 155 μM; 160 μM; 165 μM; 170 μM; 175 μM; 180 μM; 185 μM; 190 μM; 195 μM; 200 μM or more.

In some cases, the cerium, praseodymium, and/or neodymium in the media can be diluted to turn on expression of the one or more cerium, praseodymium, and/or neodymium repressed genes. For example, in some cases, the dilution of cerium, praseodymium, and/or neodymium containing media can be 1:1 (1 part cerium, praseodymium, and/or neodymium containing media to 1 part non-cerium, praseodymium, and/or neodymium containing media). In some cases, the dilution can be at least 1:2; 1:3; 1:4; 1:5; 1:7.5; 1:10; 1:15; 1:20; 1:25; 1:30; 1:35; 1:40; 1:45; 1:50; 1:75; 1:100; 1:200; 1:300; 1:400; 1:500; 1:1,000; or 1:10,000.

In some cases, the microorganism can be grown in media comprising cerium, praseodymium, and/or neodymium. The media can then be diluted to effectively turn on the expression of the cerium, praseodymium, and/or neodymium repressed genes. The microorganism can be then grown to produce desired products, such as the multicarbon products (or others disclosed throughout).

### Scandium, Yttrium, Samrium, Europium, Gadolinium, Terbium, Dysprosium, Holmium, Erbium, Thulium, and Yterribium

In certain cases, a Scandium, Yttrium, Samrium, Europium, Gadolinium, Terbium, Dysprosium, Holmium, Erbium, Thulium, and/or Yterribium switch can be used to repress or induce the expression of one or more of the genes described herein. In some cases, the media can comprise Scandium, Yttrium, Samrium, Europium, Gadolinium, Terbium, Dysprosium, Holmium, Erbium, Thulium, and/or Yterribium, which will repress or induce expression of the one or more genes under the control of the switch. In the case of Scandium, Yttrium, Samrium, Europium, Gadolinium, Terbium, Dysprosium, Holmium, Erbium, Thulium, and/or Yterribium any one of the following concentrations can effectively repress expression of the one or more genes the switch: 10 μM; 20 μM; 30 μM; 40 μM; 50 μM; 60 μM; 70 μM; 80 μM; 90 μM; 100 μM; 120 μM; 140 μM; 150 μM; 175 μM; 200 μM or more.

In some cases, the Scandium, Yttrium, Samrium, Europium, Gadolinium, Terbium, Dysprosium, Holmium, Erbium, Thulium, and/or Yterribium in the media can be diluted to reverse the effect of Scandium, Yttrium, Samrium, Europium, Gadolinium, Terbium, Dysprosium, Holmium, Erbium, Thulium, and/or Yterribium. For example, in some cases, the dilution of Scandium, Yttrium, Samrium, Europium, Gadolinium, Terbium, Dysprosium, Holmium, Erbium, Thulium, and/or Yterribium containing media can be 1:1 (1 part Scandium, Yttrium, Samrium, Europium, Gadolinium, Terbium, Dysprosium, Holmium, Erbium, Thulium, and/or Yterribium containing media to 1 part non-Scandium, Yttrium, Samrium, Europium, Gadolinium, Terbium, Dysprosium, Holmium, Erbium, Thulium, and/or Yterribium containing media). In some cases, the dilution can be at least 1:2; 1:3; 1:4; 1:5; 1:7.5; 1:10; 1:15; 1:20; 1:25; 1:30; 1:35; 1:40; 1:45; 1:50; 1:75; 1:100; 1:200; 1:300; 1:400; 1:500; 1:1,000; or 1:10,000.

After dilution, the microorganism can be then grown to produce desired products, such as the multicarbon products (or others disclosed throughout).

### Other Switches

### Arabinose

In some cases, an arabinose switch can be used to induce/repress the expression of one or more of the genes described herein. In these cases, the media can comprise arabinose, which will in some cases, induce the expression of the one or more genes under the control of the switch. In the case of arabinose any one of the following concentrations can effectively induce/repress expression of the one or more genes: 0.1 g/L; 0.5 g/L; 1 g/L; 2 g/L; 3 g/L; 4 g/L; 5 g/L; 6 g/L; 7 g/L; 8 g/L; 9 g/L; 10 g/L; 11 g/L; 12 g/L; 13 g/L; 14 g/L; 15 g/L; 16 g/L; 17 g/L; 18 g/L; 19 g/L; g/L; 20 g/L; 25 g/L; 30 g/L; 35 g/L; 40 g/L; 45 g/L; 50 g/L; 55 g/L; 60 g/L; 65 g/L; 70 g/L; 75 g/L; 80 g/L; 85 g/L; 90 g/L; 95 g/L; 100 g/L or more.

In some cases, any one of the following concentrations of arabinose can effectively induce/repress gene expression of the one or more genes controlled by an arabinose switch: 0.1 mM; 0.2 mM; 0.3 mM; 0.4 mM; 0.5 mM; 0.6 mM; 0.7 mM; 0.8 mM; 0.9 mM; 1 mM; 1.5 mM; 2 mM; 2.5 mM; 3 mM; 3.5 mM; 4 mM; 4.5 mM; 5 mM; 5.5 mM; 6 mM; 6.6 mM; 7 mM; 7.5 mM; 8 mM; 8.5 mM; 9 mM; 9.5 mM; 10 mM; 12.5 mM; 15 mM; 17.5 mM; 20 mM; 25 mM; 50 mM; 100 mM or more.

In some cases, the arabinose in the media can be diluted to turn on/off the expression of the one or more arabinose repressed/induced genes. For example, in some cases, the dilution of arabinose containing media can be 1:1 (1 part arabinose containing media to 1 part non-arabinose containing media). In some cases, the dilution can be at least 1:2; 1:3; 1:4; 1:5; 1:7.5; 1:10; 1:15; 1:20; 1:25; 1:30; 1:35; 1:40; 1:45; 1:50; 1:75; 1:100; 1:200; 1:300; 1:400; 1:500; 1:1,000; or 1:10,000.

### Isopropyl β-D-1-Thiogalactopyranoside

In certain cases, an IPTG sensitive switch can be used to induce or repress the expression of one or more of the genes described herein. In some cases, the media can comprise IPTG, which can in some instances induce expression of the one or more genes under the control of the switch. In the case of IPTG any one of the following concentrations can effectively induce or repress expression of the one or more genes: 1 μM; 2 μM; 3 μM; 4 μM; 5 μM; 6 μM; 7 μM; 8 μM; 9 μM; 10 μM; 12.5 μM; 15 μM; 17.5 μM; 20 μM; 25 μM; 30 μM; 35 μM; 40 μM; 45 μM; 50 μM; 55 μM; 60 μM; 65 μM; 70 μM; 75 μM; 80 μM; 85 μM; 90 μM; 95 μM; 100 μM; 105 μM; 110 μM; 115 μM; 120 μM; 125 μM; 130 μM; 135 μM; 140 μM; 145 μM; 150 μM; 155 μM; 160 μM; 165 μM; 170 μM; 175 μM; 180 μM; 185 μM; 190 μM; 195 μM; 200 μM or more.

In some cases, the IPTG in the media can be diluted to turn on or off expression of the one or more IPTG induced or repressed genes. For example, in some cases, the dilution of IPTG containing media can be 1:1 (1 part IPTG containing media to 1 part non-IPTG containing media). In some cases, the dilution can be at least 1:2; 1:3; 1:4; 1:5; 1:7.5; 1:10; 1:15; 1:20; 1:25; 1:30; 1:35; 1:40; 1:45; 1:50; 1:75; 1:100; 1:200; 1:300; 1:400; 1:500; 1:1,000; or 1:10,000.

In some cases, the microorganism can be grown in media comprising IPTG. IPTG can be added to the media to turn on the expression of IPTG induced genes. The microorganism can be then grown to produce desired products, such as the multicarbon products (or others disclosed throughout). The media can then be diluted to effectively turn off the expression of the IPTG induced genes.

### Genes

The vectors described throughout can comprise a nucleic acid sequence of one or more of the following genes: (i) acetolactate synthase; (ii) ketol-acid reductoisomerase; (iii) dihydroxy-acid dehydratase; and/or (iv) 2-keto acid decarboxylase. In the case of alcohol production, the vector can also contain a nucleic acid sequence of an (v) alcohol dehydrogenase. For example, the vector can comprise an acetolactate synthase gene. The vector can comprise a ketol-acid reductoisomerase gene. The vector can comprise a dihydroxy-acid dehydratase gene. The vector can comprise an 2-keto acid decarboxylase gene. The vector can comprise an alcohol dehydrogenase gene. These genes can be heterologous to the microorganism in which the vector is contacted with (and eventually transformed with).

In some instances, the vector can comprise two or more of the following genes: (i) acetolactate synthase; (ii) ketol-acid reductoisomerase; (iii) dihydroxy-acid dehydratase; (iv) 2-keto acid decarboxylase; and/or (v) alcohol dehydrogenase. In one situation, the vector can comprise at least three or more of the genes. In another case, the vector can comprise at least four or more of the genes. In another instance, the vector can comprise all five of the genes. The vector with all five genes in most cases, will be used for alcohol (e.g., isobutanol) producing strains, unless the alcohol dehydrogenase gene is non-functional at the genetic or protein level. One or more of the genes can be heterologous to the microorganism in which the vector is contacted with (and eventually transformed with).

In some cases, when an acetolactate synthase is desired, the acetolactate synthase gene can be from a bacteria (e.g., a gram positive bacterium), such as from the genus Bacillus, or the species Bacillus subtilis.

The acetolactate synthase gene can comprise a nucleotide sequence that is substantially similar to SEQ ID NO: 1 or 100. For example, the acetolactate synthase gene can comprise a nucleotide sequence that is at least 60% identical to SEQ ID NO: 1 or 100. In some cases, the acetolactate synthase gene can comprise a nucleotide sequence that is at least 65% identical to SEQ ID NO: 1 or 100. In some cases, the acetolactate synthase gene can comprise a nucleotide sequence that is at least 70% identical to SEQ ID NO: 1 or 100. In some cases, the acetolactate synthase gene can comprise a nucleotide sequence that is at least 75% identical to SEQ ID NO: 1 or 100. In some cases, the acetolactate synthase gene can comprise a nucleotide sequence that is at least 80% identical to SEQ ID NO: 1 or 100. In some cases, the acetolactate synthase gene can comprise a nucleotide sequence that is at least 85% identical to SEQ ID NO: 1 or 100. In some cases, the acetolactate synthase gene can comprise a nucleotide sequence that is at least 90% identical to SEQ ID NO: 1 or 100. In some cases, the acetolactate synthase gene can comprise a nucleotide sequence that is at least 91% identical to SEQ ID NO: 1 or 100. In some cases, the acetolactate synthase gene can comprise a nucleotide sequence that is at least 92% identical to SEQ ID NO: 1 or 100. In some cases, the acetolactate synthase gene can comprise a nucleotide sequence that is at least 93% identical to SEQ ID NO: 1 or 100. In some cases, the acetolactate synthase gene can comprise a nucleotide sequence that is at least 94% identical to SEQ ID NO: 1 or 100. In some cases, the acetolactate synthase gene can comprise a nucleotide sequence that is at least 95% identical to SEQ ID NO: 1 or 100. In some cases, the acetolactate synthase gene can comprise a nucleotide sequence that is at least 96% identical to SEQ ID NO: 1 or 100. In some cases, the acetolactate synthase gene can comprise a nucleotide sequence that is at least 97% identical to SEQ ID NO: 1 or 100. In some cases, the acetolactate synthase gene can comprise a nucleotide sequence that is at least 98% identical to SEQ ID NO: 1 or 100. In some cases, the acetolactate synthase gene can comprise a nucleotide sequence that is at least 99% identical to SEQ ID NO: 1 or 100. In some cases, the acetolactate synthase gene can comprise a nucleotide sequence that is SEQ ID NO: 1 or 100.

When a ketol-acid reductoisomerase is desired, the ketol-acid reductoisomerase gene can be from a bacteria (e.g., a gram negative bacterium), such as from the genus Escherichia, or from the species Escherichia coli.

The ketol-acid reductoisomerase gene can comprise a nucleotide sequence that is substantially similar to SEQ ID NO: 3. For example, the ketol-acid reductoisomerase gene can comprise a nucleotide sequence that is at least 60% identical to SEQ ID NO: 3. In some cases, the ketol-acid reductoisomerase gene can comprise a nucleotide sequence that is at least 65% identical to SEQ ID NO: 3. In some cases, the ketol-acid reductoisomerase gene can comprise a nucleotide sequence that is at least 70% identical to SEQ ID NO: 3. In some cases, the ketol-acid reductoisomerase gene can comprise a nucleotide sequence that is at least 75% identical to SEQ ID NO: 3. In some cases, the ketol-acid reductoisomerase gene can comprise a nucleotide sequence that is at least 80% identical to SEQ ID NO: 3. In some cases, the ketol-acid reductoisomerase gene can comprise a nucleotide sequence that is at least 85% identical to SEQ ID NO: 3. In some cases, the ketol-acid reductoisomerase gene can comprise a nucleotide sequence that is at least 90% identical to SEQ ID NO: 3. In some cases, the ketol-acid reductoisomerase gene can comprise a nucleotide sequence that is at least 91% identical to SEQ ID NO: 3. In some cases, the ketol-acid reductoisomerase gene can comprise a nucleotide sequence that is at least 92% identical to SEQ ID NO: 3. In some cases, the ketol-acid reductoisomerase gene can comprise a nucleotide sequence that is at least 93% identical to SEQ ID NO: 3. In some cases, the ketol-acid reductoisomerase gene can comprise a nucleotide sequence that is at least 94% identical to SEQ ID NO: 3. In some cases, the ketol-acid reductoisomerase gene can comprise a nucleotide sequence that is at least 95% identical to SEQ ID NO: 3. In some cases, the ketol-acid reductoisomerase gene can comprise a nucleotide sequence that is at least 96% identical to SEQ ID NO: 3. In some cases, the ketol-acid reductoisomerase gene can comprise a nucleotide sequence that is at least 97% identical to SEQ ID NO: 3. In some cases, the ketol-acid reductoisomerase gene can comprise a nucleotide sequence that is at least 98% identical to SEQ ID NO: 3. In some cases, the ketol-acid reductoisomerase gene can comprise a nucleotide sequence that is at least 99% identical to SEQ ID NO: 3. In some cases, the ketol-acid reductoisomerase gene can comprise a nucleotide sequence that is SEQ ID NO: 3.

When a dihydroxy-acid dehydratase is desired, the dihydroxy-acid dehydratase gene can be a gene from a bacterium (e.g., a gram negative bacterium) or a methanotroph, such as from the genus Escherichia and/or Methylococcus, or from the species Escherichia coli and/or Methylococcus capsulatus.

The dihydroxy-acid dehydratase gene can comprise a nucleotide sequence that is substantially similar to SEQ ID NO: 5 or 7. For example, the dihydroxy-acid dehydratase gene can comprise a nucleotide sequence that is at least 60% identical to SEQ ID NO: 5 or 7. In some cases, the dihydroxy-acid dehydratase gene can comprise a nucleotide sequence that is at least 65% identical to SEQ ID NO: 5 or 7. In some cases, the dihydroxy-acid dehydratase gene can comprise a nucleotide sequence that is at least 70% identical to SEQ ID NO: 5 or 7. In some cases, the dihydroxy-acid dehydratase gene can comprise a nucleotide sequence that is at least 75% identical to SEQ ID NO: 5 or 7. In some cases, the dihydroxy-acid dehydratase gene can comprise a nucleotide sequence that is at least 80% identical to SEQ ID NO: 5 or 7. In some cases, the dihydroxy-acid dehydratase gene can comprise a nucleotide sequence that is at least 85% identical to SEQ ID NO: 5 or 7. In some cases, the dihydroxy-acid dehydratase gene can comprise a nucleotide sequence that is at least 90% identical to SEQ ID NO: 5 or 7. In some cases, the dihydroxy-acid dehydratase gene can comprise a nucleotide sequence that is at least 91% identical to SEQ ID NO: 5 or 7. In some cases, the dihydroxy-acid dehydratase gene can comprise a nucleotide sequence that is at least 92% identical to SEQ ID NO: 5 or 7. In some cases, the dihydroxy-acid dehydratase gene can comprise a nucleotide sequence that is at least 93% identical to SEQ ID NO: 5 or 7. In some cases, the dihydroxy-acid dehydratase gene can comprise a nucleotide sequence that is at least 94% identical to SEQ ID NO: 5 or 7. In some cases, the dihydroxy-acid dehydratase gene can comprise a nucleotide sequence that is at least 95% identical to SEQ ID NO: 5 or 7. In some cases, the dihydroxy-acid dehydratase gene can comprise a nucleotide sequence that is at least 96% identical to SEQ ID NO: 5 or 7. In some cases, the dihydroxy-acid dehydratase gene can comprise a nucleotide sequence that is at least 97% identical to SEQ ID NO: 5 or 7. In some cases, the dihydroxy-acid dehydratase gene can comprise a nucleotide sequence that is at least 98% identical to SEQ ID NO: 5 or 7. In some cases, the dihydroxy-acid dehydratase gene can comprise a nucleotide sequence that is at least 99% identical to SEQ ID NO: 5 or 7. In some cases, the dihydroxy-acid dehydratase gene can comprise a nucleotide sequence that is SEQ ID NO: 5 or 7.

When a 2-keto acid decarboxylase (KDC) gene is desired, the KDC can be a gene from a bacterium (e.g., a gram positive bacterium) or a methanotroph, such as from the genus Carnobacterium and/or Methylococcus, or from the species Carnobacterium divergens and/or Methylococcus capsulatus.

The KDC gene can be from other bacterium, such as those listed in Table 4. For example, the KDC gene can be from the genus Methylocaldum, Methylosarcina, Methylomonas, Methylohalobius, Methylobacter, Lamprocystis, Andreprevotia, Lactococcus, Streptococcus, Enterococcus, Brochothrix, Carnobacterium, Helicobacter, Staphylococcus, and/or Fictibacillus. For example, KDCs from the following species can be particularly useful: Methylocaldum szegediense, Methylosarcina lacus, Methylomonas denitrificans, Methylomonas methanica, Methylohalobius crimeensis, Methylobacter marinus, Methylobacter luteus, Lamprocystis purpurea, Andreprevotia chitinilytica, Lactococcus lactis, Streptococcus didelphis, Enterococcus caccae, Enterococcus haemoperoxidus, Enterococcus moraviensis, Carnobacterium maltaromaticum, Brochothrix thermosphacta, Carnobacterium gallinarum, Carnobacterium divergens, Helicobacter bizzozeronii, Staphylococcus aureus subsp. aureus CIG290, and/or Fictibacillus macauensis.

The 2-keto acid decarboxylase gene can comprise a nucleotide sequence that is substantially similar to any one of SEQ ID NOs: 9, 11, 56, 58, 60, 62, 64, 66, 68, 70, 72, 74, 76, 78, 80, 82, 84, 86, 88, 90, 92, 94, 96, or 98. For example, the 2-keto acid decarboxylase gene can comprise a nucleotide sequence that is at least 60% identical to any one of SEQ ID NOs: 9, 11, 56, 58, 60, 62, 64, 66, 68, 70, 72, 74, 76, 78, 80, 82, 84, 86, 88, 90, 92, 94, 96, or 98. In some cases, the 2-keto acid decarboxylase gene can comprise a nucleotide sequence that is at least 65% identical to any one of SEQ ID NOs: 9, 11, 56, 58, 60, 62, 64, 66, 68, 70, 72, 74, 76, 78, 80, 82, 84, 86, 88, 90, 92, 94, 96, or 98. In some cases, the 2-keto acid decarboxylase gene can comprise a nucleotide sequence that is at least 70% identical to any one of SEQ ID NOs: 9, 11, 56, 58, 60, 62, 64, 66, 68, 70, 72, 74, 76, 78, 80, 82, 84, 86, 88, 90, 92, 94, 96, or 98. In some cases, the 2-keto acid decarboxylase gene can comprise a nucleotide sequence that is at least 75% identical to any one of SEQ ID NOs: 9, 11, 56, 58, 60, 62, 64, 66, 68, 70, 72, 74, 76, 78, 80, 82, 84, 86, 88, 90, 92, 94, 96, or 98. In some cases, the 2-keto acid decarboxylase gene can comprise a nucleotide sequence that is at least 80% identical to any one of SEQ ID NOs: 9, 11, 56, 58, 60, 62, 64, 66, 68, 70, 72, 74, 76, 78, 80, 82, 84, 86, 88, 90, 92, 94, 96, or 98. In some cases, the 2-keto acid decarboxylase gene can comprise a nucleotide sequence that is at least 85% identical to any one of SEQ ID NOs: 9, 11, 56, 58, 60, 62, 64, 66, 68, 70, 72, 74, 76, 78, 80, 82, 84, 86, 88, 90, 92, 94, 96, or 98. In some cases, the 2-keto acid decarboxylase gene can comprise a nucleotide sequence that is at least 90% identical to any one of SEQ ID NOs: 9, 11, 56, 58, 60, 62, 64, 66, 68, 70, 72, 74, 76, 78, 80, 82, 84, 86, 88, 90, 92, 94, 96, or 98. In some cases, the 2-keto acid decarboxylase gene can comprise a nucleotide sequence that is at least 91% identical to any one of SEQ ID NOs: 9, 11, 56, 58, 60, 62, 64, 66, 68, 70, 72, 74, 76, 78, 80, 82, 84, 86, 88, 90, 92, 94, 96, or 98. In some cases, the 2-keto acid decarboxylase gene can comprise a nucleotide sequence that is at least 92% identical to any one of SEQ ID NOs: 9, 11, 56, 58, 60, 62, 64, 66, 68, 70, 72, 74, 76, 78, 80, 82, 84, 86, 88, 90, 92, 94, 96, or 98. In some cases, the 2-keto acid decarboxylase gene can comprise a nucleotide sequence that is at least 93% identical to any one of SEQ ID NOs: 9, 11, 56, 58, 60, 62, 64, 66, 68, 70, 72, 74, 76, 78, 80, 82, 84, 86, 88, 90, 92, 94, 96, or 98. In some cases, the 2-keto acid decarboxylase gene can comprise a nucleotide sequence that is at least 94% identical to any one of SEQ ID NOs: 9, 11, 56, 58, 60, 62, 64, 66, 68, 70, 72, 74, 76, 78, 80, 82, 84, 86, 88, 90, 92, 94, 96, or 98. In some cases, the 2-keto acid decarboxylase gene can comprise a nucleotide sequence that is at least 95% identical to any one of SEQ ID NOs: 9, 11, 56, 58, 60, 62, 64, 66, 68, 70, 72, 74, 76, 78, 80, 82, 84, 86, 88, 90, 92, 94, 96, or 98. In some cases, the 2-keto acid decarboxylase gene can comprise a nucleotide sequence that is at least 96% identical to any one of SEQ ID NOs: 9, 11, 56, 58, 60, 62, 64, 66, 68, 70, 72, 74, 76, 78, 80, 82, 84, 86, 88, 90, 92, 94, 96, or 98. In some cases, the 2-keto acid decarboxylase gene can comprise a nucleotide sequence that is at least 97% identical to any one of SEQ ID NOs: 9, 11, 56, 58, 60, 62, 64, 66, 68, 70, 72, 74, 76, 78, 80, 82, 84, 86, 88, 90, 92, 94, 96, or 98. In some cases, the 2-keto acid decarboxylase gene can comprise a nucleotide sequence that is at least 98% identical to any one of SEQ ID NOs: 9, 11, 56, 58, 60, 62, 64, 66, 68, 70, 72, 74, 76, 78, 80, 82, 84, 86, 88, 90, 92, 94, 96, or 98. In some cases, the 2-keto acid decarboxylase gene can comprise a nucleotide sequence that is at least 99% identical to any one of SEQ ID NOs: 9, 11, 56, 58, 60, 62, 64, 66, 68, 70, 72, 74, 76, 78, 80, 82, 84, 86, 88, 90, 92, 94, 96, or 98. In some cases, the 2-keto acid decarboxylase gene can comprise a nucleotide sequence that is any one of SEQ ID NOs: 9, 11, 56, 58, 60, 62, 64, 66, 68, 70, 72, 74, 76, 78, 80, 82, 84, 86, 88, 90, 92, 94, 96, or 98.

In some cases, two or more KDC genes can be used. In some cases, two KDC genes can be used to increase the production of aldehydes, such as isobutyraldehyde, and/or alcohols, such as isobutanol. In other cases, three KDC genes can be used to increase the production of isobutyraldehyde and/or isobutanol. In some cases, four, five, or six KDC genes can be used to increase the production of isobutyraldehyde and/or isobutanol.

When an alcohol dehydrogenase is desired, the alcohol dehydrogenase gene can be from a bacterium (e.g., a gram negative or gram positive bacterium) or a yeast, such as from the genus Escherichia or Saccharomyces, or from the species Escherichia coli or Saccharomyces cerevisiae.

Other ADH genes that can be used can be from the genus Clostridium, Geobacillus, Lactococcus, Oenococcus, Pectobacterium, and/or Psychrobacter. For example, ADH genes from the following species can be particularly useful: Clostridium acetobutylicum, Geobacillus stearothermophilus, Geobacillus thermoglucosidas, Lactococcus lactis, Oenococcus oeni, Pectobacterium atrosepticum, and/or Psychrobacter cryohalolentis.

The alcohol dehydrogenase gene can comprise a nucleotide sequence that is substantially similar to any one of SEQ ID NOs: 13, 15, 17, 24, 26, 28, 30, 32, 34, 36, 38, 40, 42, 44, 46, 48, 50, 52, or 54. For example, the alcohol dehydrogenase gene can comprise a nucleotide sequence that is at least 60% identical to any one of SEQ ID NOs: 13, 15, 17, 24, 26, 28, 30, 32, 34, 36, 38, 40, 42, 44, 46, 48, 50, 52, or 54. In some cases, the alcohol dehydrogenase gene can comprise a nucleotide sequence that is at least 65% identical to any one of SEQ ID NOs: 13, 15, 17, 24, 26, 28, 30, 32, 34, 36, 38, 40, 42, 44, 46, 48, 50, 52, or 54. In some cases, the alcohol dehydrogenase gene can comprise a nucleotide sequence that is at least 70% identical to any one of SEQ ID NOs: 13, 15, 17, 24, 26, 28, 30, 32, 34, 36, 38, 40, 42, 44, 46, 48, 50, 52, or 54. In some cases, the alcohol dehydrogenase gene can comprise a nucleotide sequence that is at least 75% identical to any one of SEQ ID NOs: 13, 15, 17, 24, 26, 28, 30, 32, 34, 36, 38, 40, 42, 44, 46, 48, 50, 52, or 54. In some cases, the alcohol dehydrogenase gene can comprise a nucleotide sequence that is at least 80% identical to any one of SEQ ID NOs: 13, 15, 17, 24, 26, 28, 30, 32, 34, 36, 38, 40, 42, 44, 46, 48, 50, 52, or 54. In some cases, the alcohol dehydrogenase gene can comprise a nucleotide sequence that is at least 85% identical to any one of SEQ ID NOs: 13, 15, 17, 24, 26, 28, 30, 32, 34, 36, 38, 40, 42, 44, 46, 48, 50, 52, or 54. In some cases, the alcohol dehydrogenase gene can comprise a nucleotide sequence that is at least 90% identical to any one of SEQ ID NOs: 13, 15, 17, 24, 26, 28, 30, 32, 34, 36, 38, 40, 42, 44, 46, 48, 50, 52, or 54. In some cases, the alcohol dehydrogenase gene can comprise a nucleotide sequence that is at least 91% identical to any one of SEQ ID NOs: 13, 15, 17, 24, 26, 28, 30, 32, 34, 36, 38, 40, 42, 44, 46, 48, 50, 52, or 54. In some cases, the alcohol dehydrogenase gene can comprise a nucleotide sequence that is at least 92% identical to any one of SEQ ID NOs: 13, 15, 17, 24, 26, 28, 30, 32, 34, 36, 38, 40, 42, 44, 46, 48, 50, 52, or 54. In some cases, the alcohol dehydrogenase gene can comprise a nucleotide sequence that is at least 93% identical to any one of SEQ ID NOs: 13, 15, 17, 24, 26, 28, 30, 32, 34, 36, 38, 40, 42, 44, 46, 48, 50, 52, or 54. In some cases, the alcohol dehydrogenase gene can comprise a nucleotide sequence that is at least 94% identical to any one of SEQ ID NOs: 13, 15, 17, 24, 26, 28, 30, 32, 34, 36, 38, 40, 42, 44, 46, 48, 50, 52, or 54. In some cases, the alcohol dehydrogenase gene can comprise a nucleotide sequence that is at least 95% identical to any one of SEQ ID NOs: 13, 15, 17, 24, 26, 28, 30, 32, 34, 36, 38, 40, 42, 44, 46, 48, 50, 52, or 54. In some cases, the alcohol dehydrogenase gene can comprise a nucleotide sequence that is at least 96% identical to any one of SEQ ID NOs: 13, 15, 17, 24, 26, 28, 30, 32, 34, 36, 38, 40, 42, 44, 46, 48, 50, 52, or 54. In some cases, the alcohol dehydrogenase gene can comprise a nucleotide sequence that is at least 97% identical to any one of SEQ ID NOs: 13, 15, 17, 24, 26, 28, 30, 32, 34, 36, 38, 40, 42, 44, 46, 48, 50, 52, or 54. In some cases, the alcohol dehydrogenase gene can comprise a nucleotide sequence that is at least 98% identical to any one of SEQ ID NOs: 13, 15, 17, 24, 26, 28, 30, 32, 34, 36, 38, 40, 42, 44, 46, 48, 50, 52, or 54. In some cases, the alcohol dehydrogenase gene can comprise a nucleotide sequence that is at least 99% identical to any one of SEQ ID NOs: 13, 15, 17, 24, 26, 28, 30, 32, 34, 36, 38, 40, 42, 44, 46, 48, 50, 52, or 54. In some cases, the alcohol dehydrogenase gene can comprise a nucleotide sequence that is any one of SEQ ID NOs: 13, 15, 17, 24, 26, 28, 30, 32, 34, 36, 38, 40, 42, 44, 46, 48, 50, 52, or 54.

In some cases, two or more ADH genes can be used. In some cases, two ADH genes can be used to increase the production of alcohols, such as isobutanol. In other cases, three ADH genes can be used to increase the production of isobutanol. In some cases, four, five, or six ADH genes can be used to increase the production of isobutanol.

Additional genes can be placed inside the microorganism in order to make other desired end products by fermentation.

For example, a sugar permease gene can be place within the microorganism in order to increase production of the desired end product, such as an aldehyde, e.g., isobutyraldehyde, or an alcohol, e.g., isobutanol. In some cases, the sugar permease gene can be from a bacterium (e.g., a gram negative bacterium) such as from the genus Escherichia. More specifically, the sugar permease can be from the species Escherichia coli.

The sugar permease gene can comprise a nucleotide sequence that is substantially similar to SEQ ID NO: 19. For example, the sugar permease gene can comprise a nucleotide sequence that is at least 60% identical to SEQ ID NO: 19. In some cases, the sugar permease gene can comprise a nucleotide sequence that is at least 65% identical to SEQ ID NO: 19. In some cases, the sugar permease gene can comprise a nucleotide sequence that is at least 70% identical to SEQ ID NO: 19. In some cases, the sugar permease gene can comprise a nucleotide sequence that is at least 75% identical to SEQ ID NO: 19. In some cases, the sugar permease gene can comprise a nucleotide sequence that is at least 80% identical to SEQ ID NO: 19. In some cases, the sugar permease gene can comprise a nucleotide sequence that is at least 85% identical to SEQ ID NO: 19. In some cases, the sugar permease gene can comprise a nucleotide sequence that is at least 90% identical to SEQ ID NO: 19. In some cases, the sugar permease gene can comprise a nucleotide sequence that is at least 91% identical to SEQ ID NO: 19. In some cases, the sugar permease gene can comprise a nucleotide sequence that is at least 92% identical to SEQ ID NO: 19. In some cases, the sugar permease gene can comprise a nucleotide sequence that is at least 93% identical to SEQ ID NO: 19. In some cases, the sugar permease gene can comprise a nucleotide sequence that is at least 94% identical to SEQ ID NO: 19. In some cases, the sugar permease gene can comprise a nucleotide sequence that is at least 95% identical to SEQ ID NO: 19. In some cases, the sugar permease gene can comprise a nucleotide sequence that is at least 96% identical to SEQ ID NO: 19. In some cases, the sugar permease gene can comprise a nucleotide sequence that is at least 97% identical to SEQ ID NO: 19. In some cases, the sugar permease gene can comprise a nucleotide sequence that is at least 98% identical to SEQ ID NO: 19. In some cases, the sugar permease gene can comprise a nucleotide sequence that is at least 99% identical to SEQ ID NO: 19. In some cases, the sugar permease gene can comprise a nucleotide sequence that is SEQ ID NO: 19.

Another enzyme that can be place within the microorganism in order to increase production of the desired end product, such as an aldehyde, e.g., isobutyraldehyde, or an alcohol, e.g., isobutanol, is an arabinose operon regulatory protein (AraC). In some cases, the arabinose operon regulatory protein gene can be from a bacterium (e.g., a gram negative bacterium) such as from the genus Escherichia. More specifically, the arabinose operon regulatory protein gene can be from the species Escherichia coli.

The arabinose operon regulatory protein gene can comprise a nucleotide sequence that is substantially similar to SEQ ID NO: 21. For example, the arabinose operon regulatory protein gene can comprise a nucleotide sequence that is at least 60% identical to SEQ ID NO: 21. In some cases, the arabinose operon regulatory protein gene can comprise a nucleotide sequence that is at least 65% identical to SEQ ID NO: 21. In some cases, the arabinose operon regulatory protein gene can comprise a nucleotide sequence that is at least 70% identical to SEQ ID NO: 21. In some cases, the arabinose operon regulatory protein gene can comprise a nucleotide sequence that is at least 75% identical to SEQ ID NO: 21. In some cases, the arabinose operon regulatory protein gene can comprise a nucleotide sequence that is at least 80% identical to SEQ ID NO: 21. In some cases, the arabinose operon regulatory protein gene can comprise a nucleotide sequence that is at least 85% identical to SEQ ID NO: 21. In some cases, the arabinose operon regulatory protein gene can comprise a nucleotide sequence that is at least 90% identical to SEQ ID NO: 21. In some cases, the arabinose operon regulatory protein gene can comprise a nucleotide sequence that is at least 91% identical to SEQ ID NO: 21. In some cases, the arabinose operon regulatory protein gene can comprise a nucleotide sequence that is at least 92% identical to SEQ ID NO: 21. In some cases, the arabinose operon regulatory protein gene can comprise a nucleotide sequence that is at least 93% identical to SEQ ID NO: 21. In some cases, the arabinose operon regulatory protein gene can comprise a nucleotide sequence that is at least 94% identical to SEQ ID NO: 21. In some cases, the arabinose operon regulatory protein gene can comprise a nucleotide sequence that is at least 95% identical to SEQ ID NO: 21. In some cases, the arabinose operon regulatory protein gene can comprise a nucleotide sequence that is at least 96% identical to SEQ ID NO: 21. In some cases, the arabinose operon regulatory protein gene can comprise a nucleotide sequence that is at least 97% identical to SEQ ID NO: 21. In some cases, the arabinose operon regulatory protein gene can comprise a nucleotide sequence that is at least 98% identical to SEQ ID NO: 21. In some cases, the arabinose operon regulatory protein gene can comprise a nucleotide sequence that is at least 99% identical to SEQ ID NO: 21. In some cases, the arabinose operon regulatory protein gene can comprise a nucleotide sequence that is SEQ ID NO: 21.

The amino acid sequences can also be optimized based on the microorganism in which the enzymes will be expressed. In other words, conservative amino acids substitutions can be made based on whether the respective microorganism typically uses a specific amino acid or how much of that particular amino acid is available for use within the microorganism.

The nucleotide sequence (or more specifically the codons that are encoded by the nucleotide sequences) can be optimized based on the microorganism in which the nucleotide sequences will be expressed. The nucleotide sequences can be codon optimized based on the amount of tRNA available within each individual microorganism. In other words, conservative codon substitutions can be made based on whether the respective microorganism typically uses a specific codon or how much of a particular tRNA is available within the microorganism.

In some instances, there can be more than one copy of one of the genes described throughout, for example, one or more copy of the following genes: (i) acetolactate synthase; (ii) ketol-acid reductoisomerase; (iii) dihydroxy-acid dehydratase; (iv) 2-keto acid decarboxylase; (v) alcohol dehydrogenase; (vi) sugar permease (LacY); and/or (vii) arabinose operon regulatory protein (AraC). These copies of the genes can come from a single organism, e.g., an E. coli, or from multiple organisms, e.g., one copy from an E. coli and one copy from S. cerevisiae, etc.

### Isolated Nucleic Acids

The genes described herein can be in the form of an isolated polynucleic acid. In other words, the genes can be in forms that do not exist in nature, isolated from a chromosome or other endogenous structure. The isolated polynucleic acids can comprise a nucleic acid sequence of one or more of the following genes: (i) acetolactate synthase; (ii) ketol-acid reductoisomerase; (iii) dihydroxy-acid dehydratase; (iv) 2-keto acid decarboxylase; (v) alcohol dehydrogenase; (vi) sugar permease; and/or (vii) arabinose operon regulatory protein. For example, the isolated polynucleic acid can comprise an acetolactate synthase gene. The isolated polynucleic acid can comprise a ketol-acid reductoisomerase gene. The isolated polynucleic acid can comprise a dihydroxy-acid dehydratase gene. The isolated polynucleic acid can comprise a 2-keto acid decarboxylase gene. The isolated polynucleic acid can comprise an alcohol dehydrogenase gene. The isolated polynucleic acid can comprise a sugar permease gene. The isolated polynucleic acid can comprise an arabinose operon regulatory protein gene.

In some cases, the isolated polynucleic acid can encode for an acetolactate synthase. For example, the isolated polynucleic acid can comprise a nucleotide sequence that is substantially similar to SEQ ID NO: 1 or 100. In some cases, the isolated polynucleic acid can comprise a nucleotide sequence that is at least 60% identical to SEQ ID NO: 1 or 100. In some cases, the isolated polynucleic acid can comprise a nucleotide sequence that is at least 65% identical to SEQ ID NO: 1 or 100. In some cases, the isolated polynucleic acid can comprise a nucleotide sequence that is at least 70% identical to SEQ ID NO: 1 or 100. In some cases, the isolated polynucleic acid can comprise a nucleotide sequence that is at least 75% identical to SEQ ID NO: 1 or 100. In some cases, the isolated polynucleic acid can comprise a nucleotide sequence that is at least 80% identical to SEQ ID NO: 1 or 100. In some cases, the isolated polynucleic acid can comprise a nucleotide sequence that is at least 81% identical to SEQ ID NO: 1 or 100. In some cases, the isolated polynucleic acid can comprise a nucleotide sequence that is at least 82% identical to SEQ ID NO: 1 or 100. In some cases, the isolated polynucleic acid can comprise a nucleotide sequence that is at least 83% identical to SEQ ID NO: 1 or 100. In some cases, the isolated polynucleic acid can comprise a nucleotide sequence that is at least 84% identical to SEQ ID NO: 1 or 100. In some cases, the isolated polynucleic acid can comprise a nucleotide sequence that is at least 85% identical to SEQ ID NO: 1 or 100. In some cases, the isolated polynucleic acid can comprise a nucleotide sequence that is at least 86% identical to SEQ ID NO: 1 or 100. In some cases, the isolated polynucleic acid can comprise a nucleotide sequence that is at least 87% identical to SEQ ID NO: 1 or 100. In some cases, the isolated polynucleic acid can comprise a nucleotide sequence that is at least 88% identical to SEQ ID NO: 1 or 100. In some cases, the isolated polynucleic acid can comprise a nucleotide sequence that is at least 89% identical to SEQ ID NO: 1 or 100. In some cases, the isolated polynucleic acid can comprise a nucleotide sequence that is at least 90% identical to SEQ ID NO: 1 or 100. In some cases, the isolated polynucleic acid can comprise a nucleotide sequence that is at least 91% identical to SEQ ID NO: 1 or 100. In some cases, the isolated polynucleic acid can comprise a nucleotide sequence that is at least 92% identical to SEQ ID NO: 1 or 100. In some cases, the isolated polynucleic acid can comprise a nucleotide sequence that is at least 93% identical to SEQ ID NO: 1 or 100. In some cases, the isolated polynucleic acid can comprise a nucleotide sequence that is at least 94% identical to SEQ ID NO: 1 or 100. In some cases, the isolated polynucleic acid can comprise a nucleotide sequence that is at least 95% identical to SEQ ID NO: 1 or 100. In some cases, the isolated polynucleic acid can comprise a nucleotide sequence that is at least 96% identical to SEQ ID NO: 1 or 100. In some cases, the isolated polynucleic acid can comprise a nucleotide sequence that is at least 97% identical to SEQ ID NO: 1 or 100. In some cases, the isolated polynucleic acid can comprise a nucleotide sequence that is at least 98% identical to SEQ ID NO: 1 or 100. In some cases, the isolated polynucleic acid can comprise a nucleotide sequence that is at least 99% identical to SEQ ID NO: 1 or 100. In some cases, the isolated polynucleic acid can comprise a nucleotide sequence that is SEQ ID NO: 1 or 100.

In some cases, the isolated polynucleic acid can encode for a ketol-acid reductoisomerase. For example, the isolated polynucleic acid can comprise a nucleotide sequence that is substantially similar to SEQ ID NO: 3. In some cases, the isolated polynucleic acid can comprise a nucleotide sequence that is at least 60% identical to SEQ ID NO: 3. In some cases, the isolated polynucleic acid can comprise a nucleotide sequence that is at least 65% identical to SEQ ID NO: 3. In some cases, the isolated polynucleic acid can comprise a nucleotide sequence that is at least 70% identical to SEQ ID NO: 3. In some cases, the isolated polynucleic acid can comprise a nucleotide sequence that is at least 75% identical to SEQ ID NO: 3. In some cases, the isolated polynucleic acid can comprise a nucleotide sequence that is at least 80% identical to SEQ ID NO: 3. In some cases, the isolated polynucleic acid can comprise a nucleotide sequence that is at least 81% identical to SEQ ID NO: 3. In some cases, the isolated polynucleic acid can comprise a nucleotide sequence that is at least 82% identical to SEQ ID NO: 3. In some cases, the isolated polynucleic acid can comprise a nucleotide sequence that is at least 83% identical to SEQ ID NO: 3. In some cases, the isolated polynucleic acid can comprise a nucleotide sequence that is at least 84% identical to SEQ ID NO: 3. In some cases, the isolated polynucleic acid can comprise a nucleotide sequence that is at least 85% identical to SEQ ID NO: 3. In some cases, the isolated polynucleic acid can comprise a nucleotide sequence that is at least 86% identical to SEQ ID NO: 3. In some cases, the isolated polynucleic acid can comprise a nucleotide sequence that is at least 87% identical to SEQ ID NO: 3. In some cases, the isolated polynucleic acid can comprise a nucleotide sequence that is at least 88% identical to SEQ ID NO: 3. In some cases, the isolated polynucleic acid can comprise a nucleotide sequence that is at least 89% identical to SEQ ID NO: 3. In some cases, the isolated polynucleic acid can comprise a nucleotide sequence that is at least 90% identical to SEQ ID NO: 3. In some cases, the isolated polynucleic acid can comprise a nucleotide sequence that is at least 91% identical to SEQ ID NO: 3. In some cases, the isolated polynucleic acid can comprise a nucleotide sequence that is at least 92% identical to SEQ ID NO: 3. In some cases, the isolated polynucleic acid can comprise a nucleotide sequence that is at least 93% identical to SEQ ID NO: 3. In some cases, the isolated polynucleic acid can comprise a nucleotide sequence that is at least 94% identical to SEQ ID NO: 3. In some cases, the isolated polynucleic acid can comprise a nucleotide sequence that is at least 95% identical to SEQ ID NO: 3. In some cases, the isolated polynucleic acid can comprise a nucleotide sequence that is at least 96% identical to SEQ ID NO: 3. In some cases, the isolated polynucleic acid can comprise a nucleotide sequence that is at least 97% identical to SEQ ID NO: 3. In some cases, the isolated polynucleic acid can comprise a nucleotide sequence that is at least 98% identical to SEQ ID NO: 3. In some cases, the isolated polynucleic acid can comprise a nucleotide sequence that is at least 99% identical to SEQ ID NO: 3. In some cases, the isolated polynucleic acid can comprise a nucleotide sequence that is SEQ ID NO: 3.

In some cases, the isolated polynucleic acid can comprise a nucleotide sequence that encodes for a dihydroxy-acid dehydratase. In some cases, the isolated polynucleic acid can comprise a nucleotide sequence that is substantially similar to SEQ ID NO: 5 or 7. For example, the isolated polynucleic acid can comprise a nucleotide sequence that is at least 60% identical to SEQ ID NO: 5 or 7. In some cases, the isolated polynucleic acid can comprise a nucleotide sequence that is at least 65% identical to SEQ ID NO: 5 or 7. In some cases, the isolated polynucleic acid can comprise a nucleotide sequence that is at least 70% identical to SEQ ID NO: 5 or 7. In some cases, the isolated polynucleic acid can comprise a nucleotide sequence that is at least 75% identical to SEQ ID NO: 5 or 7. In some cases, the isolated polynucleic acid can comprise a nucleotide sequence that is at least 80% identical to SEQ ID NO: 5 or 7. In some cases, the isolated polynucleic acid can comprise a nucleotide sequence that is at least 81% identical to SEQ ID NO: 5 or 7. In some cases, the isolated polynucleic acid can comprise a nucleotide sequence that is at least 82% identical to SEQ ID NO: 5 or 7. In some cases, the isolated polynucleic acid can comprise a nucleotide sequence that is at least 83% identical to SEQ ID NO: 5 or 7. In some cases, the isolated polynucleic acid can comprise a nucleotide sequence that is at least 84% identical to SEQ ID NO: 5 or 7. In some cases, the isolated polynucleic acid can comprise a nucleotide sequence that is at least 85% identical to SEQ ID NO: 5 or 7. In some cases, the isolated polynucleic acid can comprise a nucleotide sequence that is at least 86% identical to SEQ ID NO: 5 or 7. In some cases, the isolated polynucleic acid can comprise a nucleotide sequence that is at least 87% identical to SEQ ID NO: 5 or 7. In some cases, the isolated polynucleic acid can comprise a nucleotide sequence that is at least 88% identical to SEQ ID NO: 5 or 7. In some cases, the isolated polynucleic acid can comprise a nucleotide sequence that is at least 89% identical to SEQ ID NO: 5 or 7. In some cases, the isolated polynucleic acid can comprise a nucleotide sequence that is at least 90% identical to SEQ ID NO: 5 or 7. In some cases, the isolated polynucleic acid can comprise a nucleotide sequence that is at least 91% identical to SEQ ID NO: 5 or 7. In some cases, the isolated polynucleic acid can comprise a nucleotide sequence that is at least 92% identical to SEQ ID NO: 5 or 7. In some cases, the isolated polynucleic acid can comprise a nucleotide sequence that is at least 93% identical to SEQ ID NO: 5 or 7. In some cases, the isolated polynucleic acid can comprise a nucleotide sequence that is at least 94% identical to SEQ ID NO: 5 or 7. In some cases, the isolated polynucleic acid can comprise a nucleotide sequence that is at least 95% identical to SEQ ID NO: 5 or 7. In some cases, the isolated polynucleic acid can comprise a nucleotide sequence that is at least 96% identical to SEQ ID NO: 5 or 7. In some cases, the isolated polynucleic acid can comprise a nucleotide sequence that is at least 97% identical to SEQ ID NO: 5 or 7. In some cases, the isolated polynucleic acid can comprise a nucleotide sequence that is at least 98% identical to SEQ ID NO: 5 or 7. In some cases, the isolated polynucleic acid can comprise a nucleotide sequence that is at least 99% identical to SEQ ID NO: 5 or 7. In some cases, the isolated polynucleic acid can comprise a nucleotide sequence that is SEQ ID NO: 5 or 7.

In some cases, the isolated polynucleic acid can comprise a nucleotide sequence that encodes for a 2-keto acid decarboxylase. In some cases, the isolated polynucleic acid can comprise a nucleotide sequence that is substantially similar to any one of SEQ ID NOs: 9, 11, 56, 58, 60, 62, 64, 66, 68, 70, 72, 74, 76, 78, 80, 82, 84, 86, 88, 90, 92, 94, 96, or 98. For example, the isolated polynucleic acid can comprise a nucleotide sequence that is at least 60% identical to any one of SEQ ID NOs: 9, 11, 56, 58, 60, 62, 64, 66, 68, 70, 72, 74, 76, 78, 80, 82, 84, 86, 88, 90, 92, 94, 96, or 98. In some cases, the isolated polynucleic acid can comprise a nucleotide sequence that is at least 65% identical to any one of SEQ ID NOs: 9, 11, 56, 58, 60, 62, 64, 66, 68, 70, 72, 74, 76, 78, 80, 82, 84, 86, 88, 90, 92, 94, 96, or 98. In some cases, the isolated polynucleic acid can comprise a nucleotide sequence that is at least 70% identical to any one of SEQ ID NOs: 9, 11, 56, 58, 60, 62, 64, 66, 68, 70, 72, 74, 76, 78, 80, 82, 84, 86, 88, 90, 92, 94, 96, or 98. In some cases, the isolated polynucleic acid can comprise a nucleotide sequence that is at least 75% identical to any one of SEQ ID NOs: 9, 11, 56, 58, 60, 62, 64, 66, 68, 70, 72, 74, 76, 78, 80, 82, 84, 86, 88, 90, 92, 94, 96, or 98. In some cases, the isolated polynucleic acid can comprise a nucleotide sequence that is at least 80% identical to any one of SEQ ID NOs: 9, 11, 56, 58, 60, 62, 64, 66, 68, 70, 72, 74, 76, 78, 80, 82, 84, 86, 88, 90, 92, 94, 96, or 98. In some cases, the isolated polynucleic acid can comprise a nucleotide sequence that is at least 81% identical to any one of SEQ ID NOs: 9, 11, 56, 58, 60, 62, 64, 66, 68, 70, 72, 74, 76, 78, 80, 82, 84, 86, 88, 90, 92, 94, 96, or 98. In some cases, the isolated polynucleic acid can comprise a nucleotide sequence that is at least 82% identical to any one of SEQ ID NOs: 9, 11, 56, 58, 60, 62, 64, 66, 68, 70, 72, 74, 76, 78, 80, 82, 84, 86, 88, 90, 92, 94, 96, or 98. In some cases, the isolated polynucleic acid can comprise a nucleotide sequence that is at least 83% identical to any one of SEQ ID NOs: 9, 11, 56, 58, 60, 62, 64, 66, 68, 70, 72, 74, 76, 78, 80, 82, 84, 86, 88, 90, 92, 94, 96, or 98. In some cases, the isolated polynucleic acid can comprise a nucleotide sequence that is at least 84% identical to any one of SEQ ID NOs: 9, 11, 56, 58, 60, 62, 64, 66, 68, 70, 72, 74, 76, 78, 80, 82, 84, 86, 88, 90, 92, 94, 96, or 98. In some cases, the isolated polynucleic acid can comprise a nucleotide sequence that is at least 85% identical to any one of SEQ ID NOs: 9, 11, 56, 58, 60, 62, 64, 66, 68, 70, 72, 74, 76, 78, 80, 82, 84, 86, 88, 90, 92, 94, 96, or 98. In some cases, the isolated polynucleic acid can comprise a nucleotide sequence that is at least 86% identical to any one of SEQ ID NOs: 9, 11, 56, 58, 60, 62, 64, 66, 68, 70, 72, 74, 76, 78, 80, 82, 84, 86, 88, 90, 92, 94, 96, or 98. In some cases, the isolated polynucleic acid can comprise a nucleotide sequence that is at least 87% identical to any one of SEQ ID NOs: 9, 11, 56, 58, 60, 62, 64, 66, 68, 70, 72, 74, 76, 78, 80, 82, 84, 86, 88, 90, 92, 94, 96, or 98. In some cases, the isolated polynucleic acid can comprise a nucleotide sequence that is at least 88% identical to any one of SEQ ID NOs: 9, 11, 56, 58, 60, 62, 64, 66, 68, 70, 72, 74, 76, 78, 80, 82, 84, 86, 88, 90, 92, 94, 96, or 98. In some cases, the isolated polynucleic acid can comprise a nucleotide sequence that is at least 89% identical to any one of SEQ ID NOs: 9, 11, 56, 58, 60, 62, 64, 66, 68, 70, 72, 74, 76, 78, 80, 82, 84, 86, 88, 90, 92, 94, 96, or 98. In some cases, the isolated polynucleic acid can comprise a nucleotide sequence that is at least 90% identical to any one of SEQ ID NOs: 9, 11, 56, 58, 60, 62, 64, 66, 68, 70, 72, 74, 76, 78, 80, 82, 84, 86, 88, 90, 92, 94, 96, or 98. In some cases, the isolated polynucleic acid can comprise a nucleotide sequence that is at least 91% identical to any one of SEQ ID NOs: 9, 11, 56, 58, 60, 62, 64, 66, 68, 70, 72, 74, 76, 78, 80, 82, 84, 86, 88, 90, 92, 94, 96, or 98. In some cases, the isolated polynucleic acid can comprise a nucleotide sequence that is at least 92% identical to any one of SEQ ID NOs: 9, 11, 56, 58, 60, 62, 64, 66, 68, 70, 72, 74, 76, 78, 80, 82, 84, 86, 88, 90, 92, 94, 96, or 98. In some cases, the isolated polynucleic acid can comprise a nucleotide sequence that is at least 93% identical to any one of SEQ ID NOs: 9, 11, 56, 58, 60, 62, 64, 66, 68, 70, 72, 74, 76, 78, 80, 82, 84, 86, 88, 90, 92, 94, 96, or 98. In some cases, the isolated polynucleic acid can comprise a nucleotide sequence that is at least 94% identical to any one of SEQ ID NOs: 9, 11, 56, 58, 60, 62, 64, 66, 68, 70, 72, 74, 76, 78, 80, 82, 84, 86, 88, 90, 92, 94, 96, or 98. In some cases, the isolated polynucleic acid can comprise a nucleotide sequence that is at least 95% identical to any one of SEQ ID NOs: 9, 11, 56, 58, 60, 62, 64, 66, 68, 70, 72, 74, 76, 78, 80, 82, 84, 86, 88, 90, 92, 94, 96, or 98. In some cases, the isolated polynucleic acid can comprise a nucleotide sequence that is at least 96% identical to any one of SEQ ID NOs: 9, 11, 56, 58, 60, 62, 64, 66, 68, 70, 72, 74, 76, 78, 80, 82, 84, 86, 88, 90, 92, 94, 96, or 98. In some cases, the isolated polynucleic acid can comprise a nucleotide sequence that is at least 97% identical to any one of SEQ ID NOs: 9, 11, 56, 58, 60, 62, 64, 66, 68, 70, 72, 74, 76, 78, 80, 82, 84, 86, 88, 90, 92, 94, 96, or 98. In some cases, the isolated polynucleic acid can comprise a nucleotide sequence that is at least 98% identical to any one of SEQ ID NOs: 9, 11, 56, 58, 60, 62, 64, 66, 68, 70, 72, 74, 76, 78, 80, 82, 84, 86, 88, 90, 92, 94, 96, or 98. In some cases, the isolated polynucleic acid can comprise a nucleotide sequence that is at least 99% identical to any one of SEQ ID NOs: 9, 11, 56, 58, 60, 62, 64, 66, 68, 70, 72, 74, 76, 78, 80, 82, 84, 86, 88, 90, 92, 94, 96, or 98. In some cases, the isolated polynucleic acid can comprise a nucleotide sequence that is any one of SEQ ID NOs: 9, 11, 56, 58, 60, 62, 64, 66, 68, 70, 72, 74, 76, 78, 80, 82, 84, 86, 88, 90, 92, 94, 96, or 98.

In some cases, the isolated polynucleic acid can comprise a nucleotide sequence that encodes for an alcohol dehydrogenase. In some cases, the isolated polynucleic acid can comprise a nucleotide sequence that is substantially similar to any one of SEQ ID NOs: 13, 15, 17, 24, 26, 28, 30, 32, 34, 36, 38, 40, 42, 44, 46, 48, 50, 52, or 54. For example, the isolated polynucleic acid can comprise a nucleotide sequence that is at least 60% identical to any one of SEQ ID NOs: 13, 15, 17, 24, 26, 28, 30, 32, 34, 36, 38, 40, 42, 44, 46, 48, 50, 52, or 54. In some cases, the isolated polynucleic acid can comprise a nucleotide sequence that is at least 65% identical to any one of SEQ ID NOs: 13, 15, 17, 24, 26, 28, 30, 32, 34, 36, 38, 40, 42, 44, 46, 48, 50, 52, or 54. In some cases, the isolated polynucleic acid can comprise a nucleotide sequence that is at least 70% identical to any one of SEQ ID NOs: 13, 15, 17, 24, 26, 28, 30, 32, 34, 36, 38, 40, 42, 44, 46, 48, 50, 52, or 54. In some cases, the isolated polynucleic acid can comprise a nucleotide sequence that is at least 75% identical to any one of SEQ ID NOs: 13, 15, 17, 24, 26, 28, 30, 32, 34, 36, 38, 40, 42, 44, 46, 48, 50, 52, or 54. In some cases, the isolated polynucleic acid can comprise a nucleotide sequence that is at least 80% identical to any one of SEQ ID NOs: 13, 15, 17, 24, 26, 28, 30, 32, 34, 36, 38, 40, 42, 44, 46, 48, 50, 52, or 54. In some cases, the isolated polynucleic acid can comprise a nucleotide sequence that is at least 81% identical to any one of SEQ ID NOs: 13, 15, 17, 24, 26, 28, 30, 32, 34, 36, 38, 40, 42, 44, 46, 48, 50, 52, or 54. In some cases, the isolated polynucleic acid can comprise a nucleotide sequence that is at least 82% identical to any one of SEQ ID NOs: 13, 15, 17, 24, 26, 28, 30, 32, 34, 36, 38, 40, 42, 44, 46, 48, 50, 52, or 54. In some cases, the isolated polynucleic acid can comprise a nucleotide sequence that is at least 83% identical to any one of SEQ ID NOs: 13, 15, 17, 24, 26, 28, 30, 32, 34, 36, 38, 40, 42, 44, 46, 48, 50, 52, or 54. In some cases, the isolated polynucleic acid can comprise a nucleotide sequence that is at least 84% identical to any one of SEQ ID NOs: 13, 15, 17, 24, 26, 28, 30, 32, 34, 36, 38, 40, 42, 44, 46, 48, 50, 52, or 54. In some cases, the isolated polynucleic acid can comprise a nucleotide sequence that is at least 85% identical to any one of SEQ ID NOs: 13, 15, 17, 24, 26, 28, 30, 32, 34, 36, 38, 40, 42, 44, 46, 48, 50, 52, or 54. In some cases, the isolated polynucleic acid can comprise a nucleotide sequence that is at least 86% identical to any one of SEQ ID NOs: 13, 15, 17, 24, 26, 28, 30, 32, 34, 36, 38, 40, 42, 44, 46, 48, 50, 52, or 54. In some cases, the isolated polynucleic acid can comprise a nucleotide sequence that is at least 87% identical to any one of SEQ ID NOs: 13, 15, 17, 24, 26, 28, 30, 32, 34, 36, 38, 40, 42, 44, 46, 48, 50, 52, or 54. In some cases, the isolated polynucleic acid can comprise a nucleotide sequence that is at least 88% identical to any one of SEQ ID NOs: 13, 15, 17, 24, 26, 28, 30, 32, 34, 36, 38, 40, 42, 44, 46, 48, 50, 52, or 54. In some cases, the isolated polynucleic acid can comprise a nucleotide sequence that is at least 89% identical to any one of SEQ ID NOs: 13, 15, 17, 24, 26, 28, 30, 32, 34, 36, 38, 40, 42, 44, 46, 48, 50, 52, or 54. In some cases, the isolated polynucleic acid can comprise a nucleotide sequence that is at least 90% identical to any one of SEQ ID NOs: 13, 15, 17, 24, 26, 28, 30, 32, 34, 36, 38, 40, 42, 44, 46, 48, 50, 52, or 54. In some cases, the isolated polynucleic acid can comprise a nucleotide sequence that is at least 91% identical to any one of SEQ ID NOs: 13, 15, 17, 24, 26, 28, 30, 32, 34, 36, 38, 40, 42, 44, 46, 48, 50, 52, or 54. In some cases, the isolated polynucleic acid can comprise a nucleotide sequence that is at least 92% identical to any one of SEQ ID NOs: 13, 15, 17, 24, 26, 28, 30, 32, 34, 36, 38, 40, 42, 44, 46, 48, 50, 52, or 54. In some cases, the isolated polynucleic acid can comprise a nucleotide sequence that is at least 93% identical to any one of SEQ ID NOs: 13, 15, 17, 24, 26, 28, 30, 32, 34, 36, 38, 40, 42, 44, 46, 48, 50, 52, or 54. In some cases, the isolated polynucleic acid can comprise a nucleotide sequence that is at least 94% identical to any one of SEQ ID NOs: 13, 15, 17, 24, 26, 28, 30, 32, 34, 36, 38, 40, 42, 44, 46, 48, 50, 52, or 54. In some cases, the isolated polynucleic acid can comprise a nucleotide sequence that is at least 95% identical to any one of SEQ ID NOs: 13, 15, 17, 24, 26, 28, 30, 32, 34, 36, 38, 40, 42, 44, 46, 48, 50, 52, or 54. In some cases, the isolated polynucleic acid can comprise a nucleotide sequence that is at least 96% identical to any one of SEQ ID NOs: 13, 15, 17, 24, 26, 28, 30, 32, 34, 36, 38, 40, 42, 44, 46, 48, 50, 52, or 54. In some cases, the isolated polynucleic acid can comprise a nucleotide sequence that is at least 97% identical to any one of SEQ ID NOs: 13, 15, 17, 24, 26, 28, 30, 32, 34, 36, 38, 40, 42, 44, 46, 48, 50, 52, or 54. In some cases, the isolated polynucleic acid can comprise a nucleotide sequence that is at least 98% identical to any one of SEQ ID NOs: 13, 15, 17, 24, 26, 28, 30, 32, 34, 36, 38, 40, 42, 44, 46, 48, 50, 52, or 54. In some cases, the isolated polynucleic acid can comprise a nucleotide sequence that is at least 99% identical to any one of SEQ ID NOs: 13, 15, 17, 24, 26, 28, 30, 32, 34, 36, 38, 40, 42, 44, 46, 48, 50, 52, or 54. In some cases, the isolated polynucleic acid can comprise a nucleotide sequence that is any one of SEQ ID NOs: 13, 15, 17, 24, 26, 28, 30, 32, 34, 36, 38, 40, 42, 44, 46, 48, 50, 52, or 54.

In some cases, the isolated polynucleic acid can comprise a nucleotide sequence that encodes for a sugar permease. In some cases, the isolated polynucleic acid can comprise a nucleotide sequence that is substantially similar to SEQ ID NO: 19. For example, the isolated polynucleic acid can comprise a nucleotide sequence that is at least 60% identical to SEQ ID NO: 19. In some cases, the isolated polynucleic acid can comprise a nucleotide sequence that is at least 65% identical to SEQ ID NO: 19. In some cases, the isolated polynucleic acid can comprise a nucleotide sequence that is at least 70% identical to SEQ ID NO: 19. In some cases, the isolated polynucleic acid can comprise a nucleotide sequence that is at least 75% identical to SEQ ID NO: 19. In some cases, the isolated polynucleic acid can comprise a nucleotide sequence that is at least 80% identical to SEQ ID NO: 19. In some cases, the isolated polynucleic acid can comprise a nucleotide sequence that is at least 81% identical to SEQ ID NO: 19. In some cases, the isolated polynucleic acid can comprise a nucleotide sequence that is at least 82% identical to SEQ ID NO: 19. In some cases, the isolated polynucleic acid can comprise a nucleotide sequence that is at least 83% identical to SEQ ID NO: 19. In some cases, the isolated polynucleic acid can comprise a nucleotide sequence that is at least 84% identical to SEQ ID NO: 19. In some cases, the isolated polynucleic acid can comprise a nucleotide sequence that is at least 85% identical to SEQ ID NO: 19. In some cases, the isolated polynucleic acid can comprise a nucleotide sequence that is at least 86% identical to SEQ ID NO: 19. In some cases, the isolated polynucleic acid can comprise a nucleotide sequence that is at least 87% identical to SEQ ID NO: 19. In some cases, the isolated polynucleic acid can comprise a nucleotide sequence that is at least 88% identical to SEQ ID NO: 19. In some cases, the isolated polynucleic acid can comprise a nucleotide sequence that is at least 89% identical to SEQ ID NO: 19. In some cases, the isolated polynucleic acid can comprise a nucleotide sequence that is at least 90% identical to SEQ ID NO: 19. In some cases, the isolated polynucleic acid can comprise a nucleotide sequence that is at least 91% identical to SEQ ID NO: 19. In some cases, the isolated polynucleic acid can comprise a nucleotide sequence that is at least 92% identical to SEQ ID NO: 19. In some cases, the isolated polynucleic acid can comprise a nucleotide sequence that is at least 93% identical to SEQ ID NO: 19. In some cases, the isolated polynucleic acid can comprise a nucleotide sequence that is at least 94% identical to SEQ ID NO: 19. In some cases, the isolated polynucleic acid can comprise a nucleotide sequence that is at least 95% identical to SEQ ID NO: 19. In some cases, the isolated polynucleic acid can comprise a nucleotide sequence that is at least 96% identical to SEQ ID NO: 19. In some cases, the isolated polynucleic acid can comprise a nucleotide sequence that is at least 97% identical to SEQ ID NO: 19. In some cases, the isolated polynucleic acid can comprise a nucleotide sequence that is at least 98% identical to SEQ ID NO: 19. In some cases, the isolated polynucleic acid can comprise a nucleotide sequence that is at least 99% identical to SEQ ID NO: 19. In some cases, the isolated polynucleic acid can comprise a nucleotide sequence that is SEQ ID NO: 19.

In some cases, the isolated polynucleic acid can comprise a nucleotide sequence that encodes for an arabinose operon regulatory protein. In some cases, the isolated polynucleic acid can comprise a nucleotide sequence that is substantially similar to SEQ ID NO: 21. For example, the isolated polynucleic acid can comprise a nucleotide sequence that is at least 60% identical to SEQ ID NO: 21. In some cases, the isolated polynucleic acid can comprise a nucleotide sequence that is at least 65% identical to SEQ ID NO: 21. In some cases, the isolated polynucleic acid can comprise a nucleotide sequence that is at least 70% identical to SEQ ID NO: 21. In some cases, the isolated polynucleic acid can comprise a nucleotide sequence that is at least 75% identical to SEQ ID NO: 21. In some cases, the isolated polynucleic acid can comprise a nucleotide sequence that is at least 80% identical to SEQ ID NO: 21. In some cases, the isolated polynucleic acid can comprise a nucleotide sequence that is at least 81% identical to SEQ ID NO: 21. In some cases, the isolated polynucleic acid can comprise a nucleotide sequence that is at least 82% identical to SEQ ID NO: 21. In some cases, the isolated polynucleic acid can comprise a nucleotide sequence that is at least 83% identical to SEQ ID NO: 21. In some cases, the isolated polynucleic acid can comprise a nucleotide sequence that is at least 84% identical to SEQ ID NO: 21. In some cases, the isolated polynucleic acid can comprise a nucleotide sequence that is at least 85% identical to SEQ ID NO: 21. In some cases, the isolated polynucleic acid can comprise a nucleotide sequence that is at least 86% identical to SEQ ID NO: 21. In some cases, the isolated polynucleic acid can comprise a nucleotide sequence that is at least 87% identical to SEQ ID NO: 21. In some cases, the isolated polynucleic acid can comprise a nucleotide sequence that is at least 88% identical to SEQ ID NO: 21. In some cases, the isolated polynucleic acid can comprise a nucleotide sequence that is at least 89% identical to SEQ ID NO: 21. In some cases, the isolated polynucleic acid can comprise a nucleotide sequence that is at least 90% identical to SEQ ID NO: 21. In some cases, the isolated polynucleic acid can comprise a nucleotide sequence that is at least 91% identical to SEQ ID NO: 21. In some cases, the isolated polynucleic acid can comprise a nucleotide sequence that is at least 92% identical to SEQ ID NO: 21. In some cases, the isolated polynucleic acid can comprise a nucleotide sequence that is at least 93% identical to SEQ ID NO: 21. In some cases, the isolated polynucleic acid can comprise a nucleotide sequence that is at least 94% identical to SEQ ID NO: 21. In some cases, the isolated polynucleic acid can comprise a nucleotide sequence that is at least 95% identical to SEQ ID NO: 21. In some cases, the isolated polynucleic acid can comprise a nucleotide sequence that is at least 96% identical to SEQ ID NO: 21. In some cases, the isolated polynucleic acid can comprise a nucleotide sequence that is at least 97% identical to SEQ ID NO: 21. In some cases, the isolated polynucleic acid can comprise a nucleotide sequence that is at least 98% identical to SEQ ID NO: 21. In some cases, the isolated polynucleic acid can comprise a nucleotide sequence that is at least 99% identical to SEQ ID NO: 21. In some cases, the isolated polynucleic acid can comprise a nucleotide sequence that is SEQ ID NO: 21.

### Exemplary Vector Sequences

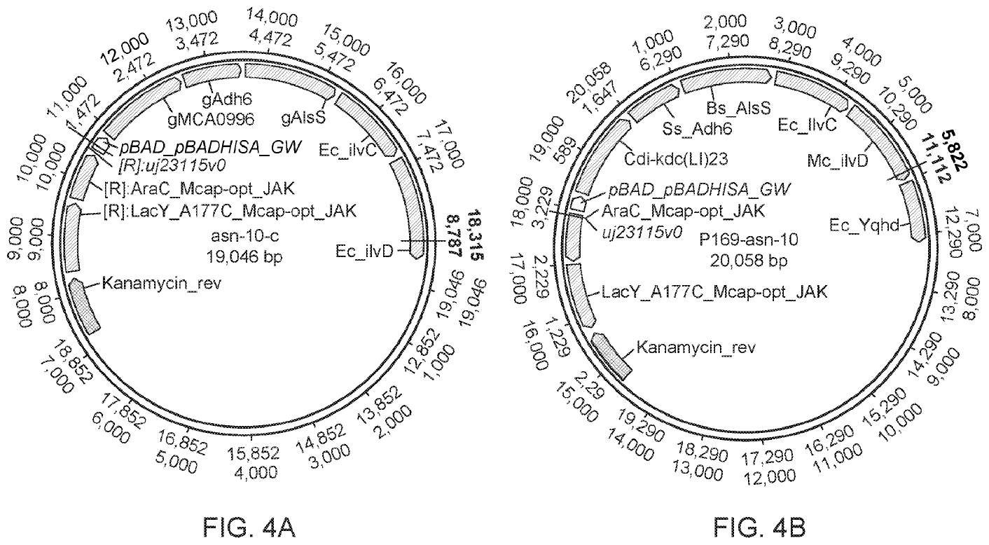

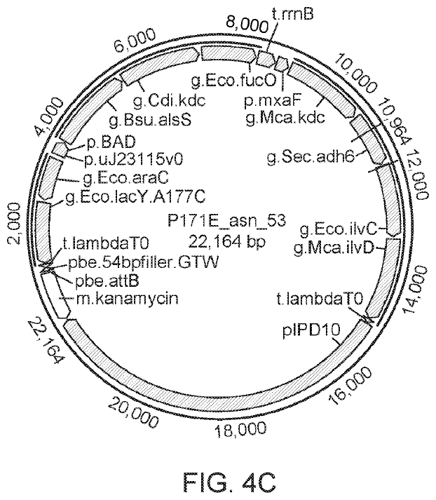

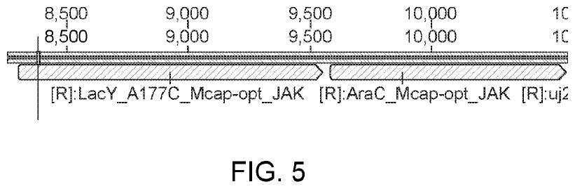

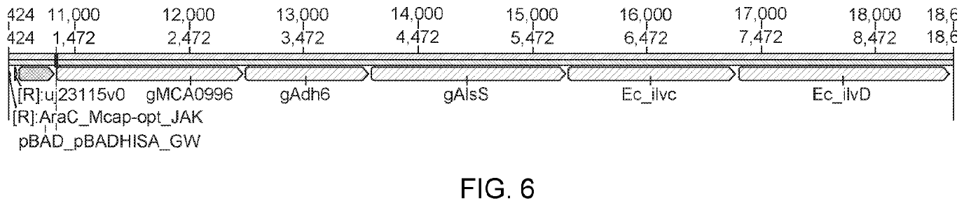

Vectors that can be integrated into various microorganisms, such as methanotrophs, are disclosed herein (see e.g., FIGS. 4A-4C, 5, and 6). In some cases, minor changes can be made to the vectors without significant changes in the effectiveness of the vectors or the amount of enzymes the vectors are able to produce.

In some cases, the expression plasmid as disclosed in FIG. 4A or 4B, can be contacted with (and inserted into) a microorganism. These expression plasmids comprise a Kanamycin marker, an arabinose induction machinery (LacY and AraC driven by a J23115 promoter) and a BAD promoter (e.g., a pBAD promoter) driving the expression of enzymes useful in the isobutanol pathway ((MCA0996=Kdc), Adh6, AlsS, IlvC and IlvD).

In other cases, the expression plasmid as disclosed in FIG. 4C can be contacted with (and inserted into) a microorganism. This expression plasmid comprises a Kanamycin marker, an arabinose induction machinery (LacY and AraC driven by a J23115 promoter) and two operons with two different promoters (a BAD promoter (pBAD) and a pMxaF promoter) driving the expression of enzymes useful in the isobutanol pathway Kdc, Adh, AlsS, IlvC and IlvD.

### II. Method of Making the Genetically Modified Microorganisms

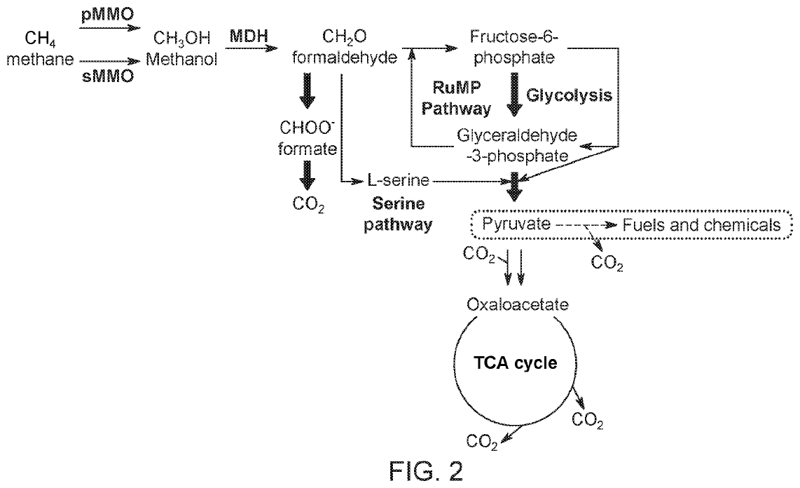

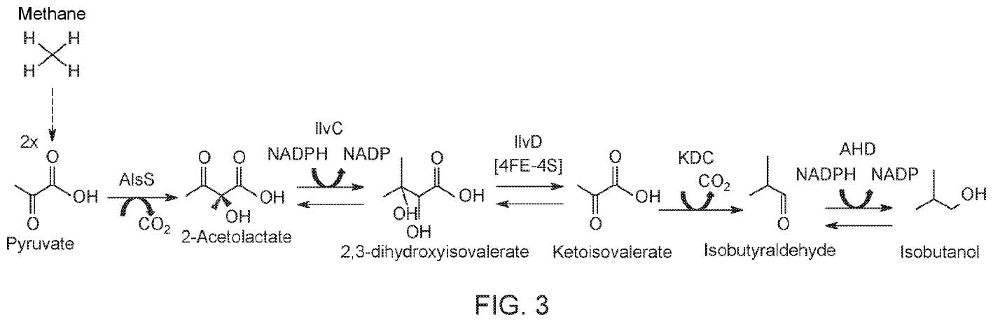

The genetically modified microorganisms above can be made by a variety of ways. A microorganism can be modified (e.g., genetically engineered) by any method to comprise and/or express one or more polynucleotides encoding for enzymes in a pathway that catalyze a conversion of a fermentable carbon source to one or more intermediates in a pathway for the production of a desired product, such as isobutanol. For example, the genetically modified microorganism can comprise one or more nucleic acids encoding for an enzyme capable of catalyzing one or more of the reactions: i) methane to methanol; ii) methanol to formaldehyde; and/or iii) formaldehyde to pyruvate. For example, the genetically modified microorganism can comprise one or more genes including but not limited to pMMO; sMMO; and/or methanol dehydrogenase (MDH). Such enzymes can include any of those enzymes as set forth in FIG. 2 or 3. For example, one or more of any of the genes above can be inserted into a microorganism. The genes can be inserted by an expression vector. The one or more genes can also be stably integrated into the genome of the microorganism.

The microorganism used in this method can be any described above, including but not limited to a prokaryote. Other microorganisms such as bacteria, yeast, or algae can be used. One microorganism of particular interest is a methanotroph, such as a methanotroph from the genera Methylobacter, Methylomicrobium, Methylomonas, Methylocaldum, Methylococcus, Methylosoma, Methylosarcina, Methylothermus, Methylohalobius, Methylogaea, Methylovulum, Crenothrix, Clonothrix, Methylosphaera, Methylocapsa, Methylocella, Methylosinus, Methylocystis, Methyloferula, Methylomarinum, or Methyloacidoiphilum. One desired species can include a Methylococcus capsulatus.

An exemplary method of making a genetically modified microorganism disclosed herein is contacting (or transforming) a microorganism with a nucleic acid that expresses at least one heterologous gene from: (i) acetolactate synthase; (ii) ketol-acid reductoisomerase; (iii) dihydroxy-acid dehydratase; (iv) 2-keto acid decarboxylase; (v) alcohol dehydrogenase; or (vi) any combination thereof. For example, in order to make a microorganism that produces an aldehyde, e.g., isobutyraldehyde, (i) acetolactate synthase; (ii) ketol-acid reductoisomerase; (iii) dihydroxy-acid dehydratase; and/or (iv) 2-keto acid decarboxylase can be transformed into a microorganism. Additionally, should a microorganism that produces an alcohol be desired, an additional gene encoding for an (v) alcohol dehydrogenase can be transformed into the microorganism. One or more of these enzymes can be heterologous to the microorganism. Additionally, one or more of these enzymes can be endogenous to the microorganism. Further, one or more of these enzymes can be overexpressed in the microorganism. The microorganism can be any microorganism that is capable of converting a carbon source into a desired product. In some cases, the product is isobutanol. In some cases, the product is isobutyraldehyde.

The acetolactate synthase; ketol-acid reductoisomerase; dihydroxy-acid dehydratase; 2-keto acid decarboxylase; and/or alcohol dehydrogenase, used in the method can be any of the variations described throughout. For example, the alcohol dehydrogenase can be from yeast such as from the genus Saccharomyces or a bacterium (e.g., a gram negative or gram positive bacterium) such as from the genus Escherichia. Other bacterial genera that can be used include Clostridium, Escherichia, Geobacillus, Lactococcus, Oenococcus, Pectobacterium, and/or Psychrobacter. More specifically, the alcohol dehydrogenase can be from the species Saccharomyces cerevisiae, Escherichia coli, Clostridium acetobutylicum, Escherichia coli, Geobacillus stearothermophilus, Geobacillus thermoglucosidas, Lactococcus lactis, Oenococcus oeni, Pectobacterium atrosepticum, and/or Psychrobacter cryohalolentis. Further, multiple alcohol dehydrogenases can be used. For example, one or more bacterial alcohol dehydrogenases and one or more yeast alcohol dehydrogenases can be expressed within a microorganism.

The one or more genes that are inserted into a microorganism can be heterologous to the microorganism itself. For example, if the microorganism is a methanotroph, the one or more genes that are inserted can be from yeast, a bacterium, or a different species of methanotroph. Further, the one or more genes can be endogenously part of the genome of the microorganism. When endogenous genes are used, they can be overexpressed or they can be modified so that expression is altered compared to the unmodified endogenous gene. For example, the endogenous gene can be made to be under the control of a different promoter, such as an inducible promoter.

### Techniques for Genetic Modification

The microorganisms disclosed herein can be genetically engineered by using classic microbiological techniques. Some of such techniques are generally disclosed, for example, in Sambrook et al., 1989, Molecular Cloning: A Laboratory Manual, Cold Spring Harbor Labs Press.

The genetically modified microorganisms disclosed herein can include a polynucleotide that has been inserted, deleted or modified (i.e., mutated; e.g., by insertion, deletion, substitution, and/or inversion of nucleotides), in such a manner that such modifications provide the desired effect of expression (e.g., over-expression) of one or more enzymes as provided herein within the microorganism. Genetic modifications which result in an increase in gene expression or function can be referred to as amplification, overproduction, overexpression, activation, enhancement, addition, or up-regulation of a gene. Addition of a gene to increase gene expression can include maintaining the gene(s) on replicating plasmids or integrating the cloned gene(s) into the genome of the production microorganism. Furthermore, increasing the expression of desired genes can include operatively linking the cloned gene(s) to native or heterologous transcriptional control elements.

Where desired, the expression of one or more of the enzymes provided herein is under the control of a regulatory sequence that controls directly or indirectly the enzyme expression in a time-dependent fashion during the fermentation. Inducible promoters can be used to achieve this.

In some cases, a microorganism is transformed or transfected with a genetic vehicle, such as an expression vector comprising a heterologous polynucleotide sequence coding for the enzymes are provided herein.

To facilitate insertion and expression of different genes coding for the enzymes as disclosed herein from the constructs and expression vectors, the constructs can be designed with at least one cloning site for insertion of any gene coding for any enzyme disclosed herein. The cloning site can be a multiple cloning site, e.g., containing multiple restriction sites.

### Transfection

Standard transfection techniques can be used to insert genes into a microorganism. As used herein, the term “transfection” or “transformation” can refer to the insertion of an exogenous nucleic acid or polynucleotide into a host cell. The exogenous nucleic acid or polynucleotide can be maintained as a non-integrated vector, for example, a plasmid, or alternatively, can be integrated into the host cell genome. The term transfecting or transfection is intended to encompass all conventional techniques for introducing nucleic acid or polynucleotide into microorganisms. Examples of transfection techniques include, but are not limited to, calcium phosphate precipitation, DEAE-dextran-mediated transfection, lipofection, electroporation, microinjection, rubidium chloride or polycation mediated transfection, protoplast fusion, and sonication. The transfection method that provides optimal transfection frequency and expression of the construct in the particular host cell line and type is favored. For stable transfectants, the constructs are integrated so as to be stably maintained within the host chromosome. In some cases, the transfection is a stable transfection.

### Transformation

Expression vectors or other nucleic acids can be introduced to selected microorganisms by any of a number of suitable methods. For example, vector constructs can be introduced to appropriate cells by any of a number of transformation methods for plasmid vectors. Standard calcium-chloride-mediated bacterial transformation is still commonly used to introduce naked DNA to bacteria (see, e.g., Sambrook et al., 1989, Molecular Cloning, A Laboratory Manual, Cold Spring Harbor Laboratory Press, Cold Spring Harbor, N.Y.), but electroporation and conjugation can also be used (see, e.g., Ausubel et al., 1988, Current Protocols in Molecular Biology, John Wiley & Sons, Inc., NY, N.Y.).

For the introduction of vector constructs to yeast or other fungal cells, chemical transformation methods can be used (e.g., Rose et al, 1990, Methods in Yeast Genetics, Cold Spring Harbor Laboratory Press, Cold Spring Harbor, N.Y.). Transformed cells can be isolated on selective media appropriate to the selectable marker used. Alternatively, or in addition, plates or filters lifted from plates can be scanned for GFP fluorescence to identify transformed clones.

For the introduction of vectors comprising differentially expressed sequences to certain types of cells, the method used can depend upon the form of the vector. Plasmid vectors can be introduced by any of a number of transfection methods, including, for example, lipid-mediated transfection (“lipofection”), DEAE-dextran-mediated transfection, electroporation or calcium phosphate precipitation (see, e.g., Ausubel et al., 1988, Current Protocols in Molecular Biology, John Wiley & Sons, Inc., N.Y., N.Y.).

Lipofection reagents and methods suitable for transient transfection of a wide variety of transformed and non-transformed or primary cells are widely available, making lipofection an attractive method of introducing constructs to eukaryotic, and particularly mammalian cells in culture. Many companies offer kits and ways for this type of transfection.

The host cell can be capable of expressing the construct encoding the desired protein, processing the protein and transporting a secreted protein to the cell surface for secretion. Processing includes co- and post-translational modification such as leader peptide cleavage, GPI attachment, glycosylation, ubiquitination, and disulfide bond formation.

Microorganisms can be transformed or transfected with the above-described expression or vectors for production of one or more enzymes as disclosed herein or with polynucleotides coding for one or more enzymes as disclosed herein and cultured in nutrient media modified as appropriate for the specific microorganism, inducing promoters, selecting transformants, or amplifying the genes encoding the desired sequences.

For example, within the context of a methanotroph, electroporation methods can be used to deliver an expression vector.

Expression of a vector (and the gene contained in the vector) can be verified by an expression assay, for example, qPCR or by measuring levels of RNA. Expression level can be indicative also of copy number. For example, if expression levels are extremely high, this can indicate that more than one copy of a gene was integrated in a genome. Alternatively, high expression can indicate that a gene was integrated in a highly transcribed area, for example, near a highly expressed promoter. Expression can also be verified by measuring protein levels, such as through Western blotting.

### CRISPR/Cas System

Methods that require any of the genes described herein can take advantage of pinpoint insertion of genes or the deletion of genes (or parts of genes). Methods described herein can take advantage of a CRISPR/Cas system. For example, double-strand breaks (DSBs) can be generated using a CRISPR/Cas system, e.g., a type II CRISPR/Cas system. A Cas enzyme used in the methods disclosed herein can be Cas9, which catalyzes DNA cleavage. Enzymatic action by Cas9 derived from Streptococcus pyogenes or any closely related Cas9 can generate double stranded breaks at target site sequences which hybridize to 20 nucleotides of a guide sequence and that have a protospacer-adjacent motif (PAM) following the 20 nucleotides of the target sequence.

A vector can be operably linked to an enzyme-coding sequence encoding a CRISPR enzyme, such as a Cas protein. Cas proteins that can be used include class 1 and class 2. Non-limiting examples of Cas proteins include Cas1, Cas1B, Cas2, Cas3, Cas4, Cas5, Cas5d, Cas5t, Cas5h, Cas5a, Cas6, Cas7, Cas8, Cas9 (also known as Csn1 or Csx12), Cas10, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Csd1, Csd2, Cst1, Cst2, Csh1, Csh2, Csa1, Csa2, Csa3, Csa4, Csa5, C2c1, C2c2, C2c3, Cpf1, CARF, DinG, homologues thereof, or modified versions thereof. An unmodified CRISPR enzyme can have DNA cleavage activity, such as Cas9. A CRISPR enzyme can direct cleavage of one or both strands at a target sequence, such as within a target sequence and/or within a complement of a target sequence. For example, a CRISPR enzyme can direct cleavage of one or both strands within 1, 2, 3, 4, 5, 6, 7, 8, 9, 10, 15, 20, 25, 50, 100, 200, 500, or more base pairs from the first or last nucleotide of a target sequence. A vector that encodes a CRISPR enzyme that is mutated with respect to a corresponding wild-type enzyme such that the mutated CRISPR enzyme lacks the ability to cleave one or both strands of a target polynucleotide containing a target sequence can be used.

A vector that encodes a CRISPR enzyme comprising one or more nuclear localization sequences (NLSs) can be used. For example, there can be 1, 2, 3, 4, 5, 6, 7, 8, 9, or 10 NLSs used. A CRISPR enzyme can comprise the NLSs at or near the amino-terminus, e.g., 1, 2, 3, 4, 5, 6, 7, 8, 9, or 10 NLSs at or near the carboxy-terminus, or any combination of these (e.g., one or more NLS at the ammo-terminus and one or more NLS at the carboxy terminus). When more than one NLS is present, each can be selected independently of others, such that a single NLS can be present in more than one copy and/or in combination with one or more other NLSs present in one or more copies.

CRISPR enzymes used in the methods can comprise at most 6 NLSs. An NLS is considered near the N- or C-terminus when the nearest amino acid to the NLS is within 50 amino acids along a polypeptide chain from the N- or C-terminus, e.g., within 1, 2, 3, 4, 5, 10, 15, 20, 25, 30, 40, or 50 amino acids.

### Guide RNA

As used herein, the term “guide RNA” and its grammatical equivalents can refer to an RNA which can be specific for a target DNA and can form a complex with Cas protein. An RNA/Cas complex can assist in “guiding” Cas protein to a target DNA.

A method disclosed herein also can comprise introducing into a cell or embryo at least one guide RNA or nucleic acid, e.g., DNA encoding at least one guide RNA. A guide RNA can interact with an RNA-guided endonuclease to direct the endonuclease to a specific target site, at which site the 5′ end of the guide RNA base pairs with a specific protospacer sequence in a chromosomal sequence.

A guide RNA can comprise two RNAs, e.g., CRISPR RNA (crRNA) and transactivating crRNA (tracrRNA). A guide RNA can sometimes comprise a single-chain RNA, or single guide RNA (sgRNA) formed by fusion of a portion (e.g., a functional portion) of crRNA and tracrRNA. A guide RNA can also be a dualRNA comprising a crRNA and a tracrRNA. Furthermore, a crRNA can hybridize with a target DNA.

As discussed above, a guide RNA can be an expression product. For example, a DNA that encodes a guide RNA can be a vector comprising a sequence coding for the guide RNA. A guide RNA can be transferred into a cell or microorganism by transfecting the cell or microorganism with an isolated guide RNA or plasmid DNA comprising a sequence coding for the guide RNA and a promoter. A guide RNA can also be transferred into a cell or microorganism in other way, such as using virus-mediated gene delivery.

A guide RNA can be isolated. For example, a guide RNA can be transfected in the form of an isolated RNA into a cell or microorganism. A guide RNA can be prepared by in vitro transcription using any in vitro transcription system known in the art. A guide RNA can be transferred to a cell in the form of isolated RNA rather than in the form of plasmid comprising encoding sequence for a guide RNA.

A guide RNA can comprise three regions: a first region at the 5′ end that can be complementary to a target site in a chromosomal sequence, a second internal region that can form a stem loop structure, and a third 3′ region that can be single-stranded. A first region of each guide RNA can also be different such that each guide RNA guides a fusion protein to a specific target site. Further, second and third regions of each guide RNA can be identical in all guide RNAs.

A first region of a guide RNA can be complementary to sequence at a target site in a chromosomal sequence such that the first region of the guide RNA can base pair with the target site. In some cases, a first region of a guide RNA can comprise from 10 nucleotides to 25 nucleotides (e.g., from 10 nts to 25 nts; or from 15 nts to 25 nts; or from 10 nts to 20 nts; or from 15 nts to 20 nts) or more. For example, a region of base pairing between a first region of a guide RNA and a target site in a chromosomal sequence can be 10, 11, 12, 13, 14, 15, 16, 17, 18, 19, 20, 22, 23, 24, 25, or more nucleotides in length. Sometimes, a first region of a guide RNA can be 19, 20, or 21 nucleotides in length.

A guide RNA can also comprises a second region that forms a secondary structure. For example, a secondary structure formed by a guide RNA can comprise a stem (or hairpin) and a loop. A length of a loop and a stem can vary. For example, a loop can range from 3 to 10 nucleotides in length, and a stem can range from 6 to 20 base pairs in length. A stem can comprise one or more bulges of 1 to 10 nucleotides. The overall length of a second region can range from 16 to 60 nucleotides in length. For example, a loop can be 4 nucleotides in length and a stem can be 12 base pairs.

A guide RNA can also comprise a third region at the 3′ end that can be essentially single-stranded. For example, a third region is sometimes not complementarity to any chromosomal sequence in a cell of interest and is sometimes not complementarity to the rest of a guide RNA. Further, the length of a third region can vary. A third region can be more than 4 nucleotides in length. For example, the length of a third region can range from 5 to 60 nucleotides in length.

A guide RNA can be introduced into a cell or embryo as an RNA molecule. For example, an RNA molecule can be transcribed in vitro and/or can be chemically synthesized. An RNA can be transcribed from a synthetic DNA molecule, e.g., a gBlocks® gene fragment. A guide RNA can then be introduced into a cell or embryo as an RNA molecule. A guide RNA can also be introduced into a cell or embryo in the form of a non-RNA nucleic acid molecule, e.g., DNA molecule. For example, a DNA encoding a guide RNA can be operably linked to promoter control sequence for expression of the guide RNA in a cell or embryo of interest. An RNA coding sequence can be operably linked to a promoter sequence that is recognized by RNA polymerase III (Pol III). Plasmid vectors that can be used to express guide RNA include, but are not limited to, px330 vectors and px333 vectors. In some cases, a plasmid vector (e.g., px333 vector) can comprise two guide RNA-encoding DNA sequences.

A DNA sequence encoding a guide RNA can also be part of a vector. Further, a vector can comprise additional expression control sequences (e.g., enhancer sequences, Kozak sequences, polyadenylation sequences, transcriptional termination sequences, etc.), selectable marker sequences (e.g., antibiotic resistance genes), origins of replication, and the like. A DNA molecule encoding a guide RNA can also be linear. A DNA molecule encoding a guide RNA can also be circular.

When DNA sequences encoding an RNA-guided endonuclease and a guide RNA are introduced into a cell, each DNA sequence can be part of a separate molecule (e.g., one vector containing an RNA-guided endonuclease coding sequence and a second vector containing a guide RNA coding sequence) or both can be part of a same molecule (e.g., one vector containing coding (and regulatory) sequence for both an RNA-guided endonuclease and a guide RNA).

### Site Specific Insertion

Inserting one or more genes in any microorganisms used in the of the methods disclosed throughout can be site-specific. For example, one or more genes can be inserted adjacent to a promoter.

Modification of a targeted locus of a microorganism can be produced by introducing DNA into microorganisms, where the DNA has homology to the target locus. DNA can include a marker gene, allowing for selection of cells comprising the integrated construct. Homologous DNA in a target vector can recombine with DNA at a target locus. A marker gene can be flanked on both sides by homologous DNA sequences, a 3′ recombination arm, and a 5′ recombination arm.

A variety of enzymes can catalyze insertion of foreign DNA into a microorganism genome. For example, site-specific recombinases can be clustered into two protein families with distinct biochemical properties, namely tyrosine recombinases (in which DNA is covalently attached to a tyrosine residue) and serine recombinases (where covalent attachment occurs at a serine residue). In some cases, recombinases can comprise Cre, (Φ31 integrase (a serine recombinase derived from Streptomyces phage (Φ31), or bacteriophage derived site-specific recombinases (including Flp, lambda integrase, bacteriophage HK022 recombinase, bacteriophage R4 integrase and phage TP901-1 integrase).

The CRISPR/Cas system can be used to perform site specific insertion. For example, a nick on an insertion site in the genome can be made by CRISPR/Cas to facilitate the insertion of a transgene at the insertion site.

The techniques which can be used to allow a DNA or RNA construct entry into a host cell in the methods described herein include, but are not limited to, calcium phosphate/DNA coprecipitation, microinjection of DNA into a nucleus, electroporation, bacterial protoplast fusion with intact cells, transfection, lipofection, infection, particle bombardment, sperm mediated gene transfer, or any other technique.

Certain aspects disclosed herein can utilize vectors (including the ones described above). Any plasmids and vectors can be used as long as they are replicable and viable in a selected host microorganism. Vectors known in the art and those commercially available (and variants or derivatives thereof) can be engineered to include one or more recombination sites for use in the methods herein. Vectors that can be used include, but are not limited to eukaryotic expression vectors such as pFastBac, pFastBacHT, pFastBacDUAL, pSFV, and pTet-Splice (Invitrogen), pEUK-C1, pPUR, pMAM, pMAMneo, pBI101, pBI121, pDR2, pCMVEBNA, and pYACneo (Clontech), pSVK3, pSVL, pMSG, pCH110, and pKK232-8 (Pharmacia, Inc.), pXT1, pSG5, pPbac, pMbac, pMClneo, and pOG44 (Stratagene, Inc.), and pYES2, pAC360, pBlueBa-cHis A, B, and C, pVL1392, pBlueBac111, pCDM8, pcDNA1, pZeoSV, pcDNA3, pREP4, pCEP4, and pEBVHis (Invitrogen, Corp.), and variants or derivatives thereof.

These vectors can be used to express a gene or portion of a gene of interest. A gene or a portion of a gene can be inserted by using known methods, such as restriction enzyme-based techniques.

### III. Other Methods

### Making Useful Chemicals

The genetically modified microorganisms described herein can be used to make chemicals that are useful, including but not limited to 2-acetolactate; 2,3-dihydroxyisovalerate; ketoisovalerate; isobutyraldehyde; and isobutanol. Other useful products or chemicals that can be made with the methods and microorganisms described throughout can include amino acids such as lysine and isoleucine, sugar/glycogen, acetate, pyruvate, lactate, citrate, isovaleraldehyde, isopentanol, acetylated isobutanol or acetylated isopentanol (isobutrylacetate and isoamylacetate), and pentadecanoic acid (rare odd chain fatty acid over produced by the genetically modified microorganisms disclosed throughout but not produced at high level in wild-type strains).

Further, some of these chemicals can be used to produce other useful products including but not limited, to isobutyl acetate, isobutyl esters such as diisobutyl phthalate (DIBP), methyl methacrylate (MMA), isobutene, para-xylene, paint solvents, varnish remover, ink ingredients, paint additives, gasoline additives, gasoline alternatives, automotive polish additives, automotive paint cleaner additives, and chemical extractants in the production of organic compounds.

The microorganism can be any of the microorganisms discussed throughout including but not limited to a prokaryote, such as a methanotroph.

The carbon substrate can be any carbon substrate discussed throughout including but not limited to methane.

2-acetolactate

With regards to 2-acetolactate, one method disclosed herein is a method of making 2-acetolactate comprising: (a) contacting a genetically modified microorganism with a carbon substrate, where the microorganism comprises a heterologous gene encoding for acetolactate synthase; and (b) growing the microorganism to produce 2-acteolactate. In some cases, the acetolactate synthase gene can be from the genus Bacillus, such as the species Bacillus subtilis and/or Bacillus lichenformis. For example, an acetolactate synthase that can be used can be encoded by a nucleic acid having substantial similarity to SEQ ID NO: 1 or 100. The microorganisms that can be used in this method are described throughout (such as a methanotroph). Additionally, the microorganism can be transformed with or made to express one or more of the genes to make other products such as 2,3-butanediol (“2,3-BDO”) and/or isobutanol and/or isobutyraldehyde (such as the genes that are described throughout). Further, the carbon sources can be any carbon source described throughout, such as a C₁ carbon source. The 2-acetolactate produced can be substantially pure. The 2-acetolactate produced can be recovered.

The 2-acetolactate can be further processed through the use of one or more enzymatic reactions. For example, 2-acetolactate can be processed into 2,3-BDO by contacting it with alpha-acetolactate (budA) or acetoin reductase (butA). In some cases, the 2-acetolactate can be contacted with a 2,3-butanediol dehydrogenase.

### 2,3-Butanediol (“2,3-BDO”)

With regards to 2,3-BDO, one method disclosed herein is a method of making 2,3-BDO comprising: (a) contacting a genetically modified microorganism with a carbon substrate, where the microorganism comprises a heterologous gene encoding for acetolactate synthase; and (b) growing the microorganism to produce 2,3-BDO. The microorganisms that can be used in this method are described throughout (such as a methanotroph). Additionally, the microorganism can be transformed with or made to express one or more of the genes (such as a gene encoding for an alpha-acetolactate (budA) or acetoin reductase (butA)) or other genes as disclosed throughout. Further, the carbon sources can be any carbon source described throughout, such as a C₁ carbon source. The 2,3-BDO produced can be substantially pure. The 2,3-BDO produced can be recovered.

The 2,3-BDO can be further processed through the use of one or more catalysts. For example, 2,3-BDO can be processed into methyl ethyl ketone (MEK) by contacting the 2,3-BDO with a dehydrating catalyst (such as alumina, direct reaction with sulfuric acid, Cu, AlO₃, and/or zeolite (or other solid acid catalysts)). 2,3-BDO can also be processed into 1,3-butadiene by contacting the 2,3-BDO with a catalyst capable of producing a hydride shift, such as alumina or sulfuric acid. 2,3-BDO can also be processed into butene by contacting the 2,3-BDO with a HBr following by Zn. MEK, 1,3-butadiene and butene can be converted into a variety of different products such as synthetic rubbers or solvents.

The 2,3-BDO can also be further processed by a diol dehydratase (B12). This enzymatic reaction can produce butan-2-one (also known as methyl ethyl ketone or MEK). Thus, disclosed is a method of making butan-2-one comprising: (a) contacting a genetically modified microorganism with a carbon substrate, where the microorganism comprises a heterologous gene encoding for diol dehydratase; and (b) growing the microorganism to produce butan-2-one. The microorganism can also comprise an acetolactate synthase (AlsS), alpha-acetolactate (budA), and/or acetoin reductase (butA).

In some cases, the butan-2-one can be further processed by an alcohol dehydrogenase. This enzymatic reaction can produce butan-2-ol (also known as 2-butanol). Thus, disclosed is a method of making butan-2-ol comprising: (a) contacting a genetically modified microorganism with a carbon substrate, where the microorganism comprises a heterologous gene encoding for an alcohol dehydrogenase; and (b) growing the microorganism to produce butan-2-ol. The microorganism can also comprise an acetolactate synthase (AlsS), alpha-acetolactate (budA), acetoin reductase (butA), and/or diol dehydratase (B12).

### Diacetyl

Diacetyl (also known as butanedione) can also be produced from 2-acetolactate. Disclosed herein is a method of making 2-acetolactate comprising: (a) contacting a genetically modified microorganism with a carbon substrate, where the microorganism comprises a heterologous gene encoding for acetolactate synthase; (b) growing the microorganism to produce 2-acetolactate; and (c) further processing 2-acetolactate into diacetyl. In general, diacetyl can be made by the spontaneous oxidative decarboxylation of acetolactate. Milne, N., et al., “Excessive by-product formation: A key contributor to low isobutanol yields of engineered Saccharomyces cerevisiae strains,” Metabolic Engineering Communications 3:39-51 (2016). Diacetyl can be produced during fermentation as a byproduct of valine synthesis, when 2-acetolactate escapes the cell and is spontaneously decarboxylated into diacetyl.

The microorganisms that can be used in this method are described throughout (such as a methanotroph). Additionally, the microorganism can be transformed with or made to express one or more of the genes as disclosed throughout. Further the carbon sources can be any carbon source described throughout, such as a C₁ carbon source. The diacetyl produced can be substantially pure. The diacetyl that is produced can be recovered.

Diacetyl can be used in food products as it has buttery characteristics. Therefore, diacetyl can be used in the manufacture of foods in which a buttery taste is desired, such as artificial butter flavoring, margarines or similar oil-based products (along with acetoin and beta-carotene) to make the final product butter-flavored. Diacetyl can also be used in electronic cigarette liquids for flavoring.

Diacetyl can also be used in alcoholic beverages. At low levels, diacetyl provides a slipperiness to the feel of the alcoholic beverage in the mouth. As diacetyl levels increase, it imparts a buttery or butterscotch flavor. For example, diacetyl can be contained in beer and wines. For example, concentrations from 0,005 mg/L to 1.7 mg/L were measured in chardonnay wines, and the amount needed for the flavor to be noticed is at least 0.2 mg/L.

2,3-dihydroxy-2-methylbutanoic acid

2,3-dihydroxy-2-methylbutanoic acid can be produced from the methods and microorganisms discussed herein. For example, disclosed is a method of making 2,3-dihydroxy-2-methylbutanoic acid comprising: (a) contacting a genetically modified microorganism with a carbon substrate, where the microorganism comprises a heterologous gene encoding for acetolactate synthase; (b) growing the microorganism to produce 2-acetolactate; and (c) contacting the 2-acetolactate with an enzyme that is capable of converting 2-acetolactate to 2,3-dihydroxy-2-methylbutanoic acid. In some cases the enzyme has an EC number 1.1.1.86. In some cases, the enzyme is a ketol-acid reductoisomerase.

The microorganisms that can be used in this method are described throughout (such as a methanotroph). Additionally, the microorganisms can be transformed with or made to express one or more of the genes as disclosed throughout. Further, the carbon sources can be any carbon source described throughout, such as a C₁ carbon source. The 2,3-dihydroxy-2-methylbutanoic acid produced can be substantially pure. The 2,3-dihydroxy-2-methylbutanoic acid that is produced can be recovered.

2,3-dihydroxy-2-methylbutanoic acid can also be used in alcoholic beverages. 2,3-dihydroxy-2-methylbutanoic acid can be contained in alcoholic beverages such as beer and wines. For example, concentrations from 0.26 ppm were measured in some German beers.

2,3-dihydroxyisovalerate

With regards to 2,3-dihydroxyisovalerate, one method disclosed herein is a method of making 2,3-dihydroxyisovalerate comprising (a) contacting a genetically modified microorganism with a carbon substrate, where the microorganism comprises at least one heterologous gene encoding for: (i) acetolactate synthase and/or (ii) ketol-acid reductoisomerase; and (b) growing the microorganism to produce 2,3-dihydroxyisovalerate. The microorganisms that can be used in this method are described throughout (such as a methanotroph). Additionally, the microorganisms can be transformed with or made to express one or more of the genes as disclosed throughout. Further the carbon sources can be any carbon source described throughout, such as a C₁ carbon source. The 2,3-dihydroxyisovalerate produced can be substantially pure. The 2,3-dihydroxyisovalerate that is produced can be recovered.

The 2,3-dihydroxyisovalerate can be further processed by the same microorganism, a different microorganism, or outside a microorganism (i.e., in vitro) through the use of additional enzymes such as a dihydroxy-acid dehydratase. The same microorganism can comprise a dihydroxy-acid dehydratase. In other instances, a different microorganism can comprise a dihydroxy-acid dehydratase or a dihydroxy-acid dehydratase is isolated from a cell and used in vitro. If the dihydroxy-acid dehydratase is in a different microorganism or is isolated from a cell, the microorganism/isolated enzyme can convert 2,3-dihydroxyisovalerate that is in the culture media (either by supplemental addition or by secretion by 2,3-dihydroxyisovalerate producing microorganism). The conversion of 2,3-dihydroxyisovalerate by dihydroxy-acid dehydratase can produce some desired products such as ketoisovalerate or isobutanol.

### Amino Acids

Amino acids can be made using the methods and microorganisms disclosed throughout. For example, disclosed herein is a method of making amino acids comprising (a) contacting a genetically modified microorganism with a carbon substrate, where the microorganism comprises at least one heterologous gene encoding for: (i) acetolactate synthase and/or (ii) ketol-acid reductoisomerase; (b) growing the microorganism to produce 2,3-dihydroxyisovalerate; and (c) contacting the 2,3-dihydroxyisovalerate with one or more enzymes that are capable of converting 2,3-dihydroxyisovalerate into an amino acid. The microorganisms that can be used in this method are described throughout (such as a methanotroph). Additionally, the microorganisms can be transformed with or made to express one or more of the genes as disclosed throughout. Further, the carbon sources can be any carbon source described throughout, such as a C₁ carbon source. The amino acids produced can be substantially pure. The amino acids that are produced can be recovered. The amino acids can be any one of valine, leucine, isoleucine, or any combination thereof.

The one or more enzymes that are capable of converting 2,3-dihydroxyisovalerate into an amino acid can be one or more of the following: dihydroxy-acid dehydratase; branched-chain amino acid transaminase (BAT2); branched-chain amino acid aminotransferase (BAT1); alpha-isopropylmalate synthase (LEU9, LEU4), isopropylmalate isomerase (LEU1), and/or beta-IPM dehydrogenase (LEU2).

For example, biosynthesis of valine includes steps of converting 2,3-dihydroxyisovalerate to 2-keto-isovalerate by dihydroxy-acid dehydratase, and conversion of 2-keto-isovalerate to valine by branched-chain amino acid transaminase (BAT2) and branched-chain amino acid aminotransferase (BAT1). Further, biosynthesis of leucine includes the same steps to 2-keto-isovalerate, followed by conversion of 2-keto-isovalerate to alpha-isopropylmalate by alpha-isopropylmalate synthase (LEU9, LEU4), conversion of alpha-isopropylmalate to beta-isopropylmalate by isopropylmalate isomerase (LEU1), conversion of beta-isopropylmalate to alpha-ketoisocaproate by beta-IPM dehydrogenase (LEU2), and finally conversion of alpha-ketoisocaproate to leucine by branched-chain amino acid transaminase (BAT2) and branched-chain amino acid aminotransferase (BAT1).

### Ketoisovalerate

With regards to ketoisovalerate, one method disclosed herein is a method of making ketoisovalerate comprising (a) contacting a genetically modified microorganism with a carbon substrate, where the microorganism comprises at least one heterologous gene encoding for: (i) acetolactate synthase, (ii) ketol-acid reductoisomerase, (iii) dihydroxy-acid dehydratase, or (iv) any combination thereof; and (b) growing the microorganism to produce ketoisovalerate. The microorganisms that can be used in this method are described throughout (such as a methanotroph). Additionally, the microorganisms can be transformed with or made to express one or more of the genes as disclosed throughout. Further the carbon sources can be any carbon source described throughout, such as a C₁ carbon source. The ketoisovalerate produced can be substantially pure. The ketoisovalerate that is produced can be recovered.

The ketoisovalerate can be further processed by the same microorganism, a different microorganism, or outside a microorganism (i.e., in vitro) through the use of additional enzymes such as a 2-keto acid decarboxylase. The same microorganism can comprise a 2-keto acid decarboxylase. In other instances, a different microorganism can comprise a 2-keto acid decarboxylase or a 2-keto acid decarboxylase is isolated from a cell and used in vitro. If the 2-keto acid decarboxylase is in a different microorganism or is isolated from a cell, the microorganism/isolated enzyme can convert ketoisovalerate that is in the culture media (either by supplemental addition or by secretion by ketoisovalerate producing microorganism). The conversion of ketoisovalerate by 2-keto acid decarboxylase can produce some desired products such as isobutyraldehyde or isobutanol.

### Isobutyraldehyde

With regards to isobutyraldehyde, one method disclosed herein is a method of making isobutyraldehyde comprising (a) contacting a genetically modified microorganism with a carbon substrate, where the microorganism comprises at least one heterologous gene encoding for: (i) acetolactate synthase, (ii) ketol-acid reductoisomerase, (iii) dihydroxy-acid dehydratase, (iv) 2-keto acid decarboxylase, or (v) any combination thereof; and (b) growing the microorganism to produce isobutyraldehyde. The microorganisms that can be used in this method are described throughout (such as a methanotroph). Additionally, the microorganisms can be transformed with or made to express one or more of the genes as disclosed throughout. Further, the carbon sources can be any carbon source described throughout, such as a C1 carbon source. The isobutyraldehyde produced can be substantially pure. The isobutyraldehyde that is produced can be recovered.

The isobutyraldehyde can be further processed by the same microorganism, a different microorganism, or outside a microorganism (i.e., in vitro) through the use of additional enzymes such as an alcohol dehydrogenase. The same microorganism can comprise an alcohol dehydrogenase. In other instances, a different microorganism can comprise an alcohol dehydrogenase or an alcohol dehydrogenase is isolated from a cell and used in vitro. If the alcohol dehydrogenase is in a different microorganism or is isolated from a cell, the microorganism/isolated enzyme can convert isobutyraldehyde that is in the culture media (either by supplemental addition or by secretion by isobutyraldehyde producing microorganism). The conversion of isobutyraldehyde by alcohol dehydrogenase can produce some desired products such as isobutanol or other products such as methyl methacrylate.

### Isobutyrate

With regards to isobutyrate, one method disclosed herein is a method of making isobutyrate comprising (a) contacting a genetically modified microorganism with a carbon substrate, where the microorganism comprises at least one heterologous gene encoding for: (i) acetolactate synthase, (ii) ketol-acid reductoisomerase, (iii) dihydroxy-acid dehydratase, (iv) 2-keto acid decarboxylase, or (v) any combination thereof; and (b) growing the microorganism to produce isobutyrate. In some cases, isobutyraldehyde produced by the microorganism can be oxidized. This oxidization can produce isobutyrate. The oxidization can be performed by catalysts or an enzyme. In some cases, when enzymatic oxidization is required, a promiscuous phenylacetaldehyde dehydrogenase (PadA) can be used (e.g., an enzyme having an EC number of 1.2.1.39). The PadA can be from a microorganism such as E. coli. The microorganisms that can be used in this method are described throughout (such as a methanotroph). Additionally, the microorganisms can be transformed with or made to express one or more of the genes as disclosed throughout. Further, the carbon sources can be any carbon source described throughout, such as a C₁ carbon source. The isobutyrate produced can be substantially pure. The isobutyrate that is produced can be recovered.

The isobutyrate produced can be made into other products such as methyl methacrylate (MMA). Isobutyrate can also be combined with many other chemicals, which can in turn be used for a variety of purposes. For example, phenoxy ethyl isobutyrate or styralyl isobutyrate have pleasant scents and can be used in a variety of perfumes.

### Methyl Methacrylate (MMA)

With regards to methyl methacrylate (MMA), disclosed herein is a method of making MMA comprising (a) contacting a genetically modified microorganism with a carbon substrate, where the microorganism comprises at least one heterologous gene encoding for: (i) acetolactate synthase, (ii) ketol-acid reductoisomerase, (iii) dihydroxy-acid dehydratase, (iv) 2-keto acid decarboxylase, or (v) any combination thereof; (b) growing the microorganism to produce isobutyraldehyde; and (c) contacting the isobutyraldehyde with one or more catalysts capable of converting isobutyraldehyde to MMA. The microorganisms that can be used in this method are described throughout (such as a methanotroph). Additionally, the microorganisms can be transformed with or made to express one or more of the genes as disclosed throughout. Further, the carbon sources can be any carbon source described throughout, such as a C₁ carbon source. The MMA from this method can be substantially pure. The MMA produced can be recovered. The MMA can be converted into polymethyl methacrylate acrylic plastics.

Isobutyraldehyde can be oxidized (sometimes in liquid phase) to form isobutyric acid. The isobutyric acid can be dehydrogenated (e.g., in a gas phase) into methacrylic acid. The catalyst that can be used for dehydrogenation can be a heteropoly acid catalyst (e.g., 12-tungstosilicate and 12-molybdophosphate). The methacrylic acid can be then esterified to form methacrylates. Otake, M., and Onoda, T., “A New Route to Methacrylates from Isobutyraldehyde,” Studies in Surface Science and Catalysis, Vol. 7, Part B: 780-791 (1981).

MMA is used primarily for the manufacture of polymethyl methacrylate acrylic plastics (PMMA). Methyl methacrylate can also be used for the production of the co-polymer methyl methacrylate-butadiene-styrene (MBS), which is used as a modifier for PVC. Another application of MMA is as cement used in total hip replacements as well as total knee replacements. MMA is also a raw material for the manufacture of other methacrylates. These derivatives include ethyl methacrylate (EMA), butyl methacrylate (BMA) and 2-ethyl hexyl methacrylate (2-EHMA). Methacrylic acid (MAA) is used as a chemical intermediate as well as in the manufacture of coating polymers, construction chemicals and textile applications.

### Isovaleraldehyde

With regards to isovaleraldehyde, disclosed herein is a method of making isovaleraldehyde comprising (a) contacting a genetically modified microorganism with a carbon substrate, where the microorganism comprises at least one heterologous gene encoding for: (i) acetolactate synthase, (ii) ketol-acid reductoisomerase, (iii) dihydroxy-acid dehydratase, (iv) 2-keto acid decarboxylase, or (v) any combination thereof; and (b) growing the microorganism to produce isovaleraldehyde. The microorganisms that can be used in this method are described throughout (such as a methanotroph). Additionally, the microorganisms can be transformed with or made to express one or more of the genes as disclosed throughout. Further, the carbon sources can be any carbon source described throughout, such as a C₁ carbon source. The isovaleraldehyde from this method can be substantially pure. The isovaleraldehyde produce can be recovered. Isovaleraldehyde can be produced as a significant product during the fermentation of pyruvate to isobutyraldehyde.

The amino acids produced by the methods and microorganisms described throughout can be converted into isovaleraldehyde. Isovaleraldehyde can be made when leucine is broken down. However, isovaleraldehyde can also be made by the hydroformylation of isobutene.

The isovaleraldehyde made by the methods and microorganisms described herein can be converted into different products. Isovaleraldehyde can be used as a flavoring in many different types of foods, such as beer, cheese, coffee, chicken, fish, chocolate, olive oil, and tea.

Isovaleraldehyde can also be used as a reactant in the synthesis of a number of compounds. For example, isovaleraldehyde can be used to synthesize 2,3-dimnethyl-2-butene. 2,3-dimethyl-2-butene can then be converted to 2,3-dimethylbutane-2,3-diol and methyltert-butylketone, better known as pinacolone. Pinacolone can then be used in the synthesis of pesticides, Additionally, a range of pharmaceuticals, such as butizide, are synthesized from isovaleraldehyde and its corresponding acid.

### Isovalerate

With regards to isovalerate, disclosed herein is a method of making isovalerate comprising (a) contacting a genetically modified microorganism with a carbon substrate, where the microorganism comprises at least one heterologous gene encoding for: (i) acetolactate synthase, (ii) ketol-acid reductoisomerase, (iii) dihydroxy-acid dehydratase, (iv) 2-keto acid decarboxylase, or (v) any combination thereof; and (b) growing the microorganism to produce isovalerate. The microorganisms that can be used in this method are described throughout (such as a methanotroph). Additionally, the microorganisms can be transformed with or made to express one or more of the genes as disclosed throughout. Further, the carbon sources can be any carbon source described throughout, such as a C₁ carbon source. The isovalerate from this method can be substantially pure. The isovalerate produced can be recovered. Isovalerate can be produced as a significant product during the fermentation of pyruvate to isobutyraldehyde. The amino acids produced by the methods and microorganisms described throughout can be converted into isovalerate. Isovalerate can be made when leucine is broken down.

The isovalerate made by the methods and microorganisms described herein can be converted into different products. Isovalerate esters can be used in perfumes as it has a pleasing scent. Isovaleric acid has also been used to synthesize β-hydroxyisovaleric acid.

### Isopentanol

With regards to isopentanol (also known as isoamyl alcohol or isopentyl alcohol), one method disclosed herein is a method of making isopentanol comprising (a) contacting a genetically modified microorganism with a carbon substrate, where the microorganism comprises at least one heterologous gene encoding for: (i) acetolactate synthase, (ii) ketol-acid reductoisomerase, (iii) dihydroxy-acid dehydratase, or (iv) any combination thereof; and (b) growing the microorganism to produce isopentanol. The microorganisms that can be used in this method are described throughout (such as a methanotroph). Additionally, the microorganisms can be transformed with or made to express one or more of the genes as disclosed throughout. Further, the carbon sources can be any carbon source described throughout, such as a C₁ carbon source. The isopentanol produced can be substantially pure. The isopentanol that is produced can be recovered. The isopentanol can also be acetylated. Therefore, the microorganism can produce acetylated isopentanol.

Isopentanol is a main ingredient in the production of banana oil, an ester found in nature. Isopentanol is also produced as a flavoring for the food industry. Isopentanol is also one of the components of the aroma produced by black truffles. Isopentanol is also the main ingredient of Kovac's reagent, used for the bacterial diagnostic indole test. Isopentanol can also be used as an antifoaming agent in a Chloroform:Isomyl Alcohol reagent. Isopentanol is used in a phenol-chloroform extraction mixed with the chloroform to further inhibit RNase activity and prevent solubility of RNAs with long tracts of poly-adenine.

### Isoamyl Acetate

With regards to isoamyl acetate, one method disclosed herein is a method of making isoamyl acetate comprising (a) contacting a genetically modified microorganism with a carbon substrate, where the microorganism comprises at least one heterologous gene encoding for: (i) acetolactate synthase, (ii) ketol-acid reductoisomerase, (iii) dihydroxy-acid dehydratase, or (iv) any combination thereof; and (b) growing the microorganism to produce isoamyl acetate. The microorganisms that can be used in this method are described throughout (such as a methanotroph). Additionally, the microorganisms can be transformed with or made to express one or more of the genes as disclosed throughout. Further, the carbon sources can be any carbon source described throughout, such as a C₁ carbon source. The isoamyl acetate produced can be substantially pure. The isoamyl acetate that is produced can be recovered.

Isoamyl acetate can be formed by contact of isoamyl alcohol (e.g., isopentanol) and an acid catalyst (e.g., a lacial acetic acid or sulfuric acid). Sulfuric acid or an acidic ion exchange resin can be used as a catalyst.

Isoamyl acetate is used to confer banana flavor in foods. Pear oil commonly refers to a solution of isoamyl acetate in ethanol that is used as an artificial flavor. Isoamyl acetate can be used as a solvent for some varnishes and nitrocellulose lacquers. Isoamyl acetate can also be used in thermometers.

### Pentadecanoic Acid

With regards to pentadecanoic acid, one method disclosed herein is a method of making pentadecanoic acid comprising (a) contacting a genetically modified microorganism with a carbon substrate, where the microorganism comprises at least one heterologous gene encoding for: (i) acetolactate synthase, (ii) ketol-acid reductoisomerase, (iii) dihydroxy-acid dehydratase, (iv) 2-keto acid decarboxylase, (v) alcohol dehydrogenase, or (vi) any combination thereof; and (b) growing the microorganism to produce pentadecanoic acid. The microorganisms that can be used in this method are described throughout (such as a methanotroph). Additionally, the microorganisms can be transformed with or made to express one or more of the genes as disclosed throughout. Further, the carbon sources can be any carbon source described throughout, such as a C₁ carbon source.

The starter unit (propionyl-CoA) for pentadecanoic acid biosynthesis can be produced through amino acid degradation, such as isoleucine. The pentadecanoic acid that is produced can be recovered. The pentadecanoic acid produced can be substantially pure.

Pentadecanoic acid is rare in nature. It is a fatty acid of exogenous (primarily ruminant) origin. Many “odd” length long chain amino acids are derived from the consumption of dairy fats (milk and meat). The butterfat in cow's milk is its major dietary source and it is used as a marker for butterfat consumption. Pentadecanoic acid can decrease mother-to-child transmission of HIV through breastfeeding.

### Isobutanol

With regards to isobutanol, one method disclosed herein is a method of making isobutanol comprising (a) contacting a genetically modified microorganism with a carbon substrate, where the microorganism comprises at least one heterologous gene encoding for: (i) acetolactate synthase, (ii) ketol-acid reductoisomerase, (iii) dihydroxy-acid dehydratase, (iv) 2-keto acid decarboxylase, (v) alcohol dehydrogenase, or (vi) any combination thereof; and (b) growing the microorganism to produce isobutanol. The microorganisms that can be used in this method are described throughout (such as a methanotroph). Additionally, the microorganisms can be transformed with or made to express one or more of the genes as disclosed throughout. Further, the carbon sources can be any carbon source described throughout, such as a C₁ carbon source. The isobutanol produced can be substantially pure. The isobutanol that is produced can be recovered.

The isobutanol can be further processed by the same microorganism, a different microorganism, or outside a microorganism (i.e., in vitro) through the use of additional enzymes or catalysts. The isobutanol can be made into different products such as isobutene.

### Isobutene (aka Isobutylene)

With regards to isobutene, one method disclosed herein is a method of making isobutene comprising (a) contacting a genetically modified microorganism with a carbon substrate, where the microorganism comprises at least one heterologous gene encoding for: (i) acetolactate synthase, (ii) ketol-acid reductoisomerase, (iii) dihydroxy-acid dehydratase, (iv) 2-keto acid decarboxylase, (v) alcohol dehydrogenase, or (vi) any combination thereof; (b) growing the microorganism to produce isobutanol; and (c) dehydrating the isobutanol to form isobutene. The microorganisms that can be used in this method are described throughout (such as a methanotroph). Additionally, the microorganisms can be transformed with or made to express one or more of the genes as disclosed throughout. Further, the carbon sources can be any carbon source described throughout, such as a C₁ carbon source. The isobutene produced can be substantially pure. The isobutene that is produced can be recovered.

In some instances, the dehydration of isobutanol can occur enzymatically. For example, an oleate hydratase can be used to convert the isobutanol produced herein to make isobutene. Should an enzymatic dehydration be desired, the genetically modified can comprises one or more oleate hydratases.

Isobutene can be further converted into different products. For example, isobutene can be used as an intermediate in the production of a variety of products. It is reacted with methanol and ethanol in the manufacture of the gasoline oxygenates methyl tert-butyl ether (MTBE) and ethyl tert-butyl ether (ETBE), respectively. Alkylation with butane produces isooctane, another fuel additive. Isobutene is also used in the production of methacrolein. Polymerization of isobutene produces butyl rubber (polyisobutylene). Antioxidants such as butylated hydroxytoluene (BHT) and butylated hydroxyanisole (BHA) are produced by Friedel-Crafts alkylation of phenols using isobutene.

Para-Xylene (p-Xylene)

With regards to para-xylene (p-xylene), one method disclosed herein is a method of making p-xylene comprising (a) contacting a genetically modified microorganism with a carbon substrate, where the microorganism comprises at least one heterologous gene encoding for: (i) acetolactate synthase, (ii) ketol-acid reductoisomerase, (iii) dihydroxy-acid dehydratase, (iv) 2-keto acid decarboxylase, (v) alcohol dehydrogenase, or (vi) any combination thereof; (b) growing the microorganism to produce isobutanol; and (c) contacting the isobutanol with a catalyst capable of converting isobutanol into p-xylene. The microorganisms that can be used in this method are described throughout (such as a methanotroph). Additionally, the microorganisms can be transformed with or made to express one or more of the genes as disclosed throughout. Further, the carbon sources can be any carbon source described throughout, such as a C₁ carbon source. The catalyst of (c) can be a catalyst that is capable of dehydrating isobutanol. The dehydration of isobutanol can form a C₄ alkene, such as isobutene. The C₄ alkene can subsequently be dimerized by an oligomerization catalyst to form a C₈ alkene, such as 2,4,4-trimethylpentenes or 2,5-dimethylhexene. The C₈ alkene can be dehydrocyclized by a dehydrocyclization catalyst to form p-xylene. The p-xylene that is produced can be recovered. The p-xylene produced can be substantially pure.

In some cases, the dehydration catalyst can be an organic or inorganic acid, or a metal salt, for example, an acidic γ-alumina catalyst. In some cases, the oligomerization catalyst can be a heterogeneous acidic catalyst. For example, the oligomerization catalyst can be an acidic zeolite, solid phosphoric acid, or a sulfonic acid resin. In some cases, the dehydrocyclization catalyst is a heterogeneous metal-containing dehydrogenation catalyst. In some cases, the dehydrocyclization catalyst is a supported chromium-containing compound. The dehydrocyclization catalyst can also be a chromium-oxide treated alumina; platinum- and tin-containing zeolites; or alumina, cobalt- or molybdenum-containing alumina.

p-xylene is an important chemical feedstock. Among other industrial applications, it is a raw material in the large scale synthesis of various polymers, such as for the production of terephthalic acid to make polyesters such as polyethylene terephthalate. It also can be polymerized directly to produce parylene. p-xylene is converted into either TPA or TPA esters by oxidation over a transition metal-containing catalyst. For example, p-xylene can be oxidized in air or oxygen (or air or oxygen diluted with other gases) over a catalyst containing nickel, manganese, and cobalt. p-xylene produced can be made into a renewable polyester by contacting TPA with ethylene glycol, propylene glycol, or butylene glycol in the presence of an acidic polymerization catalyst, such as antimony (III) oxide.

### IV. Fermentation

In general, the microorganisms disclosed herein should be used in fermentation conditions that are appropriate to convert a carbon (such as methane) to isobutanol (or other desired product). Reaction conditions that should be considered include, but are not limited to, temperature, media flow rate, pH, media redox potential, agitation rate (if using a continuous stirred tank reactor), inoculum level, maximum substrate concentrations and rates of introduction of the substrate to the bioreactor to ensure that substrate level does not become limiting, and maximum product concentrations to avoid product inhibition.

The optimum reaction conditions will depend partly on the particular microorganism of used. However, in general, it is preferred that the fermentation be performed at a pressure higher than ambient pressure. Operating at increased pressures allows a significant increase in the rate of carbon transfer (e.g., methane) from the gas phase to the liquid phase where it can be taken up by the microorganism as a carbon source for the production of isobutanol (or other desired products). This in turn means that the retention time (defined as the liquid volume in the bioreactor divided by the input gas flow rate) can be reduced when bioreactors are maintained at elevated pressure rather than atmospheric pressure.

The use of pressurized systems can greatly reduce the volume of the bioreactor required, and consequently the capital cost of the fermentation equipment. In some cases, reactor volume can be reduced in linear proportion to increases in reactor operating pressure, i.e., bioreactors operated at 10 atmospheres of pressure need only be one tenth the volume of those operated at 1 atmosphere of pressure.

It is also desirable that the rate of introduction of the gaseous carbon substrate (such as methane) is such as to ensure that the concentration of gaseous carbon substrate (such as methane) in the liquid phase does not become limiting. This is because a consequence of carbon substrate (e.g., methane) limited conditions can be that the isobutanol (or other desired product) is consumed by the culture.

### Fermentation Conditions

pH can be optimized based on the microorganism used. For example, the pH used during the methanotroph fermentation of methane to a desired product can be from 4 to 10. In other instances, the pH can be from 5 to 9; 6 to 8; 6.1 to 7.9; 6.2 to 7.8; 6.3 to 7.7; 6.4 to 7.6; or 6.5 to 7.5. For example, the pH can be from 6.6 to 7.4. In some cases, the pH can be from 5 to 9. In some cases, the pH can be from 6 to 8. In some cases, the pH can be from 6.1 to 7.9. In some cases, the pH can be from 6.2 to 7.8. In some cases, the pH can be from 6.3 to 7.7. In some cases, the pH can be from 6.4 to 7.6. In some cases, the pH can be from 6.5 to 7.5. In some cases, the pH used for the fermentation of methanotrophs can be greater than 6.

Temperature can also be adjusted based on the microorganism used. For example, the temperature used during the methanotroph fermentation of methane to a desired product can be from 30° C. to 45° C. In other instances, the temperature of the fermentation can be from 30° C. to 45° C.; 31° C. to 44° C.; 32° C. to 43° C.; 33° C. to 42° C.; 34° C. to 41° C.; 35° C. to 40° C. For example, the temperature can be from 36° C. to 39° C. (e.g., 36° C., 37° C., 38° C., or 39° C.). In some cases, the temperature can be from 30° C. to 45° C. (e.g., 30° C., 31° C., 32° C., 33° C., 34° C., 35° C., 36° C., 37° C., 38° C., 39° C., 40° C., 41° C., 42° C., 43° C., 44° C., or 45° C.). In some cases, the temperature can be from 31° C. to 44° C. (e.g., 31° C., 32° C., 33° C., 34° C., 35° C., 36° C., 37° C., 38° C., 39° C., 40° C., 41° C., 42° C., 43° C., or 44° C.). In some cases, the temperature can be from 32° C. to 43° C. In some cases, the temperature can be from 33° C. to 42° C. (e.g., 33° C., 34° C., 35° C., 36° C., 37° C., 38° C., 39° C., 40° C., 41° C., or 42° C.). In some cases, the temperature can be from 34° C. to 41° C. (e.g., 34° C., 35° C., 36° C., 37° C., 38° C., 39° C., 40° C., or 41° C.). In some cases, the temperature can be from 35° C. to 40° C. (e.g., 35° C., 36° C., 37° C., 38° C., 39° C., or 40° C.).

In some cases, the temperatures can be within one tenth of a degree. For example, in some cases, the temperature of fermentation can be 37.0° C., 37.1° C., 37.2° C., 37.3° C., 37.4° C., 37.5° C., 37.6° C., 37.7° C., 37.8° C., 37.9° C., 38.0° C., 38.1° C., 38.2° C., 38.3° C., 38.4° C., 38.5° C., 38.6° C., 38.7° C., 38.8° C., 38.9° C., 39.0° C., 39.1° C., 39.2° C., 39.3° C., 39.4° C., 39.5° C., 39.6° C., 39.7° C., 39.8° C., 39.9° C., 40.0° C., 40.1° C., 40.2° C., 40.3° C., 40.4° C., 40.5° C., 40.6° C., 40.7° C., 40.8° C., 40.9° C., 41.0° C., 41.1° C., 41.2° C., 41.3° C., 41.4° C., 41.5° C., 41.6° C., 41.7° C., 41.8° C., 41.9° C., 42.0° C., 42.1° C., 42.2° C., 42.3° C., 42.4° C., 42.5° C., 42.6° C., 42.7° C., 42.8° C., 42.9° C., 43.0° C., 43.1° C., 43.2° C., 43.3° C., 43.4° C., 43.5° C., 43.6° C., 43.7° C., 43.8° C., 43.9° C., 44.0° C., 44.1° C., 44.2° C., 44.3° C., 44.4° C., 44.5° C., 44.6° C., 44.7° C., 44.8° C., 44.9° C., 45.0° C., 45.1° C., 45.2° C., 45.3° C., 45.4° C., 45.5° C., 45.6° C., 45.7° C., 45.8° C., 45.9° C., 46.0° C., 46.1° C., 46.2° C., 46.3° C., 46.4° C., 46.5° C., 46.6° C., 46.7° C., 46.8° C., 46.9° C., 47.0° C., 47.1° C., 47.2° C., 47.3° C., 47.4° C., 47.5° C., 47.6° C., 47.7° C., 47.8° C., or 47.9° C.

In some cases, the temperature of fermentation can be from 37.0° C. to 47.9° C. In some cases, the temperature of fermentation can be from 37.1° C. to 47.8° C. In some cases, the temperature of fermentation can be from 37.2° C. to 47.7° C. In some cases, the temperature of fermentation can be from 37.3° C. to 47.6° C. In some cases, the temperature of fermentation can be from 37.4° C. to 47.5° C. In some cases, the temperature of fermentation can be from 37.5° C. to 47.4° C. In some cases, the temperature of fermentation can be from 37.6° C. to 47.3° C. In some cases, the temperature of fermentation can be from 37.7° C. to 47.2° C. In some cases, the temperature of fermentation can be from 37.8° C. to 47.1° C. In some cases, the temperature of fermentation can be from 37.9° C. to 47.0° C. In some cases, the temperature of fermentation can be from 38.0° C. to 46.9° C. In some cases, the temperature of fermentation can be from 38.1° C. to 46.8° C. In some cases, the temperature of fermentation can be from 38.2° C. to 46.7° C. In some cases, the temperature of fermentation can be from 38.3° C. to 46.6° C. In some cases, the temperature of fermentation can be from 38.4° C. to 46.5° C. In some cases, the temperature of fermentation can be from 38.5° C. to 46.4° C. In some cases, the temperature of fermentation can be from 38.6° C. to 46.3° C. In some cases, the temperature of fermentation can be from 38.7° C. to 46.2° C. In some cases, the temperature of fermentation can be from 38.8° C. to 46.1° C. In some cases, the temperature of fermentation can be from 38.9° C. to 46.0° C. In some cases, the temperature of fermentation can be from 39.0° C. to 45.9° C. In some cases, the temperature of fermentation can be from 39.1° C. to 45.8° C. In some cases, the temperature of fermentation can be from 39.2° C. to 45.7° C. In some cases, the temperature of fermentation can be from 39.3° C. to 45.6° C. In some cases, the temperature of fermentation can be from 39.4° C. to 45.5° C. In some cases, the temperature of fermentation can be from 39.5° C. to 45.4° C. In some cases, the temperature of fermentation can be from 39.6° C. to 45.3° C. In some cases, the temperature of fermentation can be from 39.7° C. to 45.2° C. In some cases, the temperature of fermentation can be from 39.8° C. to 45.1° C. In some cases, the temperature of fermentation can be from 39.9° C. to 45.0° C. In some cases, the temperature of fermentation can be from 40.0° C. to 44.9° C. In some cases, the temperature of fermentation can be from 40.1° C. to 44.8° C. In some cases, the temperature of fermentation can be from 40.2° C. to 44.7° C. In some cases, the temperature of fermentation can be from 40.3° C. to 44.6° C. In some cases, the temperature of fermentation can be from 40.4° C. to 44.5° C. In some cases, the temperature of fermentation can be from 40.5° C. to 44.4° C. In some cases, the temperature of fermentation can be from 40.6° C. to 44.3° C. In some cases, the temperature of fermentation can be from 40.7° C. to 44.2° C. In some cases, the temperature of fermentation can be from 40.8° C. to 44.1° C. In some cases, the temperature of fermentation can be from 40.9° C. to 44.0° C. In some cases, the temperature of fermentation can be from 41.0° C. to 43.9° C. In some cases, the temperature of fermentation can be from 41.1° C. to 43.8° C. In some cases, the temperature of fermentation can be from 41.2° C. to 43.7° C. In some cases, the temperature of fermentation can be from 41.3° C. to 43.6° C. In some cases, the temperature of fermentation can be from 41.4° C. to 43.5° C. In some cases, the temperature of fermentation can be from 41.5° C. to 43.4° C. In some cases, the temperature of fermentation can be from 41.6° C. to 43.3° C. In some cases, the temperature of fermentation can be from 41.7° C. to 43.2° C. In some cases, the temperature of fermentation can be from 41.8° C. to 43.1° C. In some cases, the temperature of fermentation can be from 41.9° C. to 43.0° C. In some cases, the temperature of fermentation can be from 42.0° C. to 42.9° C. In some cases, the temperature of fermentation can be from 42.1° C. to 42.8° C. In some cases, the temperature of fermentation can be from 42.2° C. to 42.7° C. In some cases, the temperature of fermentation can be from 42.3° C. to 42.6° C. In some cases, the temperature of fermentation can be from 42.4° C. to 42.5° C.

Availability of oxygen and other gases such as gaseous C₁ carbon substrates (such as methane) can affect overall microorganism biomass accumulation. For example, when considering oxygen availability, the percent of dissolved oxygen (DO) within the media can be from 10% to 40%. In certain instances, the DO concentration can be from 10% to 20%; 15% to 25%; 17% to 30%; 20% to 35%; 25% to 40%. For example, in some cases the DO concentration can be from 10% to 20%. In other cases, the DO can be from 15% to 25%. In some instances, the DO can be from 17% to 30%. In some cases, the DO can be from 20% to 35%. In some cases, the DO can be from 25% to 40%. In some cases, the DO can be 15%. In some cases, the DO can be 20%. In some cases, the DO can be 25%. In some cases, these DO concentrations can be used to grow the number of methanotrophs, e.g., increase overall biomass.

When using a methanotroph, the type of methane substances can have an effect on yield and fermentation rates. For example, natural gas can be used, which typically has a methane content of above 85% (e.g., above 90%) methane. Other components within natural gas can include but are not limited to, ethane, propane, iso-butane, normal-butane, iso-pentane, normal pentane, hexanes plus, nitrogen, carbon dioxide, oxygen, hydrogen, and hydrogen sulfides.

“Pure” methane can be used as well. In these cases, the methane typically comes from a tank. The methane contained within these tanks can range from 90% or greater methane content and the remaining gas are other gases (such as carbon dioxide). For example, gas having a methane content, of greater than 90% can be used during the fermentation process. In certain instances, the methane concentration can be greater than 90%; 91%; 92%; 93%; 94%; 95%; 96%; 97%; 98%; 99%; or 99.9%. In some instances, the methane concentration can be 90% methane and 10% are other gases (such as carbon dioxide). In other instances, the methane concentration can be 91% methane and 9% are other gases (such as carbon dioxide). In some instances, the methane concentration can be 92% methane and 8% are other gases (such as carbon dioxide). In some instances, the methane concentration can be 93% methane and 7% are other gases (such as carbon dioxide). In some instances, the methane concentration can be 94% methane and 6% are other gases (such as carbon dioxide). In some instances, the methane concentration can be 95% methane and 5% are other gases (such as carbon dioxide). In other instances, the methane concentration can be 96% methane and 4% are other gases (such as carbon dioxide). In some instances, the methane concentration can be 97% methane and 3% are other gases (such as carbon dioxide). In some instances, the methane concentration can be 98% methane and 2% are other gases (such as carbon dioxide). In some instances, the methane concentration can be 99% methane and 1% is other gases (such as carbon dioxide). In some instances, the methane concentration can be 99.9% methane and 0.1% is other gases (such as carbon dioxide).

In cases where a switch is used, the media can comprise the molecule that induces or represses the switch. For example, when a lanthanum switch is used to repress the expression of one or more of the genes described herein, the media can comprise lanthanum, which will repress expression of the one or more genes under the control of the switch. In the case of lanthanum any one of the following concentrations can effectively repress expression of the one or more genes: 0.1 μM; 0.5 μM; 1 μM; 2 μM; 3 μM; 4 μM; 5 μM; 6 μM; 7 μM; 8 μM; 9 μM; 10 μM; 12.5 μM; 15 μM; 17.5 μM; 20 μM; 25 μM; 50 μM; 100 μM or more. In one case, 0.1 μM lanthanum can be used to repression expression of the one or more genes under the control of a lanthanum switch. In other cases, at least 0.5 μM lanthanum can be used. In other cases, at least 1 μM lanthanum can be used. In other cases, at least 2 μM lanthanum can be used. In other cases, at least 3 μM lanthanum can be used. In other cases, at least 4 μM lanthanum can be used. In other cases, at least 5 μM lanthanum can be used. In other cases, at least 6 μM lanthanum can be used. In other cases, at least 7 μM lanthanum can be used. In other cases, at least 8 μM lanthanum can be used. In other cases, at least 9 μM lanthanum can be used. In other cases, at least 10 μM lanthanum can be used. In other cases, at least 12.5 μM lanthanum can be used. In other cases, at least 15 μM lanthanum can be used. In other cases, at least 17.5 μM lanthanum can be used. In other cases, at least 20 μM lanthanum can be used. In other cases, at least 25 μM lanthanum can be used. In other cases, at least 50 μM lanthanum can be used. In other cases, at least 100 μM lanthanum can be used. In some cases, a range of 0.5 μM lanthanum to 100 μM lanthanum will effectively repress gene expression. In some cases, a range of 0.5 μM lanthanum to 50 μM lanthanum will repress gene expression. In other cases, a range of 1 μM lanthanum to 20 μM lanthanum will repress gene expression. In some cases, a range of 2 μM lanthanum to 15 μM lanthanum will repress gene expression. In some cases, a range of 3 μM lanthanum to 12.5 μM lanthanum will repress gene expression. In some cases, a range of 4 μM lanthanum to 12 μM lanthanum will repress gene expression. In some cases, a range of 5 μM lanthanum to 11.5 μM lanthanum will repress gene expression. In some cases, a range of 6 μM lanthanum to 11 μM lanthanum will repress gene expression. In some cases, a range of 7 μM lanthanum to 10.5 μM lanthanum will repress gene expression. In some cases, a range of 8 μM lanthanum to 10 μM lanthanum will repress gene expression.

In some cases, the lanthanum in the media can be diluted to turn on expression of the one or more lanthanum repressed genes. For example, in some cases, the dilution of lanthanum containing media can be 1:1 (1 part lanthanum containing media to 1 part non-lanthanum containing media). In some cases, the dilution can be at least 1:2; 1:3; 1:4; 1:5; 1:7.5; 1:10; 1:15; 1:20; 1:25; 1:30; 1:35; 1:40; 1:45; 1:50; 1:75; 1:100; 1:200; 1:300; 1:400; 1:500; 1:1,000; or 1:10,000. For example, in some cases, a 1:2 dilution can be used. In some cases, at least a 1:3 dilution can be used. In some cases, at least a 1:4 dilution can be used. In some cases, at least a 1:5 dilution can be used. In some cases, at least a 1:7.5 dilution can be used. In some cases, at least a 1:10 dilution can be used. In some cases, at least a 1:15 dilution can be used. In some cases, at least a 1:20 dilution can be used. In some cases, at least a 1:25 dilution can be used. In some cases, at least a 1:30 dilution can be used. In some cases, at least a 1:35 dilution can be used. In some cases, at least a 1:40 dilution can be used. In some cases, at least a 1:45 dilution can be used. In some cases, at least a 1:50 dilution can be used. In some cases, at least a 1:75 dilution can be used. In some cases, at least a 1:100 dilution can be used. In some cases, at least a 1:200 dilution can be used. In some cases, at least a 1:300 dilution can be used. In some cases, at least a 1:400 dilution can be used. In some cases, at least a 1:500 dilution can be used. In some cases, at least a 1:1,000 dilution can be used. In some cases, at least a 1:10,000 dilution can be used.

In some cases, the microorganism can be grown in media comprising lanthanum. The media can then be diluted to effectively turn on the expression of the lanthanum repressed genes. The microorganism can be then grown in conditions to promote the production of desired products, such as 2,3-BDO and acetoin (or others disclosed throughout).

In some cases, other rare earth metals can be used. For example, other rare earth metals such as cerium (Ce), dysprosium (Dy), erbium (Er), europium (Eu), gadolinium (Gd), holmium (Ho), lutetium (Lu), neodymium (Nd), praseodymium (Pr), promethium (Pm), samarium (Sm), scandium (Sc), terbium (Tb), thulium (Tm), ytterbium (Yb), yttrium (Y), or any combination thereof, can be used to repress or activate a molecular switch.

### Bioreactor

Fermentation reactions can be carried out in any suitable bioreactor. In some embodiments of the invention, the bioreactor can comprise a first, growth reactor in which the microorganisms are cultured, and a second, fermentation reactor, to which broth from the growth reactor is fed and in which most of the fermentation product (isobutyraldehyde and/or isobutanol, for example) is produced.

### Product Recovery

The fermentation of the microorganisms disclosed herein can produce a fermentation broth comprising a desired product (e.g., isobutyraldehyde and/or isobutanol) and/or one or more by-products as well as the microorganisms (e.g., a genetically modified methanotroph), in the growth/fermentation medium.

The microorganisms and the methods herein can produce isobutyraldehyde and/or isobutanol at surprisingly high efficiency, more so than other known fermentation processes. For example, the microorganisms and the methods disclosed herein can convert a carbon substrate (such as methane) at a rate of greater than 40% of the theoretical maximum. This means that at least 40% of the available carbon within the system is converted into product, such as isobutyraldehyde and/or isobutanol. In some cases, the conversion of a carbon substrate into isobutyraldehyde and/or isobutanol can be at least 41%, 42%, 43%, 44%, 45%, 46%, 47%, 48%, 49%, 50%, 51%, 52%, 53%, 54%, 55%, 56%, 57%, 58%, 59%, or 60%. In some cases, the conversion of a C₁ carbon substrate into isobutyraldehyde and/or isobutanol can be at least 40% of the theoretical maximum. In some cases, the conversion of a C₁ carbon substrate into isobutyraldehyde and/or isobutanol can be at least 41% of the theoretical maximum. In some cases, the conversion of a C₁ carbon substrate into isobutyraldehyde and/or isobutanol can be at least 42% of the theoretical maximum. In some cases, the conversion of a C₁ carbon substrate into isobutyraldehyde and/or isobutanol can be at least 43% of the theoretical maximum. In some cases, the conversion of a C₁ carbon substrate into isobutyraldehyde and/or isobutanol can be at least 43% of the theoretical maximum. In some cases, the conversion of a C₁ carbon substrate into isobutyraldehyde and/or isobutanol can be at least 44% of the theoretical maximum. In some cases, the conversion of a C₁ carbon substrate into isobutyraldehyde and/or isobutanol can be at least 45% of the theoretical maximum. In some cases, the conversion of a C₁ carbon substrate into isobutyraldehyde and/or isobutanol can be at least 46% of the theoretical maximum. In some cases, the conversion of a C₁ carbon substrate into isobutyraldehyde and/or isobutanol can be at least 47% of the theoretical maximum. In some cases, the conversion of a C₁ carbon substrate into isobutyraldehyde and/or isobutanol can be at least 48% of the theoretical maximum. In some cases, the conversion of a C₁ carbon substrate into isobutyraldehyde and/or isobutanol can be at least 49% of the theoretical maximum. In some cases, the conversion of a C₁ carbon substrate into isobutyraldehyde and/or isobutanol can be at least 50% of the theoretical maximum. In some cases, the conversion of a C₁ carbon substrate into isobutyraldehyde and/or isobutanol can be at least 51% of the theoretical maximum. In some cases, the conversion of a C₁ carbon substrate into isobutyraldehyde and/or isobutanol can be at least 52% of the theoretical maximum. In some cases, the conversion of a C₁ carbon substrate into isobutyraldehyde and/or isobutanol can be at least 53% of the theoretical maximum. In some cases, the conversion of a C₁ carbon substrate into isobutyraldehyde and/or isobutanol can be at least 54% of the theoretical maximum. In some cases, the conversion of a C₁ carbon substrate into isobutyraldehyde and/or isobutanol can be at least 55% of the theoretical maximum. In some cases, the conversion of a C₁ carbon substrate into isobutyraldehyde and/or isobutanol can be at least 56% of the theoretical maximum. In some cases, the conversion of a C₁ carbon substrate into isobutyraldehyde and/or isobutanol can be at least 57% of the theoretical maximum. In some cases, the conversion of a C₁ carbon substrate into isobutyraldehyde and/or isobutanol can be at least 58% of the theoretical maximum. In some cases, the conversion of a C₁ carbon substrate into isobutyraldehyde and/or isobutanol can be at least 59% of the theoretical maximum. In some cases, the conversion of a C₁ carbon substrate into isobutyraldehyde and/or isobutanol can be at least 60% of the theoretical maximum.

In certain methods when producing isobutyraldehyde, the overall amount isobutyraldehyde produced can be at least 1 g/L after 72 hours (or other time frame such as 48, 60, 84, 96, 108, or 120 hours). For example, the overall amount of isobutyraldehyde after 72 hours (or other time frame) produced can be at least 3 g/L to 7 g/L, 4 g/L to 8 g/L, 5 g/L to 9 g/L, 6 g/L to 10 g/L, 7 g/L to 11 g/L, 8 g/L to 12 g/L, 9 g/L to 13 g/L, 10 g/L to 14 g/L, 11 g/L to 15 g/L, 12 g/L to 16 g/L, 13 g/L to 17 g/L, 14 g/L to 18 g/L, 15 g/L to 19 g/L, 16 g/L to 20 g/L, 17 g/L to 21 g/L, or 18 g/L to 22 g/L. In some cases, the overall amount of isobutyraldehyde produced can be at least 7 g/L after 72 hours (or other time frame). In some cases, the overall amount of isobutyraldehyde produced can be at least 9 g/L after 72 hours (or other time frame). In some cases, the overall amount of isobutyraldehyde produced can be at least 12 g/L after 72 hours (or other time frame). In some cases, the overall amount of isobutyraldehyde produced can be at least 15 g/L after 72 hours (or other time frame). In some cases, the overall amount of isobutyraldehyde produced can be at least 18 g/L after 72 hours (or other time frame). In some cases, the overall amount of isobutyraldehyde produced can be at least 20 g/L after 72 hours (or other time frame).

In certain methods when producing isobutanol, the amount of isobutanol produced can be at least 1 g/L after 72 hours (or other time frame). For example, the amount of isobutanol produced can be at least 1 g/L to 5 g/L, 2 g/L to 6 g/L, 3 g/L to 7 g/L, or 4 g/L to 8 g/L, 5 g/L to 9 g/L, 6 g/L to 10 g/L, 7 g/L to 11 g/L, 8 g/L to 12 g/L, 9 g/L to 13 g/L, 10 g/L to 14 g/L, 11 g/L to 15 g/L, 12 g/L to 16 g/L, 13 g/L to 17 g/L, 14 g/L to 18 g/L, 15 g/L to 19 g/L, 16 g/L to 20 g/L, 17 g/L to 21 g/L, or 18 g/L to 22 g/L after 72 hours (or other time frame). In some cases, the amount of isobutanol produced can be at least 8 g/L after 72 hours (or other time frame). In some cases, the amount of isobutanol produced can be at least 7 g/L after 72 hours (or other time frame). In some cases, the amount of isobutanol produced can be at least 6 g/L after 72 hours (or other time frame). In some cases, the amount of isobutanol produced can be from 1 g/L to 5 g/L after 72 hours (or other time frame). In some cases, the amount of isobutanol produced can be from 2 g/L to 6 g/L after 72 hours (or other time frame). In some cases, the amount of isobutanol produced can be from 3 g/L to 7 g/L after 72 hours (or other time frame). In some cases, the amount of isobutanol produced can be from 4 g/L to 8 g/L after 72 hours (or other time frame). In some cases, the amount of isobutanol produced can be from 5 g/L to 9 g/L after 72 hours (or other time frame). In some cases, the amount of isobutanol produced can be from 6 g/L to 10 g/L after 72 hours (or other time frame). In some cases, the amount of isobutanol produced can be from 7 g/L to 11 g/L after 72 hours (or other time frame). In some cases, the amount of isobutanol produced can be from 8 g/L to 12 g/L after 72 hours (or other time frame). In some cases, the amount of isobutanol produced can be from 9 g/L to 13 g/L after 72 hours (or other time frame). In some cases, the amount of isobutanol produced can be from 10 g/L to 14 g/L after 72 hours (or other time frame). In some cases, the amount of isobutanol produced can be from 11 g/L to 15 g/L after 72 hours (or other time frame). In some cases, the amount of isobutanol produced can be from 12 g/L to 16 g/L after 72 hours (or other time frame). In some cases, the amount of isobutanol produced can be from 13 g/L to 17 g/L after 72 hours (or other time frame). In some cases, the amount of isobutanol produced can be from 14 g/L to 18 g/L after 72 hours (or other time frame). In some cases, the amount of isobutanol produced can be from 15 g/L to 19 g/L after 72 hours (or other time frame). In some cases, the amount of isobutanol produced can be from 16 g/L to 20 g/L after 72 hours (or other time frame). In some cases, the amount of isobutanol produced can be from 17 g/L to 21 g/L after 72 hours (or other time frame). In some cases, the amount of isobutanol produced can be from 18 g/L to 22 g/L after 72 hours (or other time frame).

In some cases, when methods such as “stripping” are used to isolate isobutanol (or isobutyraldehyde) continuously during fermentation, the amount of isobutanol (or isobutyraldehyde) present in the fermentation broth can be less than 10 g/L. For example, in some cases, the fermentation broth can comprise less than 9 g/L, 8 g/L, 7 g/L, 6 g/L, 5, g/L, 4 g/L, 3 g/L, 2 g/L, or 1 g/L of isobutanol (or isobutyraldehyde) during continuous fermentation. In some cases the fermentation broth titer can be less than 1 g/L of isobutanol (or isobutyraldehyde) during continuous fermentation.

In other cases, when microorganisms are used that normally produce at least some isobutyraldehyde and/or isobutanol, after genetic modification and fermentation, the genetically modified microorganism can produce isobutyraldehyde and/or isobutanol in concentrations that are at least 1.1× the amount that is normally produced. In some cases, the genetically modified microorganism can produce at least 2×, 3×, 4×, 5×, 10×, 25×, 50×, and or 100× the amount that is normally produced. In some cases, the genetically modified microorganism can produce at least 2× the amount that is normally produced. In some cases, the genetically modified microorganism can produce at least 3× the amount that is normally produced. In some cases, the genetically modified microorganism can produce at least 4× the amount that is normally produced. In some cases, the genetically modified microorganism can produce at least 5× the amount that is normally produced. In some cases, the genetically modified microorganism can produce at least 10× the amount that is normally produced. In some cases, the genetically modified microorganism can produce at least 25× the amount that is normally produced. In some cases, the genetically modified microorganism can produce at least 50× the amount that is normally produced. In some cases, the genetically modified microorganism can produce at least 100× the amount that is normally produced.

As discussed above, in certain embodiments the isobutyraldehyde and/or isobutanol produced in the fermentation reaction is converted to other desired products directly from the fermentation broth. In other embodiments, the isobutyraldehyde and/or isobutanol is first recovered from the fermentation broth before conversion to other desired products.

In some cases, isobutyraldehyde and/or isobutanol can be continuously removed from a portion of broth and recovered as purified isobutyraldehyde and/or isobutanol. In particular embodiments, the recovery of isobutyraldehyde and/or isobutanol includes passing the removed portion of the broth containing isobutyraldehyde and/or isobutanol through a separation unit to separate the microorganisms (e.g., genetically modified methanotroph) from the broth, to produce a cell-free isobutyraldehyde and/or isobutanol containing permeate, and returning the microorganisms to the bioreactor. The cell-free isobutyraldehyde and/or isobutanol-containing permeate can then can be stored or be used for subsequent conversion to other desired products.

The recovering of isobutyraldehyde and/or isobutanol and/or one or more other products or by-products produced in the fermentation reaction can comprise continuously removing a portion of the broth and recovering separately isobutyraldehyde and/or isobutanol and one or more other products from the removed portion of the broth. In some embodiments the recovery of isobutyraldehyde and/or isobutanol and/or one or more other products includes passing the removed portion of the broth containing isobutyraldehyde and/or isobutanol and/or one or more other products through a separation unit to separate microorganisms from the isobutyraldehyde and/or isobutanol and/or one or more other products, to produce cell-free isobutyraldehyde and/or isobutanol and one or more other product-containing permeate, and returning the microorganisms to the bioreactor.

In the above embodiments, the recovery of isobutyraldehyde and/or isobutanol and one or more other products can include first removing isobutyraldehyde and/or isobutanol from the cell-free permeate followed by removing the one or more other products from the cell-free permeate. The cell-free permeate can then be returned to the bioreactor.

Isobutyraldehyde and/or isobutanol, or a mixed product stream containing isobutyraldehyde and/or isobutanol, can be recovered from the fermentation broth. For example, methods that can be used can include but are not limited to, fractional distillation or evaporation, pervaporation, and extractive fermentation. For example, stripping, adsorption, pervaporation, membrane solvent extraction, and liquid-liquid extraction can be used.

In liquid-liquid extraction, an extractant is contacted with the fermentation broth to partition the isobutyraldehyde and/or isobutanol between the fermentation broth and the extractant phase. The isobutyraldehyde and/or isobutanol and the extractant are recovered by a separation process, for example by distillation. In the recovery process, the isobutyraldehyde and/or isobutanol can also be separated from any water, non-condensable gas, and/or fermentation by-products which can have been removed from the fermentation broth through use of the extractant.

Pervaporation or vacuum membrane distillation can be used to concentrate isobutyraldehyde and/or isobutanol (Qureshi, N., et al., “Recovery of 2,3-Butanediol by Vacuum Membrane Distillation,” Separation Science and Technology 29:13 (1994)) in water as an extract from the fermentation broth. A microporous polytetrafluoroethylene (PTFE) membrane is used in the integrated process, while a silicone membrane is usually used in pervaporative ethanol or butanol fermentations.

In certain cases, isobutyraldehyde and/or isobutanol and by-products are recovered from the fermentation broth by continuously removing a portion of the broth from the bioreactor, separating microbial cells from the broth (conveniently by filtration, for example), and recovering isobutyraldehyde and/or isobutanol and optionally other alcohols and acids from the broth. Alcohols can conveniently be recovered for example by distillation, and acids can be recovered for example by adsorption on activated charcoal. The separated microbial cells can be returned to the fermentation bioreactor. The cell free permeate remaining after the isobutyraldehyde and/or isobutanol have been removed is returned to the fermentation bioreactor. Additional nutrients can be added to the cell free permeate to replenish the nutrient medium before it is returned to the bioreactor.

Also, if the pH of the broth was adjusted during recovery of isobutyraldehyde and/or isobutanol and/or by-products, the pH should be re-adjusted to a similar pH to that of the broth in the fermentation bioreactor, before being returned to the bioreactor.

In certain embodiments, the isobutyraldehyde and/or isobutanol is continuously recovered from the fermentation broth or bioreactor and fed directly for chemical conversion to one or more desired products, such as gasoline additive or polymers. For example, the isobutyraldehyde and/or isobutanol can be fed directly through a conduit to one or more vessel suitable for chemical synthesis of one or more of the desired products.

### Biomass

After the product is recovered from fermentation media, the remaining material can be spun down and harvested as biomass. This biomass can be cleaned in some cases, and then can be dried. The biomass can then be used as feed for fish, pigs, cows, and other animals. In some instances, the biomass is not dried, and can be used as a wetcake. The wetcake can also be used as animal feed.

In some cases, the biomass can comprises one or more of the microorganisms that are disclosed throughout. In some cases, the biomass can comprise homogeneous microorganisms. In some cases, the biomass can comprise a heterogeneous mix of microorganisms.

In some cases, the microorganism used in the biomass can comprise a methanotroph. In some cases, the methanotroph can be a Methylococcus. In some cases, the Methylococcus can be a Methylococcus capsulatus.

While some embodiments have been shown and described herein, such embodiments are provided by way of example only. Numerous variations, changes, and substitutions will now occur to those skilled in the art without departing from the invention. It should be understood that various alternatives to the embodiments of the invention described herein will be employed in practicing the invention.

### EXAMPLES

### Example 1: Genetic Engineering of Methanotrophs

To engineer a methanotroph to produce isobutyraldehyde or isobutanol, M. capsulatus was used as a starting point several isobutanol biosynthetic genes from a variety of sources. The various plasmids used are disclosed throughout. Generally however, the genes of the isobutanol pathway were expressed or overexpressed within a methanotroph. For example, various different combinations of α-acetolactate synthase (AlsS); ketol-acid reductoisomerase (IlvC); dihydroxy-acid dehydratase (IlvD); 2-keto acid decarboxylase (KDC); and alcohol dehydrogenase (ADH) were transformed into a methanotroph.

In order to produce methanotroph strains that can make isobutyraldehyde from methane, various different combinations of AlsS, ilvCs, ilvDs, and KDCs were transformed into a methanotroph and tested under conditions that promote isobutyraldehyde fermentation. In order to produce methanotroph strains that can produce isobutanol, the isobutyraldehyde strains were additionally transformed with various combinations of ADHs.

### Example 2: Isobutanol Productivity

The various plasmids were transformed into transformation competent methanotroph strains, and the resulting strains (including biological replicate strains) were evaluated for isobutanol production in small scale microtiter plate fermentation or 1 L or 2 L fermentations, using methane as the carbon source. The various strains that were tested are found below.

### Example 3: 2-Keto Acid Decarboxylase (KDCs)

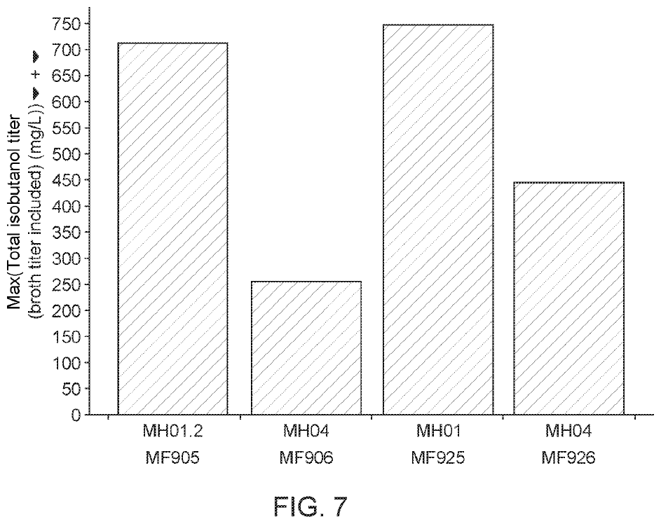

In order to improve isobutanol production, the effects of different KDCs on isobutanol production were compared using the methanotroph system disclosed herein. The isobutanol pathway in E. coli (see e.g., Atsumi et al. 2008 and Atsumi et al. 2010) was compared with the isobutanol pathway designed herein using an M. capsulatus KDC. The data in FIG. 7 shows that KivD (used in the E. coli pathway by Atsumi et al.) does not produce nearly as much isobutanol compared to the M. capsulatus KDC when expressed in a methanotroph. M. capsulatus KDC was found to be better in the context of the full pathway from pyruvate to isobutanol. For reference, MH04 comprises KivD, and MH01 comprises M. capsulatus KDC.

In order to test the effect different KDCs (e.g., Carnobacterium divergens v. Methylococcus capsulatus) have on the production of isobutanol in our strains, several different plasmids (Table 1, below) were transformed into a competent methanotroph strain and the resulting isobutanol production levels were evaluated.

TABLE 1

| | |
|---|---|
| JB140 | p.BAD>g.Cdi.kdc-g.Sce.adh6-Bsu.alsS- |
| | g.Eco.ilvC-g.Mca.ilvD-g.Eco.yqhD |
| SW1357 | p.BAD>Cdi.kdc_Sc.Adh6_Bs.AlsS-Mc.ilvC_Mc_ilvD |
| SW351 | p.BAD>g.Mca.kdc-g.Sce.adh6_g.Bsu.alsS_Ec.ilvC_Ec.ilvD |

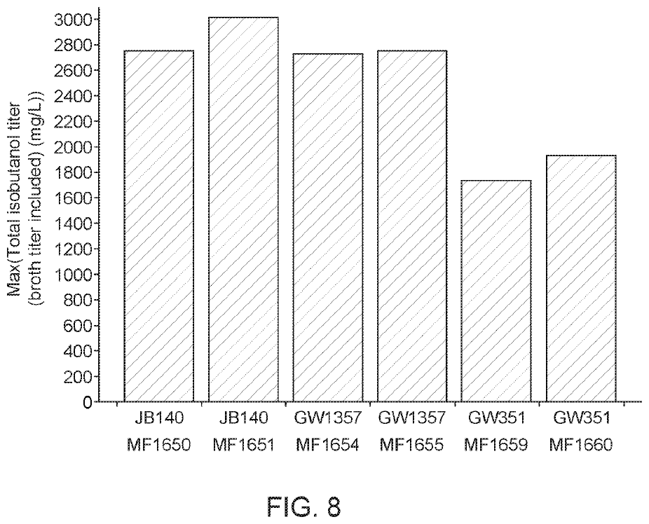

As shown in FIG. 8, methanotrophs transformed with plasmids expressing a KDC from Carnobacterium divergens resulted in an approximate 40% increase over methanotrophs that expressed an endogenous Methylococcus capsulatus KDC.

The difference of overexpression of KDC from Methylococcus capsulatus and Lactococcus lactis was tested. Plasmids comprising isobutanol pathway genes, and KDCs from Methylococcus capsulatus or Lactococcus lactis were constructed and expressed. See Table 2. Methylococcus capsulatus transformants were produced with these plasmids and tested for the ability of the transformants to produce isobutanol.

TABLE 2

| | |
|---|---|
| MH01 | p.BAD>g.Mca.kdc_g.Sce.adh6_g.Bsu.alsS_g.Eco.ilvC_g.Eco.ilvD |
| MH04 | p.BAD>g.Lla.kivD_g.Sce.adh6_g.Bsu.alsS_g.Eco.ilvC_g.Eco.ilvD |

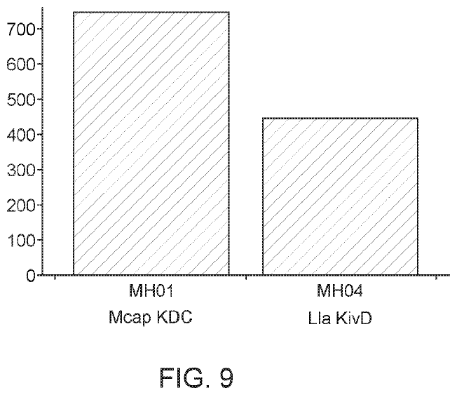

As seen in FIG. 9, KDC from Methylococcus capsulatus showed a vast improvement of isobutanol production compared to Lactococcus lactis KDC. The improvement was approximately a 40% increase.

### Example 4: Alcohol Dehydrogenases (ADHs)

It was determined that not all the aldehydes produced by the genetically modified methanotrophs were converted into alcohols. In order to efficiently produce more alcohols such as isobutanol, different alcohol dehydrogenases were tested in the methanotroph model disclosed herein.

In order to test the effect alcohol dehydrogenases had on the production of isobutanol, several different plasmids were transformed into competent methanotroph strains and the resulting isobutanol production levels were evaluated.

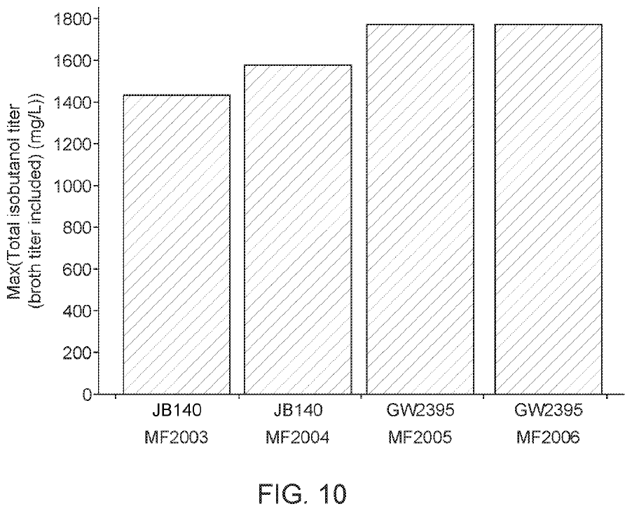

FIG. 10 shows isobutanol titers when methanotrophs are transformed with plasmids expressing either an Ec.fucO (MR2005; MF2006) or an Ec.YqdD (MF2003; MF2004) alcohol dehydrogenase. The methanotrophs expressing the Ec.fucO ADH showed increased isobutanol titers.

Another set of experiments testing different ADHs was performed. Constructs expressing different types of ADHs were created (Table 3, below). All methanotrophs expressed p.BAD>g.Mca.kdc variable ADH_g.Bsu.alsS_g.Eco.ilvC_g.Eco.ilvD. The ADH genes listed in Table 3 below were substituted into the plasmid listed as “variable ADH.” The methanotrophs were then grown up in fermenters (2 L) and isobutanol production was tested.

TABLE 3

| | SEQ | | | |
|---|---|---|---|---|
| | ID NO: | SEQ | | |
| | (Amino | ID NO: | | |
| Ref. # | Acid) | (Nucleotide) | ADH gene | Species |
| 1 | 23 | 24 | Ca_BdhA | C. acetobutylicum |
| 2 | 25 | 26 | Ca_BdhB | C. acetobutylicum |
| 3 | 27 | 28 | Ec_AdhP | E. coli |
| 4 | 29 | 30 | Ec_ahr | E. coli |
| 5 | 31 | 32 | Ec_FucO | E. coli |
| 6 | 33 | 34 | Ec_YjgB | E. coli |
| 7 | 35 | 36 | Ec_YqhD | E. coli |
| 8 | 37 | 38 | Gs_adh | G. stearothermophilus |
| 9 | 39 | 40 | Gs_adh2 | G. stearothermophilus |
| 10 | 41 | 42 | Gt_3237 | G. thermoglucosidas |
| 11 | 43 | 44 | Gt_3823 | G. thermoglucosidas |
| 12 | 45 | 46 | Ll_AdhA.29CB | L. lactis |
| 13 | 47 | 48 | Ll_AdhA | L. lactis |
| 14 | 49 | 50 | Oo_Adh3 | O. oeni |
| 15 | 51 | 52 | Pa_YqhD | P. atrosepticum |
| 16 | 53 | 54 | Psy_MadH | P. cryohalolentis |

Four of the top isobutanol producing plasmids were used that comprised: SL324 (p.BAD>g.Mca.kdc_g.Sce.adh6_g.Bsu.alsS_g.Eco.ilvC_g.Eco.ilvD); JB03 (p.BAD>g.Mca.kdc_g.Cac.BdhB_g.Bsu.alsS_g.Eco.ilvC_g.Eco.ilvD); JB07 (p.BAD>g.Mca.kdc_g.Eco.FucO_g.Bsu.alsS_g.Eco.ilvC_g.Eco.ilvD); and JB09 (p.BAD>g.Mca.kdc_g.Eco.YqhD_g.Bsu.alsS_g.Eco.ilvC_g.Eco.ilvD).

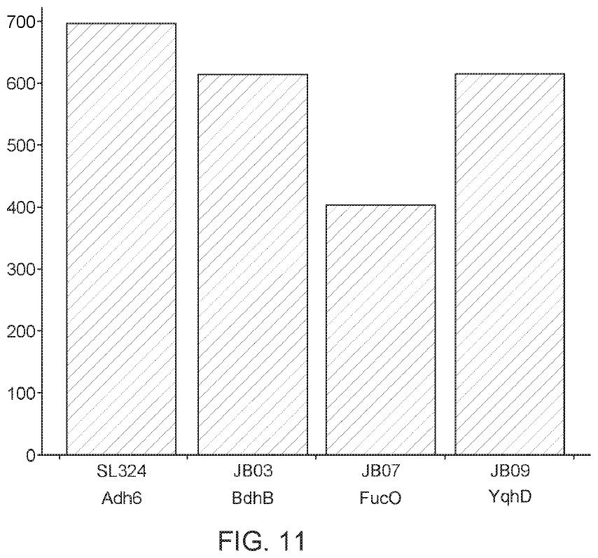

As shown in FIG. 11, Adh6 produced the most isobutanol when testing in a fermenter (2 L). BdhB and YqhD produced similar amounts of isobutanol.

### Example 5: Additional KDCs

Even with the optimization of ADHs, side products from the branch chain amino acid pathway were observed. Therefore, 21 additional KDCs listed in Table 4 were tested.

TABLE 4

| | SEQ | | | |
|---|---|---|---|---|
| | ID NO: | SEQ | | |
| Ref. | (Amino | ID NO: | | |
| No | Acid) | (Nucleotide) | KDC gene | Species |
| 1 | 55 | 56 | Msz.KDC | Methylocaldum szegediense |
| 2 | 57 | 58 | Mla.KDC | Methylosarcina lacus |
| 3 | 59 | 60 | Mde.KDC | Methylomonas denitrificans |
| 4 | 61 | 62 | Mme.KDC | Methylomonas methanica |
| 5 | 63 | 64 | Mcr.KDC | Methylohalobius crimeensis |
| 6 | 65 | 66 | Mma.KDC | Methylobacter marinus |
| 7 | 67 | 68 | Mlu.KDC | Methylobacter luteus |
| 8 | 69 | 70 | Lpu.KDC | Lamprocystis purpurea |
| 9 | 71 | 72 | Ach.KDC | Andreprevotia chitinilytica |
| 10 | 73 | 74 | Lla.KDC2 | Lactococcus lactis |
| 11 | 75 | 76 | Lla2.KDC2 | Lactococcus lactis |
| 12 | 77 | 78 | Sdi.KDC | Streptococcus didelphis |
| 13 | 79 | 80 | Eca.KDC | Enterococcus caccae |
| 14 | 81 | 82 | Eha.KDC | Enterococcus |
| | | | | haemoperoxidus |
| 15 | 83 | 84 | Emo.KDC | Enterococcus moraviensis |
| 16 | 85 | 86 | Cma.KDC | Carnobacterium |
| | | | | maltaromaticum |
| 17 | 87 | 88 | Bth.KDC | Brochothrix thermosphacta |
| 18 | 89 | 90 | Cga.KDC | Carnobacterium gallinarum |
| 19 | 91 | 92 | Cdi.KDC | Carnobacterium divergens |
| 20 | 93 | 94 | Hbi.KDC | Helicobacter bizzozeronii |
| 21 | 95 | 96 | Sau.KDC | Staphylococcus aureus |
| | | | | subsp. aureus CIG290 |
| 22 | 97 | 98 | Fma.KDC | Fictibacillus macauensis |

The following constructs in Table 5 were made and tested in 2 L fermenters:

TABLE 5

| Name | Genes |
|---|---|
| GW1035 | pBAD>Ach.KDC-Sc_Adh6-Bs_AlsS-Mc_IlvC-Mc_IlvD |
| GW1049 | pBAD>Cdi.KDC-Sc_Adh6-Bs_AlsS-Mc_IlvC-Mc_IlvD |
| GW1137 | pBAD>Mc.KDC-Adh6-Bs_AlsS-Mc_IlvC-Mc_IlvD |
| GW1145 | pBAD>Mde.KDC-Sc_Adh6-Bs_AlsS-Mc_IlvC-Mc_IlvD |
| GW1151 | pBAD>Mma-KDC-Sc_Adh6-Bs_AlsS-Mc_IlvC-Mc_IlvD |

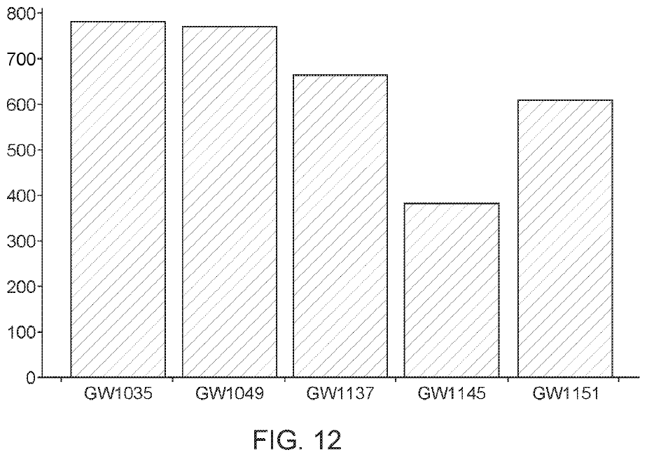

As shown in FIG. 12 KDCs from Andreprevotia chitinilytica or Carnobacterium divergens showed increased ability to produce isobutanol titers at high titers, approximately 0.8 g/L of isobutanol. Andreprevotia chitinilytica or Carnobacterium divergens KDCs produced approximately double that of Methylomonas denitrificans KDC.

### Example 6: Isobutyraldehyde Productivity

In order to increase isobutyraldehyde, strains that did not have any heterologous alcohol dehydrogenases were designed. The same combinations of acetolactate synthase; ketol-acid reductoisomerase; dihydroxy-acid dehydratase; and 2-keto acid decarboxylase were generated and tested for isobutyraldehyde production. Data from two of the following strains are shown: GW692 (pBAD>Mc.KDC_Bs.AlsS_Ec.ILVC_Ec.ILVD); and SL691 (pBAD>Bsu.alsS-g.Cdi.kdc; p.mxaF>g.Mca.kdc-g.Eco.ilvC-g.Mca.ilvD).

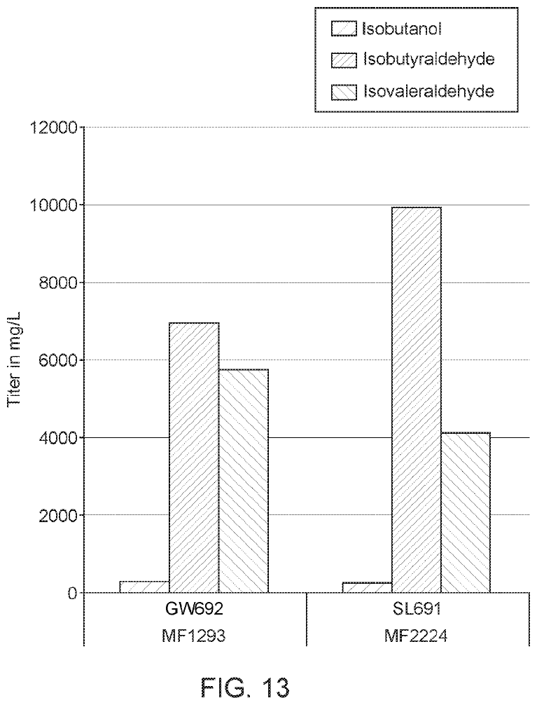

As shown in FIG. 13, the production titers of isobutyraldehyde from the two strains were increased significantly. For example, strain GW692 produced approximately 7 g/L of isobutyraldehyde, whereas strain SL691 produced close to 10 g/L. Strain GW692 also produced approximately 5.8 g/L of isovaleraldehyde, whereas strain SL691 produced approximately 4.1 g/L. Negligible amounts of isobutanol were produced by both strains.

### Example 7: Acetolactate Synthase

In order to increase the levels of 2-acteolactate, several strains were generated in which the acetolactate synthase were optimized. The plasmids described in Table 6 (below), were transformed into a methanotroph. The resulting strains were tested for the ability to utilize any increases of 2-acteolactate. Since 2,3-butanediol titers directly correlate to increases in 2-acetolactate in these strains, 2,3-BDO titers were measured as an indicator of increased 2-acteolactate production. Thus, if additional 2-acetolactate were produced by the differences in acetolactate synthase, there would be a correlating difference in 2,3-BDO titers.

TABLE 6

| | | Average | % diff. |
|---|---|---|---|
| | | Titer | vs. |
| Strain | Strain Genotype | (mg/L) | XZ58 |
| XZ58 | p.BAD>g.Bsu.alsS>(rbs.GTW0001)g. | 372 | 0.0% |
| | Kpn.BudA>p.mxaF>g.Cau.ButA | | |
| XZ557 | p.BAD>g.Blic.alsS->(rbs.GTW0001)g. | 421 | 16.1% |
| | Kpn.BudA-p.mxaF>g.Cau.ButA | | |
| XZ546 | p.BAD>g.Bsu.alsS-(rbs.Mca.MxaF)g. | 373 | 0.3% |
| | Kpn.BudA-p.mxaF>g.Cau.ButA | | |
| XZ562 | p.BAD>g.Blic.alsS-(rbs.Mca.MxaF)g. | 538 | 44.6% |
| | Kpn.BudA-p.mxaF>g.Cau.ButA | | |

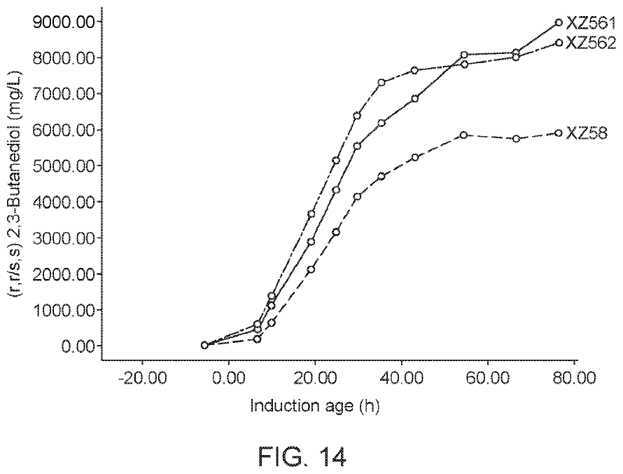

The resulting strains from Table 6 were grown in a small scale microtiter plate fermentation using methane as the carbon source. As shown in Table 6 and in FIG. 14, the strains that expressed Bacillus lichenformis AlsS gene, showed better 2,3-BDO production titers than the strains that expressed Bacillus subtilis AlsS. In one example, a strain that has a substitution of only the AlsS gene (e.g., strains XZ557) exhibited an increase of 2,3-BDO production titer of up to 16.1% compared to strain XZ58. Strain XZ546, a strain having a substitution of only the ribosome binding site for the Kpn.BudA gene, showed virtually no increase of 2,3-BDO titers compared with strain XZ58. However, remarkably, a strain that contained rbs.Mca.MxaF for the Kpn.BudA, instead of a rbs.GTW0001 and expressed a Bacillus licheniformis AlsS gene (e.g., strain XZ562), exhibited a significant increase in 2,3-BDO titers, up to 44.6% compared to strain XZ58. This data indicates that methanotroph strains expressing Bacillus licheniformis AlsS, produce greatly increased levels of 2-acetolactate compared to those expressing Bacillus subtilis AlsS.

### Example 8: Regulating Gene Expression by Using Rare Earth Metals

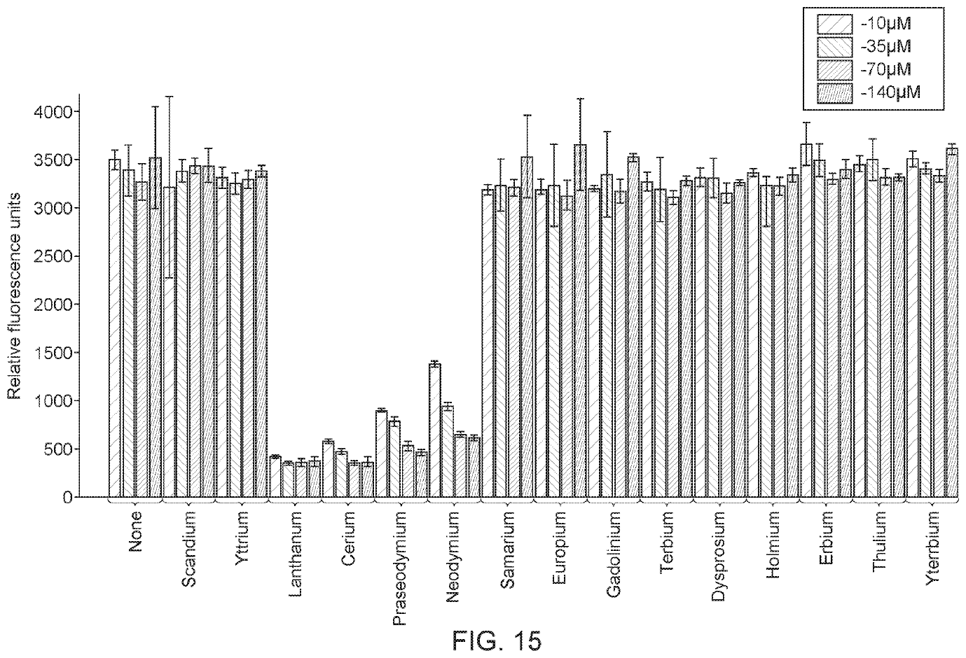

In order to determine whether rare earth metals can be used to modify the expression of genes of the isobutyraldehyde and/or isobutanol pathways, different rare earth metals (at four different concentrations: 10, 35, 70, and 140 μM) were placed into the media in the presence of a Methylococcus capsulatus having a pMxaF promoter driving mCherry expression. The cultures were treated with for 24 hours with the respective rare earth metal. As seen in FIG. 15, lanthanum (La), cerium (Ce), praseodymium (Pr), and neodymium (Nd), repressed the pMxaF promoter. The other rare earth metals did not have an observable repressive effect on the pMxaF promoter.

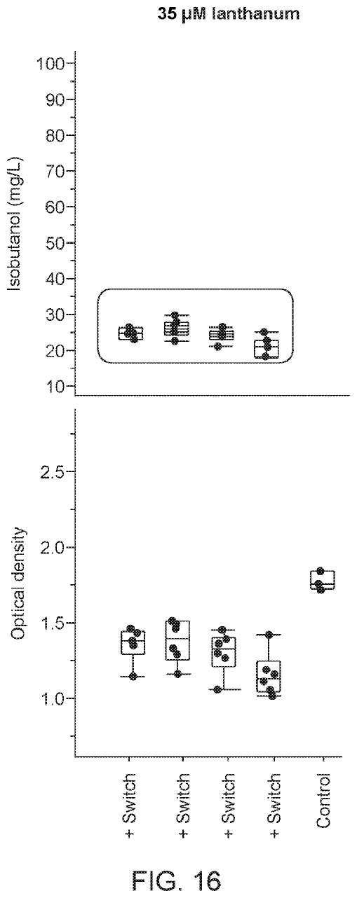

Methylococcus capsulatus expressing isobutanol pathway genes under the control of a rare earth metal switch were grown up in the presence of 35 μM lanthanum in shake bottles. After 24 hours, lanthanum was diluted out and the strain was allowed to ferment isobutanol. As shown in FIG. 16, the strains expressing isobutanol pathway genes under the control of a rare earth metal switch were able to produce isobutanol. The control strain without isobutanol gene did not produce any isobutanol.

### Example 9: Integration of Alcohol Dehydrogenase

In order to examine whether an integrated alcohol dehydrogenase gene could be used to produce isobutanol, a single copy of ADH6 was integrated into a Methylococcus capsulatus strain. Further, a strain expressing an ADH on a plasmid as well as having an integrated ADH was made. The strains were tested for their ability to form isobutanol.

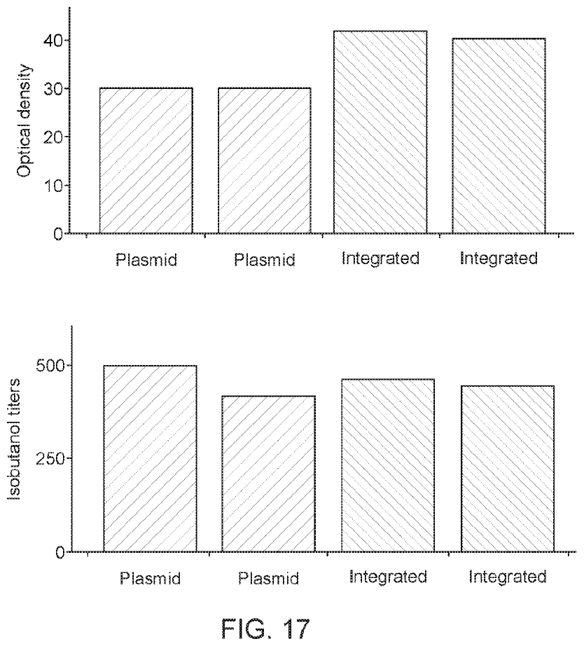

We observed a significant amount of alcohol dehydrogenase activity from the integrated ADH. The integrated ADH functioned as well as non-integrated ADH, as there was no difference in total carbon, alcohol and aldehyde in these strains. (See FIG. 17) Strains expressing both integrated ADH and a plasmid expressing ADH resulted in a better conversion of aldehydes to alcohols.

1. A genetically modified microorganism capable of converting a C₁ carbon source to a multicarbon product, the microorganism comprising heterologous genes encoding:
(a) an acetolactate synthase;
(b) a ketol-acid reductoisomerase;
(c) a dihydroxy-acid dehydratase; and
(d) a 2-keto acid decarboxylase having at least 80% identity to SEQ ID NO: 10.

2. The genetically modified microorganism of claim 1, wherein one or more of the genes is under the control of a rare earth metal switch.

3. The genetically modified microorganism of claim 2, wherein the rare earth metal switch is a lanthanum switch.

4-10. (canceled)

11. The genetically modified microorganism of claim 1, further comprising a gene encoding an alcohol dehydrogenase.

12-23. (canceled)

24. The genetically modified microorganism of claim 1, further comprising a sugar permease gene.

25. The genetically modified microorganism of claim 24, wherein the sugar permease gene is a LacY gene.

26-43. (canceled)

44. The genetically modified microorganism of claim 1, wherein the genetically modified microorganism is a methanotroph.

45. The genetically modified microorganism of claim 44, wherein the methanotroph is from the genus Methylobacter, Methylomicrobium, Methylomonas, Methylocaldum, Methylococcus, Methylosoma, Methylosarcina, Methylothermus, Methylohalobius, Methylogaea, Methylovulum, Crenothrix, Clonothrix, Methylosphaera, Methylocapsa, Methylocella, Methylosinus, Methylocystis, Methyloferula, Methylomarinum, or Methyloacidoiphilum.

46-49. (canceled)

50. The genetically modified microorganism of claim 1, wherein one or more of the genes is overexpressed.

51. The genetically modified microorganism of claim 1, comprising multiple copies of one or more of the genes.

52-78. (canceled)

79. A vector comprising nucleic acids encoding:
(a) an acetolactate synthase;
(b) a ketol-acid reductoisomerase;
(c) a dihydroxy-acid dehydratase; and
(d) a 2-keto acid decarboxylase having at least 80% identity to SEQ ID NO: 10.

80-83. (canceled)

84. The vector of claim 79, wherein one or more of the genes is under the control of a rare earth metal switch.

85. The vector of claim 84, wherein the rare earth metal switch is a lanthanum switch.

86. The vector of claim 79, further comprising a sugar permease gene.

87-88. (canceled)

89. The vector of claim 79, further comprising two or more copies of: the gene encoding the acetolactate synthase; the gene encoding the ketol-acid reductoisomerase; the gene encoding the dihydroxy-acid dehydratase; and/or the gene encoding the 2-keto acid decarboxylase.

90. (canceled)

91. A method of making a genetically modified microorganism capable of converting a C₁ carbon source to a multicarbon product, the method comprising contacting a microorganism with the vector of claim 79.

92-107. (canceled)

108. A method of making an aldehyde from a C₁ carbon source, the method comprising:
(a) contacting a C₁ carbon with the genetically modified microorganism of claim 1; and
(b) growing the genetically modified microorganism to produce the aldehyde.

109-136. (canceled)

137. A method of making an alcohol from a C₁ carbon, the method comprising:
(a) contacting a C₁ carbon with the genetically modified microorganism of claim 11; and
(b) growing the genetically modified microorganism to produce the alcohol.

138-183. (canceled)

184. The genetically modified microorganism of claim 1, wherein the 2-keto acid decarboxylase has at least 90% identity to SEQ ID NO: 10.

185. The vector of claim 79, wherein the 2-keto acid decarboxylase has at least 90% identity to SEQ ID NO: 10.


SEQUENCE LISTING (100 sequences)

SEQ ID NO: 1 - DNA, 1713 bp, Bacillus subtilis
  source 1..1713: mol_type = other DNA
atgaccaaggccaccaaggaacagaaaagcctggtcaagaaccgcggtgctgaactggttgtggactgcctcgtggaacagggcgtgacccatgtcttcggcatcccgggcgccaagatcgacgccgtcttcgacgccctgcaggataaaggtccggaaatcatcgtggcacgccatgagcagaacgcagccttcatggcccaggccgtcggtcggctgacgggtaagcccggcgtggtgctggtcacctccggtccgggagcctcgaacctggccacgggactgctcaccgccaacaccgaaggcgacccggtggtcgccctggccggtaatgtcatccgggcggatcgcctgaagcgcacccatcagtccctggataacgcggccctgttccagccaatcaccaaatatagtgtcgaagtgcaggatgtgaagaacatcccggaagccgtcaccaatgcgttccgaatcgcgtccgccggccaagcaggggcagcattcgtgagcttcccccaggacgtggtcaatgaagtgaccaacaccaaaaacgtcagagccgtagccgccccgaagctgggccctgcagcagatgacgccatctccgctgccatcgcgaagatccagaccgcaaagctgccggtcgtgctggtcggaatgaagggcggacgcccggaggccatcaaggccgtgcgtaaactgctgaagaaggtgcagctaccgttcgtggaaacctaccaggccgccggcaccctgagtcgggacttggaagaccagtatttcggccgtatcggcctgttccgcaaccagccgggcgacctgctcctggaacaagccgatgtggtgctgaccatcggctacgacccgatcgaatatgacccgaagttctggaacatcaatggcgaccgcacgatcatccatctggacgaaatcatcgccgacatcgaccatgcctatcagccggacctggaactgatcggcgacatcccgagcaccatcaaccacatcgaacacgatgccgtgaaggtggaatttgccgaacgcgaacagaagatcctgtcggacctgaagcagtatatgcatgagggcgaacaggtgcctgccgactggaagtcggacagagcccatccgctggaaatcgtgaaggaactgcgtaacgccgtcgacgaccatgtcaccgtcacctgcgatatcggcagccatgccatttggatgagccgctacttccggagctatgaaccgctgaccctgatgatctccaacggtatgcagaccctcggcgtcgccctcccgtgggccatcggcgcaagtctggtgaagccgggcgaaaaagtggtcagcgtgtccggcgacggcggcttcctgttctccgctatggaactggaaaccgcggtccgcctgaaggccccgatcgtgcatatcgtgtggaacgacagcacctacgacatggtcgccttccagcagctgaaaaagtacaaccgcaccagcgccgtggacttcggcaatatcgacatcgtgaagtatgccgaatccttcggagccaccggactgcgcgtggaatccccggaccagctggcggacgttctgcgtcagggcatgaatgccgaaggtcccgtgattatcgatgtgcccgtcgactacagcgacaacatcaacctggcctcggacaaattgccgaaggagttcggcgaactgatgaaaacaaaagcactataa

SEQ ID NO: 2 - protein, 570 aa, Bacillus subtilis
MTKATKEQKSLVKNRGAELVVDCLVEQGVTHVFGIPGAKIDAVFDALQDKGPEIIVARHEQNAAFMAQAVGRLTGKPGVVLVTSGPGASNLATGLLTANTEGDPVVALAGNVIRADRLKRTHQSLDNAALFQPITKYSVEVQDVKNIPEAVTNAFRIASAGQAGAAFVSFPQDVVNEVTNTKNVRAVAAPKLGPAADDAISAAIAKIQTAKLPVVLVGMKGGRPEAIKAVRKLLKKVQLPFVETYQAAGTLSRDLEDQYFGRIGLFRNQPGDLLLEQADVVLTIGYDPIEYDPKFWNINGDRTIIHLDEIIADIDHAYQPDLELIGDIPSTINHIEHDAVKVEFAEREQKILSDLKQYMHEGEQVPADWKSDRAHPLEIVKELRNAVDDHVTVTCDIGSHAIWMSRYFRSYEPLTLMISNGMQTLGVALPWAIGASLVKPGEKVVSVSGDGGFLFSAMELETAVRLKAPIVHIVWNDSTYDMVAFQQLKKYNRTSAVDFGNIDIVKYAESFGATGLRVESPDQLADVLRQGMNAEGPVIIDVPVDYSDNINLASDKLPKEFGELMKTKAL

SEQ ID NO: 3 - DNA, 1476 bp, Escherichia coli
  source 1..1476: mol_type = other DNA
atggctaactacttcaacaccctgaacttgcgtcagcagctggcccagctgggtaagtgccggttcatgggccgtgatgagttcgccgatggcgccagctacctgcagggcaaaaaggtggtgatcgtgggctgcggagcccagggcctgaaccagggcctgaatatgcgcgatagcggcctggacatctcctatgctctgcgcaaggaagcgatcgcggaaaagcgggcatcctggcgcaaggccaccgaaaacggtttcaaagtgggcacctacgaagaactgatcccgcaggccgatttggtcataaacctgaccccggacaagcagcattccgatgtggttcgcaccgtccagccgctgatgaaggacggggcagccctgggttactcccacggcttcaacatcgtggaagtcggcgaacagatccgcaaggacatcaccgtcgtcatggtcgcaccgaagtgtccgggcaccgaagtccgggaagaatataagcgcggattcggcgtaccgaccctgatcgccgtccatcccgaaaacgacccgaagggcgaaggcatggccatcgccaaggcctgggctgccgccaccggaggccatcgcgctggcgtgctggaaagctcgttcgtcgccgaagtgaagagcgacctgatgggcgaacagaccatcctgtgcggcatgctgcaggccggtagcctgctgtgtttcgacaagctggtcgaagaaggcaccgaccctgcgtatgccgaaaagctgatccagttcggctgggaaaccatcaccgaagcgctgaaacagggcggtatcaccctgatgatggaccgcctgtcgaaccctgccaagttacgtgcctatgccctgagcgaacagctgaaggaaatcatggcgcctctgttccagaaacatatggacgatatcatcagcggcgagttcagctccggcatgatggcggactgggcgaatgacgacaagaagctgctgacctggcgggaagaaaccggcaagacggccttcgaaaccgcaccgcagtacgaaggcaaaatcggcgaacaggagtacttcgacaaaggcgtcctgatgatcgcgatggtcaaggcgggagtcgaactggccttcgaaacaatggtcgatagcggcatcatcgaggaatccgcctactacgaaagcctgcatgaactcccgctgatcgccaataccatagcccgcaagcggctgtacgaaatgaacgtcgtgatctccgacactgccgaatacggcaattatctcttctcctatgcctgcgtgccgctcctgaagcccttcatggccgaactgcagccaggcgacctggggaaggcgatccccgaaggcgctgtcgacaacggccagctgcgcgatgtcaacgaagccattcgctcccatgccatcgagcaggtcggcaagaagctgcgtggctatatgacggacatgaagcgcatcgcagtagccggataa

SEQ ID NO: 4 - protein, 491 aa, Escherichia coli
MANYFNTLNLRQQLAQLGKCRFMGRDEFADGASYLQGKKVVIVGCGAQGLNQGLNMRDSGLDISYALRKEAIAEKRASWRKATENGFKVGTYEELIPQADLVINLTPDKQHSDVVRTVQPLMKDGAALGYSHGFNIVEVGEQIRKDITVVMVAPKCPGTEVREEYKRGFGVPTLIAVHPENDPKGEGMAIAKAWAAATGGHRAGVLESSFVAEVKSDLMGEQTILCGMLQAGSLLCFDKLVEEGTDPAYAEKLIQFGWETITEALKQGGITLMMDRLSNPAKLRAYALSEQLKEIMAPLFQKHMDDIISGEFSSGMMADWANDDKKLLTWREETGKTAFETAPQYEGKIGEQEYFDKGVLMIAMVKAGVELAFETMVDSGIIEESAYYESLHELPLIANTIARKRLYEMNVVISDTAEYGNYLFSYACVPLLKPFMAELQPGDLGKAIPEGAVDNGQLRDVNEAIRSHAIEQVGKKLRGYMTDMKRIAVAG

SEQ ID NO: 5 - DNA, 1851 bp, Escherichia coli
  source 1..1851: mol_type = other DNA
atgccgaagtatcggtcagccaccactacacatggccgcaacatggcaggcgctcgtgccctgtggcgtgctaccggcatgaccgatgccgacttcggcaagccgatcatcgccgtggtcaactctttcacccagttcgtcccagggcacgttcatctgcgcgacctgggcaagctggtggccgaacagatcgaggccgcaggtggcgtcgcgaaagagttcaacaccatcgccgtcgacgatggcatcgctatgggccacggtggcatgctgtatagcctgccgtcccgcgaactgatcgccgatagcgtcgaatatatggtgaacgcccattgcgccgatgctatggtgtgcatcagcaactgcgacaagatcacaccggggatgctgatggccagcctgcggctgaacatcccggtgatcttcgtgagcggtggcccgatggaagccggcaagaccaagctgtcggatcagatcatcaagctggatctggtcgacgccatgatccaaggtgccgatccgaaggtgagcgactcccagtccgatcaggtggaacggagcgcctgcccgacttgcggctcatgcagcggcatgttcaccgccaactccatgaattgcctgacggaagccctgggcctgtcccagcccggtaacgggagcctgttggcgacccatgccgaccgcaagcagctgttcctgaatgccggcaagcgcatcgtggaactgaccaagcgctattatgaacagaacgacgaatccgccctgccccgtaatatcgcctcaaaagccgccttcgaaaacgccatgaccctggacatcgctatgggtggcagcaccaacaccgtgctgcacctgctggctgccgctcaggaagccgagatcgacttcaccatgtccgacatcgacaagctgagtcggaaggtgccgcagctgtgcaaggtggcaccgtccacccagaagtatcatatggaagacgtgcatcgcgcaggcggtgtgatcggcatcctgggcgaactggatcgcgctggcctgctgaatcgcgatgtgaagaacgtcctgggcctgaccctgccgcagaccctggaacagtacgacgttatgctgacccaggatgatgccgtcaagaatatgttccgcgcaggccctgccggcattcgcaccacccaagccttcagccaggactgccggtgggataccctggatgacgatcgcgccaatggctgcatccgtagcctggaacatgcctattccaaggatggcggtctggccgttctgtatggcaacttcgcggaaaacggctgcatcgtcaagaccgctggcgtggatgattcgatcctgaagttcaccggaccggccaaagtctacgaaagccaggacgatgccgtcgaagccatcctgggaggcaaggtcgtcgccggagatgtcgtggtgatccgctatgaaggcccgaaaggcggtccgggcatgcaggagatgctgtatccgacctccttcttaaagagcatgggcttgggcaaagcgtgtgcgctcatcaccgatggccgcttcagtggcggcaccagcggcctgtccatcggccatgtctcgccggaagccgccagtggcggcagcatcggcctcatcgaagacggcgatctgatcgccatcgatatcccgaatcgtggcatccagctgcaggtgtccgacgccgaactagccgcacggagggaagcgcaggatgcccgaggcgacaaggcctggaccccgaagaaccgtgaacgtcaggtgtccttcgcgcttcgcgcctatgccagcctggccaccagcgccgataagggtgccgtgcgcgacaagagcaaactaggaggataa

SEQ ID NO: 6 - protein, 616 aa, Escherichia coli
MPKYRSATTTHGRNMAGARALWRATGMTDADFGKPIIAVVNSFTQFVPGHVHLRDLGKLVAEQIEAAGGVAKEFNTIAVDDGIAMGHGGMLYSLPSRELIADSVEYMVNAHCADAMVCISNCDKITPGMLMASLRLNIPVIFVSGGPMEAGKTKLSDQIIKLDLVDAMIQGADPKVSDSQSDQVERSACPTCGSCSGMFTANSMNCLTEALGLSQPGNGSLLATHADRKQLFLNAGKRIVELTKRYYEQNDESALPRNIASKAAFENAMTLDIAMGGSTNTVLHLLAAAQEAEIDFTMSDIDKLSRKVPQLCKVAPSTQKYHMEDVHRAGGVIGILGELDRAGLLNRDVKNVLGLTLPQTLEQYDVMLTQDDAVKNMFRAGPAGIRTTQAFSQDCRWDTLDDDRANGCIRSLEHAYSKDGGLAVLYGNFAENGCIVKTAGVDDSILKFTGPAKVYESQDDAVEAILGGKVVAGDVVVIRYEGPKGGPGMQEMLYPTSFLKSMGLGKACALITDGRFSGGTSGLSIGHVSPEAASGGSIGLIEDGDLIAIDIPNRGIQLQVSDAELAARREAQDARGDKAWTPKNRERQVSFALRAYASLATSADKGAVRDKSKLGG

SEQ ID NO: 7 - DNA, 1689 bp, Methylococcus capsulatus
  source 1..1689: mol_type = other DNA
atgaccgacaagcacccccgtccccattcgtcccaggtcgtcgacggcatggagcgggccccgagccgcgcgatgctgcacgccgtcggcttcgccgatgccgacttcgccaaaccgcagatcggcatcgcttccacctgggcgatggtgacgccgtgcaacatgcacatcaacaagctcgccgaggacgcagcacgcggcgtcgacggcggcggcggcaaggcagtgatcttcaacaccatcaccatttccgacggcatctcgatgggcaccgaaggaatgaaatactccctcgtgtcgcgggaagtcatcgccgactcgatcgaaaccgtggtggcctgtcagggttatgacggcgtggtcgccatcggcggctgcgacaagaacatgcccggctgcctgatcgccctcgcccgcctcaaccgtccggcggtgttcgtctatggcggcaccatcctgccgggctgccacgacggcaagaagctggacgtggtgtcggtgttcgaagcggtcggcgcccgcgccaaccaccgcatcgacgatgccgaactgcacgccatcgaatccaatgccatccccggtccgggctcctgcggtggcatgtataccgcgaacacgatggcctccgccatcgaggcattagggatgagcctgccgggcagttcggcccaggtggccatttcccgcgccaaggaactggattgcgagcgggccggcgcccaggtcctcaagctcctggacctggggctcaaaccccgcgacatcatgaccaagaaggcgttcgagaacgccatcacggtggtgatcgccctgggcggctccaccaacgccgtgctgcacctcctggccatggccaacgcctgcggcgtcgacctgaagctcgacgatttcacccgcatcgggcgcaaagtgccgatgctggcggatctgaaacccagcggcagatactcgatggccgaactggtggaaatcggcggcatccagccgctgatgaagaccttgctggacgcgggactcctgcacggcgactgcatgaccgtaaccggcaagaccctggaagaaaacctggccgacgcgcccgactacccggccggacaagacatgatccggtcgctggacaaccccatcaaaaaggacagccatctggtgatcctcaagggcaacctggcgccggaaggcgcggtcgccaagatcaccggcaaggaaggactgagcttcaccggcaccgcccgcgtattcgactgcgaggaagcggcgctcacggccatcctcgacggcacgatcgtgaaaggcgacgtaatcgtcatccgctatgaaggccccaagggcggccccggcatgcgcgagatgctctcgccgacctcggcggtcatgggcaagggattgggcaaggaggtcgccctcatcaccgacggccgcttttccggcggcacccacggcttcgtggtcggccacatcacgccggaagcctacaccggcggccccctggcgatcgtccgggacggcgataccatcaccatcgacgccgaaacccgcgaattgagcctgcacgtcaccgacgatgaaatcggccggcgcctggcgcagtggactcaaccggcgccgcgctacaccaagggcgtgctggccaaatacgccaggttggtgagcccggcctcggaaggcgccgtcaccgacgacggcctctga

SEQ ID NO: 8 - protein, 562 aa, Methylococcus capsulatus
MTDKHPRPHSSQVVDGMERAPSRAMLHAVGFADADFAKPQIGIASTWAMVTPCNMHINKLAEDAARGVDGGGGKAVIFNTITISDGISMGTEGMKYSLVSREVIADSIETVVACQGYDGVVAIGGCDKNMPGCLIALARLNRPAVFVYGGTILPGCHDGKKLDVVSVFEAVGARANHRIDDAELHAIESNAIPGPGSCGGMYTANTMASAIEALGMSLPGSSAQVAISRAKELDCERAGAQVLKLLDLGLKPRDIMTKKAFENAITVVIALGGSTNAVLHLLAMANACGVDLKLDDFTRIGRKVPMLADLKPSGRYSMAELVEIGGIQPLMKTLLDAGLLHGDCMTVTGKTLEENLADAPDYPAGQDMIRSLDNPIKKDSHLVILKGNLAPEGAVAKITGKEGLSFTGTARVFDCEEAALTAILDGTIVKGDVIVIRYEGPKGGPGMREMLSPTSAVMGKGLGKEVALITDGRFSGGTHGFVVGHITPEAYTGGPLAIVRDGDTITIDAETRELSLHVTDDEIGRRLAQWTQPAPRYTKGVLAKYARLVSPASEGAVTDDGL

SEQ ID NO: 9 - DNA, 1647 bp, Carnobacterium divergens
  source 1..1647: mol_type = other DNA
atgtataccgtgggcgactatctcctggagcggctctcggaactgggcatcaaagagatcttcggcgtgccgggcgactacaacctgaagttcctggatcacatcgtggagcatccgaacctgaagtggatcggcaacgcgaatgaactcaacgcggcgtatgccgccgacggctacgcccgcacgaagggcgtctccgcgctggtgaccaccttcggcgtcggcgagctctccgccatcaacggcatcgccggctcgtatgccgagaaagtcccggtcatccagatcgtgggcagccccacgatggcggtgcagaatgcccataagctggtgcatcataccctgggcgatggcaaattcgaccacttcgagaacatgcatgagtccgtcaccgaagccatcggcagcctcaccaaggagaacgcggtgaccgagatcgatcgcgtgctgcgggccgccgtgctcaaacggcgcccggtgtatctgaacctcccgatcgacgtggccgaaatggtcgtcgaaaaaccgtcgggccccctgctgcccaagcaggcgagcctgagcgcccgcgaggtcgaactcgtgcatgagctggagaaggccctgcagcaggcgaagaacccggtggtcctggcgggcaacgagctggcgtcgttccacctcgaaacgtacctcgccgacttcatccacaagttcaacctccccatcacgaccctccccttcggcaagggcgtcttcaacgaggaagacgagcattatctgggcgtctatgcgggctcgccgaccgaagaaggcctgcggaagcgcgtcgatacggcggacctggtcgtggcgctgggcgcgaagctgacggactccgccacctccggcttctcgtacgacttctccgaaaaacagctcttcagcctggcgtccgacgaagtcatcgtcaaagaggaacacctcgaaggcatccatctgccggccgtcatgaaggcgctgacgagcatcgactaccagggctaccagggcgacatccagccgatggcccggctgaagagcatcaaacccaccaaccaggtgctgacccagcgccacttctgggaggccatcgaaggcttcctggaaaagggcgacaccgccgtcgcggagcagggcacgagcttcttcggcctctcgaccgtgccgctgaagagcgaaatgtcgttcatcggccagccgctgtggggctccatcggctatacgttcccggcgatgctgggcagccagctcgccaacccgtccagccggcacctcctgttcatcggcgacggcagcctgcagctgacgatccaggagctcggcatggccctccgcgaaaaactcaccccgatcgtgttcgtcatcaacaataacggctatacggtcgaacgggaaatccacggcccgaatgaaatctataacgacatcccgatgtgggactaccagaaactcccgctcgtcttcggcggctccgagcagtcggtcatcacctataaagtgacgaccgaactggaactggcgaacgcgctcaaggcggcccggctggacaacaaccgcctgcagtggatcgaagtggtgatggaccagaccgatgcgccggagctcctcatgaagctgggcaagatcttcgcgaagcagaatagctga

SEQ ID NO: 10 - protein, 548 aa, Carnobacterium divergens
MYTVGDYLLERLSELGIKEIFGVPGDYNLKFLDHIVEHPNLKWIGNANELNAAYAADGYARTKGVSALVTTFGVGELSAINGIAGSYAEKVPVIQIVGSPTMAVQNAHKLVHHTLGDGKFDHFENMHESVTEAIGSLTKENAVTEIDRVLRAAVLKRRPVYLNLPIDVAEMVVEKPSGPLLPKQASLSAREVELVHELEKALQQAKNPVVLAGNELASFHLETYLADFIHKFNLPITTLPFGKGVFNEEDEHYLGVYAGSPTEEGLRKRVDTADLVVALGAKLTDSATSGFSYDFSEKQLFSLASDEVIVKEEHLEGIHLPAVMKALTSIDYQGYQGDIQPMARLKSIKPTNQVLTQRHFWEAIEGFLEKGDTAVAEQGTSFFGLSTVPLKSEMSFIGQPLWGSIGYTFPAMLGSQLANPSSRHLLFIGDGSLQLTIQELGMALREKLTPIVFVINNNGYTVEREIHGPNEIYNDIPMWDYQKLPLVFGGSEQSVITYKVTTELELANALKAARLDNNRLQWIEVVMDQTDAPELLMKLGKIFAKQNS

SEQ ID NO: 11 - DNA, 1650 bp, Methylococcus capsulatus
  source 1..1650: mol_type = other DNA
atgggcacggttgagcctggcgctatcggacaacatctgctcgcctgcctttaccaggcgggcgtcgggcacatcttcggcgttcccggcgattacgtgctgggcttctatgatctgatggccaaaggtcccgtccggcatatcgggaccacgcgggaggacaccgccgccttcgccgccgacggctatgcccgctgccggggcatgggcgccctggcggtgacttacggggtcggtgcgctcaacaccgtcaacgccgtcgccggcgcctatgcggaatcctcgccggtggtggtcatcagcggtgcgccgggggtgcgcgagcaaagggaagacccgttgatccaccaccgcttcgggccgttccggttccagcgcgagatattcgaacggatcacctgcgccgccgtggtgctggacgatccggtgatcgccttccggcaggtggagcgtgcgctcgcagccgcccgtcagcactgcaagccggtgtacatcgagattcccgccgaccgggtgatggcgccgggatatccgattccacaggaaaccccggaaacgccttccagcgacgattcggccctggcggaggcggtcgccgaggccgcggagctcctgggccgtgcggtgtcgccggtgatccttgcaggcgtcgagttgcaccggcgagggctccaggacgccctcgtcggcctcgtcgagcaggcccgcctgccggtggcggcgaccttgaccggcaagtcggtgttcgccgagcgccatcccgcctatctgggggtgtacgagggtgcgatgagcacggaaaacgcccgctacatggtcgagcagtccgacctcctgctgatgctcggggtcacgctgaacgatgtcgacacgggcatctacacggcgcgtctcgatccgcagcgcatcgtccgcgcagcccagaacgaggtcgtgattcgccatcaccgctatccccgcgtcctgctcgcggacttcgtcacggccctggcgcggtccgtcaaggcccggggcgaggcgtttccgatgccggcggggccggaaccgtgggactttcccgcgccggaccggccgatgacgatcgcccggctggtggagcggctcgaccgcgccctgacctccgacatgatcgtagtgtgcgatgtcggcgactgcctgttcgcagccaccgacctgcgcgtgcacgagcgcagcgaatttctggcgtccgccttctatacctcgatggggttcgcggtgcccgccgccctcggggcccagatcgcccgtccggaccaccgggcgctgatcctggtcggcgacggtgccttccagatgaccggaacggagctgtcgacccatgcccgtctcggcctggcgcccatcgtggtggtgctcgacaatcgcggttacagcaccgagcgcttcatcctcgacggagccttcaacgacatcgccgactggcgcttccaccggctgggcgaggtgttcggccccctacagggctacgacgcgcccgacgaagcggcgttcgaaaacgcgctcagcgaagcgctggtcaaccgaaacatgccgagcctcatcaacgtccgtctttcccccggcgatgcctcgatagccatgaagcgtctcgccgggcatctgcagtgccgggtcaagggcgagggctaa

SEQ ID NO: 12 - protein, 549 aa, Methylococcus capsulatus
MGTVEPGAIGQHLLACLYQAGVGHIFGVPGDYVLGFYDLMAKGPVRHIGTTREDTAAFAADGYARCRGMGALAVTYGVGALNTVNAVAGAYAESSPVVVISGAPGVREQREDPLIHHRFGPFRFQREIFERITCAAVVLDDPVIAFRQVERALAAARQHCKPVYIEIPADRVMAPGYPIPQETPETPSSDDSALAEAVAEAAELLGRAVSPVILAGVELHRRGLQDALVGLVEQARLPVAATLTGKSVFAERHPAYLGVYEGAMSTENARYMVEQSDLLLMLGVTLNDVDTGIYTARLDPQRIVRAAQNEVVIRHHRYPRVLLADFVTALARSVKARGEAFPMPAGPEPWDFPAPDRPMTIARLVERLDRALTSDMIVVCDVGDCLFAATDLRVHERSEFLASAFYTSMGFAVPAALGAQIARPDHRALILVGDGAFQMTGTELSTHARLGLAPIVVVLDNRGYSTERFILDGAFNDIADWRFHRLGEVFGPLQGYDAPDEAAFENALSEALVNRNMPSLINVRLSPGDASIAMKRLAGHLQCRVKGEG

SEQ ID NO: 13 - DNA, 1083 bp, Saccharomyces cerevisiae
  source 1..1083: mol_type = other DNA
atgagctatcccgagaagttcgaggggatcgccatccagagccacgaggactggaagaacccgaaaaagaccaagtatgatccgaagcccttctacgatcacgacatcgacatcaagatcgaggcctgcggcgtctgcggcagcgatatccattgtgcggctggccactggggcaacatgaagatgccgttggtcgtcggccacgagatcgtgggcaaggtcgtgaagttaggcccgaaaagcaacagcggcttgaaggtgggccagcgcgtgggtgtgggtgcgcaggtcttcagctgtctggagtgcgaccgttgcaagaacgacaacgaaccgtactgcaccaagttcgtcaccacctactcgcagccctacgaggacggctacgtctcgcagggcggttacgccaactatgtccgagtccacgaacacttcgtggtgcccatcccggaaaatatccccagccatctggcggctcccctgctgtgcggtggcttgaccgtctacagccccctcgtccgcaatggctgcggtcccggcaagaaggtgggtatcgtgggcctcggcggtataggctctatgggcacgctgatctcgaaagcgatgggcgcagaaacgtacgtgatctcgcgttcctcgcgcaagcgcgaggatgcgatgaagatgggtgcggaccactacatcgccacgctggaggagggtgactggggtgagaagtacttcgacacgttcgacctcatcgtggtgtgcgcgagttccctgacggacatcgacttcaatatcatgcccaaggcgatgaaggtcggagggcgcatcgtctccatctcgatcccggagcagcacgaaatgctgtcgctgaagccctacggcctgaaagccgtctccattagctactcggcgctcggtagtatcaaggagctcaaccagctgttgaagttggtttccgaaaaggacatcaagatctgggtggaaacgctcccggtgggcgaagccggtgtgcacgaggcctttgagcggatggagaagggggatgtccgttatcggtttacactcgtcggctacgataaagagttctcggattaa

SEQ ID NO: 14 - protein, 360 aa, Saccharomyces cerevisiae
MSYPEKFEGIAIQSHEDWKNPKKTKYDPKPFYDHDIDIKIEACGVCGSDIHCAAGHWGNMKMPLVVGHEIVGKVVKLGPKSNSGLKVGQRVGVGAQVFSCLECDRCKNDNEPYCTKFVTTYSQPYEDGYVSQGGYANYVRVHEHFVVPIPENIPSHLAAPLLCGGLTVYSPLVRNGCGPGKKVGIVGLGGIGSMGTLISKAMGAETYVISRSSRKREDAMKMGADHYIATLEEGDWGEKYFDTFDLIVVCASSLTDIDFNIMPKAMKVGGRIVSISIPEQHEMLSLKPYGLKAVSISYSALGSIKELNQLLKLVSEKDIKIWVETLPVGEAGVHEAFERMEKGDVRYRFTLVGYDKEFSD

SEQ ID NO: 15 - DNA, 1164 bp, Escherichia coli
  source 1..1164: mol_type = other DNA
atgaacaactttaatctgcacaccccaacccgcattctgtttggtaaaggcgcaatcgctggtttacgcgaacaaattcctcacgatgctcgcgtattgattacctacggcggcggcagcgtgaaaaaaaccggcgttctcgatcaagttctggatgccctgaaaggcatggacgtgctggaatttggcggtattgagccaaacccggcttatgaaacgctgatgaacgccgtgaaactggttcgcgaacagaaagtgactttcctgctggcggttggcggcggttctgtactggacggcaccaaatttatcgccgcagcggctaactatccggaaaatatcgatccgtggcacattctgcaaacgggcggtaaagagattaaaagcgccatcccgatgggctgtgtgctgacgctgccagcaaccggttcagaatccaacgcaggcgcggtgatctcccgtaaaaccacaggcgacaagcaggcgttccattctgcccatgttcagccggtatttgccgtgctcgatccggtttatacctacaccctgccgccgcgtcaggtggctaacggcgtagtggacgcctttgtacacaccgtggaacagtatgttaccaaaccggttgatgccaaaattcaggaccgtttcgcagaaggcattttgctgacgctaatcgaagatggtccgaaagccctgaaagagccagaaaactacgatgtgcgcgccaacgtcatgtgggcggcgactcaggcgctgaacggtttgattggcgctggcgtaccgcaggactgggcaacgcatatgctgggccacgaactgactgcgatgcacggtctggatcacgcgcaaacactggctatcgtcctgcctgcactgtggaatgaaaaacgcgataccaagcgcgctaagctgctgcaatatgctgaacgcgtctggaacatcactgaaggttccgatgatgagcgtattgacgccgcgattgccgcaacccgcaatttctttgagcaattaggcgtgccgacccacctctccgactacggtctggacggcagctccatcccggctttgctgaaaaaactggaagagcacggcatgacccaactgggcgaaaatcatgacattacgttggatgtcagccgccgtatatacgaagccgcccgctaa

SEQ ID NO: 16 - protein, 387 aa, Escherichia coli
MNNFNLHTPTRILFGKGAIAGLREQIPHDARVLITYGGGSVKKTGVLDQVLDALKGMDVLEFGGIEPNPAYETLMNAVKLVREQKVTFLLAVGGGSVLDGTKFIAAAANYPENIDPWHILQTGGKEIKSAIPMGCVLTLPATGSESNAGAVISRKTTGDKQAFHSAHVQPVFAVLDPVYTYTLPPRQVANGVVDAFVHTVEQYVTKPVDAKIQDRFAEGILLTLIEDGPKALKEPENYDVRANVMWAATQALNGLIGAGVPQDWATHMLGHELTAMHGLDHAQTLAIVLPALWNEKRDTKRAKLLQYAERVWNITEGSDDERIDAAIAATRNFFEQLGVPTHLSDYGLDGSSIPALLKKLEEHGMTQLGENHDITLDVSRRIYEAAR

SEQ ID NO: 17 - DNA, 1149 bp, Escherichia coli
  source 1..1149: mol_type = other DNA
atggcgaatcggatgatcctcaatgaaacggcctggttcggccgcggcgcggtcggcgccctcaccgatgaggtcaagcggcggggctaccagaaggccctgatcgtcacggataaaaccctggtgcagtgcggcgtcgtcgccaaggtgaccgacaagatggatgcggccggcctggcctgggcgatctacgacggcgtggtgcccaaccccaccatcaccgtggtgaaggaaggcctgggcgtgttccagaactcgggcgcggattatctcatcgcgatcggcggcggcagcccccaggacacctgcaaggccatcggcatcatctcgaacaaccccgagttcgcggacgtgcgctccctggagggcctgtcgccgacgaacaagccctccgtcccgatcctcgccatcccgacgacggccggcaccgcggccgaggtgaccatcaattacgtcatcaccgacgaggaaaagcggcgcaagttcgtgtgtgtggacccccatgacatcccccaggtcgccttcatcgacgccgacatgatggatggcatgccccccgccctcaaggccgcgacgggcgtggacgcgctgacgcatgccatcgaaggctacatcacccggggcgcctgggccctgacggatgccctgcatatcaaggccatcgaaatcatcgccggcgccctgcgcggctccgtggccggcgacaaggatgcgggcgaggagatggcgctgggccagtacgtggccggcatgggcttctccaatgtgggcctgggcctggtgcatggcatggcccatccgctcggcgccttctacaacacgccgcatggcgtcgcgaacgcgatcctcctgccgcatgtcatgcgctacaatgcggacttcacgggcgagaaataccgcgatatcgcccgggtcatgggcgtgaaggtcgagggcatgtcgctggaagaggcgcggaacgccgcggtcgaagccgtcttcgccctgaaccgggatgtgggcatcccgccgcacctgcgcgatgtcggcgtccgcaaggaagacatccccgcgctggcgcaggccgccctggacgatgtgtgcaccggcggcaacccccgcgaggcgacgctggaagacatcgtcgaactctaccataccgcgtggtga

SEQ ID NO: 18 - protein, 382 aa, Escherichia coli
MANRMILNETAWFGRGAVGALTDEVKRRGYQKALIVTDKTLVQCGVVAKVTDKMDAAGLAWAIYDGVVPNPTITVVKEGLGVFQNSGADYLIAIGGGSPQDTCKAIGIISNNPEFADVRSLEGLSPTNKPSVPILAIPTTAGTAAEVTINYVITDEEKRRKFVCVDPHDIPQVAFIDADMMDGMPPALKAATGVDALTHAIEGYITRGAWALTDALHIKAIEIIAGALRGSVAGDKDAGEEMALGQYVAGMGFSNVGLGLVHGMAHPLGAFYNTPHGVANAILLPHVMRYNADFTGEKYRDIARVMGVKVEGMSLEEARNAAVEAVFALNRDVGIPPHLRDVGVRKEDIPALAQAALDDVCTGGNPREATLEDIVELYHTAW

SEQ ID NO: 19 - DNA, 1254 bp, Escherichia coli
  source 1..1254: mol_type = other DNA
atgtactacctgaagaacaccaatttctggatgttcggcctgttcttcttcttctatttcttcatcatgggcgcctacttcccgttcttcccgatctggctgcacgatatcaatcacatcagcaagtcggataccggcatcatcttcgccgccatcagcctgttcagcctgctgttccagccgctcttcggcctcctgagcgacaagctcggcctgcggaagtacctgctgtggatcatcacgggcatgctggtgatgttcgcccccttcttcatcttcatcttcggcccgctcctgcagtacaacatcctcgtgggcagcatcgtgggcggcatctacctgggcttctgcttcaatgcgggcgcccccgccgtcgaggccttcatcgagaaagtgagccggcgctccaacttcgagttcggccgggcccggatgttcggctgcgtcggctgggcgctgtgcgcgagcatcgtgggcatcatgttcacgatcaacaaccagttcgtgttctggctgggctccggctgttgtctcatcctggccgtcctgctgttcttcgccaagacggacgccccgagctccgcgaccgtcgccaacgcggtcggcgccaatcactccgcgttctcgctgaagctggcgctggagctgttccgccagccgaagctgtggttcctgagcctctacgtgatcggcgtctcgtgcacgtacgatgtcttcgatcagcagttcgccaacttcttcacctcgttcttcgccaccggcgagcagggcacgcgcgtcttcggctatgtgacgacgatgggcgagctgctgaacgccagcatcatgttcttcgccccgctcatcatcaaccgcatcggcggcaaaaacgcgctgctgctcgccggcaccatcatgtcggtgcggatcatcggctcctccttcgccacctcggccctggaagtcgtcatcctgaagaccctgcacatgttcgaagtcccgttcctgctcgtcggctgtttcaagtacatcacctcccagttcgaggtgcgcttcagcgccacgatctacctggtgtgcttctgcttcttcaagcagctggcgatgatcttcatgtccgtgctggcgggcaatatgtatgagtcgatcggcttccagggcgcgtatctggtcctgggcctcgtggccctgggcttcaccctgatctccgtgttcacgctgagcggcccgggcccgctgtcgctgctgcggcgccaggtgaatgaggtcgcctga

SEQ ID NO: 20 - protein, 417 aa, Escherichia coli
MYYLKNTNFWMFGLFFFFYFFIMGAYFPFFPIWLHDINHISKSDTGIIFAAISLFSLLFQPLFGLLSDKLGLRKYLLWIITGMLVMFAPFFIFIFGPLLQYNILVGSIVGGIYLGFCFNAGAPAVEAFIEKVSRRSNFEFGRARMFGCVGWALCASIVGIMFTINNQFVFWLGSGCCLILAVLLFFAKTDAPSSATVANAVGANHSAFSLKLALELFRQPKLWFLSLYVIGVSCTYDVFDQQFANFFTSFFATGEQGTRVFGYVTTMGELLNASIMFFAPLIINRIGGKNALLLAGTIMSVRIIGSSFATSALEVVILKTLHMFEVPFLLVGCFKYITSQFEVRFSATIYLVCFCFFKQLAMIFMSVLAGNMYESIGFQGAYLVLGLVALGFTLISVFTLSGPGPLSLLRRQVNEVA

SEQ ID NO: 21 - DNA, 879 bp, Escherichia coli
  source 1..879: mol_type = other DNA
atggccgaggcgcagaacgatccgctcctgccgggctatagcttcaatgcccatctggtcgcgggcctgaccccgatcgaggcgaatggctacctggacttcttcatcgaccgcccgctgggcatgaagggctacatcctcaatctgaccatccggggccagggcgtcgtgaagaatcagggccgcgagttcgtgtgccgccccggcgacatcctcctgttcccgcccggcgaaatccatcattatggccgccatccggaagcccgcgagtggtatcatcagtgggtctatttccggccccgcgcgtattggcatgagtggctgaactggccctcgatcttcgccaacaccggcttcttccggccggacgaggcccaccagccccatttctccgacctgttcggccagatcatcaacgccggccagggcgaaggccgctattcggaactgctggccatcaacctgctcgaacagctcctcctgcggcggatggaagccatcaacgaatccctgcatcccccgatggacaaccgcgtgcgggaagcgtgccagtacatcagcgaccacctggcggactcgaatttcgatatcgcgtccgtggcccagcatgtctgcctgagcccgagccggctgtcccatctcttccgccagcagctgggcatctccgtgctctcgtggcgcgaagaccagcggatctcccaggccaagctgctgctctccaccacccgcatgcccatcgccaccgtcggccgcaacgtcggcttcgacgaccagctgtatttctcgcgggtgttcaagaagtgtacgggcgcctcgccctcggagttccgcgccggctgcgaggagaaagtcaacgacgtggccgtcaaactcagctga

SEQ ID NO: 22 - protein, 292 aa, Escherichia coli
MAEAQNDPLLPGYSFNAHLVAGLTPIEANGYLDFFIDRPLGMKGYILNLTIRGQGVVKNQGREFVCRPGDILLFPPGEIHHYGRHPEAREWYHQWVYFRPRAYWHEWLNWPSIFANTGFFRPDEAHQPHFSDLFGQIINAGQGEGRYSELLAINLLEQLLLRRMEAINESLHPPMDNRVREACQYISDHLADSNFDIASVAQHVCLSPSRLSHLFRQQLGISVLSWREDQRISQAKLLLSTTRMPIATVGRNVGFDDQLYFSRVFKKCTGASPSEFRAGCEEKVNDVAVKLS

SEQ ID NO: 23 - protein, 389 aa, Clostridium acetobutylicum
MLSFDYSIPTKVFFGKGKIDVIGEEIKKYGSRVLIVYGGGSIKRNGIYDRATAILKENNIAFYELSGVEPNPRITTVKKGIEICRENNVDLVLAIGGGSAIDCSKVIAAGVYYDGDTWDMVKDPSKITKVLPIASILTLSATGSEMDQIAVISNMETNEKLGVGHDDMRPKFSVLDPTYTFTVPKNQTAAGTADIMSHTFESYFSGVEGAYVQDGIAEAILRTCIKYGKIAMEKTDDYEARANLMWASSLAINGLLSLGKDRKWSCHPMEHELSAYYDITHGVGLAILTPNWMEYILNDDTLHKFVSYGINVWGIDKNKDNYEIAREAIKNTREYFNSLGIPSKLREVGIGKDKLELMAKQAVRNSGGTIGSLRPINAEDVLEIFKKSY

SEQ ID NO: 24 - DNA, 1170 bp, Clostridium acetobutylicum
  source 1..1170: mol_type = other DNA
atgctctcgttcgactacagcatccccaccaaggtcttcttcggcaaaggcaagatcgacgtgatcggcgaagagatcaaaaagtacggctcccgcgtgctgatcgtctacggcggcggctcgatcaaacgcaacggcatctatgaccgggccacggcgatcctgaaggaaaacaacatcgccttctacgagctgtccggcgtggagcccaacccgcggatcaccacggtcaagaagggcatcgaaatctgtcgcgaaaacaacgtcgacctggtgctggccatcggcggcggcagcgcgatcgattgctccaaggtgatcgccgccggcgtctattatgacggcgacacctgggacatggtcaaagaccccagcaagatcaccaaagtgctgccgatcgcctccatcctcaccctgagcgcgacgggcagcgaaatggatcagatcgccgtgatctcgaacatggagacgaacgaaaagctcggcgtgggccacgacgatatgcggccgaagttctcggtcctcgatccgacgtataccttcacggtgccgaagaaccagaccgccgccggcacggcggacatcatgtcgcataccttcgaatcgtatttcagcggcgtcgaaggcgcgtatgtccaggacggcatcgcggaagccatcctccgcacctgcatcaagtatggcaagatcgcgatggaaaagaccgacgactacgaggcccgcgcgaatctgatgtgggcctcgtccctggccatcaatggcctgctgagcctcggcaaggatcggaaatggtcgtgccacccgatggagcacgagctgagcgcctattacgacatcacccacggcgtgggcctggccatcctgacccccaactggatggaatatatcctgaacgacgacacgctgcataaattcgtgtcgtacggcatcaacgtctggggcatcgataagaacaaggacaactacgagatcgcccgcgaagccatcaaaaatacgcgggagtacttcaacagcctgggcatcccgtcgaagctgcgcgaggtcggcatcggcaaagataaactggagctgatggccaagcaggcggtgcgcaactcgggcggcacgatcggcagcctccgccccatcaacgcggaggatgtgctggagatcttcaagaagagctattga

SEQ ID NO: 25 - protein, 390 aa, Clostridium acetobutylicum
MVDFEYSIPTRIFFGKDKINVLGRELKKYGSKVLIVYGGGSIKRNGIYDKAVSILEKNSIKFYELAGVEPNPRVTTVEKGVKICRENGVEVVLAIGGGSAIDCAKVIAAACEYDGNPWDIVLDGSKIKRVLPIASILTIAATGSEMDTWAVINNMDTNEKLIAAHPDMAPKFSILDPTYTYTVPTNQTAAGTADIMSHIFEVYFSNTKTAYLQDRMAEALLRTCIKYGGIALEKPDDYEARANLMWASSLAINGLLTYGKDTNWSVHLMEHELSAYYDITHGVGLAILTPNWMEYILNNDTVYKFVEYGVNVWGIDKEKNHYDIAHQAIQKTRDYFVNVLGLPSRLRDVGIEEEKLDIMAKESVKLTGGTIGNLRPVNASEVLQIFKKSV

SEQ ID NO: 26 - DNA, 1173 bp, Clostridium acetobutylicum
  source 1..1173: mol_type = other DNA
atggtcgatttcgagtattcgatcccgacgcggatcttcttcggcaaggacaaaatcaacgtcctgggccgcgaactcaagaaatacggcagcaaagtgctgatcgtctacggcggcggctcgatcaagcggaacggcatctacgataaggccgtgtcgatcctggaaaagaatagcatcaagttctatgaactggcgggcgtcgaaccgaacccccgcgtgaccaccgtcgagaagggcgtcaagatctgccgggaaaacggcgtggaagtcgtgctggcgatcggcggcggctccgcgatcgactgcgccaaggtgatcgcggcggcctgcgagtacgacggcaatccctgggacatcgtcctggacggctccaagatcaagcgcgtcctcccgatcgccagcatcctgaccatcgccgcgacgggctcggaaatggacacctgggccgtcatcaacaatatggataccaacgaaaagctcatcgcggcccacccggacatggccccgaagttctcgatcctcgatcccacctacacctacaccgtcccgacgaaccagaccgcggccggcaccgccgatatcatgtcccatatcttcgaggtgtatttctccaacaccaagacggcgtacctccaggaccgcatggcggaggcgctcctccggacctgcatcaagtacggcggcatcgccctggagaagccggacgactatgaggcccgcgccaacctcatgtgggcgtcctccctggcgatcaatggcctgctgacgtacggcaaagacacgaactggtccgtgcatctcatggagcacgagctgtcggcctattatgatatcacccacggcgtgggcctcgcgatcctcacgcccaactggatggaatacatcctcaacaatgatacggtgtacaagttcgtcgagtacggcgtcaatgtctggggcatcgataaggaaaaaaatcactatgacatcgcgcatcaggcgatccagaagacgcgcgactacttcgtcaacgtgctcggcctgccctcccgcctccgcgacgtgggcatcgaagaggaaaagctggatatcatggccaaggagagcgtgaagctgaccggcggcaccatcggcaacctgcgccccgtgaacgcctccgaggtcctccagatcttcaaaaagtcggtgtga

SEQ ID NO: 27 - protein, 336 aa, Escherichia coli
MKAAVVTKDHHVDVTYKTLRSLKHGEALLKMECCGVCHTDLHVKNGDFGDKTGVILGHEGIGVVAEVGPGVTSLKPGDRASVAWFYEGCGHCEYCNSGNETLCRSVKNAGYSVDGGMAEECIVVADYAVKVPDGLDSAAASSITCAGVTTYKAVKLSKIRPGQWIAIYGLGGLGNLALQYAKNVFNAKVIAIDVNDEQLKLATEMGADLAINSHTEDAAKIVQEKTGGAHAAVVTAVAKAAFNSAVDAVRAGGRVVAVGLPPESMSLDIPRLVLDGIEVVGSLVGTRQDLTEAFQFAAEGKVVPKVALRPLADINTIFTEMEEGKIRGRMVIDFRH

SEQ ID NO: 28 - DNA, 1011 bp, Escherichia coli
  source 1..1011: mol_type = other DNA
atgaaggccgcggtcgtgaccaaggaccatcacgtcgatgtcacgtacaagacgctgcgctccctgaagcatggcgaagcgctgctgaagatggagtgctgtggcgtctgccacacggacctgcatgtgaaaaacggcgacttcggcgacaagaccggcgtcatcctcggccacgaaggcatcggcgtcgtggccgaggtgggccccggcgtcacgtccctcaagccgggcgatcgggcctcggtggcgtggttctatgagggctgcggccactgcgaatattgcaactcgggcaacgaaaccctgtgtcggtcggtgaaaaatgcgggctactccgtcgacggcggcatggcggaagaatgtatcgtggtggccgactacgccgtgaaggtcccggatggcctggacagcgccgccgcctcgtcgatcacctgcgccggcgtcaccacctataaggcggtgaaactgagcaaaatccgcccgggccagtggatcgccatctacggcctgggcggcctgggcaacctggccctgcagtacgccaagaatgtcttcaacgcgaaggtcatcgccatcgatgtcaatgatgaacagctgaagctggccacggagatgggcgcggacctggcgatcaacagccacaccgaagacgcggccaagatcgtccaggagaagaccggcggcgcccatgccgccgtggtgaccgccgtggccaaagccgccttcaattccgccgtcgacgccgtccgggccggcggccgggtcgtcgcggtgggcctgccgccggagtcgatgtccctcgacatcccgcgcctggtgctggatggcatcgaggtggtgggctccctggtcggcacccgccaggacctgaccgaagccttccagttcgccgccgaaggcaaggtcgtgcccaaggtcgccctgcggcccctcgccgacatcaacaccatcttcacggagatggaggaaggcaagatccggggccgcatggtcatcgatttccgccactga

SEQ ID NO: 29 - protein, 339 aa, Escherichia coli
MSMIKSYAAKEAGGELEVYEYDPGELRPQDVEVQVDYCGICHSDLSMIDNEWGFSQYPLVAGHEVIGRVVALGSAAQDKGLQVGQRVGIGWTARSCGHCDACISGNQINCEQGAVPTIMNRGGFAEKLRADWQWVIPLPENIDIESAGPLLCGGITVFKPLLMHHITATSRVGVIGIGGLGHIAIKLLHAMGCEVTAFSSNPAKEQEVLAMGADKVVNSRDPQALKALAGQFDLIINTVNVSLDWQPYFEALTYGGNFHTVGAVLTPLSVPAFTLIAGDRSVSGSATGTPYELRKLMRFAARSKVAPTTELFPMSKINDAIQHVRDGKARYRVVLKADF

SEQ ID NO: 30 - DNA, 1020 bp, Escherichia coli
  source 1..1020: mol_type = other DNA
atgtccatgatcaaaagctacgccgcgaaagaggcgggcggcgagctggaggtgtatgagtatgacccgggcgagctgcggccccaggacgtggaagtgcaggtcgactactgcggcatctgccattcggacctctcgatgatcgataacgagtggggcttcagccagtaccccctggtggccggccacgaggtgatcggccgcgtggtcgccctgggctcggccgcgcaggataaaggcctgcaggtcggccagcgcgtcggcatcggctggacggcccgcagctgcggccattgcgatgcctgcatcagcggcaatcagatcaattgcgaacagggcgcggtcccgaccatcatgaaccggggcggcttcgccgaaaagctgcgggccgattggcagtgggtgatcccgctgccggagaacatcgatatcgagtcggccggccccctgctgtgcggcggcatcaccgtcttcaagccgctcctgatgcatcatatcacggcgaccagccgggtcggcgtgatcggcatcggcggcctcggccacatcgcgatcaaactgctgcacgcgatgggctgcgaggtcaccgcgttctcctcgaaccccgccaaggagcaggaagtgctggcgatgggcgccgataaagtcgtgaactcgcgcgacccccaggccctcaaagccctggccggccagttcgatctcatcatcaacacggtgaacgtgtcgctggactggcagccctacttcgaagccctgacctatggcggcaacttccataccgtcggcgccgtgctgaccccgctgtccgtcccggccttcaccctgatcgccggcgaccgcagcgtgtccggcagcgccaccggcacgccgtatgagctgcgcaagctgatgcgcttcgccgcccgcagcaaggtcgccccgaccaccgagctgttccccatgtccaagatcaatgacgcgatccagcatgtccgggacggcaaggcccgctatcgcgtcgtcctcaaggcggacttctga

SEQ ID NO: 31 - protein, 382 aa, Escherichia coli; identical to SEQ ID NO: 18

SEQ ID NO: 32 - DNA, 1149 bp, Escherichia coli; identical to SEQ ID NO: 17
  source 1..1149: mol_type = other DNA

SEQ ID NO: 33 - protein, 339 aa, Escherichia coli; identical to SEQ ID NO: 29

SEQ ID NO: 34 - DNA, 1020 bp, Escherichia coli
  source 1..1020: mol_type = other DNA
atgtccatgatcaaaagctatgccgcgaaggaagccggcggcgagctggaggtctacgagtacgaccccggcgaactccgcccgcaggacgtggaggtgcaggtggattactgcggcatctgccacagcgacctgtcgatgatcgacaacgagtggggcttcagccagtacccgctggtggccggccatgaagtgatcggccgcgtcgtcgcgctgggctccgccgcccaggataaaggcctgcaggtcggccagcgcgtgggcatcggctggaccgcccggtcgtgcggccactgcgacgcctgcatctcgggcaaccagatcaattgcgagcagggcgccgtccccaccatcatgaaccgcggcggcttcgcggagaagctccgcgcggactggcagtgggtgatcccgctgccggaaaatatcgatatcgaatccgccggccccctgctgtgcggcggcatcaccgtcttcaagccgctcctgatgcatcatatcaccgccacctcccgcgtcggcgtcatcggcatcggcggcctcggccacatcgccatcaaactcctgcatgcgatgggctgtgaagtgaccgccttcagcagcaaccccgcgaaagagcaggaagtgctcgcgatgggcgcggacaaggtcgtgaacagccgcgatccccaggccctcaaagcgctggccggccagttcgatctcatcatcaacaccgtcaacgtctcgctcgactggcagccgtacttcgaagcgctgacgtacggcggcaacttccacaccgtgggcgcggtcctgacgcccctgtcggtgccggcgttcaccctgatcgccggcgatcggagcgtgtcgggctccgccaccggcaccccgtatgagctgcggaagctgatgcggttcgcggcccgcagcaaggtcgccccgacgaccgagctgttccccatgagcaagatcaacgacgccatccagcatgtgcgcgatggcaaagcccggtatcgcgtggtcctgaaagcggatttctga

SEQ ID NO: 35 - protein, 387 aa, Escherichia coli; identical to SEQ ID NO: 16

SEQ ID NO: 36 - DNA, 1164 bp, Escherichia coli
  source 1..1164: mol_type = other DNA
atgaacaatttcaacctccacaccccgacccgcatcctcttcggcaagggcgccatcgccggcctgcgcgagcagatcccgcacgacgcccgcgtcctcatcacctatggcggcggctccgtcaaaaagaccggcgtgctcgatcaggtcctggacgccctgaagggcatggacgtgctggagttcggcggcatcgagccgaacccggcctacgagacgctgatgaatgcggtgaagctggtgcgcgagcagaaggtcacgttcctgctcgcggtcggcggcggctcggtgctggacggcaccaagttcatcgccgccgcggcgaattatcccgagaacatcgatccctggcacatcctgcagacgggcggcaaggagatcaagtcggccatcccgatgggctgcgtcctgaccctccccgccaccggctcggagagcaacgccggcgccgtgatctcgcgcaaaaccaccggcgacaaacaggcgttccactccgcccatgtgcagccggtcttcgcggtgctggaccccgtctacacgtacaccctcccgccgcggcaggtcgccaacggcgtggtcgatgccttcgtgcatacggtggagcagtacgtcaccaagccggtggatgccaagatccaggaccgcttcgcggagggcatcctgctgacgctgatcgaggacggcccgaaagccctcaaggaaccggaaaactacgatgtgcgggcgaacgtcatgtgggccgcgacccaggccctgaacggcctgatcggcgccggcgtgccccaggattgggcgacgcacatgctgggccacgaactcaccgcgatgcacggcctcgaccacgcccagacgctcgccatcgtcctgccggccctgtggaatgagaagcgggacaccaagcgggcgaagctcctgcagtatgccgaacgggtgtggaacatcaccgaaggctcggacgatgaacgcatcgatgccgccatcgcggccacgcggaacttcttcgagcagctgggcgtcccgacccatctctccgactacggcctggatggctcctccatccccgcgctgctgaaaaaactggaagaacacggcatgacccagctgggcgaaaaccacgacatcaccctggatgtctcgcgccgcatctacgaagccgcccggtga

SEQ ID NO: 37 - protein, 336 aa, Bacillus stearothermophilus
MRAAVVTKDHKVSIEDKKLRALKPGEALVQTEYCGVCHTDLHVKNADFGDVTGVTLGHEGIGKVIEVAEDVESLKIGDRVSIAWMFESCGRCEYCTTGRETLCRSVKNAGYTVDGAMAEQVIVTADYAVKVPEKLDPAAASSITCAGVTTYKAVKVSNVKPGQWLGVFGIGGLGNLALQYAKNVMGAKIVAFDINDDKLAFAKELGADAIINSKDVDPVAEVMKLTDNKGLDATVVTSVAKTPFNQAVDVVKAGARVVAVGLPVDKMNLDIPRLVLDGIEVVGSLVGTRQDLREAFEFAAENKVTPKVQLRKLEEINDIFEEMENGTITGRMVIKF

SEQ ID NO: 38 - DNA, 1011 bp, Bacillus stearothermophilus
  source 1..1011: mol_type = other DNA
atgcgggcggccgtggtgaccaaggaccacaaggtcagcatcgaagataagaaactgcgggccctgaaacccggcgaggcgctggtgcagaccgaatattgtggcgtgtgtcatacggatctccatgtcaaaaacgccgatttcggcgatgtgaccggcgtgacgctcggccatgagggcatcggcaaggtgatcgaagtcgccgaagacgtggaaagcctcaagatcggcgatcgcgtgtccatcgcctggatgttcgagtcgtgtggccgctgcgagtattgcacgaccggccgggaaaccctgtgtcggagcgtcaagaatgccggctacaccgtggacggcgcgatggccgaacaggtcatcgtgacggccgactatgcggtcaaggtcccggaaaagctggacccggccgcggcgtcgtcgatcacctgcgcgggcgtcaccacctataaggccgtcaaggtgagcaatgtcaaaccgggccagtggctgggcgtcttcggcatcggcggcctgggcaacctggccctgcagtacgcgaagaatgtcatgggcgccaaaatcgtggccttcgatatcaacgatgacaagctggcgttcgccaaagaactcggcgcggatgcgatcatcaactcgaaggacgtggacccggtggccgaggtgatgaaactgacggacaacaagggcctggacgcgacggtcgtcaccagcgtcgcgaagacccccttcaatcaggcggtcgacgtggtcaaggcgggcgcccgcgtggtggccgtgggcctgccggtcgacaaaatgaacctggatatcccgcgcctcgtgctggacggcatcgaggtggtgggcagcctggtcggcacccgccaggacctgcgggaggccttcgagttcgcggccgagaataaagtgacgcccaaggtccagctccggaagctcgaagaaatcaacgatatcttcgaggagatggaaaacggcacgatcaccggccggatggtcatcaagttctga

SEQ ID NO: 39 - protein, 339 aa, Bacillus stearothermophilus
MKAAVVNEFKKALEIKEVERPKLEEGEVLVKIEACGVCHTDLHAAHGDWPIKPKLPLIPGHEGVGIVVEVAKGVKSIKVGDRVGIPWLYSACGECEYCLTGQETLCPHQLNGGYSVDGGYAEYCKAPADYVAKIPDNLDPVEVAPILCAGVTTYKALKVSGARPGEWVAIYGIGGLGHIALQYAKAMGLNVVAVDISDEKSKLAKDLGADIAINGLKEDPVKAIHDQVGGVHAAISVAVNKKAFEQAYQSVKRGGTLVVVGLPNADLPIPIFDTVLNGVSVKGSIVGTRKDMQEALDFAARGKVRPIVETAELEEINEVFERMEKGKINGRIVLKLKED

SEQ ID NO: 40 - DNA, 1020 bp, Bacillus stearothermophilus
  source 1..1020: mol_type = other DNA
atgaaggcggccgtcgtgaacgagttcaagaaggcgctggaaatcaaggaggtcgagcggcccaaactcgaagagggcgaggtcctggtgaagatcgaggcctgcggcgtgtgccataccgacctgcacgccgcccacggcgactggccgatcaagccgaaactgcccctgatcccgggccacgagggcgtgggcatcgtcgtggaagtggcgaagggcgtgaaaagcatcaaagtgggcgatcgcgtcggcatcccgtggctgtacagcgcgtgcggcgagtgcgagtactgcctgacgggccaggaaacgctctgcccgcatcagctgaatggcggctattccgtggacggcggctatgccgagtattgcaaagccccggccgactatgtcgccaagatcccggacaatctggaccccgtcgaggtcgcccccatcctgtgcgccggcgtgaccacctataaggcgctgaaagtctcgggcgcccggccgggcgagtgggtcgcgatctacggcatcggcggcctgggccacatcgccctgcagtacgccaaggcgatgggcctgaacgtggtcgcggtcgacatctccgacgagaaatcgaagctggcgaaagatctcggcgcggacatcgccatcaatggcctgaaggaagacccggtcaaggcgatccatgaccaggtcggcggcgtccatgccgccatctccgtcgcggtgaataagaaagccttcgagcaggcctatcagtccgtcaagcgcggcggcaccctggtcgtggtgggcctcccgaatgcggacctgccgatccccatcttcgatacggtgctcaacggcgtgtcggtgaagggcagcatcgtcggcacccgcaaggacatgcaggaagccctggatttcgccgcgcggggcaaggtccgccccatcgtggaaacggccgagctggaggaaatcaacgaagtgttcgagcgcatggaaaaaggcaaaatcaacggccgcatcgtcctgaagctgaaggaggattga

SEQ ID NO: 41 - protein, 346 aa, Geobacillus thermoglucosidas
MKALTYLGPGKKELMEKPKPKIEKETDAIVKMIKTTICGTDLHILSGDVPTVEEGRILGHEGVGIIEEVGSAVKNFKKGDRVLISCITSCGKCENCKKGLYAHCEDGGWILGHLIDGTQAEYVRIPHADNSLYPIPEGVDEEALVMLSDILPTGFEIGVLNGKVQPGQTVAIIGAGPVGMAALLTAQFYSPAEIIMVDLDDNRLEVAKKFGATQVVNSADGKAVEKIMELTGGKGVDVAMEAVGIPATFDICQEIVKPGGYIANIGVHGKSVEFHIEKLWIRNITLTTGLVNTTSTPMLLKTVQSKKLKPEQLITHRFAFSDIMKAYEVFGNAAKEKALKVIISNS

SEQ ID NO: 42 - DNA, 1041 bp, Geobacillus thermoglucosidas
  source 1..1041: mol_type = other DNA
atgaaagccctgacctatctgggcccgggcaaaaaagaactgatggaaaaaccgaagccgaagatcgaaaaagagacggatgccatcgtcaagatgatcaaaaccaccatctgcggcaccgacctccatatcctgtcgggcgacgtgcccaccgtggaagagggccgcatcctgggccacgagggcgtcggcatcatcgaggaagtgggctccgccgtcaagaacttcaagaaaggcgaccgggtgctgatctcgtgcatcaccagctgtggcaagtgcgagaattgcaagaagggcctgtacgcccactgcgaggacggcggctggatcctgggccatctgatcgacggcacccaggccgagtacgtgcgcatcccccatgcggacaacagcctgtacccgatccccgagggcgtcgacgaggaagccctggtcatgctgtcggatatcctgcccaccggcttcgagatcggcgtgctgaacggcaaggtccagcccggccagaccgtggcgatcatcggcgccggcccggtgggcatggccgcgctgctgaccgcccagttctacagcccggccgagatcatcatggtggacctggacgataaccgcctcgaagtggcgaagaagttcggcgcgacccaggtcgtcaacagcgcggatggcaaggcggtggagaagatcatggaactcaccggcggcaagggcgtggacgtggcgatggaagccgtcggcatcccggccaccttcgatatctgccaggagatcgtgaagccgggcggctacatcgccaacatcggcgtgcatggcaagtccgtggagttccatatcgaaaaactgtggatccgcaacatcaccctgacgacgggcctggtgaacaccacgagcacgcccatgctgctgaagacggtgcagtcgaagaagctcaagcccgaacagctcatcacccaccgcttcgccttcagcgatatcatgaaagcgtacgaagtcttcggcaacgccgccaaggagaaggcgctgaaagtcatcatcagcaattcgtga

SEQ ID NO: 43 - protein, 340 aa, Geobacillus thermoglucosidas
MKAAVVNEFKQKLEIKEVEKPKLNYGEVLVKIEACGVCHTDLHAAHGDWPVKPKLPLIPGHEGVGIVVEVAEGVKSVKVGDRVGIPWLYSACGECEYCLSGQETLCPHQLNGGYSADGGYAEYCKAPANYVAKIPEHLDPVEVAPILCAGVTTYKALKVSNAKPGEWVAIYGIGGLGHIALQYAKAMGLNVIAVDISDEKIELAKQLGADIAINGLKEDPVEAIQQNVGGAHAAISVAVTKKAFEQAYQSVRRGGCLVVVGLPNEDLPIPIFNTVLNGITVKGSIVGTRKDMQEALDFAARGKVRPIVETAPLEKINEVFERMEKGKINGRVVLTIGVNR

SEQ ID NO: 44 - DNA, 1023 bp, Geobacillus thermoglucosidas
  source 1..1023: mol_type = other DNA
atgaaagccgcggtggtcaatgagttcaagcagaaactcgaaatcaaggaagtcgaaaagccgaagctcaactacggcgaagtgctggtgaaaatcgaggcctgcggcgtctgccacaccgacctccatgcggcccacggcgactggcccgtcaagccgaagctgcccctgatcccgggccatgagggcgtgggcatcgtcgtggaagtcgcggagggcgtcaagagcgtcaaggtcggcgaccgggtgggcatcccctggctgtattccgcctgcggcgaatgcgaatactgcctgagcggccaggaaaccctgtgcccccaccagctgaacggcggctatagcgcggatggcggctatgccgagtactgtaaagcccccgccaactacgtggcgaagatcccggaacatctggaccccgtggaagtggcgcccatcctctgcgcgggcgtgaccacctataaagccctcaaggtgtccaacgccaaacccggcgagtgggtcgccatctacggcatcggcggcctcggccatatcgcgctgcagtacgccaaggcgatgggcctcaacgtcatcgccgtggatatcagcgacgagaaaatcgaactggcgaaacagctcggcgcggacatcgcgatcaacggcctgaaagaagatccggtggaagccatccagcagaacgtcggcggcgcccacgccgcgatcagcgtcgccgtgaccaagaaggcgttcgaacaggcctatcagagcgtccggcggggcggctgcctggtcgtggtcggcctgcccaacgaggacctgcccatcccgatcttcaacaccgtcctgaatggcatcaccgtcaagggctccatcgtgggcacgcggaaggatatgcaggaagcgctggatttcgcggcgcggggcaaggtgcggccgatcgtcgagacggccccgctggagaagatcaatgaggtcttcgaacgcatggagaagggcaagatcaatggccgcgtcgtgctcaccatcggcgtcaaccgctga

SEQ ID NO: 45 - protein, 340 aa, Lactococcus lactis
MKAAVVRHNPDGYADLVEKELRAIKPNEALLDMEYCGVCHTDLHVAAGDFGNKAGTVLGHEGIGIVKEIGTDVSSLQVGDRVSVAWFFEGCGHCEYCVSGNETFCREVKSAGYSVDGGMAEEAIVVADYAVKVPDGLDPIEASSITCAGVTTYKAIKVSGVKPGDWQVIFGAGGLGNLAIQYAKNVFGAKVIAVDINQDKLNLAKKIGADVTINSGDVNPVDEIKKITGGLGAQSAIVCAVARIAFEQAVASLKPMGKMVAVAVPNTEMTLSVPTVVFDGVEVAGSLVGTRLDLAEAFQFGAEGKVKPIVATRKLEEINDIIDEMKAGKIEGRMVIDFTK

SEQ ID NO: 46 - DNA, 1023 bp, Lactococcus lactis
  source 1..1023: mol_type = other DNA
atgaaggcggccgtcgtgcggcacaacccggacggctatgccgatctggtggagaaggagctccgcgcgatcaagccgaacgaggccctcctggacatggagtactgcggcgtgtgccacaccgacctgcacgtcgccgccggcgatttcggcaacaaagccggcaccgtcctgggccatgagggcatcggcatcgtgaaggaaatcggcaccgatgtgtcgtccctccaggtgggcgaccgggtcagcgtcgcctggttcttcgaaggctgcggccactgcgagtactgcgtgtccggcaacgagacgttctgccgggaagtcaagagcgcgggctacagcgtcgatggcggcatggccgaggaagccatcgtcgtcgccgactacgccgtgaaggtcccggatggcctcgatcccatcgaggccagcagcatcacctgcgcgggcgtgaccacctacaaggccatcaaggtgtccggcgtcaaaccgggcgattggcaggtgatcttcggcgccggcggcctgggcaacctcgcgatccagtacgccaaaaacgtcttcggcgcgaaggtcatcgcggtggacatcaaccaggacaagctgaacctcgcgaagaagatcggcgccgatgtcaccatcaactcgggcgatgtcaacccggtcgacgagatcaagaaaatcaccggcggcctgggcgcccagtcggccatcgtgtgcgcggtcgcccgcatcgcgttcgaacaggcggtggccagcctcaaaccgatgggcaaaatggtggcggtggccgtcccgaacaccgagatgaccctgagcgtgccgaccgtggtcttcgacggcgtcgaggtcgcgggctcgctcgtcggcacccggctggacctcgccgaggccttccagttcggcgcggagggcaaagtcaagccgatcgtcgcgacccggaagctggaggagatcaatgacatcatcgatgagatgaaggccggcaagatcgaaggccggatggtcatcgatttcaccaagtga

SEQ ID NO: 47 - protein, 340 aa, Lactococcus lactis
MKAAVVRHNPDGYADLVEKELRAIKPNEALLDMEYCGVCHTDLHVAAGDYGNKAGTVLGHEGIGIVKEIGTDVSSLQVGDRVSVAWFFEGCGHCEYCVSGNETFCREVKNAGYSVDGGMAEEAIVVADYAVKVPDGLDPIEASSITCAGVTTYKAIKVSGVKPGDWQVIFGAGGLGNLAIQYAKNVFGAKVIAVDINQDKLNLAKKIGADVIINSGDVNPVDEIKKITGGLGAQSAIVCAVARIAFEQAVASLKPMGKMVAVALPNTEMTLSVPTVVFDGVEVAGSLVGTRLDLAEAFQFGAEGKVKPIVATRKLEEINDIIDEMKAGKIEGRMVIDFTK

SEQ ID NO: 48 - DNA, 1023 bp, Lactococcus lactis
  source 1..1023: mol_type = other DNA
atgaaggcggcggtcgtgcgccataacccggacggctacgccgacctggtggaaaaggagctgcgggccatcaagccgaacgaggcgctgctcgacatggagtactgcggcgtctgccatacggacctccacgtcgccgccggcgactacggcaacaaggcgggcacggtgctgggccatgagggcatcggcatcgtgaaggaaatcggcaccgacgtgtcctccctgcaggtcggcgaccgggtcagcgtcgcctggttcttcgagggctgtggccactgcgagtattgtgtcagcggcaatgaaacgttctgtcgcgaagtcaaaaacgccggctactcggtcgatggcggcatggcggaagaagccatcgtggtcgcggactatgccgtgaaggtgccggacggcctggaccccatcgaagcgtcctcgatcacctgcgcgggcgtcacgacctacaaggcgatcaaagtgtcgggcgtcaagccgggcgactggcaggtgatcttcggcgcgggcggcctcggcaacctcgccatccagtacgccaagaacgtcttcggcgccaaagtgatcgccgtcgacatcaatcaggacaaactgaatctggcgaaaaagatcggcgccgatgtcatcatcaacagcggcgatgtgaacccggtggacgagatcaaaaagatcacgggcggcctcggcgcccagagcgcgatcgtgtgcgccgtggcccgcatcgccttcgaacaggccgtcgcgtccctgaagccgatgggcaagatggtcgccgtcgccctcccgaacacggaaatgacgctgtccgtcccgaccgtggtcttcgacggcgtggaagtggccggctcgctggtcggcacccggctggacctcgccgaggccttccagttcggcgcggaaggcaaggtcaagccgatcgtggccacgcgcaagctcgaagagatcaatgatatcatcgatgagatgaaggcgggcaagatcgaaggccgcatggtcatcgatttcaccaagtga

SEQ ID NO: 49 - protein, 401 aa, Oenococcus oeni
MRGSHHHHHHGSAERAYDFLMPSVNFFGPGVISKIGERAKMLGMKKPVIVTDKFLENLKNGAVAQTLASLKKSGVDYVVYNGVEPNPKIHNIKEVKTLYEKEDADSIITVGGGSAHDTGKGAGIIMTNGDDITKLAGIETLKNPLPPLIAVNTTAGTGSELTRHAVITNEETHLKFVVVSWRNIPLVSFNDPTLMLDIPKGLTAATGMDAFVQAVEPYVSVDHNPITDSQCIQAIKLIESSLREAVANGHNLQARTKMVEAEMLAGMAFNNANLGYVHAMAHQLGGQYDAPHGVCCALLLPYAEEYNLIADPERFAELARIMGENTDGLSTRDAAELSIKAMKQLSEDVGIPHSIKDIGAKPEDFDLMAENALKDGNAFSNPRKGTKEDIVKIFQEAYDAK

SEQ ID NO: 50 - DNA, 1206 bp, Oenococcus oeni
  source 1..1206: mol_type = other DNA
atgcgcggcagccatcatcaccatcaccacggcagcgccgaacgggcctacgatttcctgatgccctccgtcaacttcttcggcccgggcgtgatctccaagatcggcgaacgggcgaaaatgctcggcatgaagaagccggtgatcgtcaccgataagttcctggagaatctgaaaaatggcgccgtggcccagaccctggccagcctcaagaagagcggcgtcgattacgtcgtgtataacggcgtggagcccaaccccaaaatccacaacatcaaggaggtgaaaaccctgtacgaaaaggaagacgccgacagcatcatcaccgtgggcggcggctcggcccacgatacgggcaagggcgccggcatcatcatgacgaacggcgatgacatcaccaagctggccggcatcgaaaccctgaagaatcccctcccccccctgatcgccgtgaataccaccgcgggcaccggctcggaactcacgcggcacgccgtcatcacgaacgaggaaacccatctgaagttcgtcgtggtgtcctggcgcaacatcccgctggtcagcttcaatgaccccaccctgatgctggacatccccaagggcctcaccgcggccacgggcatggacgccttcgtccaggcggtcgaaccgtacgtgagcgtggatcacaatcccatcaccgactcccagtgtatccaggccatcaagctgatcgaatcgtcgctgcgggaggccgtggcgaacggccataacctgcaggcccgcaccaaaatggtggaagccgaaatgctcgcgggcatggcgttcaataacgccaacctgggctacgtccacgcgatggcccatcagctgggcggccagtacgacgccccgcatggcgtctgctgcgccctgctcctgccgtatgcggaggagtacaacctgatcgccgacccggaacgcttcgcggaactggcccgcatcatgggcgagaacaccgacggcctctcgacccgcgacgcggccgaactgtccatcaaggcgatgaaacagctgtcggaggacgtgggcatcccgcactcgatcaaggacatcggcgccaagccggaggacttcgacctgatggccgaaaatgcgctgaaggacggcaatgccttctccaacccgcgcaaaggcaccaaggaggatatcgtcaagatcttccaggaggcctatgacgccaaatga

SEQ ID NO: 51 - protein, 333 aa, Pectobacterium atrosepticum
MLNFTLHTPTKILFGEGQIAELGKEIPADARILITYGGGSVKHNGVLDQVYRALEGRNVREFSGIEPNPTYETLMKAVEVVRAEKIDFLLAVGGGSVVDGTKFIAAAADYQAAQDPWHILQTGGAEIDRGVALAAVLTLPATGSESNNGAVITRKSTNDKLAFRSPHTQPLFAVLDPVVTYTLPARQIANGVVDAFVHTVEQYLTYSVDAKVQDRFAEGLLLTLVEEGPRALAEPENYKVRANVMWSATMALNGLIGAGVPQDWSTHMLGHELTALHGLDHAQTLAIVLPAMLAARKSQKRDKLLQYAERVWNLRDGSEDQRIDGAIAATRDF

SEQ ID NO: 52 - DNA, 1002 bp, Pectobacterium atrosepticum
  source 1..1002: mol_type = other DNA
atgctgaatttcaccctccataccccgacgaagatcctgttcggcgagggccagatcgcggagctgggcaaggagatcccggccgacgcccgcatcctcatcacctacggcggcggctcggtcaaacacaacggcgtgctggatcaggtgtaccgggcgctggaaggccggaacgtgcgggagttctccggcatcgagcccaacccgacctacgaaacgctcatgaaggccgtggaggtggtccgggcggaaaagatcgatttcctgctcgccgtgggcggcggcagcgtcgtcgacggcaccaagttcatcgccgcggcggccgactaccaggccgcgcaggacccgtggcacatcctccagaccggcggcgccgaaatcgaccggggcgtggccctcgccgcggtgctgaccctgcccgccacgggcagcgaatccaataacggcgccgtcatcacccgcaaaagcaccaatgacaagctcgcgttccggtccccgcatacgcagcccctcttcgccgtcctcgacccggtggtcacgtacaccctgccggcccggcagatcgcgaatggcgtcgtcgacgccttcgtccacaccgtcgagcagtacctgacctactccgtcgacgcgaaagtccaggatcgcttcgccgagggcctgctgctcaccctggtcgaagagggcccccgggccctggccgaacccgaaaactacaaagtgcgggcgaatgtcatgtggagcgccacgatggcgctgaacggcctcatcggcgccggcgtcccccaggattggtcgacccacatgctgggccacgaactcacggccctgcacggcctcgaccacgcgcagacgctggccatcgtcctgccggccatgctggcggcccggaaatcccagaaacgggataaactcctgcagtacgccgagcgcgtctggaacctccgcgacggctcggaagatcagcggatcgacggcgccatcgccgccacgcgcgatttctga

SEQ ID NO: 53 - protein, 351 aa, Psychrobacter cryohalolentis
MANTKAYAATAPDSGLAPYAIDRRELRADDVAIEIDYCGVCHSDLHTVENDWGGSKYPVIPGHEIVGRVTAVGPEVSHFKAGDLVGVGCMVDSCRSCSACDSGLEQYCIEGSTMTYGSLDRHDGSVTHGGYSERIVVSERFVVRVPEKLDPASAAPILCAGITTYSPLKHFKVGKGHKVGVLGMGGLGHMGVKFAKALGAEVTIFTRSEAKVAEAKKQGADHVIISTDKEQMKAAADSFDFLLDTIPVAHDLNPYLKCLKYDGTHILVGLLTPIEPALQAGLLVTKRRVVAGSLIGGMPETQEVLDFCAEHDITCDIEMLDIRNINEAYVRMKKGDVKYRFVIDMKTLKEG

SEQ ID NO: 54 - DNA, 1056 bp, Psychrobacter cryohalolentis
  source 1..1056: mol_type = other DNA
atggccaataccaaggcctacgccgccacggcgccggattcgggcctggccccgtacgcgatcgaccggcgggaactgcgggccgatgacgtggcgatcgaaatcgactactgtggcgtgtgccatagcgatctccataccgtggaaaacgactggggcggctcgaagtacccggtgatcccgggccatgagatcgtcggccgggtgacggcggtgggccccgaggtcagccatttcaaggccggcgacctcgtgggcgtgggctgcatggtggattcgtgtcgctcgtgcagcgcctgcgacagcggcctcgaacagtattgcatcgagggcagcacgatgacctacggcagcctggaccgccacgatggctccgtcacccacggcggctacagcgaacgcatcgtcgtctcggaacggttcgtcgtgcgggtgcccgaaaaactggacccggcctcggccgccccgatcctgtgcgccggcatcacgacgtacagcccgctgaagcacttcaaggtgggcaagggccataaagtcggcgtcctgggcatgggcggcctgggccatatgggcgtgaagttcgccaaggccctgggcgccgaggtgacgatcttcacccggtccgaggcgaaagtggccgaagcgaagaaacagggcgccgaccatgtcatcatctcgaccgataaggagcagatgaaggccgccgccgacagcttcgatttcctcctcgacaccatcccggtggcgcacgacctgaatccgtatctgaagtgtctgaaatacgatggcacccacatcctcgtgggcctgctgacccccatcgaaccggcgctgcaggccggcctcctggtcaccaagcggcgcgtcgtggcgggcagcctgatcggcggcatgcccgagacgcaggaagtcctggacttctgcgccgaacatgacatcacctgtgatatcgagatgctcgacatccgcaacatcaacgaagcgtacgtccgcatgaaaaagggcgatgtcaagtaccgcttcgtgatcgatatgaagacgctgaaggaaggctga

SEQ ID NO: 55 - protein, 548 aa, Methylocaldus szegediense
MGTAKADSIGQYLLKRLYEAGVKHIFGVPGDYVLGFYDLMAKSPIQHVGTTREDTAAFAADGYARCRGLGALAVTYGVGALNTVNAVAGAYAESSPVIVISGAPGVREQKEDPMIHHRFGPFTFQREIFDRITCAAVTLDDPIIAFRQIDRVIAAARHSCKPVYIELPRDLVMAEGHPVPTEPPEEPASDEAALSEAVAETAELMSKSVSPTVLAGVELHRRGLQDALVELVERARLPVAATLTGKSVIAERHPAYLGVYEGAMSSENARYMVEQSDLLLMLGVTLNDIDTGVYTARLDPHRIVRAAQNEVVIRYHRYPRVTLSDFVLSLARTVKAKHETFPAPVTTPEATEFPMPERPMTIARLIERLDRALTPDMIVVSDVGDCLFAAIDLRVYERSEFLSSAFYTTMGFAVPAALGAQIARPDHRALILVGDGAFQMTGTELSTHIRFGLAPIVVVFNNCGYSTERYILDGPFNDISCWNFDRLGELFGPLNGYDAPDEESFEKALVEALANHATPSIINVHISRDDSSSAMRRLAEVLKSRVRG

SEQ ID NO: 56 - DNA, 1647 bp, Methylocaldus szegediense
  source 1..1647: mol_type = other DNA
atgggcaccgccaaggcggatagcatcggccagtacctcctgaaacgcctgtacgaggccggcgtgaaacatatcttcggcgtgcccggcgattacgtgctgggcttctacgatctgatggcgaagagcccgatccagcacgtcggcacgacccgcgaggacaccgcggccttcgccgccgacggctacgcccgctgtcgcggcctcggcgccctggcggtcacctatggcgtcggcgccctgaacaccgtgaatgccgtcgcgggcgcctatgccgaatcgagcccggtgatcgtcatcagcggcgccccgggcgtgcgcgaacagaaggaagacccgatgatccatcatcggttcggcccgttcaccttccagcgggaaatcttcgaccggatcacctgcgccgcggtcacgctggacgatcccatcatcgccttccgccagatcgaccgggtgatcgcggcggcccgccactcgtgcaaacccgtgtatatcgaactgccccgcgacctggtgatggccgaaggccatccggtcccgacggagcccccggaagagcccgcctccgatgaggcggccctgagcgaagcggtggccgaaaccgcggaactgatgtccaagtcggtgagccccaccgtcctggcgggcgtcgaactgcaccggcgcggcctgcaggacgccctggtggaactggtggaacgcgcccggctgccggtggccgccaccctcaccggcaagagcgtgatcgccgaacgccaccccgcctatctgggcgtctacgaaggcgccatgtcctcggaaaacgcccgctacatggtggaacagtccgatctcctgctcatgctgggcgtgacgctgaacgacatcgacaccggcgtctataccgcccgcctcgacccgcatcgcatcgtgcgggcggcccagaacgaggtggtgatccgctaccatcgctatccgcgggtcaccctgtcggacttcgtcctgagcctggcccgcaccgtgaaagcgaagcatgaaaccttccccgccccggtcacgacccccgaagccacggagttccccatgcccgagcgcccgatgacgatcgcccgcctcatcgaacgcctggaccgcgccctgacccccgacatgatcgtcgtgagcgatgtgggcgattgcctgttcgcggccatcgatctccgggtctacgagcggagcgagttcctctcgtcggccttctacaccacgatgggcttcgccgtcccggccgccctgggcgcccagatcgcgcggccggaccaccgggccctgatcctggtgggcgatggcgcgttccagatgacgggcaccgagctcagcacgcacatccgcttcggcctcgcgcccatcgtggtcgtgttcaacaactgtggctattcgaccgaacggtacatcctggacggcccgttcaacgacatctcgtgctggaacttcgaccggctgggcgaactgttcggcccgctgaacggctatgacgcgccggacgaggagagcttcgagaaggccctcgtcgaggccctggccaaccatgccacgcccagcatcatcaacgtccacatcagccgcgacgacagctccagcgcgatgcggcggctggccgaagtcctgaagagccgcgtccggggctga

SEQ ID NO: 57 - protein, 550 aa, Methylosarcina lacus
MNTAKFDTIGQYLLKRLYDAGVKHIFGVPGDYILGFYDLMVNSPVQHIGTTREDTAAFAADAYARCLGLGAMAVTYGVGALNTVNAVAGAYAESSPVIVISGAPGIREQREDPLIHHRFGPFTFQREIFERITCATEVLNDPVIAFRQIDRAIATARRLCKPVYIEIPRDLVMAEGYPMPDEALEPLESDETALSEALAETMELMAKSVSPMIIAGVELHRRGLQSALVNLVERAHLPVVATLSGKSVMAERHPAYLGIYEGAMSSENARYMVEQSDLLLMLGVTLNDIDTGIYTAKLDPHHMIRAAQNEVVISSHRYPRVTLSDFLTALVGLVKTRSEGFSSPPAAYEASAFPEPKRPITTARMIGRLNQALSPEMIVVCDVGDCLFAAIDLQVHEQSEFLASCYYATMGFAVPAALGAQIARPDHRLLVLVGDGAFQMTGTELSTHAYLGLNPIVVVFNNSGYGTERGILEGPFNDISSWRFDRLGEVFGPLKGYDAATEEAFEAALINSLNNRTMPSIINVHLSADDASSAMKRLAEHLKSRVKGGS

SEQ ID NO: 58 - DNA, 1653 bp, Methylosarcina lacus
  source 1..1653: mol_type = other DNA
atgaacaccgccaagttcgacaccatcggccagtacctgctcaagcggctgtacgacgcgggcgtgaaacacatcttcggcgtgccgggcgattatatcctgggcttctatgacctgatggtgaacagcccggtccagcacatcggcaccacgcgcgaggacaccgcggccttcgcggcggacgcctacgcccgctgcctgggcctcggcgcgatggcggtcacgtacggcgtgggcgccctgaataccgtgaatgccgtcgccggcgcgtatgccgaaagctcgccggtgatcgtcatctccggcgcccccggcatccgcgagcagcgggaggacccgctgatccatcatcggttcggcccgttcaccttccagcgggagatcttcgagcgcatcacctgcgcgaccgaagtgctcaacgacccggtgatcgccttccgccagatcgatcgcgcgatcgccaccgcccggcggctgtgcaagccggtgtacatcgagatcccgcgcgatctggtgatggcggaaggctaccccatgccggacgaggccctcgaacccctggagagcgatgaaaccgccctgagcgaggcgctggcggaaacgatggaactgatggcgaaaagcgtctccccgatgatcatcgccggcgtcgaactgcatcggcggggcctgcagagcgccctcgtcaacctggtggaacgcgcccatctcccggtggtcgccaccctgtcgggcaaaagcgtcatggcggaacggcacccggcctacctgggcatctacgaaggcgcgatgtcctcggaaaatgcccggtacatggtcgaacagagcgacctcctcctcatgctgggcgtcaccctcaacgacatcgataccggcatctatacggccaagctcgacccgcatcatatgatccgggcggcccagaacgaggtggtgatctcctcgcatcgctacccgcgggtcaccctctcggacttcctgacggcgctggtgggcctggtcaagacccggagcgaaggcttcagctcgccgccggccgcctacgaggccagcgccttcccggaaccgaagcggccgatcaccacggcgcggatgatcggccggctgaaccaggcgctgtcgccggaaatgatcgtggtgtgcgacgtgggcgactgcctcttcgccgccatcgatctgcaggtgcacgagcagtccgagttcctcgccagctgctactatgccacgatgggcttcgccgtgcccgcggcgctcggcgcccagatcgcgcggcccgatcaccggctgctcgtgctggtgggcgacggcgccttccagatgacgggcaccgaactgagcacccatgcctatctgggcctcaacccgatcgtcgtggtgttcaacaactccggctacggcaccgaacgcggcatcctggaaggccccttcaacgacatcagcagctggcggttcgaccgcctcggcgaagtgttcggcccgctgaagggctatgacgccgccaccgaggaggccttcgaagcggcgctcatcaactccctcaacaaccggacgatgccgagcatcatcaacgtccatctctccgccgacgacgcctcctccgccatgaagcgcctcgccgagcacctgaagagccgcgtgaagggcggctcgtga

SEQ ID NO: 59 - protein, 550 aa, Methylomonas denitrificans
MSTAKFDTIGQYLLKRLYQAGVKDIFGVPGDYVLGFYDLMIKSQVRHIGTTREDSAAFAADGYARCVGMGALAVTYGVGALNTVNAIAGAYAESSPVVLISGAPGVSEQKDDPLIHHRFGPFTFQREIFERISCASVVLNDPVIAFRQIDHAIEAARRFCKPVYIELPRDLVMAEGYPMPTETVEKFTSDEAALSEAIAETMTLLSKAVSPMIVAGVELHRRGLQGALADFVERTCLPVVATLTGKSVMSERHPAYLGIYEGAMSSEAVRDRVEKSDLLLMLGVTLNEIDTGIYTAKLNSHSTIRAALNEVVISAHRYPGIALEDFLGALASSVSLSSREVVSSPKPPESIAFPEPDRPITTARLVERLNSALSNDMIVVCDVGDCLFAAIDLRVHEQSEFLASAFYTTMGFAVPAALGAQIARPDRRALILVGDGAFQMTGTELSTHARLGLNPIVVVFNNGGYSTERCILEGPFNDINPWRFDRLGELFGPLAGYEAATEAEFEEALLNALDNHGMPSIINVHLAADDSSEAMKRLAEHLQSKIKRDA

SEQ ID NO: 60 - DNA, 1653 bp, Methylomonas denitrificans
  source 1..1653: mol_type = other DNA
atgtcgaccgcgaagttcgacaccatcggccagtatctgctgaagcggctgtatcaggccggcgtcaaggatatcttcggcgtccccggcgactacgtgctgggcttctatgacctgatgatcaagtcgcaggtgcgccatatcggcaccacgcgggaggacagcgcggccttcgcggccgacggctacgcccggtgtgtgggcatgggcgccctggcggtgacgtacggcgtcggcgccctgaacacggtgaacgccatcgccggcgcgtacgccgaatcgtcccccgtcgtcctcatcagcggcgccccgggcgtgtccgagcagaaggacgatccgctgatccatcatcgcttcggccccttcacgttccagcgcgaaatcttcgaacggatcagctgtgcctccgtcgtcctgaatgacccggtcatcgcgttccggcagatcgaccatgccatcgaggccgcccggcgcttctgcaagccggtgtatatcgaactgccgcgggacctcgtgatggcggagggctaccccatgcccaccgagacggtcgaaaagttcacctccgatgaggccgcgctgtccgaagcgatcgcggaaaccatgaccctgctcagcaaggcggtcagcccgatgatcgtggcgggcgtggagctgcatcggcgcggcctgcagggcgccctggccgacttcgtggaacggacctgtctgcccgtggtggccaccctgacgggcaagtcggtgatgtcggagcgccatcccgcctacctgggcatctacgaaggcgccatgtcctcggaagcggtgcgcgatcgggtggagaagagcgatctgctcctgatgctgggcgtgaccctcaacgaaatcgacaccggcatctacacggccaaactgaacagccatagcacgatccgcgccgcgctgaacgaggtcgtgatctccgcccaccgctatcccggcatcgcgctggaagatttcctgggcgccctggcctcgtcggtgtccctcagctcgcgcgaggtggtcagcagcccgaaacccccggagtcgatcgccttcccggaaccggaccggccgatcacgacggcccgcctggtcgaacggctcaactccgccctcagcaatgacatgatcgtcgtgtgtgacgtgggcgactgcctgttcgcggccatcgacctccgcgtgcatgagcagagcgagttcctggcctccgccttctacaccacgatgggcttcgcggtgcccgcggccctcggcgcccagatcgcccgccccgaccggcgggcgctgatcctggtcggcgacggcgcgttccagatgaccggcaccgaactgtccacgcacgcgcggctgggcctgaacccgatcgtcgtcgtgttcaataatggcggctactcgaccgaacgctgcatcctggaaggcccgttcaatgatatcaacccctggcgcttcgaccgcctgggcgagctgttcggccccctggccggctatgaggccgccacggaagccgagttcgaggaagccctgctgaacgcgctggacaaccacggcatgccgtccatcatcaatgtgcatctcgccgccgacgatagctccgaggcgatgaaacgcctggccgagcacctgcagagcaaaatcaagcgggacgcctga

SEQ ID NO: 61 - protein, 553 aa, Methylomonas methanica
MNTVKLETMGQYLLNRLYEAGVKHVFGVPGDYVLGFYDLMEKSPIQHIGTTREDTAAFAADGYARCRGLGALAVTYGVGALNTVNAVAGAYAESSPVIVISGAPGVCEQRDDPLIHHRFGPFTFQREIFERITCATAVLNDPVIAFRQIDHAIASARHYCKPVYIEIPRDLVSVEGYPMPAIAAMEPSGSDKSALSEAVAETMSLLEKSVSPMVIAGIELHRRGLQNRLLELIERARLPVTATLTGKSVIAERHPAYLGIYEGAMSSEHARYMVEQSDLLLMLGVTLNEVDTGIYTAKLDPQHTIRAALNEVVISAHRYPNIALADYLNALVDAVKPSEAGFSAKPGKPVARAFPEPDRPISINRLIERINQALEPETIVVCDVGDCLFAAIDLEVHEQSEFLASGFYTTMGFAVPAALGAQVARPGHRALILVGDGAFQMTGTELSTQARLGLDSIVIVFNNSGYSTERCILEGPFNDIARWRFDRLGEVFGPLQGFDAATEESFESALIQALNNRSMPSIINVHLASDDTSSAMRRLAEHLKSKVQGERPA

SEQ ID NO: 62 - DNA, 1662 bp, Methylomonas methanica
  source 1..1662: mol_type = other DNA
atgaacacggtcaaactggaaacgatgggccagtacctgctgaaccgcctctatgaggcgggcgtgaaacatgtcttcggcgtcccgggcgactatgtcctgggcttctacgacctgatggagaagtcccccatccagcatatcggcaccacccgcgaagataccgcggccttcgccgccgacggctacgcccgctgccgcggcctgggcgccctggccgtcacgtacggcgtcggcgccctcaacacggtgaatgcggtggcgggcgcctacgcggagtcgtcgcccgtgatcgtcatcagcggcgccccgggcgtctgtgagcagcgcgatgacccgctgatccaccaccgcttcggccccttcaccttccagcgcgagatcttcgaacggatcacctgcgccaccgcggtgctgaacgacccggtgatcgcgttccggcagatcgatcacgccatcgcctccgcccgccattattgcaagccggtctatatcgaaatcccgcgggacctcgtgtccgtggaaggctacccgatgcccgccatcgccgcgatggagccgtcgggctccgacaagtccgccctcagcgaggccgtggcggaaaccatgtcgctgctggaaaagtccgtgtccccgatggtcatcgccggcatcgagctccaccgccgcggcctgcagaaccgcctcctcgaactgatcgaacgcgcccgcctgcccgtgacggccaccctcaccggcaaaagcgtgatcgcggagcggcaccccgcgtacctgggcatctatgagggcgcgatgtccagcgaacacgcccgctatatggtcgagcagtccgacctcctcctcatgctgggcgtgaccctgaacgaggtcgacacgggcatctacaccgcgaagctggacccccagcataccatccgggcggcgctgaacgaggtggtgatctcggcccaccgctatccgaatatcgccctggccgactatctgaacgccctggtggacgcggtgaagccgtcggaggcgggcttctccgccaaaccgggcaaacccgtcgcgcgggcgttcccggagcccgaccgcccgatctcgatcaaccgcctgatcgaacgcatcaatcaggcgctggaaccggagacgatcgtggtgtgcgacgtgggcgactgtctcttcgccgccatcgatctggaggtccacgagcagtcggagttcctggcctcgggcttctataccacgatgggcttcgccgtgcccgccgccctgggcgcccaggtcgcccggcccggccaccgcgccctgatcctggtgggcgacggcgcgttccagatgaccggcacggaactctccacccaggcccggctgggcctggactcgatcgtgatcgtcttcaacaattcgggctattccacggagcggtgcatcctggagggcccgttcaacgatatcgcccgctggcgcttcgaccgcctcggcgaagtcttcggcccgctgcagggcttcgacgcggcgaccgaggagtccttcgagtcggcgctgatccaggccctgaacaaccgcagcatgcccagcatcatcaacgtccacctcgcgagcgacgacacgagcagcgcgatgcggcgcctggcggaacacctgaagtcgaaggtgcagggcgaacgccccgcctga

SEQ ID NO: 63 - protein, 552 aa, Methylohalobius crimeensis
MATRNSTTSIGEYLLQRLHEAGAHHIFGVPGDYILKFYEQISQGPVRHIGTTREDTAAFAADGYARCQGIGAMAITYGVGALNVVNAVAGAYAESSPVVVISGAPGVWEQREDPLLHHRFGPYTFQREIFDRITCATTVLDDPITAFRQIDRTIAAAQREHKPVYIELPRDRVTVAGVPLPAVAEATPQETSDAATLDEAVAETLALLAQAKSPVLIAGVEVHRCGLQDALVDLVVRAGLPVAATLTGKSVVGERHPAYIGVYEGAASSEHTRQMVERADVLIMLGVTLNDVDTGVYTANLDPHRLVRASQGEVNIRYHRYPRVQLQDFIGALARQVSPRREALPSQPFVDSGPAFPVPGQAMTTARLIARLNCALTPEMIVVSDVGDCLFAAIELRVCERSEFLASAYYTTMGFAVPAALGAQVARPDRRALILVGDGAFQMTGTELSTHARLNLAPIIIVFNNAGYSTERNILEGPFNDIAAWRFDRLGEVFGPLHGYDAKTEDAFETALARALAETGCPSLINVHLSPDDASPAMRRLTERLSHRVGNQ

SEQ ID NO: 64 - DNA, 1659 bp, Methylohalobius crimeensis
  source 1..1659: mol_type = other DNA
atggccacgcgcaactcgacgacctcgatcggcgagtatctcctccagcgcctgcacgaggccggcgcccaccacatcttcggcgtcccgggcgattatatcctgaaattctatgaacagatctcgcagggcccggtccgccacatcggcaccacgcgcgaagacaccgcggcgttcgcggcggacggctatgcccggtgccagggcatcggcgcgatggccatcacctacggcgtgggcgccctcaacgtggtgaatgccgtcgcgggcgcgtacgccgaaagctcgccggtggtcgtgatctcgggcgcccccggcgtgtgggaacagcgcgaggacccgctgctgcaccaccgcttcggcccctacacgttccagcgggagatcttcgaccggatcacctgtgccaccacggtcctggacgatccgatcacggcgttccgccagatcgatcgcacgatcgccgcggcgcagcgcgaacataagccggtctacatcgaactgccgcgcgatcgggtcacggtggccggcgtgccgctcccggccgtcgcggaggcgacgccccaggaaacgtccgacgccgccaccctggacgaggccgtggccgaaacgctggccctcctcgcccaggccaagagccccgtgctgatcgcgggcgtggaggtccaccgctgcggcctccaggatgcgctcgtcgacctggtcgtccgcgcgggcctgccggtcgcggccaccctgacgggcaaatccgtcgtgggcgaacgccacccggcgtatatcggcgtctacgagggcgccgcgtcgtccgaacacacccgccagatggtggaacgcgcggacgtgctcatcatgctcggcgtgacgctgaacgacgtggacaccggcgtctatacggcgaacctggacccccatcgcctggtccgcgcgtcccagggcgaggtgaacatccgctaccatcggtatccgcgcgtccagctgcaggacttcatcggcgccctcgcccgccaggtcagcccccgccgcgaggccctgccgtcccagcccttcgtcgatagcggccccgccttccccgtcccgggccaggcgatgaccacggcgcggctcatcgcccgcctgaactgcgccctcacccccgaaatgatcgtggtgtccgatgtgggcgattgcctgttcgccgccatcgaactccgggtctgcgaacggagcgagttcctggcctccgcctactatacgacgatgggcttcgcggtgccggccgccctgggcgcccaggtcgcgcggccggatcggcgggccctgatcctggtgggcgacggcgccttccagatgacgggcaccgagctgagcacccacgcccgcctgaacctggcgccgatcatcatcgtcttcaacaacgccggctacagcacggagcgcaatatcctggagggcccgttcaatgacatcgcggcctggcggttcgaccgcctcggcgaagtgttcggccccctgcatggctacgacgccaagacggaagatgcgttcgagacggcgctcgcccgcgccctggccgagacgggctgcccgtcgctcatcaatgtgcatctgtccccggacgacgcctcgccggccatgcgccgcctgacggagcggctgtcccatcgcgtgggcaatcagtga

SEQ ID NO: 65 - protein, 544 aa, Methylobacter marinus
MSSNPSIGHYLLTRLYESGVHHIFGVPGDYILRFYQQLSESPVQHIGTTREDTAAFATDAYARCRGLGAMAVTYGVGALNVVNAVAGAHAESSPIVVISGAPGIKERREHPLLHHRFGPFRLQREIFERITCAVAVLDDPYTAFRQIDRVLAAAREHCKPVYIELPRDRVDTEGYPIPSESLPAPASDAASLNEAVEEALQLLDEAASPVLVAGVELHRRGLQDQLLSLVDKTHLPVAATLTGKSVLGERHPCYLGIYEGAMGSPLARDRVEQADFLLMLGVTLNDVDLGIFTARLDANRIIRASQDEVIIHHHRYPQVLLRDFVSMLNERMTPRPQTGPAVAAKPAAFDFPVKGQPMKIIRLIARLNRFLTPDMVVVSDVGDCLFSAIDLRVHENSEFLASAYYTSMGFAVPAALGAQIARPTRRTLVLVGDGAFQMTGTELSTIAHLGLNPIVIVFNNKGYSTERYILDGPFNDIPAWQFERLGELFGPLTGYAASTEDEFEDCLNQALAQRSSPSLINVHLSPDDPSAAMRGLAEHLGKRV

SEQ ID NO: 66 - DNA, 1635 bp, Methylobacter marinus
  source 1..1635: mol_type = other DNA
atgagcagcaacccgtcgatcggccactacctgctcacgcggctgtacgaatccggcgtgcaccacatcttcggcgtcccgggcgactacatcctccgcttctaccagcagctgagcgagagcccggtgcagcatatcggcaccacgcgcgaagacacggccgccttcgcgaccgatgcgtatgcccgctgtcggggcctgggcgcgatggccgtgacctacggcgtcggcgccctcaatgtggtcaacgccgtggcgggcgcccatgccgagtcgagccccatcgtggtcatctccggcgcccccggcatcaaggaacgccgggaacaccccctgctgcatcaccgcttcggcccgttccgcctgcagcgcgagatcttcgaacggatcacctgcgccgtcgcggtgctggatgacccctataccgccttccggcagatcgaccgggtcctggcggcggcgcgggagcactgcaagcccgtctacatcgagctgccgcgcgatcgcgtggataccgagggctatccgatcccgtcggagtccctgccggcccccgcctccgacgccgcgagcctcaacgaagccgtggaggaagccctgcagctcctcgatgaggccgcgtcgccggtgctggtcgccggcgtggaactgcaccgccggggcctccaggatcagctcctctcgctggtggataaaacccatctgccggtcgccgccacgctcaccggcaagtccgtcctgggcgagcggcacccgtgttatctgggcatctacgagggcgcgatgggctcccccctggcgcgggaccgcgtcgagcaggccgatttcctgctgatgctgggcgtcacgctgaacgatgtggacctcggcatcttcaccgcccgcctcgacgccaatcgcatcatccgggcctcccaggacgaagtcatcatccaccaccaccgctatccgcaggtcctcctgcgggacttcgtgtccatgctgaacgaacggatgaccccgcgcccccagacgggcccggccgtcgccgcgaagccggccgccttcgatttcccggtcaaaggccagccgatgaagatcatccggctgatcgcccggctgaatcggttcctcaccccggacatggtcgtggtcagcgacgtgggcgattgcctgttcagcgcgatcgacctgcgggtgcacgaaaacagcgagttcctcgcctcggcctactacaccagcatgggcttcgcggtcccggccgcgctgggcgcccagatcgcccgccccacccggcgcacgctggtcctcgtgggcgacggcgcgttccagatgaccggcaccgagctctcgaccatcgcgcacctcggcctgaacccgatcgtgatcgtcttcaataacaagggctactcgaccgaacgctatatcctggatggcccgttcaatgatatcccggcctggcagttcgagcgcctgggcgaactcttcggcccgctgaccggctatgccgccagcaccgaggacgagttcgaagattgtctgaaccaggccctggcccagcggtcgagcccctccctcatcaacgtccacctctccccggacgatccgagcgccgccatgcggggcctcgccgaacatctgggcaagcgcgtctga

SEQ ID NO: 67 - protein, 549 aa, Methylobacter luteus
MEVHLMSSNPSIGHYLLARLYESGVHHIFGVPGDYILRFYQQLSESPIQHIGTTREDTAAFATDAYARCRGLGAMAVTYGVGALNVVNGVAGAYAESSPVVVISGAPGIKERHEHPLLHHRFGPFRLQHEIFERITCATAVLDDPYMAFRQIDRVLAAAREHCKPVYIELPRDRVDVEGYPMPSESMPAPASDAESLNEAVEETLQLLGKAASPVLIAGVELHRRGLQDKLLSLVDKTHLPVAASLTGKSVLGERHPCYLGIYEGAMGSSLARDSVEQSDFLLMLGVTMNDIDLGIFTAKLDANRIIRATQDEVIIHHHRYPHVLLRDFVTVLNERITPRPGIRPAVAAEPAAFDFPVKDQPMKILRLIERLNRFLTPDMAVVSDVGDCLFAAIDLRVHENSEFLASAYYTSMGFAVPAALGAQIANPTRRTLVLVGDGAFQMTGTELSTIARFGLNPIVIVFNNCGYSTERYILDGPFNDIACWQFERLSEVFGPLSGYSARTEDEFENCLTQAFAQQSSPSLINVHLPPDDPSAAMRGLAEHLGKRV

SEQ ID NO: 68 - DNA, 1650 bp, Methylobacter luteus
  source 1..1650: mol_type = other DNA
atggaggtgcatctgatgtcctccaacccgtccatcggccattatctgctcgcccgcctgtacgagtccggcgtgcatcacatcttcggcgtccccggcgactacatcctgcgcttctaccagcagctcagcgagtccccgatccagcacatcggcaccacgcgcgaggacaccgccgccttcgcgaccgacgcctacgcccgctgccggggcctgggcgcgatggccgtgacctacggcgtgggcgccctgaacgtcgtgaacggcgtggccggcgcgtacgcggaatcctcgccggtcgtggtgatctccggcgccccgggcatcaaggaacgccatgaacatccgctgctgcatcatcgcttcggcccgttccgcctgcagcatgaaatcttcgaacgcatcacctgcgcgacggcggtcctggacgatccctacatggcgttccggcagatcgaccgggtgctggccgccgcccgcgagcactgcaagcccgtgtatatcgagctgccgcgcgaccgggtggatgtcgaaggctatccgatgccgagcgagagcatgccggccccggcctccgacgcggaaagcctgaacgaagcggtcgaagaaacgctgcagctgctgggcaaggccgcgtcgcccgtgctgatcgcgggcgtcgaactgcaccgccgcggcctccaggacaaactgctctccctggtcgacaagacccatctcccggtggccgcctcgctgaccggcaaatcggtcctgggcgaacgccacccgtgctatctgggcatctatgaaggcgcgatgggctcgtccctggcccgggactccgtggagcagtcggacttcctcctgatgctgggcgtcaccatgaacgacatcgatctgggcatcttcaccgcgaagctggacgcgaaccgcatcatccgcgccacgcaggatgaggtcatcatccaccatcatcgctatccccacgtcctgctgcgggacttcgtcaccgtcctgaatgagcgcatcacgccccgcccgggcatccgcccggccgtcgcggcggagccggccgccttcgacttcccggtcaaggaccagcccatgaaaatcctccggctgatcgaacggctcaaccgcttcctgacccccgacatggccgtcgtctccgatgtcggcgattgcctcttcgcggcgatcgacctccgggtgcacgagaactcggagttcctggcgtcggcgtattataccagcatgggcttcgccgtgcccgcggcgctcggcgcccagatcgcgaacccgacccgccgcacgctggtgctggtcggcgacggcgccttccagatgaccggcaccgaactgtccaccatcgcccggttcggcctgaacccgatcgtgatcgtcttcaataactgtggctattcgaccgagcggtacatcctggatggccccttcaacgatatcgcctgctggcagttcgaacggctgagcgaggtgttcggcccgctctcgggctattcggcccggacggaggatgagttcgaaaactgcctgacccaggccttcgcccagcagtcctcgccgtccctcatcaacgtccacctccccccggatgacccgagcgcggccatgcgcggcctcgcggaacacctcggcaagcgcgtctga

SEQ ID NO: 69 - protein, 542 aa, Lamprocystis purpurea
MGDYLLLRLKEAGVDHCFGVPGDYVLRFYDRLCRSDIRHIGTTREDTAAFAADGYARSRGLGALAVTYGVGALNVVNAVAGANAESSPVVVISGAPGVAEQRDDPQLHHRFGPFRFQREIFERITCACAVLDDPYTALREIDRTLDAARRYSRPVYIELPRDRVDTPAFPIPHEPEEEAGSDPEALAEAVAETLALVGRAQAPVILAGVELHRRGLQDLLAGFVLKAHLPVAATLTGKSVVAERQPGYLGVYEGAMGPEGARRVVEEADLLLLLGVTPNDIDLGINTARLDPARTVRAGQEEIWVHRHRYPHVHLRDFLAALTDAVVPHPGPLPDVPGPVGAPDFPQPGQPMTMARMMARLNDFLTPDMQVVADSGDCLFASVDLRVHARSEFLASAYYTTMGFAVPAALGAQVANPGRRPLVLVGDGAFQMTGTELSTAARLGLDPIVIIGNNRGYTTERFILEGPFNDIADWRFHRLGELFGPLRGFSAPTEDAFDAALGAALAFRDGPSVIEVALRPDDCSAALTRLSERLRDVVQQSA

SEQ ID NO: 70 - DNA, 1629 bp, Lamprocystis purpurea
  source 1..1629: mol_type = other DNA
atgggcgactacctcctgctccgcctgaaggaagcgggcgtcgaccactgcttcggcgtccccggcgactacgtcctgcggttctacgatcggctctgccggtcggacatccggcacatcggcaccacccgcgaggacaccgcggccttcgcggccgacggctacgcccggtcccgcggcctgggcgccctggccgtgacgtatggcgtgggcgccctcaacgtggtcaacgcggtcgccggcgccaacgcggagtcctccccggtcgtcgtgatctccggcgccccgggcgtcgcggaacagcgcgacgatccccagctgcaccatcggttcggcccgttccggttccagcgcgagatcttcgaacgcatcacctgcgcgtgtgccgtgctcgacgacccgtacaccgccctgcgcgagatcgaccgcacgctcgacgcggcgcggcggtacagccggcccgtgtatatcgaactcccgcgcgaccgggtcgacacgcccgccttccccatcccccacgagccggaagaggaagccggcagcgacccggaggccctggccgaggcggtcgccgaaaccctggcgctcgtcggccgcgcccaggcccccgtcatcctggccggcgtggagctgcaccgccgcggcctgcaggacctcctggccggcttcgtgctgaaggcccacctcccggtggccgcgacgctgacgggcaagtcggtggtcgccgagcgccagccgggctacctgggcgtgtacgagggcgcgatgggcccggaaggcgcccgccgcgtcgtggaagaagccgacctgctgctcctgctgggcgtgaccccgaacgacatcgacctgggcatcaacaccgcccggctggaccccgcccggaccgtccgggccggccaggaagaaatctgggtccatcggcaccgctacccgcatgtgcacctgcgggatttcctggccgcgctgacggatgccgtggtgccccaccccggccccctgccggatgtccccggcccggtcggcgccccggacttcccccagccgggccagcccatgacgatggcccgcatgatggcccgcctgaacgacttcctgacccccgacatgcaggtggtggccgactccggcgactgtctgttcgcgtccgtcgacctgcgcgtccatgcccgcagcgagttcctggcctcggcctattacacgacgatgggcttcgccgtcccggccgcgctcggcgcccaggtcgccaaccccggccgccgcccgctcgtgctggtgggcgacggcgccttccagatgaccggcacggagctgtcgaccgccgcccgcctgggcctcgatccgatcgtcatcatcggcaataatcgcggctacaccaccgaacgcttcatcctggaaggcccgttcaacgacatcgccgactggcggttccatcgcctgggcgaactgttcggcccgctgcggggcttctcggcccccacggaagacgccttcgacgccgccctgggcgccgccctggccttccgggacggcccctccgtcatcgaggtggcgctgcgcccggacgactgctccgcggccctgacccggctctccgaacgcctgcgcgatgtggtgcagcagagcgcctga

SEQ ID NO: 71 - protein, 551 aa, Andreprevotia chitinilytica
MHMRETDMDTMGGYLLQALHREGVRHVFGVPGDYILRWYQLLSQSNLKHVGTSREDCAAFAADGYARCHGLGALAVTYGVGALNVVNAVAGANAESSPVVVISGAPGVAEQRQNPLLHHRFGPFCFQREIFERMTCYAAALDDPLLARRQIDRALELAQLHHKPVYLELPRDLVDAELPPALSPPTSSAPISDWDALEEAVAETLSLLAKAKSAAVLAGSELHRYQLQDELTQLVERGALPVAATLTGKSVIAERHPAYMGIYEGAMGGARTRELIERADVLLLLGATLNDVDLGIFTAKLDVQHMVQATADGVQIHHHRYTGVPLGDYVRALTAGIERSGRSLPVVEPPLAAIGFPITSQPMTVARLIGRLNDTLPQDMIVVCDTGDCLFASLELRVHARTAFLASAFYTTMGFAVPASLGAQLGSGRRPLVLVGDGAFQMTGTELATAAWKGLNPIVIVFNNAGYSTERFILDGPFNDIPSWQFHRLGELFGPLAGFDVHDEESFDSAWRSALAQTDRPSLLNVHLAPDDPSPAMRRLGEHLGKRVRAG

SEQ ID NO: 72 - DNA, 1656 bp, Andreprevotia chitinilytica
  source 1..1656: mol_type = other DNA
atgcacatgcgggagacggacatggatacgatgggcggctacctgctgcaggccctgcatcgcgagggcgtccggcatgtgttcggcgtgccgggcgactacatcctgcgctggtatcagctgctctcgcagagcaacctgaagcacgtcggcacctcgcgcgaagactgcgccgcgttcgccgccgacggctacgcgcggtgccacggcctgggcgccctggccgtcacctacggcgtcggcgccctgaacgtggtgaacgcggtggccggcgcgaacgcggagtccagccccgtggtggtcatcagcggcgccccgggcgtcgcggaacagcgccagaacccgctgctccaccaccgcttcggcccgttctgcttccagcgcgaaatcttcgaacgcatgacgtgctacgcggcggcgctcgacgatcccctcctggcgcggcgccagatcgaccgcgccctggagctggcccagctccatcacaagccggtctacctcgaactgccgcgggatctcgtggatgcggaactgccgccggccctctcgccgcccacctcctcggcgcccatctcggattgggacgcgctcgaagaagcggtcgcggaaaccctcagcctcctggcgaaggcgaaaagcgccgcggtcctcgccggctcggagctgcaccgctaccagctgcaggacgagctgacgcagctcgtggaacggggcgccctccccgtggccgccaccctgaccggcaagtccgtgatcgccgagcgccacccggcctacatgggcatctacgaaggcgcgatgggcggcgcccgcacgcgggaactgatcgagcgggcggacgtgctgctgctgctgggcgccaccctcaatgatgtggatctgggcatcttcaccgccaagctggatgtccagcacatggtgcaggcgaccgccgatggcgtccagatccaccaccaccgctacaccggcgtccccctcggcgactatgtgcgggccctgacggcgggcatcgaacgctcgggccgctccctcccggtggtggaaccccccctggcggccatcggcttcccgatcacctcgcagcccatgaccgtggcccgcctgatcggccgcctcaatgataccctgccgcaggacatgatcgtcgtctgtgacaccggcgactgcctcttcgcctccctggagctccgcgtccatgcccgcaccgccttcctggcctcggcgttctacacgacgatgggcttcgccgtgcccgcctcgctgggcgcccagctcggctcgggccggcgccccctggtgctggtgggcgatggcgccttccagatgacgggcaccgagctggccaccgccgcgtggaagggcctgaaccccatcgtcatcgtcttcaacaacgccggctactccaccgagcgcttcatcctggacggccccttcaacgacatcccgtcgtggcagttccatcgcctgggcgaactgttcggcccgctggccggcttcgatgtccacgacgaagagtcgttcgactccgcctggcgctcggccctcgcccagaccgatcgcccgtcgctgctgaacgtgcatctggcccccgacgacccctcgcccgcgatgcggcggctgggcgaacatctgggcaagcgggtgcgggcgggctga

SEQ ID NO: 73 - protein, 548 aa, Lactococcus lactis
MYTVGDYLLDRLHELGIEEIFGVPGDYNLQFLDQIISHKDMKWVGNANELNASYMADGYARTKKAAAFLTTFGVGELSAVNGLAGSYAENLPVVEIVGSPTSKVQNEGKFVHHTLADGDFKHFMKMHEPVTAARTLLTAENATVEIDRVLSALLKERKPVYINLPVDVAAAKAEKPSLPLKKENSTSNTSDQEILNKIQESLKNAKKPIVITGHEIISFGLEKTVTQFISKTKLPITTLNFGKSSVDEALPSFLGIYNGTLSEPNLKEFVESADFILMLGVKLTDSSTGAFTHHLNENKMISLNIDEGKIFNERIQNFDFESLISSLLDLSEIEYKGKYIDKKQEDFVPSNALLSQDRLWQAVENLTQSNETIVAEQGTSFFGASSIFLKSKSHFIGQPLWGSIGYTFPAALGSQIADKESRHLLFIGDGSLQLTVQELGLAIREKINPICFIINNDGYTVEREIHGPNQSYNDIPMWNYSKLPESFGATEDRVVSKIVRTENEFVSVMKEAQADPNRMYWIELILAKEGAPKVLKKMGKLFAEQNKS

SEQ ID NO: 74 - DNA, 1647 bp, Lactococcus lactis
  source 1..1647: mol_type = other DNA
atgtacaccgtaggagactacctacttgacagactccatgagctgggcatagaggaaatcttcggagtcccgggtgattacaacctgcagttcctggaccagatcatcagccacaaggacatgaagtgggtgggcaacgcgaacgaactgaacgcgagctatatggcggacggttatgcccgcaccaagaaggcagccgcgttcctcaccactttcggcgtgggcgaactcagcgccgtgaacggcttggcaggcagctatgcggaaaacctgccggtggtggaaatcgtcggctccccgacctcgaaggtgcagaacgagggcaagttcgtgcatcataccctggcggacggggacttcaagcatttcatgaagatgcacgaacccgtcaccgctgcccgcacgctgctgaccgcggaaaacgcgaccgtggagatcgaccgggtcctctccgccctgctgaaggaacggaagcccgtgtacatcaatctacccgtcgacgtagcagccgccaaggccgaaaagccctcgctcccgctgaagaaggagaactcgacgagcaacacgagtgaccaggaaatcctgaacaagatccaggagtcgttaaagaacgcgaagaagccgatcgtcatcaccggccatgagatcatcagcttcggccttgagaagaccgtgacacagttcatctccaagaccaagctgcccatcaccaccctcaacttcggcaagtccagcgtggacgaagccctgccgagcttcctgggcatctacaacggcaccctgtcggaacccaacctgaaggagttcgtcgaaagcgcggacttcatcctgatgctgggcgtgaagctgaccgactcctcgacgggagccttcacccatcacctgaacgaaaacaagatgatctccctcaacatcgatgaaggcaagatcttcaacgagcgcatccaaaacttcgacttcgaaagcctgatctcctcgctgctggacctgtccgagatcgagtacaagggcaagtacatcgacaagaagcaggaagacttcgtgccgtcgaacgcgctgctgtcgcaggaccgcctgtggcaggcggtcgaaaacctgacgcagtcaaacgaaaccatcgtcgccgaacagggaacctcgttcttcggtgcctcaagtatcttcctgaagtccaagtcccacttcatcggccagcccctgtggggctcgatcggctataccttcccggcagcgctaggctcccagatcgcggacaaggaatcgcggcacctgctcttcatcggcgacggctccctgcagctgaccgtccaggagctgggcctcgccatccgggaaaagatcaacccgatctgcttcatcattaacaacgacggctacaccgtggagcgcgagattcatggcccgaaccagagctacaacgacatccccatgtggaactactctaagctgccggaatcgtttggcgccacggaggaccgggtggtcagcaagatcgtccggacggagaacgagttcgtctcggtgatgaaggaagcgcaggcggaccccaaccggatgtattggatcgagctcatcttggccaaggagggcgctccgaaggtcctgaagaagatgggcaagctcttcgccgaacagaacaagtcgtaa

SEQ ID NO: 75 - protein, 570 aa, Lactococcus lactis
MGSSHHHHHHSSGLVPRGSHMASMYTVGDYLLDRLHELGIEEIFGVPGDYNLQFLDQIISREDMKWIGNANELNASYMADGYARTKKAAAFLTTFGVGELSAINGLAGSYAENLPVVEIVGSPTSKVQNDGKFVHHTLADGDFKHFMKMHEPVTAARTLLTAENATYEIDRVLSQLLKERKPVYINLPVDVAAAKAEKPALSLEKESSTTNTTEQVILSKIEESLKNAQKPVVIAGHEVISFGLEKTVTQFVSETKLPITTLNFGKSAVDESLPSFLGIYNGKLSEISLKNFVESADFILMLGVKLTDSSTGAFTHHLDENKMISLNIDEGIIFNKVVEDFDFRAVVSSLSELKGIEYEGQYIDKQYEEFIPSSAPLSQDRLWQAVESLTQSNETIVAEQGTSFFGASTIFLKSNSRFIGQPLWGSIGYTFPAALGSQIADKESRHLLFIGDGSLQLTVQELGLSIREKLNPICFIINNDGYTVEREIHGPTQSYNDIPMWNYSKLPETFGATEDRVVSKIVRTENEFVSVMKEAQADVNRMYWIELVLEKEDAPKLLKKMGKLFAEQNK

SEQ ID NO: 76 - DNA, 1713 bp, Lactococcus lactis
  source 1..1713: mol_type = other DNA
atgggctcgtcccatcatcatcaccaccattccagcggcctggtcccgcgcggcagccacatggcctcgatgtacaccgtgggcgattacctgctggaccggctccacgaactgggcatcgaagagatcttcggcgtcccgggcgactataacctccagttcctggatcagatcatctcccgcgaggacatgaaatggatcggcaatgcgaacgagctgaacgcctcctatatggccgacggctatgcccgcaccaaaaaagccgccgccttcctgaccacgttcggcgtgggcgaactgtcggccatcaacggcctggccggctcctacgccgagaacctgcccgtggtggaaatcgtgggctcgccgacctcgaaggtccagaacgacggcaagttcgtgcaccacacgctcgcggacggcgacttcaagcacttcatgaagatgcacgagccggtcaccgccgcccgcaccctgctgacggccgagaacgcgacctatgaaatcgaccgggtgctctcccagctcctgaaggagcggaagccggtgtacatcaacctccccgtcgatgtggccgccgccaaggccgagaaacccgcgctgagcctggagaaggagtcctccacgaccaacaccaccgaacaggtgatcctcagcaagatcgaagaatccctcaagaacgcccagaaacccgtggtgatcgcgggccatgaggtgatctcgttcggcctggagaaaaccgtcacccagttcgtgagcgaaaccaagctccccatcaccaccctcaacttcggcaagtcggccgtggacgagtccctgccgtccttcctgggcatctataatggcaaactgtcggaaatcagcctgaaaaatttcgtcgaaagcgccgacttcatcctcatgctgggcgtgaagctgaccgactcctcgaccggcgccttcacccaccacctcgatgaaaacaagatgatctccctgaacatcgacgaaggcatcatcttcaacaaggtggtcgaagacttcgacttccgggccgtcgtgtcgagcctgtccgaactgaaaggcatcgaatatgaaggccagtacatcgacaagcagtatgaagagttcatcccgtcgtcggcgccgctcagccaggatcgcctctggcaggccgtggaaagcctgacccagtccaacgaaacgatcgtcgccgaacagggcacctcgttcttcggcgccagcacgatcttcctgaagtccaactcccggttcatcggccagccgctgtggggctcgatcggctacacgttcccggccgcgctgggcagccagatcgccgacaaggaatcccgccacctcctgttcatcggcgacggctccctccagctgaccgtgcaggagctgggcctgtcgatccgcgaaaagctgaatccgatctgcttcatcatcaacaacgacggctacacggtcgaacgggagatccacggcccgacgcagtcctacaacgacatccccatgtggaactacagcaaactcccggaaaccttcggcgccaccgaagaccgcgtcgtctcgaagatcgtgcggaccgaaaacgagttcgtgtccgtgatgaaagaggcgcaggcggatgtgaaccggatgtactggatcgaactggtcctggagaaagaggacgccccgaagctcctgaagaagatgggcaagctcttcgccgagcagaacaaatga

SEQ ID NO: 77 - protein, 549 aa, Streptococcus didelphis
MYTVGDYLLDRLKEIGIDHIFGVPGDYNLQFLDQITARDDLKWVGNANELNASYMSDGYARTKKAAAFVTTFGVGELSAINGLAGSFAENVPVIEIVGSPTTKVQEAGKLVHHTLGDGNFNHFQEMHKSVTVAQVKVSAEHAQTDIDQVLLSLLKERKPVYINLPIDVAQMPAQKPESALLVEKVISEQDKIILQAIEKGLKTAKQPLIMVGHEVASFGLEATINNFIKKKKYPVTSLSLGKGIVNESPETFLGIYSGALSPQALKDYVDQADFILTLGVKLTDSVTGGFSQGFDAKQVLSLAANQASLFGENYQGYHFSDVIREIENLDIPSYSGSYIAKTKVADFEAEKGQVLSQKRFWQAMESFVQAGDTIFAEQGTSYFGASQLNLKENVAYQGQPLWGSIGYTFPAVFGSQLANPDSRHILFVGDGSLQLTVQDIGLALREQLNTIVFVINNDGYTVERKIHGPEEVYNDIPQWQYSQLPASFGGNDSQVLARKVSTEEELVEILEKARADVSRMYWIELMLPKMDAPEYLEKLGKLFAQQNKA

SEQ ID NO: 78 - DNA, 1650 bp, Streptococcus didelphis
  source 1..1650: mol_type = other DNA
atgtataccgtcggcgattatctgctggatcggctcaaggaaatcggcatcgaccatatcttcggcgtcccgggcgactacaacctccagttcctggatcagatcaccgcccgcgacgacctcaaatgggtgggcaacgccaacgaactcaacgcgagctacatgtccgacggctatgcccgcaccaagaaggccgccgccttcgtcaccaccttcggcgtgggcgaactgtcggcgatcaacggcctggccggctcgttcgcggaaaacgtccccgtgatcgagatcgtcggctcgccgacgaccaaggtgcaggaagccggcaaactggtgcaccatacgctcggcgacggcaacttcaatcacttccaggagatgcacaaatcggtcaccgtggcccaggtcaaagtcagcgccgaacatgcccagaccgacatcgaccaggtgctcctgtccctgctgaaggagcggaagccggtgtatatcaacctgcccatcgatgtggcccagatgcccgcccagaagccggaatcggccctcctggtcgagaaggtcatctccgagcaggacaagatcatcctgcaggcgatcgagaaaggcctgaagaccgccaagcagcccctcatcatggtcggccatgaagtcgccagcttcggcctggaagccaccatcaacaacttcatcaaaaagaagaagtaccccgtgacctcgctcagcctgggcaaaggcatcgtcaacgagtcgccggaaaccttcctgggcatctactccggcgccctgtccccgcaggccctgaaagactacgtcgatcaggcggacttcatcctcaccctcggcgtcaagctgaccgactccgtgacgggcggcttcagccagggcttcgatgcgaagcaggtgctgagcctggccgccaaccaggcgtcgctcttcggcgaaaactaccagggctatcacttctccgacgtgatccgcgaaatcgaaaatctggacatccccagctattcgggctcctatatcgccaagaccaaggtcgccgatttcgaagcggagaagggccaggtcctgtcccagaagcgcttctggcaggcgatggaatcgttcgtccaggccggcgacaccatcttcgccgagcagggcacctcgtacttcggcgcgagccagctcaacctgaaagagaacgtggcctaccagggccagccgctctggggcagcatcggctataccttcccggccgtcttcggcagccagctcgcgaaccccgattcgcggcatatcctgttcgtcggcgacggcagcctgcagctgaccgtccaggacatcggcctggcgctgcgcgagcagctgaacacgatcgtgttcgtcatcaacaacgacggctataccgtggagcgcaagatccacggcccggaagaggtctataatgacatcccgcagtggcagtacagccagctccccgcctccttcggcggcaacgactcgcaggtcctggcccggaaagtgagcaccgaagaggagctggtcgaaatcctggaaaaggcgcgggccgacgtgtcccggatgtattggatcgagctcatgctccccaaaatggacgcgccggagtatctggaaaagctgggcaagctgttcgcccagcagaataaagcctga

SEQ ID NO: 79 - protein, 548 aa, Enterococcus caccae
MYTVADYLLDRLKELGIDELFGVPGDYNLQFLDHITARQDLEWIGNANELNAAYMADGYARTKGISAFVTTFGVGELSAINGLAGSFAENVPVVEIVGSPTTTVQNDKKLVHHTLGDGNFLHFEKMHEEVTAAIAHLTAENALTEIDRVLIIAMIEKRPVYINLPIDIAEFKATPPLSPLSRSAEKLTDVEIAILDKVEKALSQAKNPVVIAGHEILSYHIEHQLDEFIQKFNLPITTLPLGKRAFNEEDPHYLGTYSGSTTEEPLKTRVDTADLVLLLGAKLTDSATSGFSFGFTDQQIISIGSTEVLFYGETFKAVQLDRFVSALTTLSFSRYEDEIQPVTRISNQAIKDEKLSQKQFWEMVETFLIPGDTVIGEQGTSFFGLTNVALKRNMHFIGQPLWGSIGYTFPSALGSQIANKESRHLLFIGDGSLQLTVQELGTALREKLTPIVFVINNNGYTVEREIHGATEQYNDIPMWDYQNLPLVFGGNNQTVATYKVTTAIELDEVMKTARKDTKRLQWIEVVMAQDDAPELLKKLAKIFAKQNS

SEQ ID NO: 80 - DNA, 1647 bp, Enterococcus caccae
  source 1..1647: mol_type = other DNA
atgtacaccgtcgccgattacctgctggatcgcctgaaggagctgggcatcgatgagctgttcggcgtgccgggcgactacaacctgcagttcctcgaccacatcaccgcccgccaggacctggagtggatcggcaacgccaacgaactgaacgccgcgtatatggccgacggctacgcccggaccaagggcatctcggcgttcgtgaccaccttcggcgtgggcgagctgtccgccatcaatggcctcgccggcagcttcgcggagaacgtcccggtcgtcgagatcgtgggctccccgaccaccacggtccagaacgacaagaagctggtgcatcacacgctgggcgacggcaatttcctgcatttcgagaagatgcatgaagaggtcaccgccgccatcgcgcacctcaccgccgaaaacgcgctgaccgaaatcgaccgggtgctcatcatcgccatgatcgagaagcgcccggtctatatcaacctcccgatcgacatcgccgagttcaaagccaccccgccgctcagccccctgagccggtcggccgaaaagctgacggatgtcgaaatcgccatcctcgacaaggtggaaaaggcgctgtcgcaggcgaagaaccccgtcgtgatcgccggccacgaaatcctcagctaccatatcgaacatcagctggacgagttcatccagaagttcaacctcccgatcaccacgctgcccctgggcaaacgcgcgttcaatgaggaagacccgcactatctcggcacctacagcggctccaccaccgaggagccgctgaagacccgcgtggataccgcggatctggtcctgctgctgggcgcgaagctgacggactcggccacctcgggcttctccttcggcttcaccgaccagcagatcatcagcatcggctccacggaggtcctcttctacggcgaaaccttcaaagccgtgcagctcgaccgcttcgtctcggcgctcaccaccctgagcttctcccggtacgaagatgaaatccagccggtgacccggatcagcaaccaggcgatcaaggacgagaagctgtcgcagaagcagttctgggagatggtcgagacgttcctgatcccgggcgacaccgtgatcggcgagcagggcacctcgttcttcggcctgaccaacgtcgccctgaagcggaatatgcacttcatcggccagccgctgtggggcagcatcggctatacgttcccgtcggccctcggctcgcagatcgccaacaaggaaagccggcatctgctgttcatcggcgatggctccctccagctgaccgtgcaggaactgggcaccgccctgcgcgagaagctgacgccgatcgtcttcgtcatcaacaacaatggctataccgtggaacgggagatccacggcgccaccgagcagtacaacgacatccccatgtgggattatcagaacctgccgctcgtgttcggcggcaacaaccagaccgtcgccacctacaaggtcaccaccgcgatcgaactggatgaggtcatgaaaaccgcccgcaaggacaccaagcgcctgcagtggatcgaagtggtcatggcgcaggatgacgcgccggaactgctcaagaaactcgccaaaatcttcgccaaacagaacagctga

SEQ ID NO: 81 - protein, 548 aa, Enterococcus haemoperoxidus
MYTISDYLLDRLKELGIDEVFGVPGDYNLQFLDHITAREDLKWIGNANELNAAYMADGYARTKGISAFVTTFGVGELSAVNGLAGSYAENVPVVEIIGSPTTTVQNNKKLVHHTLGDGDFLRFEKMHEEVTAAIAHLTIENATSEIDRVLTIAMTEKRPVYINLPIDIAETKTNKPNKPLQKMTERLTEAEATILSKVEKALQQAENPVIIAGHEILSYHIEHQLNEFIQKFNLPITTLPLGKGAFDEEDSHYMGTYSGSPTEEPLKSRVDNADLVLLLGAKLTDSATSGFSFGFTDKQIISIGATEVLFYGEKHEAIQLDRFVSALSTLSFSRFTGDLLPVKRISKVEFKDEQLTQKRFWKMVETFLLQGDTVVGEQGTSFFGLTNVPLKKDMHFIGQPLWGSIGYTFPSTLGSQIANKDSRHLLFIGDGSLQLTVQELGTAIREKLTPIVFVINNNGYTVEREIHGATEQYNDIPMWDYQNLPLVFGGTSQTVATYKATTEAELAEVMKSARKDTERLQWIEVVMDQEDAPLLLQKLAKIFAKQNS

SEQ ID NO: 82 - DNA, 1647 bp, Enterococcus haemoperoxidus
  source 1..1647: mol_type = other DNA
atgtacaccatcagcgactacctcctggaccggctgaaggaactcggcatcgacgaagtcttcggcgtcccgggcgactacaacctccagttcctcgatcatatcaccgcccgcgaagacctgaaatggatcggcaacgcgaatgaactcaatgccgcctacatggcggacggctatgcccgcaccaagggcatcagcgccttcgtgaccaccttcggcgtcggcgaactgagcgcggtgaacggcctggccggctcgtacgccgagaacgtgccggtggtcgagatcatcggcagccccaccaccaccgtgcagaacaacaagaaactggtccaccacacgctgggcgatggcgacttcctgcggttcgaaaagatgcacgaggaagtcaccgccgcgatcgcccatctgaccatcgagaatgccacgagcgaaatcgaccgcgtcctgacgatcgccatgaccgagaagcgccccgtgtatatcaacctgccgatcgatatcgcggaaaccaagacgaacaagccgaacaaaccgctgcagaagatgaccgagcggctcacggaagccgaagcgaccatcctgtcgaaggtcgagaaggccctccagcaggcggagaacccggtcatcatcgccggccatgagatcctgtcctaccacatcgagcaccagctgaatgagttcatccagaagttcaatctccccatcaccacgctgccgctgggcaagggcgccttcgacgaggaagactcgcactacatgggcacgtattccggctcccccacggaagagcccctgaagagccgcgtggataacgccgatctggtcctgctgctcggcgccaagctgaccgattccgcgacctccggcttctcgttcggcttcaccgacaagcagatcatcagcatcggcgcgaccgaagtcctgttctacggcgagaaacacgaggccatccagctcgatcgcttcgtgtccgccctgtccacgctctccttctcccgcttcaccggcgatctgctcccggtgaaacggatcagcaaggtcgagttcaaggacgagcagctcacccagaagcgcttctggaagatggtcgaaacgttcctgctccagggcgacaccgtcgtgggcgaacagggcaccagcttcttcggcctgaccaatgtgcccctgaagaaggatatgcacttcatcggccagccgctctggggcagcatcggctataccttccccagcaccctgggctcgcagatcgccaacaaggactcccgccacctgctgttcatcggcgatggctcgctgcagctgaccgtgcaggagctcggcacggccatccgggagaagctcacgccgatcgtcttcgtcatcaacaacaacggctacaccgtggaacgcgaaatccacggcgccaccgagcagtacaacgacatcccgatgtgggactaccagaacctccccctggtcttcggcggcacgagccagaccgtggcgacgtataaagccacgaccgaagcggagctggccgaggtcatgaagtccgcccggaaggatacggagcggctgcagtggatcgaggtggtgatggaccaggaagacgcgcccctgctgctgcagaagctggccaagatcttcgccaagcagaactcgtga

SEQ ID NO: 83 - protein, 548 aa, Enterococcus moraviensis
MYTVADYLLDRLKELGIDEVFGVPGDYNLQFLDHITARKDLEWIGNANELNAAYMADGYARTKGISALVTTFGVGELSAINGLAGSYAESIPVIEIVGSPTTTVQQNKKLVHHTLGDGDFLRFERIHEEVSAAIAHLSTENAPSEIDRVLTVAMTEKRPVYINLPIDIAEMKASAPTTPLNHTTDQLTTVETAILTKVEDALKQSKNPVVIAGHEILSYHIENQLEQFIQKFNLPITVLPFGKGAFNEEDAHYLGTYTGSTTDESMKNRVDHADLVLLLGAKLTDSATSGFSFGFTEKQMISIGSTEVLFYGEKQETVQLDRFVSALSTLSFSRFTDEMPSVKRLATPKVRDEKLTQKQFWQMVESFLLQGDTVVGEQGTSFFGLTNVPLKKDMHFIGQPLWGSIGYTFPSALGSQIANKESRHLLFIGDGSLQLTVQELGTAIREKLTPIVFVINNNGYTVEREIHGATEQYNDIPMWDYQKLPFVFGGTDQTVATYKVSTEIELDNAMTRARTDVDRLQWIEVVMDQNDAPVLLKKLAKIFAKQNS

SEQ ID NO: 84 - DNA, 1647 bp, Enterococcus moraviensis
  source 1..1647: mol_type = other DNA
atgtacacggtggcggactatctgctggaccggctgaaagagctgggcatcgacgaagtgttcggcgtgcccggcgactacaacctgcagttcctcgaccacatcacggcccggaaggacctcgaatggatcggcaatgccaacgaactgaacgccgcctacatggccgacggctacgcccgcaccaagggcatctccgcgctggtcaccaccttcggcgtcggcgaactgtcggccatcaatggcctggccggctcgtacgcggaaagcatcccggtgatcgaaatcgtgggctccccgacgacgacggtgcagcagaacaagaagctcgtgcatcatacgctgggcgacggcgatttcctgcggttcgagcgcatccacgaggaggtgtcggcggccatcgcgcacctgtccaccgagaacgccccctccgagatcgaccgcgtgctgacggtggccatgaccgaaaagcgcccggtctatatcaacctcccgatcgatatcgcggagatgaaagcgtcggcccccaccacgcccctgaaccacaccacggatcagctgacgaccgtcgagacggccatcctcaccaaggtcgaggatgcgctgaagcagtccaagaatcccgtcgtcatcgccggccacgagatcctgagctaccacatcgaaaatcagctggaacagttcatccagaagttcaacctgccgatcaccgtgctcccgttcggcaagggcgccttcaacgaagaggacgcgcattacctgggcacctatacgggcagcacgaccgacgagtccatgaagaatcgcgtcgaccatgcggacctggtcctgctgctcggcgccaagctcaccgactcggccacctcgggcttcagcttcggcttcacggagaagcagatgatctcgatcggctcgaccgaagtgctgttctatggcgagaagcaggagacggtgcagctcgaccgcttcgtgagcgccctgtcgaccctgtccttctcccgcttcaccgacgagatgccgagcgtgaaacgcctggccaccccgaaggtgcgcgatgagaagctgacccagaagcagttctggcagatggtcgagagcttcctgctccagggcgacaccgtcgtgggcgagcagggcacgagcttcttcggcctgacgaatgtgcccctgaaaaaggacatgcacttcatcggccagccgctgtggggcagcatcggctatacgttccccagcgccctgggcagccagatcgccaacaaagagtcccgccacctgctgttcatcggcgacggctcgctccagctgacggtccaggagctgggcaccgcgatccgcgaaaagctgacccccatcgtgttcgtcatcaacaacaacggctataccgtggaacgcgagatccacggcgcgaccgagcagtacaacgacatccccatgtgggactaccagaaactgccgttcgtgttcggcggcaccgatcagacggtggccacctataaggtgtccaccgaaatcgaactcgataacgcgatgacccgggcccggacggacgtggaccgcctccagtggatcgaagtcgtgatggaccagaacgacgccccggtcctgctgaagaagctcgccaagatcttcgcgaaacagaactcctga

SEQ ID NO: 85 - protein, 548 aa, Carnobacterium maltaromaticum
MYTVGNYLLDRLTELGIRDIFGVPGDYNLKFLDHVMTHKELNWIGNANELNAAYAADGYARTKGIAALVTTFGVGELSAANGTAGSYAEKVPVVQIVGTPTTAVQNSHKLVHHTLGDGRFDHFEKMQTEINGAIAHLTADNALAEIDRVLRIAVTERCPVYINLAIDVAEVVAEKPLKPLMEESKKVEEETALVLNKIEKALQDSKNPVVLIGNEIASFHLESALADFVKKFNLPVTVLPFGKGGFDEEDAHFIGVYTGAPTAESIKERVEKADLILIIGAKLTDSATAGFSYDFEDRQVISVGSDEVSFYGEIMKPVAFAQFVNGLNSLNYLGYTGEIKQVERVADIEAKASNLTQNNFWKFVEKYLSNGDTLVAEQGTSFFGASLVPLKSKMKFIGQPLWGSIGYTFPAMLGSQIANPASRHLLFIGDGSLQLTIQELGMTFREKLTPIVFVINNDGYTVEREIHGPNELYNDIPMWDYQNLPYVFGGNKGNVATYKVTTEEELVAAMSQARQDTTRLQWIEVVMGKQDSPDLLVQLGKVFAKQNS

SEQ ID NO: 86 - DNA, 1647 bp, Carnobacterium maltaromaticum
  source 1..1647: mol_type = other DNA
atgtataccgtgggcaactacctgctggaccgcctcaccgaactgggcatccgggatatcttcggcgtcccgggcgattataacctcaagttcctggaccatgtcatgacccataaggaactgaattggatcggcaacgccaacgagctgaatgcggcctatgccgccgacggctacgcgcggaccaagggcatcgcggccctggtcaccaccttcggcgtgggcgaactgagcgcggccaatggcaccgcgggctcctatgccgaaaaggtgcccgtggtgcagatcgtgggcacgcccacgacggcggtgcagaactcccacaaactggtgcaccataccctgggcgacggccgcttcgatcacttcgaaaagatgcagaccgagatcaatggcgccatcgcgcatctgaccgcggacaacgccctggcggagatcgatcgcgtgctgcggatcgccgtgaccgaacggtgcccggtctatatcaacctggccatcgatgtcgcggaggtggtggccgaaaaaccgctgaagcccctgatggaggaatcgaagaaagtcgaggaggagacggccctcgtcctcaacaagatcgaaaaggcgctccaggactccaaaaacccggtggtcctgatcggcaacgagatcgccagcttccatctggaatcggcgctggccgatttcgtcaagaagttcaacctcccggtcacggtgctgcccttcggcaagggcggcttcgacgaggaggatgcgcacttcatcggcgtctataccggcgccccgaccgccgaaagcatcaaggagcgggtggaaaaggccgacctcatcctcatcatcggcgcgaagctgaccgatagcgccaccgcgggcttctcctacgacttcgaggaccgccaggtcatcagcgtcggcagcgacgaagtgtccttctatggcgagatcatgaaacccgtggcgttcgcccagttcgtgaacggcctgaactccctgaattacctgggctacaccggcgaaatcaagcaggtggagcgggtggcggacatcgaggcgaaggcgtcgaatctcacccagaacaacttctggaagttcgtggaaaagtacctgtcgaacggcgacaccctggtggccgagcagggcaccagcttcttcggcgcctcgctcgtgccgctgaaatcgaagatgaagttcatcggccagccgctgtggggcagcatcggctatacgttccccgccatgctgggcagccagatcgcgaatcccgcgagccggcatctgctcttcatcggcgacggctccctgcagctgaccatccaggagctcggcatgaccttccgggagaaactgaccccgatcgtgttcgtcatcaacaacgatggctacaccgtcgagcgggaaatccacggcccgaacgagctctacaacgatatcccgatgtgggattatcagaacctcccgtacgtgttcggcggcaacaagggcaacgtcgccacctataaggtcaccaccgaggaagaactggtggccgccatgtcccaggcccggcaggacaccacccggctgcagtggatcgaggtcgtgatgggcaaacaggattcgccggacctcctggtccagctgggcaaggtgttcgccaagcagaacagctga

SEQ ID NO: 87 - protein, 548 aa, Brochothrix thermosphacta
MYTIGDYLLDRLNELGVEDIFGVPGDYNLTFLDHITAHPQLSWVGNANELNAAYAADGYARTKGFAALVTTFGVGELSAINGLAGSFAERVPVIEIVGSPVSTVQTDKKLVHHTLGDGDFLHFEKMHDAVTVASAHLTIQNATSEIDRVLTTALSLRRPGYINLPIDVAAAPAEKAQKKLQLKVTSPIDSTLLEKIQTAFSSAKQPVFITGHEIQSYHLEDTVAKIAAHTTVPVAALSLGKSSIDETHPQFVGIYSGALTAEPLKTYVDNADLVILLGAQLTDTATSGFSQSFSASKIIAIHPETTTVFGQDYPSNDFKELIEALTTIDYRMETSAALKTMPSTKEFIATDTLLTQNRFWEAIETNFKQNDTIVAEQGTSFFGITNTQFKKDMRLIGQPLWGSIGYTFPAALGSQLAARSKRHLLFIGDGSLQLTIQELGMALRAKLTPLIFVINNNGYTVEREIHGPNERYNDIPTWDYAQLPTVFGGTDQNVATYKVTTETELAEALVTAKADTTRLQWIEVVMDQTDAPELLKEMGRIFAKQNTH

SEQ ID NO: 88 - DNA, 1647 bp, Brochothrix thermosphacta
  source 1..1647: mol_type = other DNA
atgtatacgatcggcgactacctcctcgaccggctgaacgagctgggcgtggaagacatcttcggcgtgccgggcgactacaacctgacgttcctcgatcatatcaccgcgcatccgcagctctcctgggtcggcaatgccaacgaactcaacgccgcctacgccgccgatggctatgcgcggacgaaaggcttcgccgcgctggtgacgaccttcggcgtcggcgagctgtcggccatcaacggcctcgccggctccttcgccgaacgggtgccggtcatcgagatcgtcggcagcccggtcagcaccgtgcagaccgacaagaagctggtgcaccacaccctgggcgacggcgacttcctgcacttcgagaagatgcatgacgccgtcacggtggcctcggcgcacctcacgatccagaacgccaccagcgaaatcgaccgggtcctgacgaccgccctctcgctgcggcgccccggctatatcaacctgcccatcgacgtggcggcggccccggccgaaaaagcccagaaaaagctccagctgaaagtgaccagcccgatcgacagcacgctgctggaaaagatccagacggccttctcctcggccaagcagcccgtcttcatcacgggccatgaaatccagtcctaccacctggaggataccgtggcgaagatcgccgcgcacacgaccgtgccggtcgcggccctctcgctgggcaagagctccatcgatgaaacccatccccagttcgtcggcatctactccggcgccctgacggcggagcccctgaaaacctacgtcgataacgccgatctggtgatcctgctcggcgcccagctgaccgacacggccacctcgggcttcagccagtccttctcggccagcaaaatcatcgccatccacccggaaacgaccaccgtgttcggccaggattacccgtcgaacgatttcaaggaactgatcgaggccctcacgaccatcgattaccgcatggaaacctccgcggccctgaagacgatgccgtccaccaaggagttcatcgccaccgacacgctgctcacccagaatcgcttctgggaagccatcgaaaccaacttcaagcagaacgataccatcgtggcggaacagggcacgtcgttcttcggcatcaccaatacccagttcaagaaagatatgcgcctgatcggccagcccctgtggggctcgatcggctatacgttccccgccgccctgggctcgcagctggccgcccgctccaagcggcatctgctcttcatcggcgatggctcgctgcagctgaccatccaggaactgggcatggcgctccgcgccaaactcacccccctgatcttcgtcatcaacaacaacggctacaccgtggaacgcgagatccacggcccgaacgaacggtataatgacatcccgacctgggactatgcgcagctgccgaccgtgttcggcggcacggaccagaacgtggccacgtataaggtgacgaccgaaaccgaactggcggaggcgctcgtcaccgccaaggcggacaccacccgcctgcagtggatcgaggtggtgatggaccagacggacgccccggaactgctgaaagagatgggccgcatcttcgccaagcagaacacccattga

SEQ ID NO: 89 - protein, 548 aa, Carnobacterium gallinarum
MYTVADYLLDRLKELGINDIFGVPGDYNLKFLDHITARDDLKWIGNANELNAAYMADGYARTKGMAALVTTFGVGELSAMNGIGGSFAEKVPVIEIVGSPTTAVQNAQKLVHHTLGDGRFNHFEKMHEAITVGIGSLTKENAITEIDRILGLASEKRQPGYLNLPIDVAEMEVEKPNKPLFDTKVMEIKMEQELIKSIEKVLNSVKHPVIIAGNEIASFHLEAKLAEFIEKFNLPVTTLPFGKGVFNEEDKHYLGVYTGTPTTEPLKSYVDQADLVLLLGAKLTDSATSGFSQGFTEKQMISLASDEVIFQGEHLAGIQLPTVLDELLMINYPGYHGEIQPMSRLAEVKSSSSLVTQAYFWEAVESYLEEGDTLVAEQGTSFFGASTVPMKKGMSFIGQPLWGSIGYTFPAMLGSQIAKKGSRHLLFIGDGSLQLTVQELGMTLREKLAPIVFIINNNGYTVEREIHGPEEIYNDIPMWDYQKLPSVFGGTAENVVTYKVQTEAELATAMRKARLDSKRLQWIEVVMNQKDAPDLLVQMGKIFAKQNS

SEQ ID NO: 90 - DNA, 1647 bp, Carnobacterium gallinarum
  source 1..1647: mol_type = other DNA
atgtacacggtcgccgattacctgctcgatcggctgaaggaactgggcatcaatgacatcttcggcgtcccgggcgattataacctcaagttcctggaccacatcacggcccgcgacgacctgaaatggatcggcaacgcgaacgagctgaacgcggcgtatatggccgatggctacgcccgcaccaaaggcatggccgcgctggtcacgaccttcggcgtcggcgaactctcggccatgaacggcatcggcggctcgttcgccgagaaagtcccggtcatcgagatcgtgggctcccccaccaccgccgtccagaatgcccagaaactggtccatcatacgctgggcgacggccgcttcaaccatttcgagaagatgcatgaggcgatcaccgtgggcatcggctcgctgaccaaggagaatgccatcaccgaaatcgatcgcatcctgggcctggcgtccgagaagcggcagcccggctacctgaacctgccgatcgatgtggccgaaatggaagtggagaaaccgaacaagccgctcttcgataccaaggtcatggaaatcaaaatggaacaggagctcatcaagagcatcgagaaagtgctcaactcggtcaagcatccggtcatcatcgccggcaacgagatcgccagcttccatctggaggccaagctggccgagttcatcgagaagttcaatctccccgtcaccacgctgccgttcggcaagggcgtcttcaacgaagaagataagcattatctgggcgtgtatacgggcacccccacgacggaacccctgaagtcctacgtcgaccaggcggatctggtcctcctgctgggcgccaagctgaccgactccgccaccagcggcttcagccagggcttcaccgagaagcagatgatctcgctggcctcggacgaggtcatcttccagggcgagcacctcgccggcatccagctccccaccgtcctggatgagctgctgatgatcaactatccgggctaccacggcgagatccagccgatgtcgcggctggcggaagtgaagtcgtcctccagcctcgtcacccaggcgtacttctgggaggccgtcgagtcgtacctggaagaaggcgatacgctcgtcgccgaacagggcaccagcttcttcggcgcctccaccgtccccatgaagaagggcatgagcttcatcggccagccgctgtggggctccatcggctacacgttcccggccatgctcggctcgcagatcgccaagaagggctcgcgccatctgctgttcatcggcgacggctcgctccagctgaccgtccaggaactgggcatgacgctccgggaaaagctggcgccgatcgtgttcatcatcaacaataacggctacaccgtggaacgggaaatccacggccccgaagaaatctataacgacatcccgatgtgggactaccagaagctcccgtccgtcttcggcggcaccgccgaaaacgtggtgacctataaggtccagaccgaggcggagctggccaccgccatgcgcaaggcccgcctggactcgaagcggctgcagtggatcgaagtggtgatgaaccagaaggacgccccggacctcctggtgcagatgggcaagatcttcgccaagcagaatagctga

SEQ ID NO: 91 - protein, 548 aa, Carnobacterium divergens; identical to SEQ ID NO: 10

SEQ ID NO: 92 - DNA, 1647 bp, Carnobacterium divergens; identical to SEQ ID NO: 9
  source 1..1647: mol_type = other DNA

SEQ ID NO: 93 - protein, 174 aa, Helicobacter bizzozeronii
MIAEQGTSFFGAISMVLPSGVSFIGQPLWGSIGYTFGALLGTALASPDRRHILLIGDGSFQLVAQELSTMLRENITPIIIVINNDGYTVERCIHGPTRQYNHINMWHYSKLASFFDVHLAREVVSFQVSSVAGLREALCVAQQNSKLALIEACMDKNDAPILLKKLGALFGAQI

SEQ ID NO: 94 - DNA, 1104 bp, Helicobacter bizzozeronii
  source 1..1104: mol_type = other DNA
atgcagacgacgatcggccagtatctcctggaccggctgaagtcctacggcgtgcagcatctcttcggcgtgcccggcgactataacctggccttcctcgacctgatcgaagacgatccgcacatccagtgggtcggcaactgcaacgaactgaatgcgtcctacgccgccgacggctacgcgcggctcaagagcatgggcgccctcctgacgaccttcggcgtcggcgagctgagcgccatcaacggcatcgccggctcgtacgcggaatccgtgccggtcgtgaagatcgtcggcatgccctcccgcggcgtggtccattcccgcaagctggtgcaccacaccctgggcgacggcgagttcctcaagttctacaacatgtatgccgaagtgagcgtcgcccagacgatcctcaacaaacagaacgcccagagcgaaatcgaccgcgtcctgggcgaatgcttcctgcataaaaagccggtctacatcggcctcccggtggacgtgacgcacatcccgatcgaaacgtacgccccctcccccctggtggccaagagcgacccgaaaatcctcaacgccttcctgaaggacgcccaggagctgctgtcgaagagcaaatcccaggtggtcatggcggattatgaagtgaaccgctaccagttcaaccaggagctgacgcgcttcatcgaagccgtgaacctgcccatcgtgtcgctggcgatgggcaagggcgtcttcgatgaaacgcacccgaacttcatcggcgtgtataacggcatcctctcggacgcccgggtgagctcgctgatgaagcacgccgactgcgcgatcctggtgggcgtgaagctgacggactcgctgacggccggcttccactatatccgcgaacatcacctgtccatccagatccaccccttctactcccagatcggcgaaaagacgtacgacgatatcctcatgcaggacgtgctgaaagcgctcgcccagctgaagttccaggcctcgttcccgaaggagacgcaccccaaaacgccgcacctgaacggcaagctgacccaggacaagttcttcaagatcgactcgcgcatcctgacccccccgtga

SEQ ID NO: 95 - protein, 273 aa, Staphylococcus aureus
MKQRIGAYLIDAIHRAGVDKIFGVPGDFNLAFLDDIISNPNVDWVGNTNELNASYAADGYARLNGLAALVTTFGVGELSAVNGIAGSYAERIPVIAITGAPTRAVEQAGKYVHHSLGEGTFDDYRKMFAHITVAQGYITPENATTEIPRLINTAIAERRPVHLHLPIDVAISEIEIPTPFEVTAAKDTDASTYIELLASKLHQSKQPIIITGHEINSFHLHQELEDFVNQTQIPVAQLSLGKGAFNEENPYYMGIYDGKLPKIKYAIMWTTAI

SEQ ID NO: 96 - DNA, 822 bp, Staphylococcus aureus
  source 1..822: mol_type = other DNA
atgaaacagcgcatcggcgcctacctgatcgatgccatccaccgcgccggcgtggacaagatcttcggcgtcccgggcgatttcaacctcgccttcctggacgatatcatcagcaacccgaacgtggattgggtcggcaacaccaacgagctgaacgcctcgtatgcggccgatggctatgcccgcctcaacggcctggcggccctggtcaccaccttcggcgtgggcgaactgtcggcggtgaatggcatcgcgggcagctatgccgagcgcatcccggtgatcgccatcaccggcgcccccacccgcgccgtcgagcaggccggcaagtatgtgcatcatagcctgggcgaaggcacgttcgatgactaccggaagatgttcgcccatatcaccgtggcccagggctacatcacgcccgagaatgcgacgaccgaaatcccccgcctcatcaacacggccatcgccgagcgccgcccggtgcatctccacctgcccatcgatgtggcgatctcggagatcgagatccccaccccgttcgaggtgacggcggcgaaagacacggacgcctcgacctatatcgagctgctggccagcaaactgcaccagagcaagcagcccatcatcatcacgggccatgagatcaactccttccatctgcaccaggaactggaagatttcgtcaatcagacccagatccccgtggcgcagctctcgctgggcaaaggcgccttcaacgaggaaaacccgtactatatgggcatctacgatggcaagctgcccaaaatcaagtatgcgatcatgtggaccacggcgatctga

SEQ ID NO: 97 - protein, 554 aa, Fictibacillus macauensis
MNNHYTVGTYLLHRLSELGVRHMFGVPGDYNLTFLDDVIDFEGMEWIGNCNELNAAYAADGYARINGMAALVTTFGVGELSAINGIAGSYAEKVPVVKITGMPTTNVMNQNLYVHHTLGDGNFQHFGNMFQEVTAAQTMLTQENAAQEIDRVLLACWHEKRPVHINLPIDVYNKPVNPPEHSLLERGISSNATALEQMLTTVIPTIKEATSPVILADYEVYRYQAQEALMLLAEKTGFPVATLSMGKGVFNETHPQFIGVYNGDLSSDYVKNMVDHADCILSIGVKLTDSITGGFSHEFTEEQVIDISPYSVSKKALKWAPITMLDALGAITDALEQKPTPATTARLAAYSNESSFTATNTTLTQERFFDQVSHFLQEGDVILAEQGTSFFGAATMPLPKGATFIGQPLWGSIGYTLPALLGSQLADESRRNLLLIGDGSFQLTAQELSTMLRQRIAPIIFLINNDGYTVERAIHGENQVYNDIQMWDYSKLPAVFGAADASVTYKVRTEEELEAALHSAQNSSQLVFIEVMMEKNDTPELLTALSKRFANQNN

SEQ ID NO: 98 - DNA, 1665 bp, Fictibacillus macauensis
  source 1..1665: mol_type = other DNA
atgaacaaccattataccgtcggcacctatctgctgcatcgcctgtccgagctgggcgtccgccatatgttcggcgtgcccggcgactataatctgacgttcctggacgatgtcatcgacttcgaaggcatggaatggatcggcaactgtaacgagctcaacgccgcctacgcggccgacggctatgcccgcatcaacggcatggccgccctggtgaccaccttcggcgtcggcgagctgtcggccatcaacggcatcgccggctcgtacgccgaaaaagtgccggtcgtcaaaatcacgggcatgcccaccaccaacgtgatgaaccagaatctgtacgtccatcacacgctgggcgacggcaacttccagcacttcggcaacatgttccaggaggtcaccgccgcgcagacgatgctgacccaggaaaacgcggcgcaggagatcgatcgcgtgctgctcgcctgctggcacgaaaagcgcccggtgcacatcaacctcccgatcgatgtctacaacaagccggtcaacccccccgagcattcgctcctggaacggggcatctcgtcgaacgcgaccgcgctcgaacagatgctgaccaccgtgatcccgacgatcaaggaggccacctcgcccgtgatcctggcggattatgaggtgtatcgctaccaggcccaggaagccctgatgctgctggcggaaaagaccggcttcccggtggccaccctgagcatgggcaagggcgtgttcaacgaaacccatccccagttcatcggcgtgtacaacggcgacctgtcgtccgactacgtgaagaatatggtcgaccatgccgactgtatcctctccatcggcgtcaagctgaccgacagcatcacgggcggcttcagccatgagttcaccgaggagcaggtcatcgacatctccccgtatagcgtgagcaaaaaagccctcaaatgggcgcccatcacgatgctggatgcgctgggcgccatcacggatgccctggagcagaagccgacccccgccaccaccgcgcggctcgccgcctactcgaacgagagctccttcaccgcgacgaacacgacgctgacccaggagcgcttcttcgaccaggtgtcccacttcctccaggagggcgacgtgatcctggcggaacagggcaccagcttcttcggcgcggccacgatgccgctcccgaagggcgccacgttcatcggccagccgctgtggggcagcatcggctacaccctgccggccctgctgggcagccagctggccgacgaatcccgccgcaatctcctgctcatcggcgatggctcgttccagctcaccgcccaggagctgtcgacgatgctgcgccagcggatcgcgccgatcatcttcctcatcaacaacgacggctacaccgtggaacgggcgatccacggcgagaatcaggtgtataacgacatccagatgtgggactattcgaagctgccggcggtcttcggcgcggcggacgccagcgtcacctacaaggtccggaccgaagaggagctggaggcggccctgcatagcgcccagaactcgtcccagctggtcttcatcgaagtgatgatggagaagaatgacacccccgaactgctgacggccctgagcaagcgcttcgcgaatcagaacaactga

SEQ ID NO: 99 - protein, 572 aa, Bacillus licheniformis
MNNVAAKNETLTVRGAELVVDSLIQQGVTHVFGIPGAKIDAVFDVLKDKGPELIVCRHEQNAAFMAAAVGRLTGKPGVCLVTSGPGASNLATGLVTANTEGDPVVALAGAVKRADRLKKTHQSMDNAALFQPITKYSAEVEDANNIPEAVTNAFRAAASGQAGAAFLSFPQDVTAGPATAKPVKTMPAPKLGAASDEQISAAIAKIHNANLPVVLVGMKGGRPEAIEAVRRLLRKVKLPFVETYQAAGTLSHDLEDQYFGRIGLFRNQPGDMLLEKADVVLTVGYDPIEYDPVFWNGKGERSVIHLDEIQADIDHDYQPEIELIGDIAETLNHIEHDSLPVSIDESFAPVLDYLKKALEEQSEPPKETKTDLVHPLQIVRDLRELLSDDITVTCDIGSHAIWMSRYFRTYRPHGLLISNGMQTLGVALPWAIAATLVNPGQKVVSVSGDGGFLFSAMELETAVRLKAPIVHIVWNDSTYDMVAFQQEMKYKRTSGVDFGGIDIVKYAESFGAKGLRVNSPDELAEVLKAGLDAEGPVVIDIPVDYSDNIHLADQRFPKKFEEHFNKEASKQS

SEQ ID NO: 100 - DNA, 1719 bp, Bacillus licheniformis
  source 1..1719: mol_type = other DNA
atgaataacgtcgcggccaagaacgaaaccctgaccgtccggggcgccgaactcgtggtggatagcctgatccagcagggcgtgacccatgtcttcggcatcccgggcgccaaaatcgacgcggtcttcgacgtgctgaaggataagggccccgaactgatcgtctgccgccatgagcagaacgcggccttcatggccgccgccgtcggccgcctgacgggcaagccgggcgtctgcctggtcacctccggcccgggcgcctcgaatctcgcgaccggcctggtcaccgcgaacacggaaggcgacccggtggtcgccctggcgggcgccgtgaagcgggcggatcggctgaagaagacgcaccagtcgatggataacgccgccctgttccagcccatcacgaagtacagcgcggaggtggaagacgcgaacaacatcccggaggccgtgacgaacgccttccgcgccgcggcgtccggccaggccggcgcggccttcctcagcttcccccaggatgtcaccgccggcccggccaccgccaagccggtcaaaaccatgcccgccccgaagctgggcgccgcgagcgatgaacagatctccgccgcgatcgcgaagatccacaacgcgaatctgccggtggtcctcgtgggcatgaagggcggccggccggaagccatcgaagccgtgcgccgcctgctccgcaaggtcaagctcccgttcgtggaaacctaccaggcggccggcacgctgtcgcacgatctggaggatcagtacttcggccggatcggcctgttccggaaccagccgggcgacatgctcctggaaaaggccgacgtggtcctgaccgtgggctacgacccgatcgagtacgatccggtgttctggaatggcaaaggcgaacgctcggtcatccacctcgacgaaatccaggccgatatcgatcacgactaccagcccgagatcgaactcatcggcgacatcgcggaaaccctcaatcacatcgagcatgactcgctgccggtgtccatcgacgaatccttcgcgcccgtgctcgactatctcaagaaggcgctcgaagaacagtcggagcccccgaaggaaacgaagaccgatctggtccacccgctccagatcgtgcgcgacctgcgcgagctgctctccgatgacatcaccgtcacctgcgacatcggcagccacgccatctggatgtcccgctatttccgcacctatcgcccgcatggcctcctgatctccaacggcatgcagacgctgggcgtcgccctgccgtgggcgatcgccgcgaccctggtgaacccgggccagaaggtggtgtcggtcagcggcgatggcggcttcctcttctccgcgatggaactcgaaaccgccgtccgcctcaaggcgccgatcgtgcacatcgtgtggaacgactccacgtacgacatggtcgcgttccagcaggaaatgaagtacaagcgcacctccggcgtcgatttcggcggcatcgacatcgtcaagtatgcggaatccttcggcgccaaaggcctccgcgtgaatagccccgatgaactggccgaggtcctgaaggccggcctcgacgcggagggcccggtggtcatcgacatccccgtcgactactcggataacatccacctggccgaccagcgcttcccgaagaagttcgaggagcacttcaacaaggaagcgtcgaagcagtcctga